(19) United States
(12) Patent Application Publication
(10) Pub. No.: US 2017/0327872 A1
(43) Pub. Date: Nov. 16, 2017

(54) DETECTION METHOD

(71) Applicant: The Secretary of State for Health, London (GB)

(72) Inventors: Neil Woodford, London (GB); Katie Hopkins, London (GB); Matthew Ellington, London (GB)

(73) Assignee: The Secretary of State for Health, London (GB)

(21) Appl. No.: 15/522,130

(22) PCT Filed: Oct. 30, 2015
(86) PCT No.: PCT/GB2015/053276

§ 371 Date: Apr. 26, 2017

(30) Foreign Application Priority Data
Oct. 30, 2014 (GB) 1419330.4

(51) Int. Cl. C12Q 1/68; C12Q 1/04

(52) U.S. Cl. CPC C12Q 1/689; C12Q 1/04; C12Q 2600/106

(57) ABSTRACT

This invention relates to the detection of carbapenem-resistant bacteria and the diagnosis of carbapenem-resistant bacteria infection. More specifically, the invention relates to new primers and probes for particular carbapenemase genes, which enable the accurate detection of carbapenem-resistant bacteria.

### FIELD OF THE INVENTION

This invention relates to the detection of carbapenem-resistant bacteria and the diagnosis of carbapenem-resistant bacteria infection. More specifically, the invention relates to new primers and probes for particular carbapenemase genes, which enable the accurate detection of carbapenem-resistant bacteria.

### BACKGROUND OF THE INVENTION

Antibiotics are an important class of medicines. However, antibiotic resistance and the spread of resistant bacteria caused by the overuse and misuse of antibiotics is a major problem. The World Health Organisation describes the problems of antibiotic resistance as one of the biggest threats to human health today.

Resistance to beta-lactam antibiotics such as penicillin has come about through the emergence of bacterial strains which produce beta-lactamases; enzymes which hydrolyse the beta-lactam ring of beta-lactam antibiotics, ablating their antibacterial properties.

Carbapenems are a powerful group of broad spectrum beta-lactam (penicillin-related) antibiotics. Carbapenems have proved particularly useful as they have a structure that renders them resistant to most beta-lactamases, such that carbapenems are effective against bacteria which may be resistant to other beta-lactam antibiotics. Thus, in many cases carbapenems are the last line of defence against multi-resistant bacterial infections, particularly multi-resistant gram-negative bacteria.

Resistance has begun to emerge to carbapenems. Bacteria now exist which encode carbapenemases; a subset of beta-lactamases that are able to cleave the modified beta-lactam ring found in carbapenems, rendering them inactive. Carbapenem resistance is not due to a single carbapenemase enzyme, instead multiple carbapenemases have emerged. Carbapenems are encoded on horizontally-transferrable plasmids and have now spread globally.

An increase in antibiotic resistance, particularly carbapenemase resistance, in Gram-negative bacteria is especially concerning due to the speed with which resistance is emerging and because there are fewer new and developmental antibiotics against Gram-negative bacteria.

As well as a need for new antibiotics to treat carbapenem-resistant bacteria, there is also a need for screening methods to enable the detection of carbapenem-resistant bacteria. Early detection of infection typically allows for a more effective therapeutic treatment with a correspondingly more favourable clinical outcome. In view of the increasing threat and global prevalence carbapenem-resistant bacteria, new strategies are required for more effective prevention, treatment, and diagnosis of carbapenem-resistant bacteria infection. Ideally, diagnosis would be made by a technique that accurately, rapidly, and simultaneously detects a plurality of different carbapenemase enzymes at a single point in time, thereby minimizing progression of infection during the time required for diagnosis.

### SUMMARY OF THE INVENTION

Previous attempts to develop new diagnostic methods for carbapenem-resistant bacteria infection have typically focused on substrate-based methods. In more detail, samples have been plated on media containing a carbapenem antibiotic and assessed for bacterial growth. Bacteria able to grow in the presence of a carbapenem antibiotic are classed as carbapenem-resistant.

More recently, there have been attempts to develop nucleic acid-based methods for detecting carbapenem-resistant bacteria. Commercial kits for use in such methods have been developed. However, these methods and kits have been focused on the detection of individual carbapenemase families (such as KPC carbapenemases), and as such it is not possible to detect multiple carbapenemase using a single method with commercially available reagents.

Furthermore, as well as the existence of multiple different carbapenemase families, sequence variation within an individual carbapenemase family also makes it difficult to reliably identify the presence of carbapenem-resistant bacteria. Even if multiple assays are conducted for different carbapenemase families, variation within individual families may mean that some carbapenem-resistant bacteria are not detected, potentially resulting in false negative results.

The present inventors have conducted a detailed analysis of the sequences of the OXA-48-like and VIM carbapenemase genes. Based on the results of this analysis, the inventors have designed new primers and probes which maximize coverage of the different OXA-48-like and VIM carbapenemase gene variants. This reduces the risk of false negatives.

Another advantage provided by the present invention is that the mode of detection of the carbapenemase genes is flexible; it can be run using real-time/quantitative PCR (qPCR) or using standard PCR depending on the equipment available.

Therefore, the present invention allows for the flexible, accurate, rapid and sensitive detection of carbapenem-resistant bacteria and the diagnosis of carbapenem-resistant bacteria infection through a measurement of the OXA-48-like and/or VIM genes in a biological sample at a single point in time.

Accordingly, the present invention provides a method for determining the presence and/or amount of one or more carbapenemase-producing bacteria in a sample comprising determining the presence and/or amount of a carbapenemase gene selected from an OXA-48-like gene and/or a VIM gene in said sample, in which the presence and/or amount of the OXA-48-like gene is determined using at least one oligonucleotide specific for the OXA-48-like gene and/or the presence and/or amount of the VIM gene is determined using at least one oligonucleotide specific for the VIM gene, wherein: (a) the at least one oligonucleotide specific for the OXA-48-like gene comprises a region of at least 24 contiguous bases from SEQ ID NO: 3, or from a nucleic acid sequence having at least 80% sequence identity to the full-length of the nucleic acid sequence of SEQ ID NO: 3 and hybridises to a target region of the OXA-48-like gene comprising bases 570 to 670 of the OXA-48-like gene; and/or (b) the at least one oligonucleotide specific for the VIM gene is 15 to 35 bases in length and hybridises to a nucleic acid sequence within a target region of the VIM gene comprising bases 235 to 400, 238 to 403 and/or 245 to 410 of the VIM gene.

In some embodiments, the at least one oligonucleotide sequence specific for the VIM gene hybridises to a target region of the VIM gene comprising bases 350 to 410 of the VIM gene. The at least one oligonucleotide sequence specific for the VIM gene may comprise a region of at least 15 contiguous bases from SEQ ID NO: 18, or from a nucleic acid sequence having at least 80% sequence identity to the full-length of the nucleic acid sequence of SEQ ID NO: 18. In some embodiments the oligonucleotide specific for the OXA-48-like gene is a probe, and/or the oligonucleotide specific for the VIM gene is a reverse primer. The presence and/or amount of the OXA-48-like gene and the VIM gene may be determined.

The at least one oligonucleotide specific for the VIM gene may hybridise to a target region of the VIM gene comprising bases: (i) 235 to 280, 238 to 283 or 240 to 290; and/or (ii) 360 to 410, 363 to 413 or 270 to 420 of the VIM gene.

One or more additional oligonucleotide specific for the OXA-48-like gene comprising a nucleic acid sequence of SEQ ID NO: 2 and/or 16, or a nucleic acid sequence having at least 80% sequence identity to the full-length of the nucleic acid sequence of SEQ ID NO: 2 and/or 16, may be used to determine the presence and/or amount of the OXA-48-like gene.

One or more additional oligonucleotide specific for the VIM gene comprising a nucleic acid sequence of SEQ ID NO: 19 and/or 17, or a nucleic acid sequence having at least 80% sequence identity to the full-length of the nucleic acid sequence of SEQ ID NO: 19 and/or 17, may be used to determine the presence and/or amount of the VIM gene.

An additional oligonucleotide specific for the OXA-48-like gene may hybridise to a target region of the OXA-48-like gene comprising: (i) bases 421 to 490 of the OXA-48-like gene; (ii) bases 650 to 717 of the OXA-48-like gene; or (iii) a first additional oligonucleotide specific for the OXA-48-like gene may hybridise to a target region of the OXA-48-like gene comprising bases 421 to 490 of the OXA-48-like gene, and a second additional oligonucleotide specific for the OXA-48-like gene may hybridise to a target region of the OXA-48-like gene comprising bases 650 to 717 of the OXA-48-like gene. In some embodiments: (i) the additional oligonucleotide specific for the OXA-48-like gene which hybridises to a target region of the OXA-48-like gene comprising bases 421 to 490 of the OXA-48-like gene is a forward primer; and/or (ii) the additional oligonucleotide specific for the OXA-48-like gene which hybridises to a target region of the OXA-48-like gene comprising bases 650 to 717 of the OXA-48-like gene a reverse primer. In some embodiments: (i) the additional oligonucleotide specific for the OXA-48-like gene which hybridises to a target region of the OXA-48-like gene comprising bases 421 to 490 of the OXA-48-like gene comprises or consists of a nucleic acid sequence of SEQ ID NO: 16, or a nucleic acid sequence having at least 80% sequence identity to the full-length of the nucleic acid sequence of SEQ ID NO: 16; and/or (ii) the additional oligonucleotide specific for the OXA-48-like gene which hybridises to a target region of the OXA-48-like gene comprising bases 650 to 717 of the OXA-48-like gene comprises or consists of a nucleic acid sequence of SEQ ID NO: 2, or a nucleic acid sequence having at least 80% sequence identity to the full-length of the nucleic acid sequence of SEQ ID NO: 2.

In some embodiments: (i) an additional oligonucleotide specific for the VIM gene hybridises to a target region of the VIM gene comprising bases 235 to 400, 238 to 403 and/or 245 to 410 of the VIM gene, preferably to a target region of the VIM gene comprising bases 235 to 270 of the VIM gene; or (ii) an additional oligonucleotide specific for the VIM gene hybridises to a target region of the VIM gene comprising bases 235 to 400, 238 to 403 and/or 245 to 410 of the VIM gene, preferably to a target region of the VIM gene comprising bases 275 to 330 of the VIM gene; or (iii) a first additional oligonucleotide specific for the VIM gene hybridises to a target region of the VIM gene comprising bases 235 to 400, 238 to 403 and/or 245 to 410 of the VIM gene, preferably to a target region of the VIM gene comprising bases 235 to 270 of the VIM gene, and a second additional oligonucleotide specific for the VIM gene hybridises to a target region of the VIM gene comprising bases 235 to 400, 238 to 403 and/or 245 to 410 of the VIM gene, preferably to a target region of the VIM gene comprising bases 275 to 330 of the VIM gene. The additional oligonucleotide specific for the VIM gene which hybridises to a target region of the VIM gene comprising bases 235 to 400, 238 to 403 and/or 245 to 410 of the VIM gene, preferably to a target region of the VIM gene comprising bases 235 to 270 of the VIM gene, may be a forward primer; and/or the additional oligonucleotide specific for the VIM gene which hybridises to a target region of the VIM gene comprising bases 235 to 400, 238 to 403 and/or 245 to 410 of the VIM gene, preferably to a target region of the VIM gene comprising bases 275 to 330 of the VIM gene, may be a probe. In some embodiments: (i) the additional oligonucleotide specific for the VIM gene which hybridises to a target region of the VIM gene comprising bases 235 to 400, 238 to 403 and/or 245 to 410 of the VIM gene, preferably to a target region of the VIM gene comprising bases 235 to 270 of the VIM gene, comprises or consists of a nucleic acid sequence of SEQ ID: 17, or a nucleic acid sequence having at least 80% sequence identity to the full-length of the nucleic acid sequence of SEQ ID NO: 17; and/or (ii) the additional oligonucleotide specific for the VIM gene which hybridises to a target region of the VIM gene comprising bases 235 to 400, 238 to 403 and/or 245 to 410 of the VIM gene, preferably to a target region of the VIM gene comprising bases 275 to 330 of the VIM gene, comprises or consists of a nucleic acid sequence of SEQ ID: 19, or a nucleic acid sequence having at least 80% sequence identity to the full-length of the nucleic acid sequence of SEQ ID NO: 19.

The method of the invention may further comprise determining the presence and/or amount of one or more additional carbapenemase gene. The one or more additional carbapenemase gene may be selected from a KPC gene, an NDM gene, an IMP gene, an IMI gene, a GES gene and an SPM gene. In some embodiments the one or more additional carbapenemase gene is: (i) a KPC gene; (ii) an NDM gene; (iii) an IMP gene; (iv) a KPC gene and an NDM gene; (v) a KPC gene and an IMP gene; (vi) an NDM gene and an IMP gene; or (vii) a KPC gene, an NDM gene and an IMP gene. In a preferred embodiment, the presence and/or amount of: (i) an OXA-48-like gene and an NDM gene; (ii) an OXA-48-like gene, a VIM gene and an NDM gene; (iii) an OXA-48-like gene, a VIM gene, a KPC gene and an NDM gene; or (iv) an OXA-48-like gene, a VIM gene, a KPC gene an NDM gene and an IMP gene; is determined.

The presence and/or amount of the one or more additional carbapenemase gene may be determined using at least one oligonucleotide specific for said one or more additional carbapenemase gene. The at least one oligonucleotide specific for the NDM gene may hybridise to a target region of the NDM gene comprising bases 108 to 320 of the NDM gene. The at least one oligonucleotide specific for the KPC gene may hybridise to a target region of the KPC gene comprising bases 580 to 800 or 600 to 810 of the KPC gene. The at least one oligonucleotide specific for the IMP gene may hybridise to a target region of the IMP gene comprising bases 337 to 515 of the IMP gene. The at least one oligonucleotide specific for the NDM gene may hybridises to a target region of the NDM gene comprising bases 108 to 150, 294 to 320 and/or 160 to 200 of the NDM gene. The at least one oligonucleotide specific for the KPC gene may hybridise to a target region of the KPC gene comprising bases: (i) 580 to 620 or 600 to 650; (ii) 745 to 800 or 760 to 815; and/or (iii) 660 to 710 or 675 to 725; of the KPC gene. The at least one oligonucleotide specific for the IMP gene may hybridise to a target region of the IMP gene comprising bases 337 to 363, 440 to 514 and/or 471 to 515 of the IMP gene. The at least one oligonucleotide specific for the NDM gene may comprise a nucleic acid sequence of SEQ ID NO: 7, 8 and/or 9, or a nucleic acid sequence having at least 80% sequence identity to the full-length of the nucleic acid sequence of SEQ ID NO: 7, 8 and/or 9. The at least one oligonucleotide specific for the KPC gene may comprise a nucleic acid sequence of SEQ ID NO: 21, 11 and/or 12, or a nucleic acid sequence having at least 80% sequence identity to the full-length of the nucleic acid sequence of SEQ ID NO: 21, 11 and/or 12. The at least one oligonucleotide specific for the IMP gene may comprise a nucleic acid sequence of SEQ ID NO: 22, 23, 24 and/or 25, or a nucleic acid sequence having at least 70% sequence identity to the full-length of the nucleic acid sequence of SEQ ID NO: 22, 23, 24 and/or 25.

The presence and/or amount of the OXA-48-like gene, the VIM gene and/or the one or more additional carbapenemase gene may be determined by PCR and/or hybridisation, preferably by real-time PCR.

In some embodiments: (i) the presence and/or amount the OXA-48-like gene is compared with the presence and/or amount of the OXA-48-like in a control sample; (ii) the presence and/or amount the VIM gene is compared with the presence and/or amount of the VIM gene in a control sample; and/or (iii) the presence and/or amount the one or more additional carbapenemase gene is compared with the presence and/or amount of the one or more additional carbapenemase gene in a control sample.

The invention further provides a method for diagnosing a carbapenem-resistant bacteria infection in an individual comprising carrying out a method of the invention on a sample obtained from the individual. Said sample may be a sample of blood, cerebral spinal fluid, saliva, urine, cells, a cellular extract, a stool sample, a tissue sample or a tissue biopsy, or a swab from an individual, such as a rectal swab, or a swab from the environment.

The invention further provides a device for use in the method of the invention, which comprises one or more oligonucleotide specific for the OXA-48-like gene, the VIM gene and/or the one or more additional carbapenemase gene. In some embodiments the one or more oligonucleotide specific for the OXA-48-like gene, the VIM gene and/or the one or more additional carbapenemase gene is an oligonucleotide as defined herein. The one or more oligonucleotide may be immobilised on a surface.

The invention also provides a method for determining the presence and/or amount of one or more carbapenemase-producing bacteria in a sample comprising determining the presence and/or amount of a carbapenemase gene selected from an OXA-48-like gene and/or a VIM gene in said sample.

In one embodiment the presence and/or amount of the OXA-48-like gene and the VIM gene is determined. The presence and/or amount of the OXA-48-like gene may be determined using at least one oligonucleotide specific for the OXA-48-like gene and/or the presence and/or amount of the VIM gene may be determined using at least one oligonucleotide specific for the VIM gene. The at least one oligonucleotide specific for the OXA-48-like gene typically hybridises to a nucleic acid sequence within a target region of the OXA-48-like gene comprising bases 421 to 717 of the OXA-48-like gene. The at least one oligonucleotide specific for the VIM gene typically hybridises to a nucleic acid sequence within a target region of the VIM gene comprising bases 235 to 400, 238 to 403 or 245 to 410 of the VIM gene. The at least one oligonucleotide specific for the OXA-48-like gene may hybridise to a target region of the OXA-48-like gene comprising bases 421 to 490, 570 to 670 and/or 650 to 717 of the OXA-48-like gene. The at least one oligonucleotide specific for the VIM gene may hybridise to a target region of the VIM gene comprising bases: (i) 235 to 280, 238 to 283 or 240 to 290; and/or (ii) 360 to 410, 363 to 413 or 270 to 420; of the VIM gene. The at least one oligonucleotide specific for the OXA-48-like gene preferably comprises a nucleic acid sequence of SEQ ID NO: 16, 2 and/or 3, or a nucleic acid sequence having at least 80% sequence identity to the nucleic acid sequence of SEQ ID NO: 16, 2 and/or 3. The at least one oligonucleotide specific for the VIM gene preferably comprises a nucleic acid sequence of SEQ ID NO: 17, 18 and/or 19, or a nucleic acid sequence having at least 80% sequence identity to the nucleic acid sequence of SEQ ID NO: 17, 18 and/or 19.

### DESCRIPTION OF FIGURES

FIG. 1: Alignment of segments of OXA48-like carbapenemase gene sequences. Forward primers are shown underlined, reverse primers are shown double-underlined and probe sequences are dotted-underlined.

FIG. 2: Alignment of segments of VIM carbapenemase gene sequences. Forward primers are shown underlined, reverse primers are shown double-underlined and probe sequences are dotted-underlined.

FIG. 3: Alignment of segments of KPC carbapenemase gene sequences. Forward primers are shown underlined, reverse primers are shown double-underlined and probe sequences are dotted-underlined.

FIG. 4: Alignment of segments of NDM carbapenemase gene sequences. Forward primers are shown underlined, reverse primers are shown double-underlined and probe sequences are dotted-underlined.

FIG. 5: Alignment of segments of IMP carbapenemase gene sequences. Forward primers are shown underlined, reverse primers are shown double-underlined and probe sequences are dotted-underlined.

### DETAILED DESCRIPTION OF THE INVENTION

The present invention allows for the flexible, accurate, rapid and sensitive detection of carbapenem-resistant bacteria and the diagnosis of carbapenem-resistant bacteria infection through a measurement of the OXA-48-like and/or VIM genes in a biological sample at a single time point (“snapshot”) or during the course of an infection.

### Carbapenemases and Carbapenem-Resistant Bacteria

Carbapenems are a critically important sub-class of beta-lactam antibiotics that possess the broadest spectrum of activity and highest activity against Gram-positive and Gram-negative bacteria, which means they are often used as a last line of defence in treating persistent infections or infections with bacteria known or suspected of being resistant to other antibiotics.

Structurally, carbapenems are very similar to penicillins, but the sulphur atom at position 1 of the beta-lactam ring has been replaced with an unsubstituted carbon atom.

Resistance to carbapenem antibiotics is emerging in both Gram-negative and Gram-positive bacteria. Examples of carbapenem-resistant Gram-negative bacteria include Pseudomonas spp., Acinetobacter spp. and Stenotrophomonas spp., as well as the Enterobacteriaceae (for example Klebsiella spp., Escherichia coli and Enterobacter spp.). Examples of carbapenem-resistant Gram-positive bacteria include Staphylococcus spp., Streptococcus spp., Enterococcus spp. and Nocardia spp.

Mechanisms of resistance to carbapenems include the production of carbapenemases, efflux pumps and mutations that alter the expression and/or function of porins and penicillin binding proteins. Carbapenemases are specific beta-lactamases with the ability to hydrolyse carbapenems. Production of carbapenemases is a common cause of carbapenem resistance. Bacteria which produce one or more carbapenemase are referred to as carbapenemase-producing bacteria. As such bacteria are either (entirely) resistant to, or less susceptible to, carbapenem antibiotics than bacteria which do not possess a mechanism of carbapenem resistance, they are also referred to herein as carbapenem-resistant bacteria. Herein the terms carbapenemase-producing bacteria and carbapenem-resistant bacteria are used interchangeably. The present invention may be used in the detection and/or diagnosis of both Gram-positive and/or Gram-negative carbapenem-resistant bacteria, including any of the bacterial species identified herein.

The production of carbapenemases is particularly associated with Gram-negative carbapenem-resistant bacteria. Typically, therefore, the present invention relates to the detection and/or diagnosis of Gram-negative carbapenemase-producing bacteria (also referred to herein as Gram-negative carbapenem-resistant bacteria), for example Pseudomonas spp., Acinetobacter spp. and Stenotrophomonas spp., and/or the Enterobacteriaceae. In a preferred embodiment, the present invention relates to the detection and/or diagnosis of carbapenem-resistant Enterobacteriaceae, such as Klebsiella spp., Escherichia coli, Citrobacter spp., Cronobacter spp., Leclercia spp., Shigella spp., Salmonella spp. and Enterobacter spp.

The present invention may be used to detect and/or diagnose at least one species of carbapenem-resistant bacteria, such as those identified herein, particularly at least one species of Gram-negative carbapenem-resistant bacteria. For example, the present invention may be used to detect at least one, at least two, at least three, at least four, at least five, at least six, at least seven, at least eight, at least nine, at least ten or more species of carbapenemase-producing bacteria (carbapenem-resistant bacteria), particularly Gram-negative carbapenem-resistant bacteria.

Beta-lactamases are grouped into four distinct categories (A to D) based on their structure. Class B beta-lactamases are Zn²⁺-dependent and are all carbapenemases. Class A, C and D beta-lactamases use serine as a nucleophile to hydrolyse the beta-lactam bond.

The present invention relates to the detection or diagnosis of carbapenemase-producing bacteria (carbapenem-resistant bacteria) by detecting the presence and/or amount of two class B carbapenemases, specifically OXA-48-like carbapenemases and/or VIM carbapenemases. The presence and/or amount of one or more OXA-48-like carbapenemase and/or one or more VIM carbapenemase may be used to detect carbapenemase-producing bacteria (carbapenem-resistant bacteria) or diagnose a carbapenem-resistant bacterial infection according to the present invention. In a preferred embodiment, the presence and/or amount of both one or more OXA-48-like carbapenemase and one or more VIM carbapenemase is determined.

The term “OXA-48-like carbapenemase” refers to a subgroup of OXA carbapenemases having a significant degree of sequence identity (at the amino acid and/or nucleic acid level) with OXA-48. An OXa-48-like carbapenemase may be defined as an OXA carbapenemase having at least 85%, at least 90%, at least 95%, at least 96%, at least 97%, at least 98%, at least 99% or more sequence identity (at the amino acid or nucleic acid level) with any OXA carbapenemase accession number or SEQ ID NO disclosed herein, or to any known OXA-48-like carbapenemase. Typically, an OXA-48-like carbapenemase according to the present invention may be defined as an OXA carbapenemase having at least 85%, at least 90%, at least 95%, at least 96%, at least 97%, at least 98%, at least 99% or more sequence identity with one or more of the following OXA carbapenemases (at the amino acid or nucleic acid level: OXA-181 (Accession No. GI 304368222, HM992946.1), OXA-232 GI 444236140, JX423831.1, 2677-3474), OXA-247 (GI 442769982, JX893517.1, 1-786), OXA-163 (GI 323817046, HQ700343.1, 1-786), OXA-204 (GI 408795221, JQ809466.1, 5375-6172), OXA-370 (GI 573006828, KF900153.1, 1-798), OXA-245 (GI 442577759, JX438001.1, 1-798), OXA-162 (GI 270312218, GU197550.1, 1-798), OXA-48 (GI AY236073.2, 2188-2985) and/or OXA-244 (GI 442577757, JX438000.1, 1-798). In a preferred embodiment, an OXA-48-like carbapenemase has at least 85%, at least 90%, at least 95%, at least 96%, at least 97%, at least 98%, at least 99% or more sequence identity with the OXA-48 carbapenemase (GI AY236073.2, 2188-2985).

The term “VIM carbapenemase” includes all subgroups and subtypes of the VIM carbapenemase family, including but not limited to VIM-1, VIM-2 and/or VIM-7 carbapenemases, such as those identified by accession number and/or SEQ ID NO herein. The term “VIM carbapenemase” also encompasses any carbapenemase having at least 85%, at least 90%, at least 95%, at least 96%, at least 97%, at least 98%, at least 99% or more sequence identity (at the amino acid or nucleic acid level) with any VIM carbapenemase accession number or SEQ ID NO disclosed herein, or to any known VIM carbapenemase.

As well as determining the presence and/or amount of one or more OXA-48-like carbapenemase and/or one or more VIM carbapenemase, the presence and/or amount of one or more additional carbapenemase may be determined. For example the presence and/or amount of at least two, at least three, at least four, at least five, at least six, at least seven, at least eight, at least nine, at least ten or more additional carbapenemase may determined in combination with determining the presence and/or amount of one or more OXA-48-like carbapenemase and/or one or more VIM carbapenemase.

The one or more additional carbapenemase is not limited, and may be a class A, B, C or D carbapenemase. For example, the one or more additional carbapenemase may be selected from K. pneumonia carbapenemase (KPC, class A), GES (class A), SME (class A), NmcA (class A), IMP (class B), New Delhi metallo-beta-lactamase (NDM or NDM-1, class B), Verona integrin-encoded metallo-beta-lactamase (VIM, class B), CphA (class B), CMY (class C), oxacillinase (OXA, class D), or others (e.g. SME, IMI, NMC, SPM, AIM, GIM, DIM and CcrA). Any combination of these additional carbapenemase may be used according to the present invention. Any member of these carbapenemase families may be used in the context of the present invention. Non-exhaustive examples of OXA carbapenemases that may be used as a one or more additional carbapenemase include OXA-10, OXA-23, OXA-24/40, OXA-48, OXA-51, OXA-55, OXA-58, OXA-143, OXA-181 and OXA-232.

Typically, the one or more additional carbapenemase is selected from a KPC carbapenemase and/or an NDM carbapenemase. In a preferred embodiment, the presence and/or amount of a KPC carbapenemase and an NDM carbapenemase is determined.

The term “NDM carbapenemase” includes all subgroups and subtypes of the NDM carbapenemase family, including but not limited to NDM-1 carbapenemases, NDM-2 carbapenemases, NDM-3 carbapenemases, NDM-4 carbapenemases and NDM-5 carbapenemases, such as those identified by accession number and/or SEQ ID NO herein, or a variant having at least 85%, at least 90%, at least 95%, at least 96%, at least 97%, at least 98%, at least 99% or more sequence identity (at the amino acid or nucleic acid level) with any NDM carbapenemase accession number or SEQ ID NO disclosed herein, or to any known NDM carbapenemase. In a preferred embodiment of the invention the presence and/or amount of an OXA-48-like carbapenemase and the presence and/or amount of an NDM carbapenemase, such as NDM-1, NDM-4 and/or NDM-5, is determined. As a non-limiting example, the presence and/or amount of OXA-48 may be determined and the presence and/or amount of NDM-1 may be determined. As another non-limiting example, the presence and/or amount of OXA-48 may be determined and the presence and/or amount of NDM-4 may be determined. As another non-limiting example, the presence and/or amount of OXA-48 may be determined and the presence and/or amount of NDM-5 may be determined. As another non-limiting example, the presence and/or amount of OXA-181 may be determined and the presence and/or amount of NDM-1 may be determined. As another non-limiting example, the presence and/or amount of OXA-181 may be determined and the presence and/or amount of NDM-4 may be determined. As another non-limiting example, the presence and/or amount of OXA-181 may be determined and the presence and/or amount of NDM-5 may be determined. As another non-limiting example, the presence and/or amount of OXA-232 may be determined and the presence and/or amount of NDM-1 may be determined. As another non-limiting example, the presence and/or amount of OXA-232 may be determined and the presence and/or amount of NDM-4 may be determined. As another non-limiting example, the presence and/or amount of OXA-232 may be determined and the presence and/or amount of NDM-5 may be determined. Any oligonucleotide specific for an OXA-48-like gene of the invention may be used in the detection of said OXA-48, OXA-181 and/or OXA-232 gene. In addition, the presence and/or amount of a VIM carbapenemase and/or one or more additional carbapenemase, such as a KPC carbapenemase (e.g. KPC-1 and/or KPC-2), may also be determined.

The term “KPC carbapenemase” includes all subgroups and subtypes of the KPC carbapenemase family, including but not limited to KPC-1 and/or KPC-2, such as those identified by accession number and/or SEQ ID NO herein, or a variant having at least 85%, at least 90%, at least 95%, at least 96%, at least 97%, at least 98%, at least 99% or more sequence identity (at the amino acid or nucleic acid level) with any KPC carbapenemase accession number or SEQ ID NO disclosed herein, or to any known KPC carbapenemase. This applies equally to other carbapenemase gene families disclosed herein. In a preferred embodiment of the invention the presence and/or amount of an OXA-48-like carbapenemase and the presence and/or amount of an KPC carbapenemase, such as KPC-1 and/or KPC-2, is determined. As a non-limiting example, the presence and/or amount of OXA-48 may be determined and the presence and/or amount of KPC-2 may be determined. Any oligonucleotide specific for an OXA-48-like gene of the invention may be used in the detection of said OXA-48 gene.

The one or more additional carbapenemase may be selected from IMP, IMI, SME, SPM and GES.

Thus, in a preferred embodiment, the present invention relates to a method for detecting the presence and/or amount of one or more carbapenemase-producing bacteria (carbapenem-resistant bacteria) comprising determining the presence and/or amount of: (i) and OXA-48-like carbapenemase and a VIM carbapenemase; (ii) an OXA-48-like carbapenemase and a NDM carbapenemase; (iii) an OXA-48-like carbapenemase, and a KPC carbapenemase; (iv) an OXA-48-like carbapenemase, a KPC carbapenemase and a NDM carbapenemase; (v) an OXA-48-like carbapenemase, a VIM carbapenemase and a KPC carbapenemase; (vi) an OXA-48-like carbapenemase, a VIM carbapenemase and a NDM carbapenemase; (vii) a VIM carbapenemase and a NDM carbapenemase; (viii) a VIM carbapenemase and a KPC carbapenemase; (ix) a VIM carbapenemase, a KPC carbapenemase and a NDM carbapenemase; or (x) an OXA-48-like carbapenemase, a VIM carbapenemase, a KPC carbapenemase and a NDM carbapenemase. In a particularly preferred embodiment, the present invention relates to a method for detecting the presence and/or amount of one or more carbapenemase-producing bacteria (carbapenem-resistant bacteria) comprising determining the presence and/or amount of an OXA-48-like carbapenemase, a VIM carbapenemase, a KPC carbapenemase and a NDM carbapenemase.

In another preferred embodiment, the present invention relates to a method for detecting the presence and/or amount of one or more carbapenemase-producing bacteria (carbapenem-resistant bacteria) comprising determining the presence and/or amount of any combination of carbapenemases as set out in (i) to (x) of the preceding paragraph, particularly detecting the presence and/or amount of an OXA-48-like carbapenemase, a VIM carbapenemase, a KPC carbapenemase, a NDM carbapenemase, wherein the presence and/or amount of at least one of an IMP carbapenemase, an IMI carbapenemase, an SME carbapenemase, an SPM carbapenemase and/or a GES carbapenemase is also determined. Said method may involve the detection of at least two, at least three, at least four, or all five of an IMP carbapenemase, an IMI carbapenemase, an SME carbapenemase, an SPM carbapenemase and/or a GES carbapenemase or any combination thereof (in addition to any of the combinations of OXA-48-like carbapenemase, a VIM carbapenemase, a KPC carbapenemase and a NDM carbapenemase). As an example, said method may involve the detection of an IMP carbapenemase and an IMI carbapenemase (in addition to any combination of an OXA-48-like carbapenemase, a VIM carbapenemase, a KPC carbapenemase, a NDM carbapenemase).

In a particularly preferred embodiment, the present invention relates to a method for detecting the presence and/or amount of one or more carbapenemase-producing bacteria (carbapenem-resistant bacteria) comprising determining the presence and/or amount of an OXA-48-like carbapenemase, a VIM carbapenemase, a KPC carbapenemase, a NDM carbapenemase, an IMP carbapenemase, an IMI carbapenemase, an SME carbapenemase, an SPM carbapenemase and a GES carbapenemase.

In embodiments of the invention wherein the presence and/or amount of both an NDM carbapenemase gene and a KPC carbapenemase gene is detection, the KPC gene may preferably be a KPC-2 gene and the NDM gene may preferably be an NDM-1 gene, and NDM-4 gene and/or an NDM-5 gene, particularly preferably an NDM-1 gene.

The carbapenemase genes as described herein may have a nucleic acid sequence as shown in the sequences in the Sequence Information section herein. The relevant sequence identifiers are also shown. The one or more carbapenemase gene of the invention may have a sequence identity of at least 80% with the corresponding nucleic acid sequence shown in the Sequence Information section. Sequence identity may be calculated as described herein. A sequence identity of at least 80% includes at least 82%, at least 84%, at least 86%, at least 88%, at least 90%, at least 91%, at least 92%, at least 93%, at least 94%, at least 95%, at least 96%, at least 97%, at least 98%, at least 99%, and 100% sequence identity (to each and every nucleic acid sequence presented herein and/or to each and every SEQ ID NO and/or to each and every accession number presented herein). For example, the OXA-48 gene may have at least 80% sequence identity with any of the OXA gene accession numbers or SEQ ID NO disclosed herein and/or the VIM gene may have at least 80% sequence identity with any of the VIM gene accession numbers or SEQ ID NO disclosed herein.

### Detection and Quantification of Carbapenem-Resistant Bacteria

The present inventors have developed new primer/probe sequences for OXA-48-like carbapenemase genes and VIM carbapenemase genes. These primers/probes have been designed to maximise coverage of different variants of the OXA-48-like and VIM carbapenemase genes respectively. Thus, the present invention provides primers/probes that enable the detection of OXA-48-like carbapenemase and/or VIM carbapenemase-producing bacteria with improved sensitivity, because they detect more OXA-48-like and VIM carbapenemase variants, and so the likelihood of false negatives is reduced.

### Decision Trees

A “decision rule” or a “decision tree” is a method used to classify individuals. This rule can take on one or more forms that are known in the art, as exemplified in Hastie et al., in “The Elements of Statistical Learning” Springer-Nerlag (Springer, New York (2001)). Analysis of biomarkers in the complex mixture of molecules within the sample generates features in a data set. A decision rule or a decision tree may be used to act on a data set of features to detect one or more carbapenemase-producing bacteria and/or diagnose a carbapenem-resistant bacterial infection.

The application of the decision rule or the decision tree does not require perfect classification. A classification may be made with at least about 60%, at least about 70%, at least about 80%, at least about 90% certainty or even more. The useful degree of certainty may vary, depending on the particular method of the present invention. “Certainty” is defined as the total number of accurately classified individuals divided by the total number of individuals subjected to classification. As used herein, “certainty” means “accuracy”.

Classification may also be characterized by its “sensitivity”. The “sensitivity” of classification relates to the percentage of samples containing carbapenemase-producing bacteria and/or carbapenem-resistant bacterial infections that were correctly identified. “Sensitivity” is defined in the art as the number of true positives divided by the sum of true positives and false negatives.

The “specificity” of a method is defined as the percentage of samples that were correctively identified as not having carbapenemase-producing bacteria and/or carbapenem-resistant bacterial infections compared with an uninfected/negative control(s). That is, “specificity” relates to the number of true negatives divided by the sum of true negatives and false positives.

Typically, the accuracy, sensitivity and/or specificity is at least about 90%, at least about 80%, at least about 70% or at least about 60%.

Diagnosing a carbapenem-resistant bacterial infection in an individual means to identify or detect the presence and/or amount of one or more carbapenemase-producing bacteria in the individual. This is achieved by determining the presence and/or amount of one more carbapenemase gene as described herein.

Because of the sensitivity of the present invention to detect a carbapenem-resistant bacterial infection before an overtly observable clinical manifestation, the diagnosis, identification or detection of a carbapenem-resistant bacterial infection includes the detection of the onset of a carbapenem-resistant bacterial infection, as defined above.

According to the present invention, a carbapenem-resistant bacterial infection may be diagnosed or detected, by determining the presence and/or amount of one or more carbapenemase-producing bacteria in a sample obtained from an individual. As used herein, “obtain” means “to come into possession of”. The present invention is particularly useful in predicting and diagnosing a carbapenem-resistant bacterial infection in an individual, who is suspected of having a carbapenem-resistant bacterial infection, or who is at risk of a carbapenem-resistant bacterial infection. The present invention may be used to confirm a clinical suspicion of a carbapenem-resistant bacterial infection.

### Controls or Reference Populations

The presence and/or amount of the one or more carbapenemase-producing bacteria in a sample may be measured relative to a control or reference population, for example relative to the corresponding carbapenemase-producing bacteria of a control or reference population. Herein the terms “control” and “reference population” are used interchangeably. The actual amount of the one or more carbapenemase produced by the one or more carbapenemase-producing bacteria, such as the mass, molar amount, concentration or molarity of the one or more carbapenemase may be assessed and compared with the corresponding value from the control or reference population. Alternatively, the amount of one or more carbapenemase may be compared with that of the control or reference population without quantifying the mass, molar amount, concentration or molarity of the one or more carbapenemase.

The control or reference population can be generated from one individual or a population of two or more individuals. The control or reference population, for example, may comprise three, four, five, ten, 15, 20, 30, 40, 50 or more individuals. Furthermore, the control or reference population and the individual's (test) sample that are compared in the methods of the present invention may be generated from the same individual, provided that the test and reference samples are taken at different time points and compared to one another. For example, a sample may be obtained from an individual at the start of a study period. A control or reference taken from that sample may then be compared to subsequent samples from the same individual. Such a comparison may be used, for example, to determine the progression of a carbapenemase-producing bacteria and/or carbapenem-resistant bacterial infection in the individual by repeated classifications over time.

The control or reference may be obtained, for example, from a population of carbapenemase-producing bacteria negative individuals (i.e. individuals negative for infection by one or more carbapenem-resistant bacteria) or carbapenemase-producing bacteria positive individuals (i.e. individuals positive for infection by one or more carbapenem-resistant bacteria).

Typically the control or reference population does not comprise one or more carbapenemase-producing bacteria and/or is not infected with one or more carbapenem-resistant bacteria (i.e. is negative for carbapenem-resistant bacterial infection). Alternatively, the control or reference population may comprise one or more carbapenemase-producing bacteria and/or be infected with one or more carbapenem-resistant bacteria (i.e. is positive for carbapenem-resistant bacterial infection) and may be subsequently diagnosed with a carbapenem-resistant bacterial infection using conventional techniques. For example, a population of carbapenem-resistant bacterial infection-positive individuals used to generate the reference or control may be diagnosed with carbapenem-resistant bacterial infection about 24, 48, 72, 96 or more hours after biological samples were taken from them for the purposes of generating a reference or control. In one embodiment, the population of carbapenem-resistant bacterial infection-positive individuals is diagnosed with carbapenem-resistant bacterial infection using conventional techniques about 0-36 hours, about 36-60 hours, about 60-84 hours, or about 84-108 hours after the biological samples were taken.

As described herein, the present invention relates to a method for determining the presence and/or amount of one or more carbapenemase-producing bacteria and/or diagnosing a carbapenem-resistant bacterial infection. Thus, in some instances it is sufficient to detect the presence of one or more carbapenemase-producing bacteria (i.e. carbapenem-resistant bacteria) in a sample. In such cases, the control or reference population may be positive or negative for carbapenemase-producing bacteria/carbapenem-resistant bacterial infection.

In other instances, the amount of the one or more carbapenemase-producing bacteria (i.e. carbapenem-resistant bacteria) is determined relative to a control or reference population. In such cases, the amount of the one or more carbapenemase-producing bacteria (i.e. carbapenem-resistant bacteria) is typically increased compared with a control or reference population, the amount may be increased by at least 10%, at least 20%, at least 30%, at least 40%, at least 50%, at least 60, at least 70%, at least 80%, at least 90%, at least 100%, at least 150%, at least 200% compared with the control or reference population.

Alternatively, if the control or reference population is positive for carbapenemase-producing bacteria and/or a carbapenem-resistant bacterial infection, the amount of the one or more carbapenemase-producing bacteria (i.e. carbapenem-resistant bacteria) may be decreased compared with the control or reference population. For example, the amount may be decreased by at least 10%, at least 20%, at least 30%, at least 40%, at least 50%, at least 60, at least 70%, at least 80%, at least 90%, at least 95%, at least 99%, up to total elimination of the one or more carbapenemase-producing bacteria (carbapenem-resistant bacteria). Such a carbapenem-resistant bacterial infection positive control or reference population may, for example, be used to monitor an individual's response to a treatment directed to the carbapenem-resistant bacterial infection, such that if the treatment is successful, the amount of carbapenemase-producing bacteria will decrease relative to the control or reference population over time.

### Measurement of the One or More Carbapenemase-Producing Bacteria

The presence and/or amount of the one or more carbapenemase-producing bacteria (carbapenem-resistant bacteria) according to the present invention is determined by determining the presence and/or amount of one or more carbapenemase gene produced by the one or more carbapenemase-producing bacteria. If more than one carbapenemase-producing bacteria is present in a sample, the different bacteria may produce the same carbapenemase gene or different carbapenemase genes. Each carbapenemase-producing bacteria according to the present invention may produce one, two, three, four, five, six, seven, eight, nine, ten or more different carbapenemase genes.

Measurements of the one or more carbapenemase gene may include, for example, measurements that indicate the presence, concentration, expression level, or any other value associated with the one or more carbapenemase gene.

The present and/or amount of said one or more carbapenemase gene may be determined by quantitative and/or qualitative analysis. Thus, the presence of a carbapenemase-producing bacteria or carbapenem-resistant bacterial infection may be determined simply by determining the expression of one or more carbapenemase gene according to the invention (qualitative analysis), with no need to determine the amount of the carbapenemase gene. Alternatively, the amount of the one or more carbapenemase gene may be determined (quantitative analysis).

The amount of the one or more one or more carbapenemase gene encompasses, but is not limited to, the mass of the one or more carbapenemase gene, the molar amount of the one or more carbapenemase gene, the concentration of the one or carbapenemase gene, the molarity of the one or more carbapenemase gene and the copy number of the one or more carbapenemase gene. This amount may be given in any appropriate units. For example, the concentration of the one or more carbapenemase gene may be given in pg/ml, ng/ml or μg/ml.

The presence and/or amount of the one or more carbapenemase gene may be measured directly or indirectly. For example, the copy number of the one or more carbapenemase gene may be determined using PCR, qPCR or qRT-PCR. The expression level of the one or more carbapenemase gene may be determined, for example using reverse transcription PCR (RT-PCR). The relative presence and/or amount of the one or more carbapenemase gene relative to a control or reference population may be determined using any appropriate technique. Suitable standard techniques are known in the art.

As used herein, “comparison” includes any means to discern at least one difference in the presence and/or amount of the one or more carbapenemase gene in the individual and the control or reference population. Thus, a comparison may include a visual inspection of chromatographic spectra or numerical data, and a comparison may include arithmetical or statistical comparisons of values assigned to expression of the one or more carbapenemase gene in the individual's sample and the control or reference. Such statistical comparisons include, but are not limited to, applying a decision rule or decision tree. If at least one internal standard is used, the comparison to discern a difference between the individual and the reference or control may also include features of these internal standards, such that the present and/or amount of the one or more carbapenemase gene in the individual's sample is correlated to the internal standards. The comparison can confirm the presence or absence of one or more carbapenemase-producing bacteria (i.e. carbapenem-resistant bacteria), and thus to detect or diagnose a carbapenem-resistant bacterial infection.

The presence and/or amount level of the one or more carbapenemase gene (and hence the one or more carbapenemase-producing bacteria) may be altered compared with a control or reference population for at least 12 hours, at least 24 hours, at least 30 hours, at least 48 hours, at least 72 hours, at least 96 hours, at least 120 hours, at least 144 hours, at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks or more.

Although the invention does not require a monitoring period to diagnose a carbapenem-resistant bacterial infection, it will be understood that repeated classifications of the individual, i.e., repeated snapshots, may be taken over time until the individual is no longer at risk. Alternatively, the presence and/or amount of one or more carbapenemase-producing bacteria in a sample obtained from the individual may be compared to presence and/or amount of one or more carbapenemase-producing bacteria in samples obtained from the same individual at different points in time.

As used herein, an “individual” is an animal, preferably a mammal, more preferably a human or non-human primate. The terms “individual,” “subject” and “patient” are used interchangeably herein. The individual can be normal, suspected of having a carbapenem-resistant bacterial infection or at risk of a carbapenem-resistant bacterial infection.

The present invention enables the rapid detection of one or more carbapenemase-producing bacteria and/or a carbapenem-resistant bacterial infection. By way of example, the method of the invention is typically completed within 3 hours, preferably within 1 to 3 hours, 1 to 2 hours or 1 to 1.5 hours.

The presence and/or amount of one or more carbapenemase-producing bacteria, as determined by determining the presence and/or amount of one or more carbapenemase genes may be detected, quantified or determined by any appropriate means.

### Samples

The presence and/or amount of the one or more carbapenemase gene may be determined in a sample obtained from a bacterial isolate or bacterial culture. The presence and/or amount of the one or more carbapenemase gene may be determined in a sample obtained from an environmental sample, for example a swab taken from any environmental site, such as a surface in a hospital. The presence and/or amount of the one or more carbapenemase gene may be determined in a sample obtained from an individual. The sample may be any suitable biological material, for example blood, plasma, saliva, serum, sputum, urine, cerebral spinal fluid, cells, a cellular extract, a tissue sample, a tissue biopsy, a stool sample, a swab from any body site and the like. Typically the sample is from a bacterial isolate/culture or a rectal swab, stool specimen, urine sample and/or blood sample. The precise biological sample that is taken from the individual may vary, but the sampling preferably is minimally invasive and is easily performed by conventional techniques. The biological sample may be taken from the individual before, during, and/or after treatment for a carbapenem-resistant bacterial infection. In one embodiment, the sample is taken after treatment for a carbapenem-resistant bacterial infection has been initiated.

When samples are taken from a patient or the environment (rather than a bacterial culture or isolate), extraction of the bacterial DNA is typically required prior to testing according to the present invention. Accordingly, the methods of the present invention may comprise a DNA extraction step. In a preferred embodiment a formal and standard DNA extraction method is used to extract the bacterial DNA. Any suitable technique for DNA extraction may be used. Suitable techniques are known in the art, for example spin column and precipitation techniques. Equally, an automated robotic system for DNA extraction may be used.

### Methodology

As described herein, the one or more carbapenemase gene of the invention is detected (quantitatively and/or qualitatively) at the nucleic acid level. Thus, the one or more carbapenemase gene may be detected as DNA and/or RNA and may be detected using any appropriate technique. The presence and/or amount of the one or more carbapenemase gene of the invention may be measured directly or indirectly.

Any appropriate agent may be used to determine the presence and/or amount of the one or more carbapenemase gene of the invention. For example, the presence and/or amount of the one or more carbapenemase gene of the invention may be determined using an agent selected from peptides and peptidomimetics, antibodies, small molecules and single-stranded DNA or RNA molecules, as described herein. The relative presence and/or amount of the one or more carbapenemase gene of the invention relative to a control or reference population (see above) may be determined using any appropriate technique. Suitable standard techniques are known in the art.

Typically the determination of the presence and/or amount of the one or more carbapenemase gene is carried out by amplifying said one or more carbapenemase gene, a target region of said one or more carbapenemase gene or a fragment of said gene or target region. Amplification may be carried out using methods and platforms known in the art, for example PCR (for example, with the use of “Fast DNA Polymerase”, Life Technologies), such as real-time or quantitative PCR (qPCR), block-based PCR, ligase chain reaction, glass capillaries, isothermal amplification methods including loop-mediated isothermal amplification, rolling circle amplification transcription mediated amplification, nucleic acid sequence-based amplification, signal mediated amplification of RNA technology, strand displacement amplification, isothermal multiple displacement amplification, helicase-dependent amplification, single primer isothermal amplification, and circular helicase-dependent amplification. If employed, amplification may be carried using any amplification platform. In a preferred embodiment, PCR is used, in a more preferred embodiment, q-PCR is used.

PCR amplification primers are typically employed to amplify approximately 100-400, for example 100-300 or 100-200 base pair regions of the target/complementary nucleic acid that contain the nucleotide targets of the one or more carbapenemase gene of the present invention. In the presence of a suitable polymerase and DNA precursors (dATP, dCTP, dGTP and dTTP), forward and reverse primers are extended in a 5′ to 3′ direction, thereby initiating the synthesis of new nucleic acid strands that are complementary to the individual strands of the target nucleic acid. The primers thereby drive amplification of target nucleic acid sequences in the one or more carbapenemase gene, thereby generating amplification products comprising said target nucleic acid sequences.

In a preferred embodiment, the oligonucleotide probes disclosed herein are used as primers to amplify the one or more carbapenemase gene, one or more target regions within said one or more carbapenemase gene or a fragment of said carbapenemase gene. All references, description and features herein described in relation to probes of the invention apply equally to primers of the invention. In this embodiment, the probes (acting as primers) are extended from their 3′ ends (i.e. in a 5′-to-′3′) direction. Such an amplification step may be employed in conjunction with a general amplification step (used to amplify all the DNA within a sample to facilitate detection).

Typically, the presence and/or amount of the one or more carbapenemase gene is determined using PCR. DNA is isolated from a sample and specific primers are used to amplify the one or more carbapenemase gene (if present in the sample). In a preferred embodiment, qPCR is used to determine the presence and/or amount of the one or more carbapenemase gene.

The presence and/or amount of the one or more carbapenemase gene may be determined by determining the presence and/or amount of a target region of the one or more carbapenemase gene and/or the presence and/or amount of one or more fragment of said carbapenemase gene or target region thereof. References herein to determining the presence and/or amount of the one or more carbapenemase gene apply equally to determining the presence and/or amount of a target region of the one or more carbapenemase gene, and/or the presence and/or amount one or more fragment of said carbapenemase gene or target region thereof.

For example, specific primer pairs as disclosed herein for the one or more carbapenemase gene may be used to amplify the one or more carbapenemase gene, a target region thereof or a fragment of the one or more carbapenemase gene or target region thereof. Typically the fragment is from a target region of the one or more carbapenemase gene. The amplification products of PCR reaction may then be visualised by any appropriate means. For example, the amplification products may be separated and visualised by agarose gel electrophoresis. As the molecular weight of the one or more carbapenemase gene or fragment thereof will be known, the presence of the one or more carbapenemase gene or fragment thereof may be readily determined by the band size of the amplification products as run on an agarose gel. This method has the advantage of requiring standard equipment that will be present in most laboratories.

Specific primer pairs as disclosed herein can also be used in qPCR to determine the presence and/or amount of the one or more carbapenemase gene, a target region thereof and/or a fragment of the one or more carbapenemase gene or a target region thereof. Non-specific fluorescent dyes may be used in qPCR according to the present invention. Standard qPCR methods using such non-specific fluorescent dyes are known in the art. Alternatively, DNA probes specific for the one or more carbapenemase gene of the invention, said probes further comprising a fluorescent reporter, may be used. Said DNA probes may comprise or consist of one or more of the oligonucleotides of the invention as described herein, further comprising a fluorescent reporter. Examples of fluorescent reporters (also referred to as fluorescent tags) are also described herein. Instruments enabling fast on-screen detection of genes by qPCR are commercially available (for example, Taqman®).

DNA is typically extracted and/or isolated from a sample prior to analysis by a method of the present invention. Preferably the DNA is purified prior to analysis. Any appropriate may be used to extract and/or isolate and/or purify the DNA. Standard techniques are known in the art and commercial kits are available.

Any other appropriate technique for determining the presence and/or amount of the one or more carbapenemase gene, a target region thereof and/or a fragment of one or more carbapenemase gene or a target region thereof may also be used. For example, the presence and/or amount of the one or more carbapenemase gene, a target region thereof and/or a fragment of one or more carbapenemase gene or a target region thereof may be determined using a method selected from nuclear magnetic resonance, nucleic acid arrays, dot blotting, slot blotting, reverse transcription amplification by reverse transcription PCR (RT-PCR) and Northern analysis.

The methods of the present invention also encompass determining the expression of the one or more carbapenemase gene of the invention, a target region thereof and/or a fragment of the one or more carbapenemase gene or a target region thereof, for example using reverse transcription PCR (RT-PCR). Typically mRNA from a sample is extracted and, optionally, purified, prior to analysis by RT-PCR. Oligonucleotides of the invention may be used as primers for the reverse transcription of the one or more carbapenemase gene, a target region thereof and/or a fragment of one or more carbapenemase gene or a target region thereof, and/or as primers for the amplification of the resulting cDNA. Either standard PCR or qPCR may be used to amplify and analyse the cDNA resulting from the reverse transcription (i.e. either RT-PCR or quantitative RT-PCR, RT-qPCR).

Other means of detection of the one or more carbapenemase gene at the nucleic acid level include: (i) carbapenemase-specific oligonucleotide DNA or RNA or any other nucleic acid derivative probes bound to a solid surface; (ii) purified RNA (labelled by any method, for example using reverse transcription and amplification) hybridised to probes; (iii) labelling the RNA in a sample by any method and hybridised to probes; (iv) purified RNA hybridised to probes and a second probe (labelled by any method) hybridised to the purified RNA; (v) hybridising probes to RNA in a sample, and a second probe (labelled by any method) which is hybridised to the RNA; (vi) obtaining purified RNA (labelled by any method), and hybridising the purified labelled RNA to probes; (vii) obtaining purified RNA and hybridising the RNA to probes, then using a second probe (labelled by any method) which hybridises to the RNA; (viii) RT-PCR using any primer/probe combination or inter-chelating fluorescent label, for example SyberGreen; (ix) end-point PCR; (x) digital PCR; (xi) sequencing; (xii) array cards (RT-PCR); (xiii) lateral flow devices/methodology; and/or (xiv) digital microfluidics. Other suitable techniques include rapid whole genome sequencing.

DNA or RNA from a sample (either purified or unpurified) may be labelled via any method (typically amplification) and used to interrogate one or more probe immobilised on a surface. The probe may be any length as defined herein. Typically, the oligonucleotide probe is 10 to 60 nucleotides in length, more preferably 10 to 50 nucleotides in length, even more preferably 10 to 40 nucleotides in length, even more preferably 12 to 40 nucleotides in length, and most preferably 15 to 35 nucleotides in length.

Alternatively, one or more probe may be immobilised on a surface and the DNA or RNA from a sample is hybridised to one or more second probe (labelled by any method). The DNA or RNA hybridised with the second (labelled) probe is then used to interrogate the one or more probe immobilised on the surface. Examples of such methodology are known in the art, including the Vantix™ system.

Any appropriate agent for the detection of and/or for the determination of the amount of the one or more carbapenemase gene of the invention, a target region thereof and/or a fragment of one or more carbapenemase gene or a target region thereof may be used. Similarly, any method that allows for the detecting of the one or more carbapenemase gene, a target region thereof and/or a fragment of one or more carbapenemase gene or a target region thereof the quantification, or relative quantification of the one or more carbapenemase gene may be used.

Agents for the detection of or for the determination of the amount of one or more carbapenemase gene may be used to determine the amount of the one or more carbapenemase gene in a sample obtained from the individual. Such agents typically bind to the one or more carbapenemase gene. Such agents may bind specifically to the one or more carbapenemase gene. The agent for the detection of or for the determination of the amount of the one or more carbapenemase gene may be a nucleic acid probe or other binding agent specific for the one or more carbapenemase gene, a target region thereof and/or a fragment of one or more carbapenemase gene or a target region thereof. By specific, it will be understood that the agent binds to the molecule of interest, in this case the one or more carbapenemase gene, with no significant cross-reactivity to any other molecule, particularly any other nucleic acid. For example, an agent or antibody that is specific for one or more OXA-48-like carbapenemase will show no significant cross-reactivity with human neutrophil elastase and/or VIM. Cross-reactivity may be assessed by any suitable method. Cross-reactivity of an agent or for the one or more carbapenemase gene with a molecule other than the one or more carbapenemase gene may be considered significant if the agent or antibody binds to the other molecule at least 5%, 10%, 15%, 20%, 25%, 30%, 35%, 40%, 45%, 50%, 55%, 60%, 65%, 70%, 75%, 80%, 85%, 90% or 100% as strongly as it binds to the one or more carbapenemase gene. An agent that is specific for the one or more carbapenemase gene may bind to another molecule such as human neutrophil elastase at less than 90%, 85%, 80%, 75%, 70%, 65%, 60%, 55%, 50%, 45%, 40%, 35%, 30%, 25% or 20% the strength that it binds to the one or more carbapenemase gene. Preferably, the agent or antibody binds to the other molecule at less than 20%, less than 15%, less than 10% or less than 5%, less than 2% or less than 1% the strength that it binds to the one or more carbapenemase gene.

The determination of the presence and/or amount of the one or more carbapenemase gene may use of one or more separation methods. For example, suitable separation methods may include a mass spectrometry method, such as electrospray ionization mass spectrometry (ESI-MS), ESI-MS/MS, ESI-MS/(MS)n (n is an integer greater than zero), matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SLMS), quadrupole time-of-flight (Q-TOF), atmospheric pressure chemical ionization mass spectrometry (APCI-MS), APCI-MS/MS, APCI-(MS)n, atmospheric pressure photoionization mass spectrometry (APPI-MS), APPI-MS/MS, and APPI-(MS)n. Other mass spectrometry methods may include, inter alia, quadrupole, fourier transform mass spectrometry (FTMS) and ion trap. Other suitable separation methods may include chemical extraction partitioning, column chromatography, ion exchange chromatography, hydrophobic (reverse phase) liquid chromatography, isoelectric focusing, one-dimensional polyacrylamide gel electrophoresis (PAGE), two-dimensional polyacrylamide gel electrophoresis (2D-PAGE) or other chromatography, such as thin-layer, gas or liquid chromatography, or any combination thereof. The sample may be fractionated prior to application of the separation method.

The determination of the presence and/or amount of the one or more carbapenemase gene may not require physical separation of the one or more carbapenemase gene. For example, nuclear magnetic resonance (NMR) spectroscopy may be used to resolve one or more carbapenemase gene from a complex mixture of molecules. An analogous use of NMR to classify tumours is disclosed in Hagberg, NMR Biomed. 11: 148-56 (1998), for example. Additional procedures include nucleic acid amplification technologies, which may be used to generate a profile of the one or more carbapenemase genes without physical separation of individual carbapenemase genes. (See Stordeur et al, J. Immunol. Methods 259: 55-64 (2002) and Tan et al, Proc. Nat'l Acad. Sci. USA 99: 11387-11392 (2002), for example.)

In one embodiment, the total mRNA from a sample of the individual is assayed, and the various mRNA species that are obtained from the sample are used interrogated for the presence of carbapenemase mRNAs, by hybridizing these mRNAs to an array of specific probes for particular carbapenemase genes, which said probes may comprise oligonucleotides or cDNAs, using standard methods known in the art. Alternatively, the mRNAs may be subjected to gel electrophoresis or blotting methods such as dot blots, slot blots or Northern analysis, all of which are known in the art. (See, e.g., Sambrook et al. in “Molecular Cloning, 3rd ed.,” Cold Spring Harbor Laboratory Press, Cold Spring Harbor, N.Y. (2001).) mRNA profiles also may be obtained by reverse transcription followed by amplification and detection of the resulting cDNAs, as disclosed by Stordeur et al, supra, for example. In another embodiment, the profile may be obtained by using a combination of methods, such as a nucleic acid array combined with mass spectroscopy.

### Probes and Primers

Any appropriate detection means can be used to determine the presence and/or amount of the one or more carbapenemase gene of the invention, a target region thereof and/or a fragment of one or more carbapenemase gene or a target region thereof, and hence determine the presence and/or amount of one or more carbapenemase-producing bacteria, as described herein.

Typically the presence of the one or more carbapenemase gene may be detected, and/or the amount of the one or more carbapenemase gene determined using at one or more oligonucleotide specific for the one or more carbapenemase gene. Typically, said oligonucleotide is an oligonucleotide probe or primer.

As well as providing methods for determining the presence and/or amount of one or more carbapenemase-producing bacteria, the invention also provides oligonucleotides, particularly primers, primer pairs and probes suitable for use in such methods. Such oligonucleotides are described in more detail herein. Any reference herein to an oligonucleotide specific for the one or more carbapenemase gene applies equally to an oligonucleotide probe or primer for the same one or more carbapenemase gene. Similarly, any reference herein to an oligonucleotide probe or primer for the one or more carbapenemase gene applies equally to an oligonucleotide specific for the same one or more carbapenemase gene. In addition, any reference or disclosure herein to an oligonucleotide probe of the invention applies equally to an oligonucleotide primer of the invention and any reference or disclosure herein to an oligonucleotide primer of the invention applies equally to an oligonucleotide probe of the invention.

An oligonucleotide specific for the one or more carbapenemase gene of the invention, or a probe or primer of the invention may have at least 80% sequence identity to the one or more carbapenemase gene of the invention, or a target region within said carbapenemase gene, measured over any appropriate length of sequence. Typically the % sequence identity is determined over a length of contiguous nucleic acid residues. An oligonucleotide specific for the one or more carbapenemase gene, or an oligonucleotide probe or primer of the invention may, for example, have at least 80% sequence identity to the one or more carbapenemase gene of the invention, or target region thereof, measured over at least 10, at least 11, at least 12, at least 13, at least 14, at least 15, at least 16, at least 14, at least 18, at least 19, at least 20, at least 21, at least 22, at least 23, at least 24, at least 25, at least 26, at least 27, at least 28, at least 29, at least 30, at least 35, at least 40, at least 50, at least 60, or more nucleic acid residues, up to the oligonucleotide specific for the one or more carbapenemase gene, or the oligonucleotide probe or primer having at least 80% sequence identity with the one or more carbapenemase gene of the invention, a target region thereof and/or a fragment of one or more carbapenemase gene or a target region thereof, over the entire length of the oligonucleotide specific for the one or more carbapenemase gene, or the oligonucleotide probe or primer. Sequence identity may be determined with respect to any accession number or SEQ ID NO of the one or more carbapenemase gene disclosed herein. For example, sequence identity of an oligonucleotide specific for an OXA-48-like carbapenemase gene, or OXA-48-like oligonucleotide probe or primer may be determined with respect to the sequence of any OXA-48-like accession number or SEQ ID NO disclosed herein. This also applies to the other carbapenemases disclosed herein, for example the sequence identity of a VIM specific oligonucleotide, probe or primer may be determined relative to the sequence of any VIM accession number or SEQ ID disclosed herein, etc. Where an accession number encompasses multiple genes, typically in the context of the present invention reference to such an accession number only refers to the carbapenemase gene within that accession number. Thus, SEQ ID NOs corresponding to the accession numbers herein typically comprise or consist of the relevant carbapenemase gene sequence.

An oligonucleotide specific for the one or more carbapenemase gene, or an oligonucleotide probe or primer of the invention may be complementary to the one or more carbapenemase gene of the invention, or a target region thereof. Typically the oligonucleotide specific for the one or more carbapenemase gene, or the oligonucleotide probe or primer of the invention is complementary over a length of contiguous nucleic acid residues. An oligonucleotide specific for the one or more carbapenemase gene, or an oligonucleotide probe or primer of the invention may, for example, be complementary to the one or more carbapenemase gene of the invention, or target region thereof, measured over at least 10, at least 11, at least 12, at least 13, at least 14, at least 15, at least 16, at least 14, at least 18, at least 19, at least 20, at least 21, at least 22, at least 23, at least 24, at least 25, at least 26, at least 27, at least 28, at least 29, at least 30, at least 35, at least 40, at least 50, at least 60, at least 70, at least 80, at least 90, or more nucleic acid residues, up to the oligonucleotide specific for the one or more carbapenemase gene, or the oligonucleotide probe or primer being complementary to the one or more carbapenemase gene of the invention, or target region thereof, over the entire length of the oligonucleotide specific for the one or more carbapenemase gene, or the oligonucleotide probe or primer.

An oligonucleotide specific for the one or more carbapenemase gene, or an oligonucleotide probe or primer of the invention may be complementary to the reverse sequence of the one or more carbapenemase gene of the invention, or a target region thereof. Typically the oligonucleotide specific for the one or more carbapenemase gene, or the oligonucleotide probe or primer of the invention is complementary over a length of contiguous nucleic acid residues of the reverse sequence. An oligonucleotide specific for the one or more carbapenemase gene, or an oligonucleotide probe or primer of the invention may, for example, be complementary to the reverse sequence of the one or more carbapenemase gene of the invention, or target region thereof, measured over at least 10, at least 11, at least 12, at least 13, at least 14, at least 15, at least 16, at least 14, at least 18, at least 19, at least 20, at least 21, at least 22, at least 23, at least 24, at least 25, at least 26, at least 27, at least 28, at least 29, at least 30, at least 35, at least 40, at least 50, at least 60, at least 70, at least 80, at least 90, or more nucleic acid residues, up to the oligonucleotide specific for the one or more carbapenemase gene, or the oligonucleotide probe or primer being complementary to the reverse sequence of the one or more carbapenemase gene of the invention, or target region thereof, over the entire length of the oligonucleotide specific for the one or more carbapenemase gene, or the oligonucleotide probe or primer.

An oligonucleotide specific for the one or more carbapenemase gene, or an oligonucleotide probe or primer of the invention may be complementary to a variant of the one or more carbapenemase gene of the invention, or a variant of a target region of said carbapenemase gene. Typically the oligonucleotide specific for the one or more carbapenemase gene, or the oligonucleotide probe or primer is complementary to a variant having at least 80% sequence identity to the one or more carbapenemase gene of the invention, or a variant having at least 80% sequence identity to the target region of said carbapenemase gene. The % sequence identity of the variant to the one or more carbapenemase gene of the invention, or a variant of a target region of said carbapenemase gene may be calculated over any appropriate length of sequence in the one or more carbapenemase gene, as described herein.

A sequence identity of at least 80% includes at least 82%, at least 84%, at least 86%, at least 88%, at least 90%, at least 91%, at least 92%, at least 93%, at least 94%, at least 95%, at least 96%, at least 97%, at least 98%, at least 99%, and 100% sequence identity (to each and every nucleic acid sequence presented herein and/or to each and every SEQ ID NO presented herein).

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22 (22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. MoI. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262 (5131) Science 208-214 (1993); Align-M, see, e.g., Ivo Van WaIIe et al., Align-M—A New Algorithm for Multiple Alignment of Highly Divergent Sequences, 20 (9) Bioinformatics: 1428-1435 (2004). Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992.

Variants of the specific sequences provided above may alternatively be defined by reciting the number of nucleotides that differ between the variant sequences and the specific reference sequences provided. These differences may result from the addition, deletion and/or substitution of one or more nucleotide position within the variant sequence compared with the reference sequence. Thus, in one embodiment, the sequence may comprise (or consist of) a nucleotide sequence that differs from the specific sequences provided at no more than ten nucleotide positions, no more than nine nucleotide positions, no more than eight nucleotide positions, no more than seven nucleotide positions, no more than six nucleotide positions, no more than five nucleotide positions, no more than four nucleotide positions, no more than three nucleotide positions, no more than two nucleotide positions or no more than one nucleotide position. Conservative substitutions are preferred. The term variants as defined herein also encompasses splice variants.

An oligonucleotide specific for the one or more carbapenemase gene, or an oligonucleotide primer of the invention may be at least ten, at least 11, at least 12, at least 13, at least 14, at least 15, at least 16, at least 17, at least 18, at least 20, at least 21, at least 22, at least 23, at least 24, at least 25, at least 26, at least 27, at least 28, at least 29, at least 30, at least 35, at least 40, at least 50 or more nucleotides in length. In a preferred embodiment, the oligonucleotide specific for the one or more carbapenemase gene, or the oligonucleotide primer is 10 to 40 nucleotides in length, more preferably 15 to 35 nucleotides in length. An oligonucleotide probe of the invention may be at least ten, at least 11, at least 12, at least 13, at least 14, at least 15, at least 16, at least 17, at least 18, at least 20, at least 21, at least 22, at least 23, at least 24, at least 25, at least 30, at least 40, at least 50 or more nucleotides in length at least 60, at least 70, at least 80, at least 90, at least 100, or more nucleotides in length. In a preferred embodiment, the oligonucleotide probe is 10 to 60 nucleotides in length, more preferably 10 to 50 nucleotides in length, even more preferably 10 to 40 nucleotides in length and even more preferably 12 to 40 nucleotides in length, and most preferably 15 to 35 nucleotides in length.

The oligonucleotides specific for the one or more carbapenemase gene, or the oligonucleotide probes and primers of the invention are typically designed to hybridise to their target nucleic acid sequence present in the one or more carbapenemase gene of the invention. In the context of the present invention, the term hybridises includes hybridising to the sense strand of a target sequence, the reverse of a target sequence, the complement of a target sequence or the reverse complement of a target sequence. Further, references herein to an oligonucleotide specific for the one or more carbapenemase gene, or the oligonucleotide probes and primers of the invention comprising a particular sequence also encompass oligonucleotides, probes and primers consisting of said sequences, as well as oligonucleotides comprising or consisting of the complement or reverse complement of said sequences. In a preferred embodiment, reverse primers and/or probes of the invention are reverse complement sequences and so are shown in this orientation. Also in the context of the present invention, the terms “hybridise” and “bind” may be used interchangeably.

Any reference herein to a target sequence (OXA-48-like or any other carbapenemase gene) defined in terms of the bases of a particular sequence applies equally to sub-ranges within the recited bases. Thus, definitions of an oligonucleotide specific to said target region may apply equally to oligonucleotides specific for a sub-range of bases within the recited target region. As a non-limiting example, a reference herein to a target region of an OXA gene comprising bases 650 to 717 of said gene applies equally to a target region of said OXA gene comprising bases 660 to 717 or 670 to 717 or 680 to 717 or 690 to 717, or any other sub-range within the 650 to 717 target region. An oligonucleotide specific for a target region of an OXA gene comprising bases 650 to 717 of said gene applies equally to a an oligonucleotide specific for a target region of said OXA gene comprising bases 660 to 717 or 670 to 717 or 680 to 717 or 690 to 717, or any other sub-range within the 650 to 717 target region. An OXA-48-like specific oligonucleotide, probe or primer of the invention may hybridise or bind to a nucleic acid sequence within a target region of an OXA gene comprising bases 421 to 780 of any of the OXA accession numbers or SEQ ID NOs disclosed herein, or the complement or reverse complement thereof (or the sequence of any other OXA gene, the accession numbers and SEQ ID NOs disclosed herein are not intended as an exhaustive list of OXA sequences). In a preferred example, the target region of the OXA gene comprises bases 421 to 717, more preferably bases 427 to 717 or 427 to 704 of any of the OXA accession numbers or SEQ ID NOs disclosed herein or the complement or reverse complement thereof. An OXA-48-like oligonucleotide or probe of the invention preferably hybridises to a target region of the OXA gene comprising bases 570 to 670, preferably bases 570 to 610 of any of the OXA accession numbers or SEQ ID NOs disclosed herein or the complement or reverse complement thereof. An OXA-48-like oligonucleotide or forward primer of the invention preferably hybridises to a target region of the OXA gene comprising bases 421 to 490, more preferably 421 to 470 any of the OXA accession numbers or SEQ ID NOs disclosed herein or the complement or reverse complement thereof. An OXA-48-like oligonucleotide or reverse primer of the invention preferably hybridises to a target region of the OXA gene comprising bases 650 to 717, preferably 660 to 717, more preferably 670 to 717, even more preferably 680 to 717, most preferably 690 to 717 of any of the OXA accession numbers or SEQ ID NOs disclosed herein or the complement or reverse complement thereof.

In a preferred embodiment, an OXA-48-like gene having at least 85%, at least 90%, at least 95%, at least 96%, at least 97%, at least 98%, at least 99% or more sequence identity with one or more of the following OXA carbapenemases (at the amino acid or nucleic acid level: OXA-181 (SEQ ID NO: 31, Accession No. GI 304368222, HM992946.1), OXA-232 (SEQ ID NO: 32, GI 444236140, JX423831.1, 2677-3474,), OXA-247 (SEQ ID NO: 33, GI 442769982, JX893517.1, 1-786), OXA-163 (SEQ ID NO: 34, GI 323817046, HQ700343.1, 1-786), OXA-204 (SEQ ID NO: 37, GI 408795221, JQ809466.1, 5375-6172), OXA-370 (SEQ ID NO: 39, GI 573006828, KF900153.1, 1-798), OXA-245 (SEQ ID NO: 40, GI 442577759, JX438001.1, 1-798), OXA-162 (SEQ ID NO: 41, GI 270312218, GU197550.1, 1-798), OXA-48 (SEQ ID NO: 43, GI AY236073.2, 2188-2985) and/or OXA-244 (SEQ ID NO: 52, GI 442577757, JX438000.1, 1-798), or the OXA-48-like gene of one of these accession numbers is used as a reference sequence to determine the positions of the target regions of the OXA-48-like gene. For example, the target sequences may be those from one or more of these accession numbers, or regions corresponding to these target regions from variants of these accession numbers or from another OXA gene (e.g. from another OXA accession number disclosed herein).

In another embodiment, the OXA gene of accession number JN714122 (SEQ ID NO: 46), JQ99150 (SEQ ID NO: 29) and/or JQ809466 (SEQ ID NO: 37) is used as a reference OXA gene sequence to determine the positions of the target regions of the OXA-48 gene. For example, the target sequences may be those from one or more of accession numbers JN714122 (SEQ ID NO: 46), JQ99150 (SEQ ID NO: 29) and/or JQ809466 (SEQ ID NO: 37), or regions corresponding to these target regions from variants of these accession numbers or from another OXA gene (e.g. from another OXA accession number disclosed herein).

According to the present invention, one or more additional oligonucleotide specific for the OXA-48-like gene may be used. Said one or more additional oligonucleotide specific for the OXA-48-like gene may hybridise to a target region of the OXA-48-like gene comprising bases 421 to 490 of the OXA-48-like gene; and/or an additional oligonucleotide specific for the OXA-48-like gene may hybridises to a target region of the OXA-48-like gene comprising bases 650 to 717 of the OXA-48-like gene; and/or a first additional oligonucleotide specific for the OXA-48-like gene may hybridise to a target region of the OXA-48-like gene comprising bases 421 to 490 of the OXA-48-like gene, and a second additional oligonucleotide specific for the OXA-48-like gene may hybridise to a target region of the OXA-48-like gene comprising bases 650 to 717 of the OXA-48-like gene. Typically the additional oligonucleotide specific for the OXA-48-like gene which hybridises to a target region of the OXA-48-like gene comprising bases 421 to 490 of the OXA-48-like gene is a forward primer and/or the additional oligonucleotide specific for the OXA-48-like gene which hybridises to a target region of the OXA-48-like gene comprising bases 650 to 717 of the OXA-48-like gene a reverse primer.

A VIM specific oligonucleotide, probe or primer of the invention may hybridise or bind to a nucleic acid sequence within a target region of a VIM gene comprising bases 235 to 410, 245 to 420, 238 to 413, preferably bases 235 to 400, 238 to 403 or 245 to 410, more preferably bases 255 to 400, 258 to 403 or 270 to 410 of any of the VIM accession numbers or SEQ ID NOs disclosed herein or the complement or reverse complement thereof (or the sequence of any other VIM gene, the accession numbers and SEQ ID NOs disclosed herein are not intended as an exhaustive list of VIM sequences).

A VIM oligonucleotide or forward primer of the invention preferably hybridises to a target region of the VIM gene comprising bases 235 to 280, 238 to 283 or 245 to 290, more preferably 235 to 270, 238 to 273 or 245 to 280, most preferably 235 to 260, 238 to 263 or 245 to 270 of any of the VIM accession numbers or SEQ ID NOs disclosed herein or the complement or reverse complement thereof. A VIM oligonucleotide or reverse primer of the invention preferably hybridises to a target region of the VIM gene comprising bases 360 to 410, 363 to 413 or 370 to 420, preferably 370 to 410, 373 to 413 or 380 to 420, more preferably 380 to 400, 383 to 403 or 390 to 410 of any of the VIM accession numbers or SEQ ID NOs disclosed herein or the complement or reverse complement thereof. A VIM oligonucleotide or probe of the invention preferably hybridises to a target region of the VIM gene comprising bases 270 to 330, 273 to 330 or 280 to 340, preferably 280 to 320, 283 to 323 or 290 to 330, or more preferably 280 to 310, 283 to 313 or 290 to 320 of any of the VIM accession numbers or SEQ ID NOs disclosed herein or the complement or reverse complement thereof. In a preferred embodiment, the VIM gene of accession number Y18050 (SEQ ID NO: 98), AF191564 (SEQ ID NO: 75) and/or AJ536835 (SEQ ID NO: 53) is used as a reference VIM gene sequence to determine the positions of the target regions of the VIM gene. For example, the target sequences may be those from one or more of accession numbers Y18050 (SEQ ID NO: 98), AF191564 (SEQ ID NO: 75) and/or AJ536835 (SEQ ID NO: 53), or regions corresponding to these target regions from variants of these accession numbers or from another VIM gene (e.g. from another VIM accession number disclosed herein).

According to the present invention, one or more additional oligonucleotide specific for the VIM gene may be used. Said one or more additional oligonucleotide specific for the VIM gene may hybridise to a target region of the VIM gene comprising bases 235 to 400, 238 to 403 and/or 245 to 410 of the VIM gene, preferably bases 235 to 280, 238 to 283 or 240 to 290; and/or 360 to 410, 363 to 413 or 270 to 420 of the VIM gene. Said one or more additional oligonucleotide specific for the VIM gene may hybridise to a target region of the VIM gene comprising bases 235 to 270 or 275 to 330 of the VIM gene. In a preferred embodiment, a first additional oligonucleotide specific for the VIM gene hybridises to a target region of the VIM gene comprising bases 235 to 400, 238 to 403 and/or 245 to 410 of the VIM gene, preferably to a target region of the VIM gene comprising bases 235 to 270 of the VIM gene, and a second additional oligonucleotide specific for the VIM gene hybridises to a target region of the VIM gene comprising bases 235 to 400, 238 to 403 and/or 245 to 410 of the VIM gene, preferably to a target region of the VIM gene comprising bases 275 to 330 of the VIM gene. Typically the additional oligonucleotide specific for the VIM gene which hybridises to a target region of the VIM gene comprising bases 235 to 400, 238 to 403 and/or 245 to 410 of the VIM gene, preferably to a target region of the VIM gene comprising bases 235 to 270 of the VIM gene, is a forward primer; and/or the additional oligonucleotide specific for the VIM gene which hybridises to a target region of the VIM gene comprising bases 235 to 400, 238 to 403 and/or 245 to 410 of the VIM gene, preferably to a target region of the VIM gene comprising bases 275 to 330 of the VIM gene, is a probe.

An NDM specific oligonucleotide, probe or primer of the invention may hybridise or bind to a nucleic acid sequence within a target region of an NDM gene comprising bases 108 to 320 of any of the NDM accession numbers or SEQ ID NOs disclosed herein or the complement or reverse complement thereof (or the sequence of any other NDM gene, the accession numbers and SEQ ID NOs disclosed herein are not intended as an exhaustive list of NDM sequences). In a preferred example, the target region of the NDM gene comprises bases 108 to 314 or 110 to 314 of any of the NDM accession numbers or SEQ ID NOs disclosed herein or the complement or reverse complement thereof. An NDM oligonucleotide or forward primer of the invention preferably hybridises to a target region of the NDM gene comprising bases 108 to 150, more preferably 108 to 140, even more preferably 108 to 130 any of the NDM accession numbers or SEQ ID NOs disclosed herein or the complement or reverse complement thereof. An NDM oligonucleotide or reverse primer of the invention preferably hybridises to a target region of the NDM gene comprising bases 294 to 320, preferably 294 to 314 of any of the NDM accession numbers or SEQ ID NOs disclosed herein or the complement or reverse complement thereof. An NDM oligonucleotide or probe of the invention preferably hybridises to a target region of the NDM gene comprising bases 150 to 210, preferably 160 to 200, more preferably 160 to 190 of any of the NDM accession numbers of SEQ ID NOs as disclosed herein or the complement or reverse complement thereof. In a preferred embodiment, the NDM gene of accession number FN396876 (SEQ ID NO: 131) is used as a reference NDM gene sequence to determine the positions of the target regions of the NDM gene. For example, the target sequences may be those from accession number FN396876 (SEQ ID NO: 131), or regions corresponding to these target regions from variants of this accession number or from another NDM gene (e.g. from another NDM accession number disclosed herein).

A KPC specific oligonucleotide, probe or primer of the invention may hybridise or bind to a nucleic acid sequence within a target region of an KPC gene comprising bases 580 to 800 or 600 to 810 of any of the KPC accession numbers or SEQ ID NOs disclosed herein or the complement or reverse complement thereof (or the sequence of any other KPC gene, the accession numbers and SEQ ID NOs disclosed herein are not intended as an exhaustive list of KPC sequences). In a preferred example, the target region of the KPC gene comprises bases 580 to 780 or 600 to 790, more preferably bases 585 to 770 or 600 to 785 of any of the KPC accession numbers or SEQ ID NOs disclosed herein or the complement or reverse complement thereof. A KPC oligonucleotide or forward primer of the invention preferably hybridises to a target region of the KPC gene comprising bases 580 to 620 or 600 to 650, more preferably 580 to 610 or 600 to 630 any of the KPC accession numbers or SEQ ID NOs disclosed herein or the complement or reverse complement thereof. A KPC oligonucleotide or reverse primer of the invention preferably hybridises to a target region of the KPC gene comprising bases 745 to 800 or 760 to 815 of any of the NDM accession numbers or SEQ ID NOs disclosed herein. A KPC oligonucleotide or probe of the invention preferably hybridises to a target region of the KPC gene comprising bases 660 to 710 or 675 to 725, preferably 670 to 700 or 685 to 715 of any of the KPC accession numbers or SEQ IDs disclosed herein or the complement or reverse complement thereof. In a preferred embodiment, the KPC gene of accession number AF297554 (SEQ ID NO: 108) is used as a reference KPC gene sequence to determine the positions of the target regions of the KPC gene. For example, the target sequences may be those from accession number AF297554 (SEQ ID NO: 108), or regions corresponding to these target regions from variants of this accession number or from another KPC gene (e.g. from another KPC accession number disclosed herein).

An IMP specific oligonucleotide, probe or primer of the invention may hybridise or bind to a nucleic acid sequence within a target region of an IMP gene comprising bases 337 to 515 of any of the IMP accession numbers or SEQ ID NOs disclosed herein or the complement or reverse complement thereof (or the sequence of any other IMP gene, the accession numbers and SEQ ID NOs disclosed herein are not intended as an exhaustive list of IMP sequences). In a preferred example, the target region of the IMP gene comprises bases 337 to 363, 440 to 514 and/or 471 to 515 of any of the IMP accession numbers or SEQ ID NOs disclosed herein or the complement or reverse complement thereof. An IMP probe of the invention preferably hybridises to a target region of the IMP gene comprising bases 440 to 514 any of the IMP accession numbers or SEQ ID NOs disclosed herein or the complement or reverse complement thereof. An IMP forward primer of the invention preferably hybridises to a target region of the IMP gene comprising bases 337 to 363 of any of the IMP accession numbers or SEQ ID NOs disclosed herein or the complement or reverse complement thereof. An IMP reverse primer of the invention preferably hybridises to a target region of the IMP gene comprising bases 471 to 515 of any of the IMP accession numbers or SEQ ID NOs disclosed herein or the complement or reverse complement thereof. In a preferred embodiment, the IMP gene of accession number S71932 (SEQ ID NO: 192) is used as a reference IMP gene sequence to determine the positions of the target regions of the IMP gene. For example, the target sequences may be those from accession number S71932 (SEQ ID NO: 192), or regions corresponding to these target regions from variants of this accession number or from another IMP gene (e.g. from another IMP accession number disclosed herein).

In one embodiment, the oligonucleotide specific for the one or more carbapenemase gene, or an oligonucleotide primer or probe of the invention is designed against a consensus target sequence from different variants of the one or more carbapenemase gene. Thus, the oligonucleotide, primer and/or probe may comprise or consist of a sequence corresponding to a consensus target sequence, or a sequence having a defined sequence identity with said consensus target sequence. Alternatively, the oligonucleotide, primer and/or probe may be complementary or reverse complementary to a consensus target sequence, or a sequence having a defined sequence identity with said consensus target sequence. Thus, the oligonucleotide, primer and/or probe may hybridise or bind to the consensus target sequence or the complement or reverse complement thereof, or the oligonucleotide, primer and/or probe may hybridise or bind to a sequence having a defined sequence identity with said consensus target sequence, or complement or reverse complement thereof.

For example, an oligonucleotide specific for an OXA-48-like carbapenemase gene, or an oligonucleotide primer or probe for an OXA-48-like carbapenemase gene may be designed against a consensus target sequence, wherein the consensus is generated across the corresponding target sequence of each member of the OXA-48-like family. Thus, an oligonucleotide specific for an OXA-48-like carbapenemase gene, or an oligonucleotide primer or probe for an OXA-48-like carbapenemase gene may bind to or hybridise not only the consensus target sequence or the complement or reverse complement thereof, but also to sequences having a defined % sequence identity with said consensus target sequence or complement or reverse complement thereof. Typically an oligonucleotide specific for an OXA-48-like carbapenemase gene, or an oligonucleotide primer or probe for an OXA-48-like carbapenemase gene will bind to or hybridise target sequences corresponding to the consensus sequence (or the complement or reverse complement thereof) in all members of the OXA-48-like carbapenemase family. This applies equally to the other carbapenemase families of the invention, including VIM, KPC, NDM, IMP, and/or other carbapenemase genes.

In more detail, an oligonucleotide specific for an OXA-48-like carbapenemase gene, or an oligonucleotide primer or probe for an OXA-48-like carbapenemase gene may bind to a target consensus sequence comprising or consisting of the nucleic acid sequence GATTATGGYAATGAGGAYATYTCGGGC (SEQ ID NO: 1), CATATCCATATTCATCGCAAAAAACCACAC (SEQ ID NO: 2), or CCATTGGCTTCGGTCAGCATGGCTTGTTT (SEQ ID NO: 3), or a consensus sequence that comprises or consists of the complement or reverse complement of any one of these sequences. Possible positions of mismatches are shown underlined. Thus, the oligonucleotide specific for an OXA-48-like carbapenemase gene, or the oligonucleotide primer or probe for an OXA-48-like carbapenemase gene may comprise or consist of any one of these nucleic acid sequences, or the complement or reverse complement thereof. An oligonucleotide specific for an OXA-48-like carbapenemase gene, or an oligonucleotide primer or probe for an OXA-48-like carbapenemase gene may comprise a maximum of three, a maximum of two, a maximum of one or no mismatches compared with any one of the above-identified consensus sequences, or the complement or reverse complement thereof. Thus, the oligonucleotide specific for an OXA-48-like carbapenemase gene, or the oligonucleotide primer or probe for an OXA-48-like carbapenemase gene may have at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more sequence identity with any one of the above-identified consensus sequences or the complement or reverse complement thereof. In a preferred embodiment the oligonucleotide primer or probe for an OXA-48-like carbapenemase gene has at least 88%, at least 89%, at least 90%, at least 91%, at least 92%, at least 93%, at least 94%, at least 95%, at least 96%, at least 97%, at least 98%, at least 99% or more sequence identity with any one of the above-identified consensus sequences or the complement or reverse complement thereof.

Based on the nucleic acid sequences of all the OXA-48-like carbapenemase genes, an oligonucleotide specific for an OXA-48-like carbapenemase gene, or the oligonucleotide primer or probe for an OXA-48-like carbapenemase gene may have a maximum of two, a maximum of one or no mismatches with any one target sequence from the OXA-48-like gene family which corresponds to the consensus sequences identified above, to the complement or reverse complement of said target sequence. Thus, typically the oligonucleotide specific for an OXA-48-like carbapenemase gene, or the oligonucleotide primer or probe for an OXA-48-like carbapenemase gene has at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more sequence identity with any one target sequence from the OXA-48-like gene family which corresponds to the consensus sequences identified above, to the complement or reverse complement of said target sequence. In a preferred embodiment the oligonucleotide primer or probe for an OXA-48-like carbapenemase gene has at least 88%, at least 89%, at least 90%, at least 91%, at least 92%, at least 93%, at least 94%, at least 95%, at least 96%, at least 97%, at least 98%, at least 99% or more sequence identity with any one target sequence from the OXA-48-like gene family which corresponds to the consensus sequences identified above, to the complement or reverse complement of said target sequence. See Table 1 below.

Typically, wherein the present invention relates to determining the presence and/or amount of an OXA-48-like gene, the at least one oligonucleotide specific for the OXA-48-like gene comprises a region of at least 24, at least 25, at least 26, at least 27, at least 28 or the full-length of 29 contiguous bases from SEQ ID NO: 3, or from a nucleic acid sequence having at least 80% sequence identity to the full-length of the nucleic acid sequence of SEQ ID NO: 3 (as defined herein) or a nucleic acid which is complementary to SEQ ID NO: 3 or a nucleic acid having at least 80% sequence identity to the full-length of the nucleic acid sequence of SEQ ID NO: 3, and hybridises to a target region of the OXA-48-like gene comprising bases 570 to 670, preferably bases 570 to 610, of the OXA-48-like gene. Typically, this oligonucleotide specific for the OXA-48-like gene is a probe.

The oligonucleotide specific for the OXA-48-like gene may be used in combination with any of the other oligonucleotides disclosed herein, including other oligonucleotides specific for the OXA-48-like gene, and/or oligonucleotides specific for one or more other carbapenemase genes, including but not limited to a VIM gene, a KPC gene, an NDM gene, an IMP gene, an IMI gene, a GES gene and/or an SPM gene.

One or more additional oligonucleotide specific for the OXA-48-like gene may be used to determine the presence and/or amount of the OXA-48-like gene in addition to the specific oligonucleotide defined above. Said one or more additional oligonucleotide specific for the OXA-48-like gene may comprise any other oligonucleotide specific for an OXA-48-like gene as described herein.

Typically, said one more additional oligonucleotide specific for the OXA-48-like gene comprises a nucleic acid sequence of SEQ ID NO: 2 and/or 16, or a nucleic acid sequence having at least 80% sequence identity (as defined herein) to the full-length of the nucleic acid sequence of SEQ ID NO: 2 and/or 16, or a nucleic acid sequence which is complementary to any of said sequences. Any combination of these three oligonucleotides may be used in accordance with the present invention. The oligonucleotide comprising the nucleic acid sequence of SEQ ID NO: 2 or a nucleic acid sequence having at least 80% sequence identity (as defined herein) to the full-length of the nucleic acid sequence of SEQ ID NO: 2, or a nucleic acid sequence which is complementary to either of said sequences may be used a reverse primer; and/or the nucleic acid sequence of SEQ ID NO: 16, or a nucleic acid sequence having at least 80% sequence identity (as defined herein), or a nucleic acid sequence which is complementary to either of said sequences to the full-length of the nucleic acid sequence of SEQ ID NO: 16 may be used a forward primer.

In a preferred embodiment all three of (i) an oligonucleotide specific for the OXA-48-like gene comprises a region of at least 24, at least 25, at least 26, at least 27, at least 28 or the full-length of 29 contiguous bases from SEQ ID NO: 3, or from a nucleic acid sequence having at least 80% sequence identity to the full-length of the nucleic acid sequence of SEQ ID NO: 3 (as defined herein) and hybridises to a target region of the OXA-48-like gene comprising bases 570 to 670, preferably bases 570 to 610, of the OXA-48-like gene; (ii) an oligonucleotide comprising the nucleic acid sequence of SEQ ID NO: 2, or a nucleic acid sequence complementary to the nucleic acid sequence of SEQ ID NO: 2, or a nucleic acid sequence having at least 80% sequence identity (as defined herein) to the full-length of the nucleic acid sequence of SEQ ID NO: 2, or a nucleic acid sequence complementary to the nucleic acid sequence of SEQ ID NO:2; and (iii) a nucleic acid sequence of SEQ ID NO: 16, or a nucleic acid sequence complementary to the nucleic acid sequence of SEQ ID NO: 16, or a nucleic acid sequence having at least 80% sequence identity (as defined herein) to the full-length of the nucleic acid sequence of SEQ ID NO: 16, or a nucleic acid sequence complementary to the nucleic acid sequence of SEQ ID NO: 16 are used according to the present invention.

The additional oligonucleotide specific for the OXA-48-like gene which hybridises to a target region of the OXA-48-like gene comprising bases 421 to 490 of the OXA-48-like gene may comprise or consist of a nucleic acid sequence of SEQ ID NO: 16, or a nucleic acid sequence complementary to the nucleic acid sequence of SEQ ID NO: 16, or a nucleic acid sequence having at least 80% sequence identity to the full-length of the nucleic acid sequence of SEQ ID NO: 16, or a nucleic acid sequence complementary to the nucleic acid sequence of SEQ ID NO: 16; and/or the additional oligonucleotide specific for the OXA-48-like gene which hybridises to a target region of the OXA-48-like gene comprising bases 650 to 717 of the OXA-48-like gene may comprise or consist of a nucleic acid sequence of SEQ ID NO: 2, or a nucleic acid sequence complementary to the nucleic acid sequence of SEQ ID NO: 2, or a nucleic acid sequence having at least 80% sequence identity to the full-length of the nucleic acid sequence of SEQ ID NO: 2, or a nucleic acid sequence complementary to the nucleic acid sequence of SEQ ID NO: 2.

The one or more additional oligonucleotide specific for the OXA-48-like gene according to the present invention may comprise any other oligonucleotide specific for an OXA-48-like gene as described herein, and may be used in any combination with any of the oligonucleotides specific for any other carbapenemase gene (examples of other carbapenemase genes and oligonucleotides specific for said other carbapenemase genes are described herein), particularly the oligonucleotides specific for the VIM and/or NDM and/or KPC carbapenemase genes as described herein.

An oligonucleotide specific for a VIM carbapenemase gene, or an oligonucleotide primer or probe for a VIM carbapenemase gene may bind to a target consensus sequence comprising or consisting of the nucleic acid sequence TTGCTTYTKATTGATACAGCKTGGGG (SEQ ID NO: 4), GTACGTYGCCACYCCAGCC (SEQ ID NO: 5), or TCGCGGAGATTGARAAGCAAATTGGACTTCC (SEQ ID NO: 6), or a consensus sequence that comprises or consists of the complement or reverse complement of any one of these sequences. Possible positions of mismatches are shown underlined. Thus, the oligonucleotide specific for a VIM carbapenemase gene, or the oligonucleotide primer or probe for a VIM carbapenemase gene may comprise or consist of any one of these nucleic acid sequences, or the complement or reverse complement thereof. An oligonucleotide specific for a VIM carbapenemase gene, or an oligonucleotide primer or probe for a VIM carbapenemase gene may comprise a maximum of three, a maximum of two, a maximum of one or no mismatches compared with any one of the above-identified consensus sequences, or the complement or reverse complement thereof. Thus, the oligonucleotide specific for a VIM carbapenemase gene, or the oligonucleotide primer or probe for a VIM carbapenemase gene may have at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more sequence identity with any one of the above-identified consensus sequences or the complement or reverse complement thereof. In a preferred embodiment the oligonucleotide primer or probe for a VIM carbapenemase gene has at least 88%, at least 89%, at least 90%, at least 91%, at least 92%, at least 93%, at least 94%, at least 95%, at least 96% or at least 97% sequence identity with any one of the above-identified consensus sequences or the complement or reverse complement thereof.

Based on the nucleic acid sequences of all the VIM carbapenemase genes, an oligonucleotide specific for a VIM carbapenemase gene, or the oligonucleotide primer or probe for a VIM carbapenemase gene may have a maximum of two, a maximum of one or no mismatches with any one target sequence from the VIM gene family which corresponds to the consensus sequences identified above, to the complement or reverse complement of said target sequence. Thus, typically the oligonucleotide specific for a VIM carbapenemase gene, or the oligonucleotide primer or probe for a VIM carbapenemase gene has at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more sequence identity with any one target sequence from the VIM gene family which corresponds to the consensus sequences identified above, to the complement or reverse complement of said target sequence. In a preferred embodiment the oligonucleotide primer or probe for a VIM carbapenemase gene has at least 88%, at least 89%, at least 90%, at least 91%, at least 92%, at least 93%, at least 94%, at least 95%, at least 96% or at least 97% sequence identity with any one target sequence from the VIM gene family which corresponds to the consensus sequences identified above, to the complement or reverse complement of said target sequence. See Table 1 below.

Typically, wherein the present invention relates to determining the presence and/or amount of a VIM gene, the at least one oligonucleotide specific for the VIM gene is 10 to 60 nucleotides in length and hybridises to a nucleic acid sequence within a target region of the VIM gene comprising bases 235 to 400, 238 to 403 and/or 245 to 410, preferably bases 350 to 410, of the VIM gene. In a preferred embodiment, said oligonucleotide is preferably 10 to 50 nucleotides in length, more preferably 10 to 40 nucleotides in length and even more preferably 12 to 40 nucleotides in length, and most preferably 15 to 35 nucleotides in length. Said least one oligonucleotide sequence specific for the VIM gene typically comprises a region of at least 15 contiguous bases from SEQ ID NO: 18, or from a nucleic acid sequence having at least 80% sequence identity to the full-length of the nucleic acid sequence of SEQ ID NO: 18, or from a nucleic acid which is complementary to SEQ ID NO: 18 or a nucleic acid having at least 80% sequence identity to the full-length of the nucleic acid sequence of SEQ ID NO: 18. Typically, this oligonucleotide specific for the VIM gene is a reverse primer.

The oligonucleotide specific for the VIM gene may be used in combination with any of the other oligonucleotides disclosed herein, including other oligonucleotides specific for the VIM gene, and/or oligonucleotides specific for one or more other carbapenemase genes, including but not limited to an OXA-48-like gene, a KPC gene, an NDM gene, an IMP gene, an IMI gene, a GES gene and/or an SPM gene.

One or more additional oligonucleotide specific for the VIM gene may be used to determine the presence and/or amount of the VIM gene in addition to the specific oligonucleotide defined above. Said one or more additional oligonucleotide specific for the VIM gene may comprise any other oligonucleotide specific for a VIM gene as described herein.

Typically, said one more additional oligonucleotide specific for the VIM gene comprises a nucleic acid sequence of SEQ ID NO: 19 and/or 17, or a nucleic acid sequence having at least 80% sequence identity (as defined herein) to the full-length of the nucleic acid sequence of SEQ ID NO: 19 and/or 17, or a nucleic acid sequence which is complementary to any of said sequences. Any combination of these oligonucleotides may be used in accordance with the present invention. The oligonucleotide comprising the nucleic acid sequence of SEQ ID NO: 19 or a nucleic acid sequence having at least 80% sequence identity (as defined herein) to the full-length of the nucleic acid sequence of SEQ ID NO: 19, or a nucleic acid sequence which is complementary to either of said sequences may be used a probe; and/or the nucleic acid sequence of SEQ ID NO: 17, or a nucleic acid sequence having at least 80% sequence identity (as defined herein), or a nucleic acid sequence which is complementary to either of said sequences to the full-length of the nucleic acid sequence of SEQ ID NO: 17 may be used a forward primer.

In a preferred embodiment all three of (i) an oligonucleotide specific for the VIM gene is 10 to 60 nucleotides in length, preferably 10 to 50 nucleotides in length, more preferably 10 to 40 nucleotides in length and even more preferably 12 to 40 nucleotides in length, and most preferably 15 to 35 nucleotides in length and hybridises to a nucleic acid sequence within a target region of the VIM gene comprising bases 235 to 400, 238 to 403 and/or 245 to 410, preferably bases 350 to 410, of the VIM gene, wherein said oligonucleotide sequence specific for the VIM gene typically comprises a region of at least 15 contiguous bases from SEQ ID NO: 18, or from a nucleic acid sequence having at least 80% sequence identity to the full-length of the nucleic acid sequence of SEQ ID NO: 18, or from a nucleic acid which is complementary to SEQ ID NO: 18 or a nucleic acid having at least 80% sequence identity to the full-length of the nucleic acid sequence of SEQ ID NO: 18; (ii) an oligonucleotide comprising the nucleic acid sequence of SEQ ID NO: 19, or a nucleic acid sequence complementary to the nucleic acid sequence of SEQ ID NO: 19, or a nucleic acid sequence having at least 80% sequence identity (as defined herein) to the full-length of the nucleic acid sequence of SEQ ID NO: 19, or a nucleic acid sequence complementary to the nucleic acid sequence of SEQ ID NO:19; and (iii) a nucleic acid sequence of SEQ ID NO: 17, or a nucleic acid sequence complementary to the nucleic acid sequence of SEQ ID NO: 17, or a nucleic acid sequence having at least 80% sequence identity (as defined herein) to the full-length of the nucleic acid sequence of SEQ ID NO: 17, or a nucleic acid sequence complementary to the nucleic acid sequence of SEQ ID NO: 17 are used according to the present invention.

The additional oligonucleotide specific for the VIM gene which hybridises to a target region of the target region of the VIM gene comprising bases 235 to 400, 238 to 403 and/or 245 to 410 of the VIM gene, preferably to a target region of the VIM gene comprising bases 235 to 270 of the VIM gene, may comprise or consist of a nucleic acid sequence of SEQ ID NO: 17, or a nucleic acid sequence complementary to the nucleic acid sequence of SEQ ID NO: 17, or a nucleic acid sequence having at least 80% sequence identity to the full-length of the nucleic acid sequence of SEQ ID NO: 17, or a nucleic acid sequence complementary to the nucleic acid sequence of SEQ ID NO: 17, and/or the additional oligonucleotide specific for the VIM gene which hybridises to a target region of the VIM gene comprising bases 235 to 400, 238 to 403 and/or 245 to 410 of the VIM gene, preferably to a target region of the VIM gene comprising bases 275 to 330 of the VIM gene, may comprise or consist of a nucleic acid sequence of SEQ ID NO: 19, or a nucleic acid sequence complementary to the nucleic acid sequence of SEQ ID NO: 19, or a nucleic acid sequence having at least 80% sequence identity to the full-length of the nucleic acid sequence of SEQ ID NO: 19, or a nucleic acid sequence complementary to the nucleic acid sequence of SEQ ID NO: 19.

The one or more additional oligonucleotide specific for the VIM gene according to the present invention may comprise any other oligonucleotide specific for an VIM gene as described herein, and may be used in any combination with any of the oligonucleotides specific for any other carbapenemase gene (examples of other carbapenemase genes and oligonucleotides specific for said other carbapenemase genes are described herein), particularly the oligonucleotides specific for the OXA-48-like and/or NDM and/or KPC carbapenemase genes as described herein.

An oligonucleotide specific for an NDM carbapenemase gene, or an oligonucleotide primer or probe for an NDM carbapenemase gene may bind to a target consensus sequence comprising or consisting of the nucleic acid sequence CCAGCAAATGGAAACTGGCGAC (SEQ ID NO: 7), ATCCAGTTGAGGATCTGGGCG (SEQ ID NO: 8), or ACCGAATGTCTGGCAGCACACTTC (SEQ ID NO: 9), or a consensus sequence that comprises or consists of the complement or reverse complement of any one of these sequences. Thus, the oligonucleotide specific for an NDM carbapenemase gene, or the oligonucleotide primer or probe for an NDM carbapenemase gene may comprise or consist of any one of these nucleic acid sequences, or the complement or reverse complement thereof. An oligonucleotide specific for an NDM carbapenemase gene, or an oligonucleotide primer or probe for an NDM carbapenemase gene may comprise a maximum of three, a maximum of two, a maximum of one or no mismatches compared with any one of the above-identified consensus sequences, or the complement or reverse complement thereof. Typically said oligonucleotide, primer or probe has no mismatches compared with any one of the above-identified consensus sequences, or the complement or reverse complement thereof. Thus, the oligonucleotide specific for an NDM carbapenemase gene, or the oligonucleotide primer or probe for an NDM carbapenemase gene may have at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more sequence identity with any one of the above-identified consensus sequences or the complement or reverse complement thereof. In a preferred embodiment the oligonucleotide primer or probe for an NDM carbapenemase gene has at least 90%, at least 91%, at least 92%, at least 93%, at least 94%, at least 95%, at least 96%, at least 97%, at least 98%, at least 99% or more, even more preferably 100% sequence identity with any one of the above-identified consensus sequences or the complement or reverse complement thereof.

Based on the nucleic acid sequences of all the NDM carbapenemase genes, an oligonucleotide specific for an NDM carbapenemase gene, or an oligonucleotide primer or probe for an NDM carbapenemase gene may have a maximum of two, a maximum of one or no mismatches with any one target sequence from the NDM gene family which corresponds to the consensus sequences identified above, or to the complement or reverse complement of said target sequence. In a preferred embodiment, said oligonucleotide, primer or probe has no mismatches compared with any one target sequence from the NDM gene family which corresponds to the consensus sequences identified above, or to the complement or reverse complement of said target sequence. Thus, typically the oligonucleotide specific for an NDM carbapenemase gene, or the oligonucleotide primer or probe for an NDM carbapenemase gene has at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more sequence identity with any one target sequence from the NDM gene family which corresponds to the consensus sequences identified above, or to the complement or reverse complement of said target sequence. In a preferred embodiment the oligonucleotide primer or probe for an NDM carbapenemase gene has at least 88%, at least 89%, at least 90%, at least 91%, at least 92%, at least 93%, at least 94%, at least 95%, at least 96% or at least 97%, or even more preferably 100% sequence identity with any one target sequence from the NDM gene family which corresponds to the consensus sequences identified above, or to the complement or reverse complement of said target sequence. See Table 1 below.

An oligonucleotide specific for a KPC carbapenemase gene, or an oligonucleotide primer or probe for a KPC carbapenemase gene may bind to a target consensus sequence comprising or consisting of the nucleic acid sequence GCAGCGGCAGCAGYTTGTTGATT (SEQ ID NO: 10), GTAGACGGCCAACACAATAGGTGC (SEQ ID NO: 11), or CAGTCGGAGACAAAACCGGAACCTGC (SEQ ID NO: 12), or a consensus sequence that comprises or consists of the complement or reverse complement of any one of these sequences. Possible positions of mismatches are shown underlined. Thus, the oligonucleotide specific for a KPC carbapenemase gene, or the oligonucleotide primer or probe for a KPC carbapenemase gene may comprise or consist of any one of these nucleic acid sequences, or the complement or reverse complement thereof. An oligonucleotide specific for a KPC carbapenemase gene, or an oligonucleotide primer or probe for a KPC carbapenemase gene may comprise a maximum of three, a maximum of two, a maximum of one or no mismatches compared with any one of the above-identified consensus sequences, or the complement or reverse complement thereof. Typically said oligonucleotide, primer or probe has a maximum of one or no mismatches compared with any one of the above-identified consensus sequences, or the complement or reverse complement thereof. Thus, the oligonucleotide specific for a KPC carbapenemase gene, or the oligonucleotide primer or probe for a KPC carbapenemase gene may have at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more sequence identity with any one of the above-identified consensus sequences or the complement or reverse complement thereof. In a preferred embodiment the oligonucleotide primer or probe for a KPC carbapenemase gene has at least 95%, at least 96%, at least 97%, at least 98%, at least 99% or more, even more preferably 100% sequence identity with any one of the above-identified consensus sequences or the complement or reverse complement thereof.

Based on the nucleic acid sequences of all the KPC carbapenemase genes, an oligonucleotide specific for a KPC carbapenemase gene, or an oligonucleotide primer or probe for a KPC carbapenemase gene may have a maximum of two, a maximum of one or no mismatches with any one target sequence from the KPC gene family which corresponds to the consensus sequences identified above, or to the complement or reverse complement of said target sequence. In a preferred embodiment, said oligonucleotide, primer or probe has a maximum of one or no mismatches compared with any one target sequence from the KPC gene family which corresponds to the consensus sequences identified above, or to the complement or reverse complement of said target sequence. Thus, typically the oligonucleotide specific for a KPC carbapenemase gene, or the oligonucleotide primer or probe for a KPC carbapenemase gene has at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more sequence identity with any one target sequence from the KPC gene family which corresponds to the consensus sequences identified above, or to the complement or reverse complement of said target sequence. In a preferred embodiment the oligonucleotide primer or probe for a KPC carbapenemase gene has at least 95%, at least 96%, at least 97%, at least 98% at least 99% or more, or even more preferably 100% sequence identity with any one target sequence from the KPC gene family which corresponds to the consensus sequences identified above, or to the complement or reverse complement of said target sequence. See Table 1 below.

An oligonucleotide specific for an IMP carbapenemase gene, or an oligonucleotide primer or probe for an IMP carbapenemase gene may bind to a target consensus sequence comprising or consisting of the nucleic acid sequence YCMACRTATGCRTCTRWRTTAACRAATG (SEQ ID NO: 13), CCAAACYASTASRTTRTCYKGAGYR (SEQ ID NO: 14) or TGMCCNGRDCCNGGRTARAAMAYTTCWATYT (SEQ ID NO: 15), or a consensus sequence that comprises or consists of the complement or reverse complement of any one of these sequences. Possible positions of mismatches are shown underlined. Thus, the oligonucleotide specific for an IMP carbapenemase gene, or the oligonucleotide primer or probe for an IMP carbapenemase gene may comprise or consist of any one of these nucleic acid sequences, or the complement or reverse complement thereof. An oligonucleotide specific for an IMP carbapenemase gene, or an oligonucleotide primer or probe for an IMP carbapenemase gene may comprise a maximum of eleven, a maximum of ten, a maximum of nine, a maximum of eight, a maximum of seven, a maximum of six, a maximum of five, a maximum of four, a maximum of three, a maximum of two, a maximum of one or no mismatches compared with any one of the above-identified consensus sequences, or the complement or reverse complement thereof. For the consensus sequence of CCAAACYASTASRTTRTCYKGAGYR (SEQ ID NO: 14), or the complement or reverse complement thereof, although there are eight variant positions in the consensus sequence for the IMP gene family, typically only seven are counted in the mismatch analysis because one mixed (degenerate) base is used in the oligonucleotide, primer or probe sequence. As the variance in the consensus sequence for the IMP family is accounted for in the oligonucleotide, probe or primer sequence, a base pair match will occur at this position in any binding/hybridisation. For the consensus sequence of TGMCCNGRDCCNGGRTARAAMAYTTCWATYT (SEQ ID NO: 15), although there is a maximum of eleven mismatches between the consensus sequence, or complement or reverse complement thereof and an oligonucleotide, probe or primer of the invention, the oligonucleotide, probe or primer may comprise the compensatory use of inosines (I, that attempt to pair with any of A, T, G or C). Thus, when these I residues are taken into account, there may be a maximum of eight, a maximum of seven a maximum of six, a maximum of five, a maximum of four, a maximum of three, a maximum of two, a maximum of one or no mismatches between an oligonucleotide specific for an IMP carbapenemase gene, or an oligonucleotide primer or probe for an IMP carbapenemase gene and the consensus sequence or complement or reverse complement thereof.

Thus, the oligonucleotide specific for an IMP carbapenemase gene, or the oligonucleotide primer or probe for an IMP carbapenemase gene may have at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 80%, at least 81%, at least 82%, at least 83%, at least 84%, t least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more sequence identity with any one of the above-identified consensus sequences or the complement or reverse complement thereof. In a preferred embodiment the oligonucleotide primer or probe for an IMP carbapenemase gene has at least 64%, at least 65%, at least 66%, at least 67%, at least 68%, at least 69%, at least 70%, at least 71%, at least 72%, at least 73%, at least 74%, at least 75%, at least 80%, sequence identity with any one of the above-identified consensus sequences or the complement or reverse complement thereof.

Based on the nucleic acid sequences of all the IMP carbapenemase genes, an oligonucleotide specific for an IMP carbapenemase gene, or an oligonucleotide primer or probe for an IMP carbapenemase gene may have a maximum of five, a maximum of four, a maximum of three, a maximum of two, a maximum of one or no mismatches with any one target sequence from the IMP gene family which corresponds to the consensus sequences identified above, or to the complement or reverse complement of said target sequence. In a preferred embodiment, said oligonucleotide, primer or probe has a maximum of two, a maximum of one or no mismatches compared with any one target sequence from the IMP gene family which corresponds to the consensus sequences identified above, or to the complement or reverse complement of said target sequence. Thus, typically the oligonucleotide specific for an IMP carbapenemase gene, or the oligonucleotide primer or probe for an NDM carbapenemase gene has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more sequence identity with any one target sequence from the IMP gene family which corresponds to the consensus sequences identified above, or to the complement or reverse complement of said target sequence. In a preferred embodiment the oligonucleotide primer or probe for an IMP carbapenemase gene has at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more sequence identity with any one target sequence from the IMP gene family which corresponds to the consensus sequences identified above, or to the complement or reverse complement of said target sequence. See Table 1 below.

An oligonucleotide specific for the one or more carbapenemase gene, or an oligonucleotide probe or primer may comprise or be complementary or reverse complementary to a nucleic acid sequence within a target nucleic acid sequence from the one or more carbapenemase gene of the invention, or to a nucleic acid sequence having at least 80% identity to said target nucleic acid sequence. Any suitable oligonucleotide specific for the one or more carbapenemase gene, or any suitable oligonucleotide probe or primer which comprises or is complementary (as defined herein) to a nucleic acid sequence within a target nucleic acid sequence of one or more carbapenemase gene of the invention may be used.

It is preferred that the binding conditions for an oligonucleotide specific for the one or more carbapenemase gene, or an oligonucleotide probe or primer hybridising to its target sequence are such that a high level of specificity is provided—i.e. hybridisation of the oligonucleotide, probe or primer occurs under “stringent conditions”. In general, stringent conditions are selected to be about 5° C. lower than the thermal melting point (Tm) for the specific sequence at a defined ionic strength and pH. The Tm is the temperature (under defined ionic strength and pH) at which 50% of the target (or complement) sequence

TABLE 1
Preferred target consensus sequences and
oligonucleotide/probe/primer sequences

| | | | | | | | Maximum |
|---|---|---|---|---|---|---|---|
| | | | | | % of | | % of |
| | | | | | primer/ | Maximum | primer |
| | | | | | probe | # | probe |
| | | | | Mis- | sequence | mismatches | sequence |
| | | | | matches | matched | in any | mis- |
| | | | | vs | to | one target | matched |
| | | | | consensus | consensus | sequence | in any |
| | | Consensus | Primer/probe | sequence | for | vs | one |
| | Length | sequence | sequence | for | the gene | primer/ | target |
| | (bp) | (SEQ ID NO) | (SEQ ID NO) | family | family | probe | sequence |
| KPC fwd | 23 | GCAGCGGCAGCAGYT | GCAGCGGCAGCAGTTT | 1 | 95.3% | 1 | 95.3% |
| | | TGTTGATT(10) | GTTGATT(21) | | | | |
| KPC rev^{$} | 24 | GTAGACGGCCAACAC | GTAGACGGCCAACACA | 0 | 100% | 0 | 100% |
| | | AATAGGTGC(11) | ATAGGTGC(11) | | | | |
| KPC probe | 26 | CAGTCGGAGACAAAA | CAGTCGGAGACAAAAC | 0 | 100% | 0 | 100% |
| | | CCGGAACCTGC(12) | CGGAACCTGC(12) | | | | |
| NDM fwd | 27 | CCAGCAAATGGAAAC | CCAGCAAATGGAAACT | 0 | 100% | 0 | 100% |
| | | TGGCGAC(7) | GGCGAC(7) | | | | |
| NDM rev^{$} | 21 | ATCCAGTTGAGGATCT | ATCCAGTTGAGGATCT | 0 | 100% | 0 | 100% |
| | | GGGCG(8) | GGGCG(8) | | | | |
| NDM probe | 24 | ACCGAATGTCTGGCA | ACCGAATGTCTGGCAG | 0 | 100% | 0 | 100% |
| | | GCACACTTC(9) | CACACTTC(9) | | | | |
| OXA48fwd | 27 | GATTATGGYAATGAG | GATTATGGTAATGAGG | 3 | 88.9% | 3 | 88.9% |
| | | GAYATYTCGGGC(1) | ACATTTCGGGC(16) | | | | |
| OXA48rev^{$} | 30 | CATATCCATATTCATC | CATATCCATATTCATCG | 0 | 100% | 0 | 100% |
| | | GCAAAAAACCACAC(2) | CAAAAAACCACAC(2) | | | | |
| OXA48probe^{$}* | 29 | CCATTGGCTTCGGTCA | CCATTGGCTTCGGTCAG | 0 | 100% | 0 | 100% |
| | | GCATGGCTTGTTT(3) | CATGGCTTGTTT(3) | | | | |
| VIMfwd | 26 | TTGCTTYTKATTGATA | TTGCTTTTGATTGATAC | 3 | 88.5% | 2 | 92.3% |
| | | CAGCKTGGGG(4) | AGCGTGGGG(17) | | | | |
| VIMrev^{$} | 19 | GTACGTYGCCACYCC | GTACGTTGCCACCCCA | 2 | 89.5% | 2 | 89.5% |
| | | AGCC(5) | GCC(18) | | | | |
| VIMprobe | 33 | TCGCGGAGATTGARAA | TCTCGCGGAGATTGAAAA | 1 | 97% | 1 | 97% |
| | | GCAAATTGGACTTCC(6) | GCAAATTGGACTTCC(19) | | | | |
| IMPfwd | 28 | YCMACRTATGCRTCT | CCCACGTATGCATCTGA | 8 | 71.4% | 2 | 92.9% |
| | | RWRTTAACRAATG(13) | ATTAACAAATG (22) | | | | |
| IMPrev^{$}** | 25 | CCAAACYASTASRTTR | CCAAACCACTACGTTAT | 8 | 68% | 3 | 88% |
| | | TCYKGAGYR(14) | CTKGAGTG(23) | (7) | (72%) | (2) | (92%) |
| IMPprobe1^{$}*** | 31 | TGMCCNGRDCCNGGR | TGICCTGGICCAGGATA | 11 | 64.5% | 4 | 87.1% |
| | | TARAAMAYTTCWATY | AAAAACTTCAATIT(24) | (8) | (74.2%) | (2) | (93.5%) |
| | | T(15) | | | | | |
| IMPprobe2^{$}*** | 31 | TGMCCNGRDCCNGGR | TGICCIGGGCCIGGATAA | 11 | 64.5% | 5 | 83.9% |
| | | TARAAMAYTTCWATY | AAAACTTCAATTT(25) | (8) | (74.2%) | (2) | (93.5%) |
| | | T(15) | | | | | |

Mismatches shown in bold
^{$}Reverse complemented sequence shown (5′-3′ sequence for the reverse strand- in the orientation that the primer/probe is used)
*mismatches vs Shewanella chromosomal (non-acquired) genes seen in alignments not counted due to no evidence of clinical relevance for those gene variants (Shewanella = environmental organism).
**There were eight variant positions in the consensus sequence for the gene family, but only seven were counted in this analysis because one mixed (degenerate base) position was used in the primer sequence. As the variance in the consensus sequence for the family is accounted for in the primer sequence a base pair match will occur in this position in the reaction.
***The total number of variant positions is shown first in each instance. The figures in parenthesis show the number of variant positions after the compensatory use of Inosines (that attempt to pair with any of A, T G or C) is taken into account.

hybridises to a perfectly matched probe or primer. In this regard, the Tm of oligonucleotides, probes or primers of the present invention, at a salt concentration of about 0.02M or less at pH 7, is for example above 60° C., such as about 70° C.

Premixed buffer solutions are commercially available (e.g. EXPRESSHYB Hybridisation Solution from CLONTECH Laboratories, Inc.), and hybridisation can be performed according to the manufacturer's instructions.

Oligonucleotides, probes and primers of the present invention may be screened to minimise self-complementarity and dimer formation (oligonucleotide-oligonucleotide, probe-probe or primer-primer binding).

In one embodiment the at least one oligonucleotide specific for the OXA-48-like gene, or OXA-48-like probe or primer comprises or consists of a nucleic acid sequence of CCATTGGCTTCGGTCAGCATGGCTTGTTT (SEQ ID NO: 3) GATTATGGTAATGAGGACATTTCGGGC (SEQ ID NO: 16), and/or CATATCCATATTCATCGCAAAAAACCACAC (SEQ ID NO: 2), or a nucleic acid sequence having at least 70%, at least 80%, at least 85%, at least 90%, at least 95%, at least 96%, at least 97%, at least 98%, at least 99% or more sequence identity to the nucleic acid sequence of SEQ ID NO: 3, 16 or 2. In a preferred embodiment, the at least one oligonucleotide specific for the OXA-48-like gene, or OXA-48-like primer or probe is at least 80%, at least 85%, at least 90%, at least 95%, at least 96%, at least 97%, at least 98%, at least 99% or more sequence identity to the nucleic acid sequence of SEQ ID NO: 3, 16 or 2. Typically the at least one oligonucleotide sequence specific for the OXA-48-like gene is a primer or probe as disclosed herein. For example, an OXA-48-like forward primer of the invention may comprise or consist of the nucleic acid sequence of GATTATGGTAATGAGGACATTTCGGGC (SEQ ID NO: 16) or a nucleic acid sequence having at least 70% or more sequence identity thereto (as defined above); an OXA-48-like reverse primer of the invention may comprise or consist of the nucleic acid sequence of CATATCCATATTCATCGCAAAAAACCACAC (SEQ ID NO: 2) or a nucleic acid sequence having at least 70% or more sequence identity thereto (as defined above); and/or an OXA-48-like probe of the invention may comprise or consist of the nucleic acid sequence of CCATTGGCTTCGGTCAGCATGGCTTGTTT (SEQ ID NO: 3) or a nucleic acid sequence having at least 70% or more sequence identity thereto (as defined above).

In one embodiment the at least one oligonucleotide specific for the VIM gene, or VIM probe or primer comprises or consists of a nucleic acid sequence of GTACGTTGCCACCCCAGCC (SEQ ID NO: 18), TTGCTTTTGATTGATACAGCGTGGGG (SEQ ID NO: 17), TCTCGCGGAGATTGAAAAGCAAATTGGACTTCC (SEQ ID NO: 19), and/or TCGCGGAGATTGARAAGCAAATTGGA (SEQ ID NO: 20), or a nucleic acid sequence having at least 70%, at least 80%, at least 85%, at least 90%, at least 95%, at least 96%, at least 97%, at least 98%, at least 99% or more sequence identity to the nucleic acid sequence of SEQ ID NO: 17, 18, 19 or 20. In a preferred embodiment, the at least one oligonucleotide specific for the VIM gene, or VIM primer or probe is at least 85%, at least 90%, at least 95%, at least 96%, at least 97%, at least 98%, at least 99% or more sequence identity to the nucleic acid sequence of SEQ ID NO: 18, 17, 19 or 20. Typically the at least one oligonucleotide sequence specific for the VIM gene is a primer or probe as disclosed herein. For example, a VIM forward primer of the invention may comprise or consist of the nucleic acid sequence of TTGCTTTTGATTGATACAGCGTGGGG (SEQ ID NO: 17) or a nucleic acid sequence having at least 70% or more sequence identity thereto (as defined above); a VIM reverse primer of the invention may comprise or consist of the nucleic acid sequence of GTACGTTGCCACCCCAGCC (SEQ ID NO: 18) or a nucleic acid sequence having at least 70% or more sequence identity thereto (as defined above); and/or a VIM probe of the invention may comprise or consist of the nucleic acid sequence of TCTCGCGGAGATTGAAAAGCAAATTGGACTTCC (SEQ ID NO: 19) or TCGCGGAGATTGARAAGCAAATTGGA (SEQ ID NO: 20), or a nucleic acid sequence having at least 70% or more sequence identity thereto (as defined above).

In one embodiment the at least one oligonucleotide specific for the KPC gene, or KPC probe or primer comprises or consists of a nucleic acid sequence of GCAGCGGCAGCAGTTTGTTGATT (SEQ ID NO: 21), GTAGACGGCCAACACAATAGGTGC (SEQ ID NO: 11), and/or CAGTCGGAGACAAAACCGGAACCTGC (SEQ ID NO: 12), or a nucleic acid sequence having at least 70%, at least 80%, at least 85%, at least 90%, at least 95%, at least 96%, at least 97%, at least 98%, at least 99% or more sequence identity to the nucleic acid sequence of SEQ ID NO: 21, 11 or 12. In a preferred embodiment, the at least one oligonucleotide specific for the KPC gene, or KPC primer or probe is at least 90%, at least 95%, at least 96%, at least 97%, at least 98%, at least 99% or more sequence identity to the nucleic acid sequence of SEQ ID NO: 21, 11 or 12. Typically the at least one oligonucleotide sequence specific for the KPC gene is a primer or probe as disclosed herein. For example, a KPC forward primer of the invention may comprise or consist of the nucleic acid sequence of GCAGCGGCAGCAGTTTGTTGATT (SEQ ID NO: 21) or a nucleic acid sequence having at least 70% or more sequence identity thereto (as defined above); a KPC reverse primer of the invention may comprise or consist of the nucleic acid sequence of GTAGACGGCCAACACAATAGGTGC (SEQ ID NO: 11) or a nucleic acid sequence having at least 70% or more sequence identity thereto (as defined above); and/or a KPC probe of the invention may comprise or consist of the nucleic acid sequence of CAGTCGGAGACAAAACCGGAACCTGC (SEQ ID NO: 12) or a nucleic acid sequence having at least 70% or more sequence identity thereto (as defined above).

In one embodiment the at least one oligonucleotide specific for the NDM gene, or NDM probe or primer comprises or consists of a nucleic acid sequence of CCAGCAAATGGAAACTGGCGAC (SEQ ID NO: 181), ATCCAGTTGAGGATCTGGGCG (SEQ ID NO: 8), and/or ACCGAATGTCTGGCAGCACACTTC (SEQ ID NO: 9), or a nucleic acid sequence having at least 70%, at least 80%, at least 85%, at least 90%, at least 95%, at least 96%, at least 97%, at least 98%, at least 99% or more sequence identity to the nucleic acid sequence of SEQ ID NO: 7, 8 or 9. In a preferred embodiment, the at least one oligonucleotide specific for the NDM gene, or NDM primer or probe is at least 90%, at least 95%, at least 96%, at least 97%, at least 98%, at least 99% or more sequence identity to the nucleic acid sequence of SEQ ID NO: 7, 8 or 9. Typically the at least one oligonucleotide sequence specific for the NDM gene is a primer or probe as disclosed herein. For example, an NDM forward primer of the invention may comprise or consist of the nucleic acid sequence of CCAGCAAATGGAAACTGGCGAC (SEQ ID NO: 7) or a nucleic acid sequence having at least 70% or more sequence identity thereto (as defined above); an NDM reverse primer of the invention may comprise or consist of the nucleic acid sequence of ATCCAGTTGAGGATCTGGGCG (SEQ ID NO: 8) or a nucleic acid sequence having at least 70% or more sequence identity thereto (as defined above); and/or an NDM probe of the invention may comprise or consist of the nucleic acid sequence of ACCGAATGTCTGGCAGCACACTTC (SEQ ID NO: 9) or a nucleic acid sequence having at least 70% or more sequence identity thereto (as defined above).

In one embodiment the at least one oligonucleotide specific for the IMP gene, or IMP probe or primer comprises or consists of a nucleic acid sequence of CCCACGTATGCATCTGAATTAACAAATG (SEQ ID NO: 22), CCAAACCACTACGTTATCTKGAGTG (SEQ ID NO: 23), TGICCTGGICCAGGATAAAAAACTTCAATIT (SEQ ID NO: 24), and/or TGICCIGGGCCIGGATAAAAAACTTCAATTT (SEQ ID NO: 25), or a nucleic acid sequence having at least 60%, at least 65%, at least 70%, at least 80%, at least 85%, at least 90%, at least 95%, at least 96%, at least 97%, at least 98%, at least 99% or more sequence identity to the nucleic acid sequence of SEQ ID NO: 22, 23, 24 or 25. In a preferred embodiment, the at least one oligonucleotide specific for the IMP gene, or IMP primer or probe is at least 90%, at least 95%, at least 96%, at least 97%, at least 98%, at least 99% or more sequence identity to the nucleic acid sequence of SEQ ID NO: 22, 23, 24 or 25. Typically the at least one oligonucleotide sequence specific for the IMP gene is a primer or probe as disclosed herein. For example, an IMP forward primer of the invention may comprise or consist of the nucleic acid sequence of CCCACGTATGCATCTGAATTAACAAATG (SEQ ID NO: 22) or a nucleic acid sequence having at least 70% or more sequence identity thereto (as defined above); an IMP reverse primer of the invention may comprise or consist of the nucleic acid sequence of CCAAACCACTACGTTATCTKGAGTG (SEQ ID NO: 23) or a nucleic acid sequence having at least 70% or more sequence identity thereto (as defined above); and/or an IMP probe of the invention may comprise or consist of the nucleic acid sequence of TGICCTGGICCAGGATAAAAAACTTCAATIT (SEQ ID NO: 24) or TGICCIGGGCCIGGATAAAAAACTTCAATTT (SEQ ID NO: 25), or a nucleic acid sequence having at least 70% or more sequence identity thereto (as defined above).

Any of the oligonucleotides, probes or primers described herein may comprise a tag and/or label. The tag and/or label may, for example, be located (independently of one another) towards the middle or towards or at the 5′ or 3′ end of the herein described oligonucleotides/probes/primers, for example at the 5′ end.

Hence, following hybridisation of tagged/labelled probe to target nucleic acid, the tag/label is associated with the target nucleic acid in the one or more carbapenemase gene or fragment thereof. Alternatively, if an amplification step is employed, the probes may act as primers during the method of the invention and the tag/label may therefore become incorporated into the amplification product as the primer is extended.

Examples of suitable labels include detectable labels such as radiolabels or fluorescent or coloured molecules, enzymatic markers or chromogenic markers—e.g. dyes that produce a visible colour change upon hybridisation of the probe or primer. By way of example, the label may be digoxygenin, fluorescein-isothiocyanate (FITC), R-phycoerythrin, Alexa 532, carboxy-X-rhodamine (ROX), carboxytetramethylrhodamine (TAMRA), 4,5-dichloro-dimethoxy-fluorescein (JOE), BHQ-1/2/3, Cy5, Cy5.5 or Cy3. The oligonucleotides, probes or primer preferably contain a Fam label (e.g. a 5′ Fam label), and/or a minor groove binder (MGB). The label may be a reporter molecule, which is detected directly, such as by exposure to photographic or X-ray film. Alternatively, the label is not directly detectable, but may be detected indirectly, for example, in a two-phase system. An example of indirect label detection is binding of an antibody to the label.

Examples of suitable tags include “complement/anti-complement pairs”. The term “complement/anti-complement pair” denotes non-identical moieties that form a non-covalently associated, stable pair under appropriate conditions. Examples of suitable tags include biotin and streptavidin (or avidin). By way of example, a biotin tag may be captured using streptavidin, which may be coated onto a substrate or support such as a bead (for example a magnetic bead) or membrane. Likewise, a streptavidin tag may be captured using biotin, which may be coated onto a substrate or support such as a bead (for example a magnetic bead) or membrane. Other exemplary complement/anti-complement pairs include receptor/ligand pairs, antibody/antigen (or hapten or epitope) pairs, and the like. Another example is a nucleic acid sequence tag that binds to a complementary sequence. The latter may itself be pre-labelled, or may be attached to a surface (e.g. a bead) which is separately labelled. An example of the latter embodiment is the well-known LuminexR bead system. Other exemplary pairs of tags and capture molecules include receptor/ligand pairs and antibody/antigen (or hapten or epitope) pairs. Where subsequent dissociation of the complement/anti-complement pair is desirable, the complement/anti-complement pair has a binding affinity of, for example, less than 10⁹ M⁻¹. One exemplary tagged oligonucleotide, probe or primer is a biotin-labelled oligonucleotide, probe or primer, which may be detected using horse-radish peroxidase conjugated streptavidin.

The oligonucleotides, probes or primers of the invention may be labelled with different labels or tags, thereby allowing separate identification of each oligonucleotide, probe or primer when used in the method of the present invention.

Any conventional method may be employed to attach nucleic acid tags to an oligonucleotide, probe or primer of the present invention (e.g. to the 5′ end of the defined binding region of the oligonucleotide, probe or primer). Alternatively, oligonucleotides, probes or primers of the invention (with pre-attached nucleic acid tags) may be constructed by commercial providers.

Detection of the one or more carbapenemase gene, a target region of the one or more carbapenemase gene or a fragment of said gene or target region may be carried out by any known means. In this regard, the probe or amplification product may be tagged and/or labelled, and the detection method may therefore comprise detecting said tag and/or label.

In one embodiment, the oligonucleotide(s), probe(s) or primer(s) may comprise a tag and/or label. Thus, in one embodiment, following hybridisation of tagged/labelled oligonucleotide/probe/primer to target nucleic acid in the one or more carbapenemase gene, the tag/label becomes associated with the target nucleic acid. Thus, in one embodiment, the assay may comprise detecting the tag/label and correlating presence of tag/label with presence of the one or more carbapenemase gene of the invention.

In one embodiment, tag and/or label may be incorporated during extension of the oligonucleotide(s), probe(s) or primer(s). In doing so, the amplification product(s) become tagged/labelled, and the assay may therefore comprise detecting the tag/label and correlating presence of tag/label with presence of amplification product, and hence the presence of one or more carbapenemase gene of the invention.

By way of example, in one embodiment, the amplification product may incorporate a tag/label (e.g. via a tagged/labelled dNTP such as biotin-dNTP) as part of the amplification process, and the assay may further comprise the use of a binding partner complementary to said tag (e.g. streptavidin) that includes a detectable tag/label (e.g. a fluorescent label, such as R-phycoerythrin). In this way, the amplified product incorporates a detectable tag/label (e.g. a fluorescent label, such as R-phycoerythrin).

In one embodiment, the oligonucleotide(s), probe(s) or primer(s) and/or the amplification product(s) may include a further tag/label (as the complement component) to allow capture of the amplification product(s).

By way of example, a “complement/anti-complement” pairing may be employed in which an anti-complement capture component binds to said further tag/label (complement component) and thereby permits capture of the probe(s) and/or amplification product(s). Examples of suitable “complement/anti-complement” partners have been described earlier in this specification, such as a complementary pair of nucleic acid sequences, a complementary antibody-antigen pair, etc. The anti-complement capture component may be attached (e.g. coated) on to a substrate or solid support—examples of suitable substrates/supports include membranes and/or beads (e.g. a magnetic or fluorescent bead). Capture methods are well known in the art. For example, LuminexR beads may be employed. Alternatively, the use of magnetic beads may be advantageous because the beads (plus captured, tagged/labelled amplification product) can easily be concentrated and separated from the sample, using conventional techniques known in the art.

Immobilisation provides a physical location for the oligonucleotides, probes, primers and/or anti-complement capture component of the invention, and may serve to fix the capture component/oligonucleotide/probe/primer at a desired location and/or facilitate recovery or separation of oligonucleotide/probe/primer. The support may be a rigid solid support made from, for example, glass, plastic or silica, such as a bead (for example a fluorescent or magnetic bead). Alternatively, the support may be a membrane, such as nylon or nitrocellulose membrane. 3D matrices are also suitable supports for use with the present invention—e.g. polyacrylamide or PEG gels. Immobilisation to a support/platform may be achieved by a variety of conventional means. By way of example, immobilisation onto a support such as a nylon membrane may be achieved by UV cross-linking. Alternatively, biotin-labelled molecules may be bound to streptavidin-coated substrates (and vice-versa), and molecules prepared with amino linkers may be immobilised on to silanised surfaces. Another means of immobilisation is via a poly-T tail or a poly-C tail, for example at the 3′ or 5′ end. Said immobilisation techniques apply equally to the probe component (and primer/primer pair component, if present) of the present invention.

In one embodiment, the oligonucleotide, probes and/or primers of the invention comprise a nucleic acid sequence tag/label (e.g. attached to each probe at the 5′ end of the defined sequence of the probe/primer that binds to target/complement nucleic acid). In more detail, each of the oligonucleotides/probes/primers is provided with a different nucleic acid sequence tag/label, wherein each of said tags/labels (specifically) binds to a complementary nucleic acid sequence present on the surface of a bead. Each of the different tags/labels binds to its complementary sequence counterpart (and not to any of the complementary sequence counterparts of the other tags), which is located on a uniquely identifiable bead. In this regard, the beads are uniquely identifiable, for example by means of fluorescence at a specific wavelength. Thus, in use, oligonucleotides/probes/primers of the invention bind to target nucleic acid (if present in the sample). Thereafter, (only) the bound probes may be extended (in the 3′ direction) in the presence of one or more labelled dNTP (e.g. biotin labelled dNTPs, such as biotin-dCTPs).

The extended primers may be contacted with a binding partner counterpart to the labelled dNTPs (e.g. a streptavidin labelled fluorophore, such as streptavidin labelled R-phycoerythrin), which binds to those labelled dNTPs that have become incorporated into the extended primers. Thereafter, the labelled extended primers may be identified by allowing them to bind to their nucleic acid counterparts present on the uniquely identifiable beads. The latter may then be “called” (e.g. to determine the type of bead present by wavelength emission) and the nature of the primer extension (and thus the type of target/complement nucleic acid present) may be determined.

Typically, probes/primers of the invention are oligonucleotides having sequence identity or complementarity with a region of the one or more carbapenemase gene (either the sense strand, the complementary strand or the reverse of either strand) of the invention as disclosed herein. One or more probe may be immobilised on a solid support, and used to interrogate mRNA or DNA obtained from a test sample. If the mRNA or DNA from the test sample contains the one or more carbapenemase gene targeted by the immobilised probe, it will bind to the probe, and may then be detected. The probes/primers of the invention may also be detected using PCR, such as real time PCR.

Any oligonucleotide with the appropriate level of sequence identity with the one or more carbapenemase gene of the invention, or with one or more target sequences within said one or more carbapenemase gene of the invention may be used as a probe or primer as described herein. Any oligonucleotide with the appropriate level of complementarity with the one or more carbapenemase gene of the invention, or with one or more target sequences within said one or more carbapenemase gene of the invention may be used as a probe or primer as described herein.

### Methods of Diagnosis

As described herein, the present invention provides a method for diagnosing a carbapenemase-producing bacterial infection in an individual, comprising determining the presence and/or amount of one or more carbapenemase-producing bacteria in a sample obtained from the individual by determining the presence and/or amount of a carbapenemase gene selected from an OXA-48-like gene and/or a VIM gene in said sample.

The presence and/or amount of one or more additional carbapenemase gene in said sample may also be determined. In particular, the presence and/or amount of one or more of the additional carbapenemases disclosed herein may be determined in a diagnostic method according to the present invention. In a preferred embodiment, the presence and/or amount of a KPC carbapenemase gene and/or a NDM carbapenemase gene in the sample is also determined. Thus, the presence and/or amount of any of the following combinations of carbapenemase genes may be determined: (i) an OXA-48-like carbapenemase and a VIM carbapenemase; (ii) an OXA-48-like carbapenemase and a NDM carbapenemase; (iii) an OXA-48-like carbapenemase, and a KPC carbapenemase; (iv) an OXA-48-like carbapenemase, a KPC carbapenemase and a NDM carbapenemase; (v) an OXA-48-like carbapenemase, a VIM carbapenemase and a KPC carbapenemase; (vi) an OXA-48-like carbapenemase, a VIM carbapenemase and a NDM carbapenemase; (vii) a VIM carbapenemase and a NDM carbapenemase; (viii) a VIM carbapenemase and a KPC carbapenemase; (ix) a VIM carbapenemase, a KPC carbapenemase and a NDM carbapenemase; or (x) an OXA-48-like carbapenemase, a VIM carbapenemase, a KPC carbapenemase and a NDM carbapenemase.

In a preferred embodiment, the presence and/or amount of an OXA-48-like carbapenemase gene, a VIM carbapenemase gene, a KPC carbapenemase gene and an NDM carbapenemase gene in the sample is determined in a diagnostic method according to the present invention.

In a more preferred embodiment, the presence and/or amount of at least one of an IMP carbapenemase gene an IMI carbapenemase gene, an SME carbapenemase gene, an SPM carbapenemase gene and/or a GES carbapenemase gene is determined in addition to the presence and/or amount of: (i) an OXA-48-like carbapenemase gene and a VIM carbapenemase gene; (ii) an OXA-48-like carbapenemase gene, a VIM carbapenemase gene and a KPC carbapenemase gene; (iii) an OXA-48-like carbapenemase gene, a VIM carbapenemase gene and an NDM carbapenemase gene; or (iv) an OXA-48-like carbapenemase gene, a VIM carbapenemase gene, a KPC carbapenemase gene and an NDM carbapenemase gene. As disclosed herein, the presence and/or amount of at least one, at least two, at least three, at least four or all five an IMP carbapenemase gene an IMI carbapenemase gene, an SME carbapenemase gene, an SPM carbapenemase gene and/or a GES carbapenemase gene, or any combination thereof may be determined in a diagnostic method according to the present invention.

In a particularly preferred embodiment, the presence and/or amount of an OXA-48-like carbapenemase gene, a VIM carbapenemase gene, a KPC carbapenemase gene, an NDM carbapenemase gene and an IMP carbapenemase gene is determined in a diagnostic method according to the present invention. In an even more preferred embodiment, the presence and/or amount of an OXA-48-like carbapenemase gene, a VIM carbapenemase gene, a KPC carbapenemase gene, an NDM carbapenemase gene, an IMP carbapenemase gene, an IMI carbapenemase gene, an SME carbapenemase gene, an SPM carbapenemase gene and a GES carbapenemase gene is determined in a diagnostic method according to the present invention.

The method may comprise determining the presence and/or amount of said carbapenemase gene(s) in a first sample taken from the individual at a single initial point in time and multiple time points thereafter to monitor the efficacy of treatment and disease resolution, and comparing the presence and/or amount of said carbapenemase gene(s) in said first sample to the presence and/or amount of said carbapenemase gene(s) in a reference or control sample. Said comparison may determine the status of carbapenem-resistant bacterial infection in the individual with an accuracy, sensitivity and/or specificity of at least about 90%, at least about 80%, at least about 70% or at least about 60. Typically the accuracy, sensitivity and/or specificity is of at least about 80% or at least about 90%.

The method may comprise determining the presence and/or amount of the one or more carbapenemase gene in a first sample from the individual; and comparing the presence or amount of the one or more carbapenemase gene in the individual's first sample to the presence and/or amount of the one or more carbapenemase gene in a sample from a reference or control population, said comparison being capable of classifying the individual as belonging to or not belonging to the reference or control population, wherein the comparison determines the status of carbapenem-resistant bacterial infection in the individual.

The method may further comprise determining the presence and/or amount of the one or more carbapenemase genes in a second sample taken from the individual; and comparing the presence and/or amount of the one or more carbapenemase genes in the individual's second sample to the presence and/or amount of the one or more carbapenemase genes in the control or reference sample, wherein the second comparison is capable of classifying the individual as belonging to or not belonging to the control or reference population, and wherein the second comparison determines the status of carbapenem-resistant bacterial infection in the individual.

The methods of the invention may be repeated at least once, at least twice, at least three times, at least four times, at least five times, or more. The presence and/or amount of the one or more carbapenemase genes can be determined in a separate sample taken from the individual each time the method is repeated.

The methods of the invention may be used to diagnose, detect and/or predict carbapenem-resistant bacterial infection and/or infection with one or more carbapenemase-producing bacteria. The methods of the invention may be used to distinguish between a carbapenem-resistant bacterial infection and the absence of such an infection. The methods of the invention may be used to identify an individual with a carbapenem-resistant bacterial infection and/or an individual uninfected with one or more carbapenem-resistant bacteria. The methods of the invention may also be used to determine the status of a carbapenem-resistant bacterial infection. Determining the status of a carbapenem-resistant bacterial infection in an individual may comprise determining the progression or resolution of a carbapenem-resistant bacterial infection. Determining the status of a carbapenem-resistant bacterial infection in an individual may comprise determining the presence of a carbapenem-resistant bacterial infection in an individual.

A carbapenem-resistant bacteria infection may be diagnosed or predicted prior to the onset of clinical symptoms, and/or as subsequent confirmation after the onset of clinical symptoms. Accordingly, the present invention allows for more effective therapeutic intervention and/or diagnosis in the pre-symptomatic stage of infection.

The invention also provides the use of one or more carbapenemase gene, oligonucleotide, probe and/or primer as defined herein in the manufacture of a diagnostic for a carbapenem-resistant bacterial infection. Said diagnostic may be for diagnosing a carbapenem-resistant bacterial infection.

### Kits and Devices

The invention also provides kits and devices that are useful in determining the presence and/or amount of one or more carbapenemase-producing bacteria, the status of a carbapenem-resistant bacterial infection, and/or diagnosing or detecting a carbapenem-resistant bacterial infection. The kits and devices of the present invention comprise at least one oligonucleotide, probe and/or primer of the invention and/or one or more agent for the detection of or for the determination of the amount of the one or more carbapenemase gene of the invention. Specific oligonucleotides, probes and/or primers and agents for the detection of said one or more carbapenemase gene useful in the present invention are set forth herein. The oligonucleotides, probes and/or primers of the kit or device can be used to determine the presence and/or amount of one or more carbapenemase-producing bacteria (by determining the presence and/or amount of one or more carbapenemase gene) according to the present invention.

Generally, the oligonucleotides, probes and/or primers of the kit will bind, with at least some specificity, to the one or more carbapenemase gene contained in the sample obtained from the individual. The oligonucleotides, probes and/or primers and/or agent(s) for the detection of the one or more carbapenemase gene may be part of an array, or the oligonucleotides, probes and/or primers and/or agent(s) may be packaged separately and/or individually. The oligonucleotides, probes and/or primers and/or agent(s) may be immobilised on an inert support.

The kit or device may also comprise at least one internal standard to be used in generating profiles of the one or more carbapenemase gene according to the present invention. Likewise, the internal standards can be any of the classes of compounds described above.

The kits and devices of the present invention also may contain reagents that can be used to detectably label the one or more carbapenemase gene contained in the biological samples from which the profiles of the one or more carbapenemase gene are generated. For this purpose, the kit or device may comprise antibodies which bind to the oligonucleotides, probes and/or primers of the invention. The antibodies themselves may be detectably labelled. The kit or device also may comprise a specific binding component, such as an aptamer.

In a preferred embodiment, a kit or device of the invention comprises one or more oligonucleotide, probe and/or primer specific for the one or more carbapenemase gene. In a more preferred embodiment, the one or more oligonucleotide, probe or primer specific for the one or more carbapenemase gene is an oligonucleotide of the invention, more preferably one or more of SEQ ID NOs: 1 to 15, even more preferably one or more of SEQ ID NOs: 2 to 3, 7 to 9, 11, 12, 16 to 19 and/or 21 to 25. Typically, the kit or device of the invention comprises at least one of SEQ ID NOs: 1 to 3 and at least one of SEQ ID NOs: 4 to 6, even more preferably at least one of SEQ ID NOs: 2, 3 and 16 and at least one of SEQ ID NOs: 17 to 19. In a preferred embodiment, the kit or device of the invention comprises SEQ ID NOs: 1, 2, 4 and 5, and optionally comprises at least one of SEQ ID NOs: 3 or 6. In a preferred embodiment, a kit or device of the invention comprises at least one of SEQ ID Nos: 3 or 18, and optionally one or more of SEQ ID Nos: 16, 2, 19 and 17. In an even more preferred embodiment, the kit or device of the invention comprises SEQ ID Nos: 2 and 16 to 18 and optionally comprises at least one of SEQ ID Nos: 3, 19 and/or 20. The kit or device of the invention may further comprise one or more of SEQ ID Nos: 7 to 13 and/or 15. In a preferred embodiment, the kit or device of the invention may further comprise one or more of SEQ ID Nos: 7 to 9, 11, 12 and/or 21 to 25. In a particularly preferred embodiment, the kit or device of the invention comprises SEQ ID Nos: 3, 18, 16, 19, 2 and 17, or oligonucleotides with at least 80% sequence identity to said SEQ ID Nos (as defined herein). The kit or device of the invention may comprise oligonucleotides having a defined level of sequence identity with any one of the above mentioned sequence, as defined herein.

The kit or device may provide one or more oligonucleotide probe that is capable of forming a duplex with the one or more carbapenemase gene or with a complementary strand of said one or more one or more carbapenemase gene. The one or more oligonucleotide probe may be detectably labelled. Typically, the one or more oligonucleotide probe used in the methods of the invention is selected from one or more of the oligonucleotide described herein. In a preferred embodiment, the one or more oligonucleotide probe is selected from an oligonucleotide probe that comprises or is complementary to at least one nucleic acid sequence having at least 80% sequence identity to the nucleic acid sequence of any one or more of SEQ ID NOs: 2 to 3, 7 to 9, 11, 12, 16 to 19 and/or 21 to 25. Typically, the oligonucleotide probe of the invention is selected from SEQ ID NOs: 3, 16 and/or 2 and 18, 19, 17 and/or 20.

The kits and devices of the present invention may also include other classes of compounds including, but not limited to, proteins (including antibodies), and fragments thereof, peptides, polypeptides, proteoglycans, glycoproteins, lipoproteins, carbohydrates, lipids, nucleic acids, organic and inorganic chemicals, and natural and synthetic polymers.

The kits and devices of the present invention may also include pharmaceutical excipients, diluents and/or adjuvants. Examples of pharmaceutical adjuvants include, but are not limited to, preservatives, wetting agents, emulsifying agents, and dispersing agents. Prevention of the action of microorganisms can be ensured by the inclusion of various antibacterial and antifungal agents, for example, paraben, chlorobutanol, phenol sorbic acid, and the like. It may also be desirable to include isotonic agents such as sugars, sodium chloride, and the like.

### Sequence Information

A non-exhaustive list of OXA-48-like carbapenemase gene of the inventions is set out below (Table 2). Thus, an OXA-48-like carbapenemase gene of the invention may have a polynucleotide sequence of any one of the accession numbers or SEQ ID NOs below, or a fragment or variant of one of said sequences, as defined herein.

| Genbank | | |
|---|---|---|
| Accession | | |
| Number | Version Number | SEQ ID NO: |
| CP000469 | CP000469.1 GI: 117610791 | 26 |
| CP000446 | CP000446.1 GI: 113883030 | 27 |
| CP000444 | CP000444.1 GI: 113886955 | 28 |
| JQ996150 | JQ996150.1 GI: 440685600 | 29 |
| JN205800 | JN205800.1 GI: 347602110 | 30 |
| HM992946 | HM992946.1 GI: 304368222 | 31 |
| JX423831 | JX423831.1 GI: 444236140 | 32 |
| JX893517 | JX893517.1 GI: 442769982 | 33 |
| HQ700343 | HQ700343.1 GI: 323817046 | 34 |
| KC902850 | KC902850.1 GI: 529273301 | 35 |
| KC902852 | KC902852.1 GI: 529273309 | 36 |
| JQ809466 | JQ809466.1 GI: 408795221 | 37 |
| JN704570 | JN704570.1 GI: 379139226 | 38 |
| KF900153 | KF900153.1 GI: 573006828 | 39 |
| JX438001 | JX438001.1 GI: 442577759 | 40 |
| GU197550 | GU197550.1 GI: 270312218 | 41 |
| HM015773 | HM015773.1 GI: 295684221 | 42 |
| AY236073 | AY236073.2 GI: 69209772 | 43 |
| JX423832 | JX423832.1 GI: 443909556 | 44 |
| HE617182 | HE617182.1 GI: 379131236 | 45 |
| JN714122 | JN714122.1 GI: 359359955 | 46 |
| JN626286 | JN626286.1 GI: 359422634 | 47 |
| KC354801 | KC354801.1 GI: 479327067 | 48 |
| KC757416 | KC757416.2 GI: 513046067 | 49 |
| KC335143 | KC335143.1 GI: 544600294 | 50 |
| KC757417 | KC757417.2 GI: 513046066 | 51 |
| JX438000 | JX438000.1 GI: 442577757 | 52 |

A non-exhaustive list of VIM carbapenemase gene of the inventions is set out below (Table 3). Thus, a VIM carbapenemase gene of the invention may have a polynucleotide sequence of any one of the accession numbers or SEQ ID Nos below, or a fragment or variant of one of said sequences, as defined herein.

| Genbank | | |
|---|---|---|
| Accession | | |
| Number | Version Number | SEQ ID NO: |
| AJ536835 | AJ536835.1 GI: 30014071 | 53 |
| DQ365886 | DQ365886.1 GI: 86279732 | 54 |
| AM778091 | AM778091.1 GI: 157310266 | 55 |
| AY524987 | AY524987.1 GI: 42406212 | 56 |
| EU090799 | EU090799.1 GI: 156628070 | 57 |
| AF300454 | AF300454.1 GI: 11037698 | 58 |
| DQ835309 | DQ835309.1 GI: 112785289 | 59 |
| AY605049 | AY605049.2 GI: 54887317 | 60 |
| DQ022682 | DQ022682.1 GI: 66734335 | 61 |
| JN982330 | JN982330.1 GI: 388253361 | 62 |
| GQ414736 | GQ414736.1 GI: 256692864 | 63 |
| EU118148 | EU118148.2 GI: 208879769 | 64 |
| EU419745 | EU419745.1 GI: 171705293 | 65 |
| EU419746 | EU419746.1 GI: 171705295 | 66 |
| GQ242167 | GQ242167.1 GI: 253684194 | 67 |
| AY524988 | AY524988.1 GI: 42406214 | 68 |
| AY524989 | AY524989.1 GI: 42406216 | 69 |
| EF522838 | EF522838.1 GI: 145321079 | 70 |
| AY165025 | AY165025.1 GI: 26892103 | 71 |
| HM855205 | HM855205.1 GI: 302340960 | 72 |
| JX982635 | JX982635.1 GI: 441494187 | 73 |
| JN129451 | JN129451.1 GI: 355526407 | 74 |
| AF191564 | AF191564.1 GI: 7381447 | 75 |
| AJ586617 | AJ586617.2 GI: 46559085 | 76 |
| KC469971 | KC469971.2 GI: 465214873 | 77 |
| AY144612 | AY144612.1 GI: 24415594 | 78 |
| DQ143913 | DQ143913.1 GI: 72536051 | 79 |
| JX013656 | JX013656.1 GI: 396586112 | 80 |
| JX982636 | JX982636.1 GI: 441494189 | 81 |
| HQ858608 | HQ858608.1 GI: 323473327 | 82 |
| JF803952 | JF803952.1 GI: 336327549 | 83 |
| FR748153 | FR748153.1 GI: 315064910 | 84 |
| FJ445404 | FJ445404.1 GI: 219872982 | 85 |
| AY635904 | AY635904.1 GI: 49035768 | 86 |
| FJ499397 | FJ499397.1 GI: 237688234 | 87 |
| FJ822963 | FJ822963.1 GI: 262180929 | 88 |
| JF900599 | JF900599.1 GI: 336169750 | 89 |
| EU581706 | EU581706.1 GI: 187960474 | 90 |
| EF693948 | EF693948.1 GI: 157411581 | 91 |
| AY135661 | AY135661.1 GI: 22758830 | 92 |
| JX311308 | JX311308.1 GI: 401716858 | 93 |
| JX258134 | JX258134.1 GI: 401713038 | 94 |
| JX982634 | JX982634.1 GI: 441494185 | 95 |
| JN676230 | JN676230.2 GI: 389656440 | 96 |
| GU724872 | GU724872.1 GI: 291586255 | 97 |
| Y18050 | Y18050.2 GI: 30840267 | 98 |
| HM750249 | HM750249.1 GI: 308746072 | 99 |
| DQ023222 | DQ023222.1 GI: 66277416 | 100 |
| FJ915116 | FJ915116.1 GI: 229560242 | 101 |
| AY456196 | AY456196.1 GI: 38456063 | 102 |

A non-exhaustive list of KPC carbapenemase gene of the inventions is set out below (Table 4). Thus, a KPC carbapenemase gene of the invention may have a polynucleotide sequence of any one of the accession numbers or SEQ ID Nos below, or a fragment or variant of one of said sequences, as defined herein.

| Accession | | |
|---|---|---|
| Number | Version Number | SEQ ID NO: |
| JF431928 | JF431928.1 GI: 343952330 | 103 |
| KC433553 | KC433553.1 GI: 451824560 | 104 |
| JX524191 | JX524191.1 GI: 409974861 | 105 |
| KC465199 | KC465199.1 GI: 478437782 | 106 |
| HQ641421 | HQ641421.1 GI: 326467060 | 107 |
| AF297554 | AF297554.1 GI: 10121874 | 108 |
| KC465200 | KC465200.1 GI: 478437784 | 109 |
| AY034847 | AY034847.1 GI: 14626419 | 110 |
| AY700571 | AY700571.1 GI: 51557251 | 111 |
| HM066995 | HM066995.1 GI: 296939581 | 112 |
| EU400222 | EU400222.2 GI: 214010993 | 113 |
| HQ342890 | HQ342890.1 GI: 327397926 | 114 |
| HQ342889 | HQ342889.1 GI: 327397924 | 115 |
| FJ624872 | FJ624872.1 GI: 223558116 | 116 |
| EU555534 | EU555534.1 GI: 172054654 | 117 |
| GQ140348 | GQ140348.1 GI: 239925270 | 118 |
| FJ234412 | FJ234412.1 GI: 209892783 | 119 |
| HM769262 | HM769262.1 GI: 312623594 | 120 |
| AF395881 | AF395881.1 GI: 15705412 | 121 |
| EU729727 | EU729727.1 GI: 189916550 | 122 |

A non-exhaustive list of NDM carbapenemase gene of the inventions is set out below (Table 5). Thus, an NDM carbapenemase gene of the invention may have a polynucleotide sequence of any one of the accession numbers or SEQ ID Nos below, or a fragment or variant of one of said sequences, as defined herein.

| Accession | | |
|---|---|---|
| Number | Version Number | SEQ ID NO: |
| KF361506 | KF361506.1 GI: 530540024 | 123 |
| AB744718 | AB744718.1 GI: 451810087 | 124 |
| JX262694 | JX262694.1 GI: 401884433 | 125 |
| JN104597 | JN104597.1 GI: 344226673 | 126 |
| JQ348841 | JQ348841.1 GI: 378405410 | 127 |
| KC999080 | KC999080.2 GI: 672816753 | 128 |
| JQ734687 | JQ734687.1 GI: 388890592 | 129 |
| JN967644 | JN967644.1 GI: 366085572 | 130 |
| FN396876 | FN396876.1 GI: 255031061 | 131 |
| JF703135 | JF703135.1 GI: 327244638 | 132 |

A non-exhaustive list of IMP carbapenemase gene of the inventions is set out below (Table 6). Thus, an IMP carbapenemase gene of the invention may have a polynucleotide sequence of any one of the accession numbers or SEQ ID Nos below, or a fragment or variant of one of said sequences, as defined herein.

| Accession | | |
|---|---|---|
| Number | Version Number | SEQ ID NO: |
| FN556190 | FN556190.1 GI: 308026240 | 133 |
| HM138673 | HM138673.1 GI: 299780310 | 134 |
| EF184215 | EF184215.1 GI: 134035513 | 135 |
| AY780674 | AY780674.2 GI: 76008227 | 136 |
| KF148593 | KF148593.1 GI: 529283985 | 137 |
| JQ002629 | JQ002629.1 GI: 403309640 | 138 |
| GQ302618 | GQ302618.2 GI: 268627877 | 139 |
| GQ302617 | GQ302617.2 GI: 268627875 | 140 |
| AY553332 | AY553332.1 GI: 62736339 | 141 |
| AB204557 | AB204557.1 GI: 59891390 | 142 |
| AB074436 | AB074436.1 GI: 17026025 | 143 |
| AB777501 | AB777501.1 GI: 459978576 | 144 |
| AB753458 | AB753458.1 GI: 408535170 | 145 |
| DQ361087 | DQ361087.2 GI: 126574581 | 146 |
| AJ584652 | AJ584652.2 GI: 37653301 | 147 |
| AY033653 | AY033653.3 GI: 194359396 | 148 |
| HM75876 | HM175876.1 GI: 300391789 | 149 |
| EU588392 | EU588392.2 GI: 194245456 | 150 |
| HQ438058 | HQ438058.1 GI: 314912176 | 151 |
| EF184216 | EF184216.1 GI: 134035518 | 152 |
| AY553333 | AY553333.1 GI: 62736340 | 153 |
| JF810083 | JF810083.1 GI: 342161745 | 154 |
| AF290912 | AF290912.1 GI: 13491138 | 155 |
| JQ407409 | JQ407409.1 GI: 383216584 | 156 |
| GQ221781 | GQ221781.1 GI: 253987477 | 157 |
| AB777500 | AB777500.1 GI: 459978567 | 158 |
| AF318077 | AF318077.1 GI: 13094933 | 159 |
| JF894248 | JF894248.1 GI: 335345650 | 160 |
| DQ307573 | DQ307573.1 GI: 83583500 | 161 |
| GQ221782 | GQ221782.1 GI: 253987479 | 162 |
| GU045307 | GU045307.1 GI: 261864919 | 163 |
| HQ875573 | HQ875573.1 GI: 345128596 | 164 |
| FJ384365 | FJ384365.1 GI: 217038355 | 165 |
| AF288045 | AF288045.2 GI: 195977007 | 166 |
| AF244145 | AF244145.1 GI: 13128916 | 167 |
| AJ420864 | AJ420864.1 GI: 25278055 | 168 |
| AJ243491 | AJ243491.1 GI: 8546572 | 169 |
| EF192154 | EF192154.1 GI: 122934823 | 170 |
| AB196988 | AB196988.1 GI: 56783616 | 171 |
| EF118171 | EF118171.1 GI: 122891986 | 172 |
| EU042136 | EU042136.1 GI: 154867189 | 173 |
| AF322577 | AF322577.2 GI: 14161935 | 174 |
| JN848782 | JN848782.2 GI: 536749749 | 175 |
| JX131372 | JX131372.1 GI: 401668653 | 176 |
| GU207399 | GU207399.1 GI: 281021955 | 177 |
| AJ550807 | AJ550807.1 GI: 33086392 | 178 |
| AJ512502 | AJ512502.1 GI: 40644310 | 179 |
| AB715422 | AB715422.2 GI: 697287983 | 180 |
| AB010417 | AB010417.1 GI: 4760642 | 181 |
| AM283490 | AM283490.1 GI: 110350566 | 182 |
| EU541448 | EU541448.1 GI: 171188393 | 183 |
| AB753456 | AB753456.1 GI: 408535166 | 184 |
| AB753457 | AB753457.1 GI: 408535168 | 185 |
| AB195637 | AB195637.1 GI: 57158242 | 186 |
| AB074433 | AB074433.1 GI: 17026019 | 187 |
| AB188812 | AB188812.1 GI: 51555859 | 188 |
| AB040994 | AB040994.1 GI: 10566736 | 189 |
| DQ522237 | DQ522237.1 GI: 102231664 | 190 |
| AB469046 | AB469046.1 GI: 211922742 | 191 |
| S71932 | S71932.1 GI: 560551 | 192 |
| JF816544 | JF816544.1 GI: 350285632 | 193 |

| SEQ ID NO: 1 | |
|---|---|
| gattatggya atgaggayat ytcgggc | 27 |
| SEQ ID NO: 2 | |
| catatccata ttcatcgcaa aaaaccacac | 30 |
| SEQ ID NO: 3 | |
| ccattggctt cggtcagcat ggcttgttt | 29 |
| SEQ ID NO: 4 | |
| ttgcttytka ttgatacagc ktgggg | 26 |
| SEQ ID NO: 5 | |
| gtacgtygcc acyccagcc | 19 |
| SEQ ID NO: 6 | |
| tcgcggagat tgaraagcaa attggacttc c | 31 |
| SEQ ID NO: 7 | |
| ccagcaaatg gaaactggcg ac | 22 |
| SEQ ID NO: 8 | |
| atccagttga ggatctgggc g | 21 |
| SEQ ID NO: 9 | |
| accgaatgtc tggcagcaca cttc | 24 |
| SEQ ID NO: 10 | |
| gcagcggcag cagyttgttg att | 23 |
| SEQ ID NO: 11 | |
| gtagacggcc aacacaatag gtgc | 24 |
| SEQ NO: 12 | |
| cagtcggaga caaaaccgga acctgc | 26 |
| SEQ NO: 13 | |
| ycmacrtatg crtctrwrtt aacraatg | 28 |
| SEQ NO: 14 | |
| ccaaacyast asrttrtcyk gagyr | 25 |
| SEQ NO: 15 | |
| tgmccngrdc cnggrtaraa mayttcwaty t | 31 |
| n is a, c, g, or t | |
| SEQ NO: 16 | |
| gattatggta atgaggacat ttcgggc | 27 |
| SEQ NO: 17 | |
| ttgcttttga ttgatacagc gtgggg | 26 |
| SEQ NO: 18 | |
| gtacgttgcc accccagcc | 19 |
| SEQ NO: 19 | |
| tctcgcggag attgaaaagc aaattggact tcc | 33 |
| SEQ NO: 20 | |
| tcgcggagat tgaraagcaa attgga | 26 |
| SEQ NO: 21 | |
| gcagcggcag cagtttgttg att | 23 |
| SEQ NO: 22 | |
| cccacgtatg catctgaatt aacaaatg | 28 |
| SEQ NO: 23 | |
| ccaaaccact acgttatctk gagtg | 25 |
| SEQ NO: 24 | |
| tgncctggnc caggataaaa aacttcaatn t | 31 |
| n- inosine | |
| SEQ NO: 25 | |
| tgnccngggc cnggataaaa aacttcaatt t | 31 |
| n- inosine | |
| SEQ NO: 26 | |
| atgcgtgcgt tagccttatc ggctgtgttg ctggtgacaa cgatgattgg catgcctgcg | 60 |
| gtggcaaagg agtggcaaga gaacaagagc tggaatgctc actttagcga acataaaacc | 120 |
| caaggcgtgg ttgtgctgtg gaacgagaat acacagcagg gttttactaa cgatcttaaa | 180 |
| cgggcaaacc aagcattttt acctgcatcg acctttaaga tcccaaacag tttaattgcc | 240 |
| ttggacttag gtgtggttaa ggatgagcat caagtcttta aatgggatgg acagacgcga | 300 |
| gatatcgccg catggaatcg cgaccatgat ttaatcaccg cgatgaagta ttcggttgtg | 360 |
| cctgtttatc aagaatttgc ccgccaaatt ggcgaggctc gtatgagtaa aatgctgcac | 420 |
| gccttcgatt atggcaatga ggatatctcg ggcaatttag acagcttttg gctcgatggt | 480 |
| ggtattcgaa tttcggctac ccagcaaatc gcttttttac gcaagctgta ccacaacaag | 540 |
| ctgcacgttt ctgagcgtag tcagcgcatc gtcaagcaag ccatgctgac cgaggcaaat | 600 |
| gccgattata ttatccgagc gaaaactggt tactcgacgc gaattgagcc gaaaattggt | 660 |
| tggtgggttg gctgggtcga actcgatgac aatgtgtggt tcttcgcgac caatatggat | 720 |
| atgccttccg ctgagggcct agggttacgt caaagcatta cgaaaacagt gctgaaacag | 780 |
| gaaaaaatta ttccttag | 798 |
| SEQ NO: 27 | |
| atgcgtgcgt tagccttatc ggctgtgttg atggtgacaa cgatgattgg catgcctgcg | 60 |
| gtggcaaagg agtggcaaga gaacaagagc tggaatgctc actttagcga acataaaacc | 120 |
| caaggcgtgg ttgtgctctg gaacgagaat acacagcagg gttttactaa cgatcttaaa | 180 |
| cgggcaaacc aagcattttt accggcatcg acctttaaga tcccaaacag tttaattgcc | 240 |
| ttggacttag gtgtggttaa ggatgagcat caggtcttta aatgggatgg acagacgcga | 300 |
| gatatcgccg catggaatcg tgaccatgat ttaatcaccg cgatgaagta ttcggttgtg | 360 |
| cctgtttatc aagaatttgc ccgccaaatt ggcgaggctc gtatgaataa aatgctgcac | 420 |
| gccttcgatt atggcaatga ggatatctcg ggcaatttgg acagtttttg gctcgatggt | 480 |
| ggtattcgca tttcggctac ccagcaaatc gcttttttac gcaagctgta ccacaacaag | 540 |
| ctgcacgttt ctgagcgtag tcagcgcatc gtgaaacaag ccatgctgac cgaggcaaat | 600 |
| gccgactata tcatccgagc gaaaaccggc tattcggtca gaattgaacc gaaaatcggt | 660 |
| tggtgggttg gctgggtcga actggatgat aatgtgtggt ttttcgcgac caatatggat | 720 |
| atgccttccg ctgagggctt agggttacgt caaagcatta cgaaagcagt gctgaaacag | 780 |
| gaaaaaatta ttccttag | 798 |
| SEQ NO: 28 | |
| atgcgtgcgt tagccttatc ggctgtgttg atggtgacaa cgatgattgg catgcctgcg | 60 |
| gtggcaaagg agtggcaaga gaacaagagt tggaatgctc actttagcga acataaaacc | 120 |
| caaggcgtgg ttgtgctctg gaacgagaat aaacagcagg gttttactaa cgatcttaaa | 180 |
| cgggcaaacc aagcattttt accggcatcg acttttaaga tcccaaacag tttaattgcc | 240 |
| ttggacttag gtgtggttaa ggatgagcat caggtcttta aatgggatgg acagacgcga | 300 |
| gatatcgccg cgtggaatcg tgaccatgac ttaattaccg cgatgaagta ctcggttgtg | 360 |
| cctgtttatc aagaatttgc ccgccaaatt ggcgaggcgc gtatgagtaa aatgctgcat | 420 |
| gccttcgatt atggcaatga ggatatctcg ggcaatttgg acagtttttg gctcgatggt | 480 |
| ggtattcgca tttcggctac ccagcaaatc gcttttttac gcaagctcta ccacaacaag | 540 |
| ctgcacgtct ctgagcgtag tcagcgcatc gtcaaacaag ccatgctgac cgaagcaaat | 600 |
| gccgactata tcattcgggc gaaaacgggt tattcggtca gaattgagcc gaaaatcggt | 660 |
| tggtgggttg gctgggtcga actcgatgac aatgtgtggt tcttcgcgac caatatggat | 720 |
| atgccttccg ctgagggctt agggttacgt caaagcatta cgaaagcagt gctaaaacag | 780 |
| gaaaaaatta ttccttag | 798 |
| SEQ NO: 29 | |
| atgcgtgtat tagccttatc ggctgtgttt ttggtggcat cgattatcgg aatgccagcg | 60 |
| gtagcaaagg aatggcaaga aaacaaaagt tggaatgctc actttactga acataaatca | 120 |
| cagggcgtag ttgtgctctg gaatgagaat aagcagcaag gatttaccaa taatcttaaa | 180 |
| cgggcgaacc aagcattttt acccgcatct acctttaaaa ttcccaatag cttgatcgcc | 240 |
| ctcgatttgg gcgtggttaa ggatgaacac caagtcttta agtgggatgg acagacgcgt | 300 |
| gatatcgccg cttggaatcg tgaccatgac ttaattaccg cgatgaagta ctcagttgtg | 360 |
| cctgtttatc aagaatttgc ccgccaaatt ggtgaggcac gtatgagtaa aatgctgcac | 420 |
| gccttcgatt atggcaatga ggatatctcg ggcaatgtag acagtttttg gctcgatggt | 480 |
| ggtattcgca tttcggctac ccagcaaatc gcttttttac gcaagctgta tcacaacaag | 540 |
| ctgcacgttt ctgagcgtag tcagcgcatc gtgaaacaag ccatgctgac cgaagccaat | 600 |
| ggcgactata ttattcgggc taaaacggga tactcgacta gaatcgaacc taagattggc | 660 |
| tggtgggttg gttgggttga acttgatgat aatgtgtggt tttttgcgat gaatatggat | 720 |
| atgcccacat cggatggttt agggctgcgc caagccatca caaaagaagt gctcaaacag | 780 |
| gagaaaatta ttccctag | 798 |
| SEQ ID NO: 30 | |
| atgcgtgtat tagccttatc ggctgtgttt ttggtggcat cgattatcgg aatgccagcg | 60 |
| gtagcaaagg aatggcaaga aaacaaaagt tggaatgctc actttactga acataaatca | 120 |
| cagggcgtag ttgtgctctg gaatgagaat aagcagcaag gatttaccaa taatcttaaa | 180 |
| cgggcgaacc aagcattttt acccgcatct acctttaaaa ttcccaatag cttgatcgcc | 240 |
| ctcgatttgg gcgtggttaa ggatgaacac caagtcttta agtgggatgg acagacgcgt | 300 |
| gatatcgccg cttggaatcg tgaccatgac ttaattaccg cgatgaagta ctcagttgtg | 360 |
| cctgtttatc aagaatttgc ccgccaaatt ggtgaggcac gtatgagtaa aatgctgcac | 420 |
| gccttcgatt atggcaatga ggatatctcg ggcaatgtag acagtttttg gctcgatggt | 480 |
| ggtattcgca tttcggctac ccagcaaatc gcttttttac gcaagctgta tcacaacaag | 540 |
| ctgcacgttt ctgagcgtag tcagcgcatc gtgaaacaag ccatgctgac cgaagccaat | 600 |
| ggcgactata ttattcgggc taaaacggga tactcgacta gaatcgaacc taagattggc | 660 |
| tggtgggttg gttgggttga acttgatgat aatgtgtggt tttttgcgat gaatatggat | 720 |
| atgcccacat cggatggttt agggctgcgc caagccatca caaaagaagt gctcaaacag | 780 |
| gagaaaatta ttccctag | 798 |
| SEQ ID NO: 31 | |
| atgcgtgtat tagccttatc ggctgtgttt ttggtggcat cgattatcgg aatgccagcg | 60 |
| gtagcaaagg aatggcaaga aaacaaaagt tggaatgctc actttactga acataaatca | 120 |
| cagggcgtag ttgtgctctg gaatgagaat aagcagcaag gatttaccaa taatcttaaa | 180 |
| cgggcgaacc aagcattttt acccgcatct acctttaaaa ttcccaatag cttgatcgcc | 240 |
| ctcgatttgg gcgtggttaa ggatgaacac caagtcttta agtgggatgg acagacgcgt | 300 |
| gatatcgccg cttggaatcg tgaccatgac ttaattaccg cgatgaagta ctcagttgtg | 360 |
| cctgtttatc aagaatttgc ccgccaaatt ggtgaggcac gtatgagtaa aatgctgcac | 420 |
| gccttcgatt atggcaatga ggatatctcg ggcaatgtag acagtttttg gctcgatggt | 480 |
| ggtattcgca tttcggctac ccagcaaatc gcttttttac gcaagctgta tcacaacaag | 540 |
| ctgcacgttt ctgagcgtag tcagcgcatc gtgaaacaag ccatgctgac cgaagccaat | 600 |
| ggcgactata ttattcgggc taaaacggga tactcgacta gaatcgaacc taagattggc | 660 |
| tggtgggttg gttgggttga acttgatgat aatgtgtggt tttttgcgat gaatatggat | 720 |
| atgcccacat cggatggttt agggctgcgc caagccatca caaaagaagt gctcaaacag | 780 |
| gagaaaatta ttccctag | 798 |
| SEQ ID NO: 32 | |
| atgcgtgtat tagccttatc ggctgtgttt ttggtggcat cgattatcgg aatgccagcg | 60 |
| gtagcaaagg aatggcaaga aaacaaaagt tggaatgctc actttactga acataaatca | 120 |
| cagggcgtag ttgtgctctg gaatgagaat aagcagcaag gatttaccaa taatcttaaa | 180 |
| cgggcgaacc aagcattttt acccgcatct acctttaaaa ttcccaatag cttgatcgcc | 240 |
| ctcgatttgg gcgtggttaa ggatgaacac caagtcttta agtgggatgg acagacgcgt | 300 |
| gatatcgccg cttggaatcg tgaccatgac ttaattaccg cgatgaagta ctcagttgtg | 360 |
| cctgtttatc aagaatttgc ccgccaaatt ggtgaggcac gtatgagtaa aatgctgcac | 420 |
| gccttcgatt atggcaatga ggatatctcg ggcaatgtag acagtttttg gctcgatggt | 480 |
| ggtattcgca tttcggctac ccagcaaatc gcttttttac gcaagctgta tcacaacaag | 540 |
| ctgcacgttt ctgagcgtag tcagcgcatc gtgaaacaag ccatgctgac cgaagccaat | 600 |
| ggcgactata ttattcgggc taaaacggga tactcgacta gtatcgaacc taagattggc | 660 |
| tggtgggttg gttgggttga acttgatgat aatgtgtggt tttttgcgat gaatatggat | 720 |
| atgcccacat cggatggttt agggctgcgc caagccatca caaaagaagt gctcaaacag | 780 |
| gagaaaatta ttccctag | 798 |
| SEQ ID NO: 33 | |
| atgcgtgtat tagccttatc ggctgtgttt ttggtggcat cgattatcgg aatgcctgcg | 60 |
| gtagcaaagg aatggcaaga aaacaaaagt tggaatgctc actttactga acataaatca | 120 |
| cagggcgtag ttgtgctctg gaatgagaat aagcagcaag gatttaccaa taatcttaaa | 180 |
| cgggcgaacc aagcattttt acccgcatct acctttaaaa ttcccaatag cttgatcgcc | 240 |
| ctcgatttgg gcgtggttaa ggatgaacac caagtcttta agtgggatgg acagacgcgc | 300 |
| gatatcgcca cttggaatcg cgatcataat ctaatcaccg cgatgaaata ttcagttgtg | 360 |
| cctgtttatc aagaatttgc ccgccaaatt ggcgaggcac gtatgagcaa gatgctacat | 420 |
| gctttcgatt atggtaatga ggacatttcg ggcaatgtag acagtttctg gctcgacggt | 480 |
| ggtattcgaa tttcggccac ggagcaaatc agctttttaa gaaagctgta tcacaataag | 540 |
| ttacacgtat cggagcgcag ccagcgtatt gtcaaacaag ccatgctgac cgaagccaat | 600 |
| ggcgactata ttattcgggc taaaactgga tccaatacta agattggctg gtgggtcggt | 660 |
| tgggttgaac ttgatgataa tgtgtggttt tttgcgatga atatggatat gcccacatcg | 720 |
| gatggtttag ggctgcgcca agccatcaca aaagaagtgc tcaaacagga aaaaattatt | 780 |
| ccctag | 786 |
| SEQ ID NO: 34 | |
| atgcgtgtat tagccttatc ggctgtgttt ttggtggcat cgattatcgg aatgcctgcg | 60 |
| gtagcaaagg aatggcaaga aaacaaaagt tggaatgctc actttactga acataaatca | 120 |
| cagggcgtag ttgtgctctg gaatgagaat aagcagcaag gatttaccaa taatcttaaa | 180 |
| cgggcgaacc aagcattttt acccgcatct acctttaaaa ttcccaatag cttgatcgcc | 240 |
| ctcgatttgg gcgtggttaa ggatgaacac caagtcttta agtgggatgg acagacgcgc | 300 |
| gatatcgcca cttggaatcg cgatcataat ctaatcaccg cgatgaaata ttcagttgtg | 360 |
| cctgtttatc aagaatttgc ccgccaaatt ggcgaggcac gtatgagcaa gatgctacat | 420 |
| gctttcgatt atggtaatga ggacatttcg ggcaatgtag acagtttctg gctcgacggt | 480 |
| ggtattcgaa tttcggccac ggagcaaatc agctttttaa gaaagctgta tcacaataag | 540 |
| ttacacgtat cggagcgcag ccagcgtatt gtcaaacaag ccatgctgac cgaagccaat | 600 |
| ggcgactata ttattcgggc taaaactgga tacgatacta agattggctg gtgggtcggt | 660 |
| tgggttgaac ttgatgataa tgtgtggttt tttgcgatga atatggatat gcccacatcg | 720 |
| gatggtttag ggctgcgcca agccatcaca aaagaagtgc tcaaacagga aaaaattatt | 780 |
| ccctag | 786 |
| SEQ ID NO: 35 | |
| atgcgtgtat tagccttatc ggctgtgttt ttggtggcat cgattatcgg aatgcctgcg | 60 |
| gtagcaaagg aatggcaaga aaacaaaagt tggaatgctc actttactga acataaatca | 120 |
| cagggcgtag ttgtgctctg gaatgagaat aagcagcaag gatttaccaa taatcttaaa | 180 |
| cgggcgaacc aagcattttt acccgcatct acctttaaaa ttcccaatag cttgatcgcc | 240 |
| ctcgatttgg gcgtggttaa ggatgaacac caagtcttta agtgggatgg acagacgcgc | 300 |
| gatatcgcca cttggaatcg cgatcataat ctaatcaccg cgatgaaata ttcagttgtg | 360 |
| cctgtttatc aagaatttgc ccgccaaatt ggcgaggcac gtatgagcaa gatgctacat | 420 |
| gctttcgatt atggtaatga ggacatttcg ggcaatgtag acagtttctg gctcgatggt | 480 |
| ggtattcgaa tttcggccac tgagcaaatc agctttttaa gaaagctgta tcacaataag | 540 |
| ttacacgtat cggagcgcag tcagcgcatc gtgaaacaag ccatgctgac cgaagccaat | 600 |
| ggcgactata ttattcgggc taaaacggga tactcgacta gaatcgaacc taagattggc | 660 |
| tggtgggtcg gttgggttga acttgatgat aatgtgtggt tttttgcgat gaatatggat | 720 |
| atgcccacat cggatggttt agggctgcgc caagccatca caaaagaagt gctcaaacag | 780 |
| gaaaaaatta ttccctag | 798 |
| SEQ ID NO: 36 | |
| atgcgtgtat tagccttatc ggctgtgttt ttggtggcat cgattatcgg aatgcctgcg | 60 |
| gtagcaaagg aatggcaaga aaacaaaagt tggaatgctc actttactga acataaatca | 120 |
| cagggcgtag ttgtgctctg gaatgagaat aagcagcaag gatttaccaa taatcttaaa | 180 |
| cgggcgaacc aagcattttt acccgcatct acctttaaaa ttcccaatag cttgatcgcc | 240 |
| ctcgatttgg gcgtggttaa ggatgaacac caagtcttta agtgggatgg acatcggcgc | 300 |
| gatatcgcca cttggaatcg cgatcataat ctaatcaccg cgatgaaata ttcagttgtg | 360 |
| cctgtttatc aagaatttgc ccgccaaatt ggcgaggcac gtatgagcaa gatgctacat | 420 |
| gctttcgatt atggtaatga ggacatttcg ggcaatgtag acagtttctg gctcgatggt | 480 |
| ggtattcgaa tttcggccac tgagcaaatc agctttttaa gaaagctgta tcacaataag | 540 |
| ttacacgtat cggagcgcag ccagcgtatt gtcaaacaag ccatgctgac cgaagccaat | 600 |
| ggcgactata ttattcgggc taaaacggga tactcgacta gaatcgaacc taagattggc | 660 |
| tggtgggtcg gttgggttga actggatgat aatgtgtggt tttttgcgat gaatatggat | 720 |
| atgcccacat cggatggttt agggctgcgc caagccatca caaaagaagt gctcaaacag | 780 |
| gaaaaaatta ttccctag | 798 |
| SEQ ID NO: 37 | |
| atgcgtgtat tagccttatc ggctgtgttt ttggtggcat cgattatcgg aatgcctgcg | 60 |
| gtagcaaagg aatggcaaga aaacaaaagt tggaatgctc actttactga acataaatca | 120 |
| cagggcgtag ttgtgctctg gaatgagaat aagcagcaag gatttaccaa taatcttaaa | 180 |
| cgggcgaacc aagcattttt acccgcatct acctttaaaa ttcccaatag cttgatcgcc | 240 |
| ctcgatttgg gcgtggttaa ggatgaacac caagtcttta agtgggatgg acatcggcgc | 300 |
| gatatcgcca cttggaatcg cgatcataat ctaatcaccg cgatgaaata ttcagttgtg | 360 |
| cctgtttatc aagaatttgc ccgccaaatt ggcgaggcac gtatgagcaa gatgctacat | 420 |
| gctttcgatt atggtaatga ggacatttcg ggcaatgtag acagtttctg gctcgatggt | 480 |
| ggtattcgaa tttcggccac tgagcaaatc agctttttaa gaaagctgta tcacaataag | 540 |
| ttacacgtat cggagcgcag ccagcgtatt gtcaaacaag ccatgctgac cgaagccaat | 600 |
| ggcgactata ttattcgggc taaaacggga tactcgacta gaatcgaacc taagattggc | 660 |
| tggtgggtcg gttgggttga actggatgat aatgtgtggt tttttgcgat gaatatggat | 720 |
| atgcccacat cggatggttt agggctgcgc caagccatca caaaagaagt gctcaaacag | 780 |
| gaaaaaatta ttccctag | 798 |
| SEQ ID NO: 38 | |
| atgcgtgtat tagccttatc ggctgtgttt ttggtggcat cgattatcgg aatgcctgcg | 60 |
| gtagcaaagg aatggcaaga aaacaaaagt tggaatgctc actttactga atataaatca | 120 |
| cagggcgtag ttgcgctctg gaatgagaat aagcagcaag gatttaccaa taatcttaaa | 180 |
| cgggcgaacc aagcattttt acccgcatct acctttaaaa ttcccaatag cttgatcgcc | 240 |
| ctcgatttgg gcgtggttaa ggatgaacac caagtcttta agtgggatgg acagacgcgc | 300 |
| gatatcgcca cttggaatcg cgatcataat ctaatcaccg cgatgaaata ttcagttgtg | 360 |
| cctgtttatc aagaatttgc ccgccaaatt ggcgaggcac gtatgagcaa gatgctacat | 420 |
| gctttcgatt atggtaatga ggacatttcg ggcaatgtag gcagtttctg gctcgacggt | 480 |
| ggtattcgaa tttcggccac tgagcaaatc agctttttaa gaaagctgta tcacaataag | 540 |
| ttacacgtat cggagcgcag ccagcgtatt gtcaaacaag ccatgctgac cgaagccaat | 600 |
| ggcgactata ttattcgggc taaaactgga tactcgacta gaatcgaacc taagattggc | 660 |
| tggtgggtcg gttgggttga acttgatgat aatgtgtggt tttttgcgat gaatatggat | 720 |
| atgcccacat cggatggttt agggctgcgc caagccatca caaaagaagt gctcaaacag | 780 |
| gaaaaaatta ttccctag | 798 |
| SEQ ID NO: 39 | |
| atgcgtgtat tagccttatc ggctgtgttt ttggtggcat cgattatcgg aatgcctgcg | 60 |
| gtagcaaagg aatggcaaga aaacaaaagt tggaatgctc actttactga acataaatca | 120 |
| cagggcgtag ttgtgctctg gaatgagaat aagcagcaag gatttaccaa taatcttaaa | 180 |
| cgggcgaacc aagcattttt acccgcatct acctttaaaa ttcccaatag cttgatcgcc | 240 |
| ctcgatttgg gcgtggttaa ggatgaacac caagtcttta agtgggatgg acagacgcgc | 300 |
| gatatcgcca cttggaatcg cgatcataat ctaatcaccg cgatgaaata ttcagttgtg | 360 |
| cctgtttatc aagaatttgc ccgccaaatt ggcgaggcac gtatgagcaa gatgctacat | 420 |
| gctttcgatt atggtaatga ggacatttcg ggcaatgtag acagtttctg gctcgacggt | 480 |
| ggtattcgaa tttcggccac ggagcaaatc agctttttaa gaaagctgta tcacaataag | 540 |
| ttacacgtat cggagcgcag ccagcgtatt gtcaaacaag ccatgctgac cgaagccaat | 600 |
| ggcgactata ttattcgggc taaaactgga tacgagacta gaatcgaacc taagattggc | 660 |
| tggtgggtcg gttgggttga acttgatgat aatgtgtggt tttttgcgat gaatatggat | 720 |
| atgcccacat cggatggttt agggctgcgc caagccatca caaaagaagt gctcaaacag | 780 |
| gaaaaaatta ttccctag | 798 |
| SEQ ID NO: 40 | |
| atgcgtgtat tagccttatc ggctgtgttt ttggtggcat cgattatcgg aatgcctgcg | 60 |
| gtagcaaagg aatggcaaga aaacaaaagt tggaatgctc actttactga acataaatca | 120 |
| cagggcgtag ttgtgctctg gaatgagaat aagcagcaag gatttaccaa taatcttaaa | 180 |
| cgggcgaacc aagcattttt acccgcatct acctttaaaa ttcccaatag cttgatcgcc | 240 |
| ctcgatttgg gcgtggttaa ggatgaacac caagtcttta agtgggatgg acagacgcgc | 300 |
| gatatcgcca cttggaatcg cgatcataat ctaatcaccg cgatgaaata ttcagttgtg | 360 |
| cctgtttatc aatactttgc ccgccaaatt ggcgaggcac gtatgagcaa gatgctacat | 420 |
| gctttcgatt atggtaatga ggacatttcg ggcaatgtag acagtttctg gctcgacggt | 480 |
| ggtattcgaa tttcggccac ggagcaaatc agctttttaa gaaagctgta tcacaataag | 540 |
| ttacacgtat cggagcgcag ccagcgtatt gtcaaacaag ccatgctgac cgaagccaat | 600 |
| ggtgactata ttattcgggc taaaactgga tactcgacta gaatcgaacc taagattggc | 660 |
| tggtgggtcg gttgggttga acttgatgat aatgtgtggt tttttgcgat gaatatggat | 720 |
| atgcccacat cggatggttt agggctgcgc caagccatca caaaagaagt gctcaaacag | 780 |
| gaaaaaatta ttccctag | 798 |
| SEQ ID NO: 41 | |
| atgcgtgtat tagccttatc ggctgtgttt ttggtggcat cgattatcgg aatgcctgcg | 60 |
| gtagcaaagg aatggcaaga aaacaaaagt tggaatgctc actttactga acataaatca | 120 |
| cagggcgtag ttgtgctctg gaatgagaat aagcagcaag gatttaccaa taatcttaaa | 180 |
| cgggcgaacc aagcattttt acccgcatct acctttaaaa ttcccaatag cttgatcgcc | 240 |
| ctcgatttgg gcgtggttaa ggatgaacac caagtcttta agtgggatgg acagacgcgc | 300 |
| gatatcgcca cttggaatcg cgatcataat ctaatcaccg cgatgaaata ttcagttgtg | 360 |
| cctgtttatc aagaatttgc ccgccaaatt ggcgaggcac gtatgagcaa gatgctacat | 420 |
| gctttcgatt atggtaatga ggacatttcg ggcaatgtag acagtttctg gctcgacggt | 480 |
| ggtattcgaa tttcggccac ggagcaaatc agctttttaa gaaagctgta tcacaataag | 540 |
| ttacacgtat cggagcgcag ccagcgtatt gtcaaacaag ccatgctgac cgaagccaat | 600 |
| ggtgactata ttattcgggc taaaactgga tactcggcta gaatcgaacc taagattggc | 660 |
| tggtgggtcg gttgggttga acttgatgat aatgtgtggt tttttgcgat gaatatggat | 720 |
| atgcccacat cggatggttt agggctgcgc caagccatca caaaagaagt gctcaaacag | 780 |
| gaaaaaatta ttccctag | 798 |
| SEQ ID NO: 42 | |
| atgcgtgtat tagccttatc ggctgtgttt ttggtggcat cgattatcgg aatgcctgcg | 60 |
| gtagcaaagg aatggcaaga aaacaaaagt tggaatgctc actttactga acataaatca | 120 |
| cagggcgtag ttgtgctctg gaatgagaat aagcagcaag gatttaccaa taatcttaaa | 180 |
| cgggcgaacc aagcattttt acccgcatct acctttaaaa ttcccaatag cttgatcgcc | 240 |
| ctcgatttgg gcgtggttaa ggatgaacac caagtcttta agtgggatgg acagacgcgc | 300 |
| gatatcgcca cttggaatcg cgatcataat ctaatcaccg cgatgaaata ttcagttgtg | 360 |
| cctgtttatc aagaatttgc ccgccaaatt ggcgaggcac gtatgagcaa gatgctacat | 420 |
| gctttcgatt atggtaatga ggacatttcg ggcaatgtag acagtttctg gctcgacggt | 480 |
| ggtattcgaa tttcggccac ggagcaaatc agctttttaa gaaagctgta tcacaataag | 540 |
| ttacacgtat cggagcgcag ccagcgtatt gtcaaacaag ccatgctgac cgaagccaat | 600 |
| ggtgactata ttattcgggc taaaactgga tactcggcta gaatcgaacc taagattggc | 660 |
| tggtgggtcg gttgggttga acttgatgat aatgtgtggt tttttgcgat gaatatggat | 720 |
| atgcccacat cggatggttt agggctgcgc caagccatca caaaagaagt gctcaaacag | 780 |
| gaaaaaatta ttccctag | 798 |
| SEQ ID NO: 43 | |
| atgcgtgtat tagccttatc ggctgtgttt ttggtggcat cgattatcgg aatgcctgcg | 60 |
| gtagcaaagg aatggcaaga aaacaaaagt tggaatgctc actttactga acataaatca | 120 |
| cagggcgtag ttgtgctctg gaatgagaat aagcagcaag gatttaccaa taatcttaaa | 180 |
| cgggcgaacc aagcattttt acccgcatct acctttaaaa ttcccaatag cttgatcgcc | 240 |
| ctcgatttgg gcgtggttaa ggatgaacac caagtcttta agtgggatgg acagacgcgc | 300 |
| gatatcgcca cttggaatcg cgatcataat ctaatcaccg cgatgaaata ttcagttgtg | 360 |
| cctgtttatc aagaatttgc ccgccaaatt ggcgaggcac gtatgagcaa gatgctacat | 420 |
| gctttcgatt atggtaatga ggacatttcg ggcaatgtag acagtttctg gctcgacggt | 480 |
| ggtattcgaa tttcggccac ggagcaaatc agctttttaa gaaagctgta tcacaataag | 540 |
| ttacacgtat cggagcgcag ccagcgtatt gtcaaacaag ccatgctgac cgaagccaat | 600 |
| ggtgactata ttattcgggc taaaactgga tactcgacta gaatcgaacc taagattggc | 660 |
| tggtgggtcg gttgggttga acttgatgat aatgtgtggt tttttgcgat gaatatggat | 720 |
| atgcccacat cggatggttt agggctgcgc caagccatca caaaagaagt gctcaaacag | 780 |
| gaaaaaatta ttccctag | 798 |
| SEQ ID NO: 44 | |
| atgcgtgtat tagccttatc ggctgtgttt ttggtggcat cgattatcgg aatgcctgcg | 60 |
| gtagcaaagg aatggcaaga aaacaaaagt tggaatgctc actttactga acataaatca | 120 |
| cagggcgtag ttgtgctctg gaatgagaat aagcagcaag gatttaccaa taatcttaaa | 180 |
| cgggcgaacc aagcattttt acccgcatct acctttaaaa ttcccaatag cttgatcgcc | 240 |
| ctcgatttgg gcgtggttaa ggatgaacac caagtcttta agtgggatgg acagacgcgc | 300 |
| gatatcgcca cttggaatcg cgatcataat ctaatcaccg cgatgaaata ttcagttgtg | 360 |
| cctgtttatc aagaatttgc ccgccaaatt ggcgaggcac gtatgagcaa gatgctacat | 420 |
| gctttcgatt atggtaatga ggacatttcg ggcaatgtag acagtttctg gctcgacggt | 480 |
| ggtattcgaa tttcggccac ggagcaaatc agctttttaa gaaagctgta tcacaataag | 540 |
| ttacacgtat cggagcgcag ccagcgtatt gtcaaacaag ccatgctgac cgaagccaat | 600 |
| ggtgactata ttattcgggc taaaactgga tactcgacta gaatcgaacc taagattggc | 660 |
| tggtgggtcg gttgggttga acttgatgat aatgtgtggt tttttgcgat gaatatggat | 720 |
| atgcccacat cggatggttt agggctgcgc caagccatca caaaagaagt gctcaaacag | 780 |
| gaaaaaatta ttccctag | 798 |
| SEQ ID NO: 45 | |
| atgcgtgtat tagccttatc ggctgtgttt ttggtggcat cgattatcgg aatgcctgcg | 60 |
| gtagcaaagg aatggcaaga aaacaaaagt tggaatgctc actttactga acataaatca | 120 |
| cagggcgtag ttgtgctctg gaatgagaat aagcagcaag gatttaccaa taatcttaaa | 180 |
| cgggcgaacc aagcattttt acccgcatct acctttaaaa ttcccaatag cttgatcgcc | 240 |
| ctcgatttgg gcgtggttaa ggatgaacac caagtcttta agtgggatgg acagacgcgc | 300 |
| gatatcgcca cttggaatcg cgatcataat ctaatcaccg cgatgaaata ttcagttgtg | 360 |
| cctgtttatc aagaatttgc ccgccaaatt ggcgaggcac gtatgagcaa gatgctacat | 420 |
| gctttcgatt atggtaatga ggacatttcg ggcaatgtag acagtttctg gctcgacggt | 480 |
| ggtattcgaa tttcggccac ggagcaaatc agctttttaa gaaagctgta tcacaataag | 540 |
| ttacacgtat cggagcgcag ccagcgtatt gtcaaacaag ccatgctgac cgaagccaat | 600 |
| ggtgactata ttattcgggc taaaactgga tactcgacta gaatcgaacc taagattggc | 660 |
| tggtgggtcg gttgggttga acttgatgat aatgtgtggt tttttgcgat gaatatggat | 720 |
| atgcccacat cggatggttt agggctgcgc caagccatca caaaagaagt gctcaaacag | 780 |
| gaaaaaatta ttccctag | 798 |
| SEQ ID NO: 46 | |
| atgcgtgtat tagccttatc ggctgtgttt ttggtggcat cgattatcgg aatgcctgcg | 60 |
| gtagcaaagg aatggcaaga aaacaaaagt tggaatgctc actttactga acataaatca | 120 |
| cagggcgtag ttgtgctctg gaatgagaat aagcagcaag gatttaccaa taatcttaaa | 180 |
| cgggcgaacc aagcattttt acccgcatct acctttaaaa ttcccaatag cttgatcgcc | 240 |
| ctcgatttgg gcgtggttaa ggatgaacac caagtcttta agtgggatgg acagacgcgc | 300 |
| gatatcgcca cttggaatcg cgatcataat ctaatcaccg cgatgaaata ttcagttgtg | 360 |
| cctgtttatc aagaatttgc ccgccaaatt ggcgaggcac gtatgagcaa gatgctacat | 420 |
| gctttcgatt atggtaatga ggacatttcg ggcaatgtag acagtttctg gctcgacggt | 480 |
| ggtattcgaa tttcggccac ggagcaaatc agctttttaa gaaagctgta tcacaataag | 540 |
| ttacacgtat cggagcgcag ccagcgtatt gtcaaacaag ccatgctgac cgaagccaat | 600 |
| ggtgactata ttattcgggc taaaactgga tactcgacta gaatcgaacc taagattggc | 660 |
| tggtgggtcg gttgggttga acttgatgat aatgtgtggt tttttgcgat gaatatggat | 720 |
| atgcccacat cggatggttt agggctgcgc caagccatca caaaagaagt gctcaaacag | 780 |
| gaaaaaatta ttccctag | 798 |
| SEQ ID NO: 47 | |
| atgcgtgtat tagccttatc ggctgtgttt ttggtggcat cgattatcgg aatgcctgcg | 60 |
| gtagcaaagg aatggcaaga aaacaaaagt tggaatgctc actttactga acataaatca | 120 |
| cagggcgtag ttgtgctctg gaatgagaat aagcagcaag gatttaccaa taatcttaaa | 180 |
| cgggcgaacc aagcattttt acccgcatct acctttaaaa ttcccaatag cttgatcgcc | 240 |
| ctcgatttgg gcgtggttaa ggatgaacac caagtcttta agtgggatgg acagacgcgc | 300 |
| gatatcgcca cttggaatcg cgatcataat ctaatcaccg cgatgaaata ttcagttgtg | 360 |
| cctgtttatc aagaatttgc ccgccaaatt ggcgaggcac gtatgagcaa gatgctacat | 420 |
| gctttcgatt atggtaatga ggacatttcg ggcaatgtag acagtttctg gctcgacggt | 480 |
| ggtattcgaa tttcggccac ggagcaaatc agctttttaa gaaagctgta tcacaataag | 540 |
| ttacacgtat cggagcgcag ccagcgtatt gtcaaacaag ccatgctgac cgaagccaat | 600 |
| ggtgactata ttattcgggc taaaactgga tactcgacta gaatcgaacc taagattggc | 660 |
| tggtgggtcg gttgggttga acttgatgat aatgtgtggt tttttgcgat gaatatggat | 720 |
| atgcccacat cggatggttt agggctgcgc caagccatca caaaagaagt gctcaaacag | 780 |
| gaaaaaatta ttccctag | 798 |
| SEQ ID NO: 48 | |
| atgcgtgtat tagccttatc ggctgtgttt ttggtggcat cgattatcgg aatgcctgcg | 60 |
| gtagcaaagg aatggcaaga aaacaaaagt tggaatgctc actttactga acataaatca | 120 |
| cagggcgtag ttgtgctctg gaatgagaat aagcagcaag gatttaccaa taatcttaaa | 180 |
| cgggcgaacc aagcattttt acccgcatct acctttaaaa ttcccaatag cttgatcgcc | 240 |
| ctcgatttgg gcgtggttaa ggatgaacac caagtcttta agtgggatgg acagacgcgc | 300 |
| gatatcgcca cttggaatcg cgatcataat ctaatcaccg cgatgaaata ttcagttgtg | 360 |
| cctgtttatc aagaatttgc ccgccaaatt ggcgaggcac gtatgagcaa gatgctacat | 420 |
| gctttcgatt atggtaatga ggacatttcg ggcaatgtag acagtttctg gctcgacggt | 480 |
| ggtattcgaa tttcggccac ggagcaaatc agctttttaa gaaagctgta tcacaataag | 540 |
| ttacacgtat cggagcgcag ccagcgtatt gtcaaacaag ccatgctgac cgaagccaat | 600 |
| ggtgactata ttattcgggc taaaactgga tactcgacta gaatcgaacc taagattggc | 660 |
| tggtgggtcg gttgggttga acttgatgat aatgtgtggt tttttgcgat gaatatggat | 720 |
| atgcccacat cggatggttt agggctgcgc caagccatca caaaagaagt gctcaaacag | 780 |
| gaaaaaatta ttccctag | 798 |
| SEQ ID NO: 49 | |
| atgcgtgtat tagccttatc ggctgtgttt ttggtggcat cgattatcgg aatgcctgcg | 60 |
| gtagcaaagg aatggcaaga aaacaaaagt tggaatgctc actttactga acataaatca | 120 |
| cagggcgtag ttgtgctctg gaatgagaat aagcagcaag gatttaccaa taatcttaaa | 180 |
| cgggcgaacc aagcattttt acccgcatct acctttaaaa ttcccaatag cttgatcgcc | 240 |
| ctcgatttgg gcgtggttaa ggatgaacac caagtcttta agtgggatgg acagacgcgc | 300 |
| gatatcgcca cttggaatcg cgatcataat ctaatcaccg cgatgaaata ttcagttgtg | 360 |
| cctgtttatc aagaatttgc ccgccaaatt ggcgaggcac gtatgagcaa gatgctacat | 420 |
| gctttcgatt atggtaatga ggacatttcg ggcaatgtag acagtttctg gctcgacggt | 480 |
| ggtattcgaa tttcggccac ggagcaaatc agctttttaa gaaagctgta tcacaataag | 540 |
| ttacacgtat cggagcgcag ccagcgtatt gtcaaacaag ccatgctgac cgaagccaat | 600 |
| ggtgactata ttattcgggc taaaactgga tactcgacta gaatcgaacc taagattggc | 660 |
| tggtgggtcg gttgggttga acttgatgat aatgtgtggt tttttgcgat gaatatggat | 720 |
| atgcccacat cggatggttt agggctgcgc caagccatca caaaagaagt gctcaaacag | 780 |
| gaaaaaatta ttccctag | 798 |
| SEQ ID NO: 50 | |
| atgcgtgtat tagccttatc ggctgtgttt ttggtggcat cgattatcgg aatgcctgcg | 60 |
| gtagcaaagg aatggcaaga aaacaaaagt tggaatgctc actttactga acataaatca | 120 |
| cagggcgtag ttgtgctctg gaatgagaat aagcagcaag gatttaccaa taatcttaaa | 180 |
| cgggcgaacc aagcattttt acccgcatct acctttaaaa ttcccaatag cttgatcgcc | 240 |
| ctcgatttgg gcgtggttaa ggatgaacac caagtcttta agtgggatgg acagacgcgc | 300 |
| gatatcgcca cttggaatcg cgatcataat ctaatcaccg cgatgaaata ttcagttgtg | 360 |
| cctgtttatc aagaatttgc ccgccaaatt ggcgaggcac gtatgagcaa gatgctacat | 420 |
| gctttcgatt atggtaatga ggacatttcg ggcaatgtag acagtttctg gctcgacggt | 480 |
| ggtattcgaa tttcggccac ggagcaaatc agctttttaa gaaagctgta tcacaataag | 540 |
| ttacacgtat cggagcgcag ccagcgtatt gtcaaacaag ccatgctgac cgaagccaat | 600 |
| ggtgactata ttattcgggc taaaactgga tactcgacta gaatcgaacc taagattggc | 660 |
| tggtgggtcg gttgggttga acttgatgat aatgtgtggt tttttgcgat gaatatggat | 720 |
| atgcccacat cggatggttt agggctgcgc caagccatca caaaagaagt gctcaaacag | 780 |
| gaaaaaatta ttccctag | 798 |
| SEQ ID NO: 51 | |
| atgcgtgtat tagccttatc ggctgtgttt ttggtggcat cgattatcgg aatgcctgcg | 60 |
| gtagcaaagg aatggcaaga aaacaaaagt tggaatgctc actttactga acataaatca | 120 |
| cagggcgtag ttgtgctctg gaatgagaat aagcagcaag gatttaccaa taatcttaaa | 180 |
| cgggcgaacc aagcattttt acccgcatct acctttaaaa ttcccaatag cttgatcgcc | 240 |
| ctcgatttgg gcgtggttaa ggatgaacac caagtcttta agtgggatgg acagacgcgc | 300 |
| gatatcgcca cttggaatcg cgatcataat ctaatcaccg cgatgaaata ttcagttgtg | 360 |
| cctgtttatc aagaatttgc ccgccaaatt ggcgaggcac gtatgagcaa gatgctacat | 420 |
| gctttcgatt atggtaatga ggacatttcg ggcaatgtag acagtttctg gctcgacggt | 480 |
| ggtattcgaa tttcggccac ggagcaaatc agctttttaa gaaagctgta tcacaataag | 540 |
| ttacacgtat cggagcgcag ccagcgtatt gtcaaacaag ccatgctgac cgaagccaat | 600 |
| ggtgactata ttattcgggc taaaactgga tactcgacta gaatcgaacc taagattggc | 660 |
| tggtgggtcg gttgggttga acttgatgat aatgtgtggt tttttgcgat gaatatggat | 720 |
| atgcccacat cggatggttt agggctgcgc caagccatca caaaagaagt gctcaaacag | 780 |
| gaaaaaatta ttccctag | 798 |
| SEQ ID NO: 52 | |
| atgcgtgtat tagccttatc ggctgtgttt ttggtggcat cgattatcgg aatgcctgcg | 60 |
| gtagcaaagg aatggcaaga aaacaaaagt tggaatgctc actttactga acataaatca | 120 |
| cagggcgtag ttgtgctctg gaatgagaat aagcagcaag gatttaccaa taatcttaaa | 180 |
| cgggcgaacc aagcattttt acccgcatct acctttaaaa ttcccaatag cttgatcgcc | 240 |
| ctcgatttgg gcgtggttaa ggatgaacac caagtcttta agtgggatgg acagacgcgc | 300 |
| gatatcgcca cttggaatcg cgatcataat ctaatcaccg cgatgaaata ttcagttgtg | 360 |
| cctgtttatc aagaatttgc ccgccaaatt ggcgaggcac gtatgagcaa gatgctacat | 420 |
| gctttcgatt atggtaatga ggacatttcg ggcaatgtag acagtttctg gctcgacggt | 480 |
| ggtattcgaa tttcggccac ggagcaaatc agctttttaa gaaagctgta tcacaataag | 540 |
| ttacacgtat cggagcgcag ccagcgtatt gtcaaacaag ccatgctgac cgaagccaat | 600 |
| ggtgactata ttattcgggc taaaactgga tactcgactg gaatcgaacc taagattggc | 660 |
| tggtgggtcg gttgggttga acttgatgat aatgtgtggt tttttgcgat gaatatggat | 720 |
| atgcccacat cggatggttt agggctgcgc caagccatca caaaagaagt gctcaaacag | 780 |
| gaaaaaatta ttccctag | 798 |
| SEQ ID NO: 53 | |
| atgtttcaaa ttcgcagctt tctggttggt atcagtgcat tcgtcatggc cgtacttgga | 60 |
| tcagcagcat attccgcaca gcctggcggt gaatatccga cagtagatga cataccggta | 120 |
| ggggaagttc ggctgtacaa gattggcgat ggcgtttggt cgcatatcgc aactcagaaa | 180 |
| ctcggtgaca cggtgtactc gtctaatgga cttatcgtcc gcgatgctga tgagttgctt | 240 |
| cttattgata cagcgtgggg ggcgaagaac acggtagccc ttctcgcgga gattgaaaag | 300 |
| caaattggac ttccagtaac gcgctcaatt tctacgcact tccatgacga tcgagtcggt | 360 |
| ggagttgatg tcctccgggc ggctggagtg gcaacgtaca cctcaccctt gacacgccag | 420 |
| ctggccgaag cggcgggaaa cgaggtgcct gcgcactctc taaaagcgct ctcctctagt | 480 |
| ggagatgtgg tgcgcttcgg tcccgtagag gttttctatc ctggtgctgc gcattcgggc | 540 |
| gacaatcttg tggtatacgt gccggccgtg cgcgtactgt ttggtggctg tgcagttcat | 600 |
| gaggcgtcac gcgaatccgc gggtaatgtt gccgatgcca atttggcaga atggcctgct | 660 |
| accattaaac gaattcaaca gcggtatccg gaagcagagg tcgtcatccc cggccacggt | 720 |
| ctaccgggcg gtctggaatt gctccaacac acaactaacg ttgtcaaaac gcacaaagta | 780 |
| cgcccggtgg ccgagtaa | 798 |
| SEQ ID NO: 54 | |
| atgttaaaag ttattagtag tttattgttc tacatgaccg cctctctaat ggctgtagct | 60 |
| agtccgttag cccattccgg ggagtcgaga ggtgagtatc cgacagtcag cgaaattccg | 120 |
| gtcggagaag ttcggctgta ccagattgac gatggtgttt ggtcgcatat cgcaacgcat | 180 |
| acgtttgatg gcgtggtgta cccgtccaat ggtctcattg tccgtgatgg cgatgagttg | 240 |
| cttttgattg atacagcttg gggtacgaaa aacacagtgg cccttctcgc ggagattgag | 300 |
| aagcaaattg gacttcccgt aacgcgttca gtctccacgc actttcatga cgaccgcgtc | 360 |
| ggcggagttg atgcccttag ggcggctgga gtggcgacgt acgcatcgcc ctcgacacgc | 420 |
| cgtctagccg aggcagaggg gaacgaggtt cccacacact ctctagaagg gctctcatcg | 480 |
| agtggggacg cagtgcgttt cggtccagta gagctcttct atcctggtgc tgcgcattcg | 540 |
| accgacaatc tggttgtata cgtcccgtca gcgaacgtgc tatacggtgg ttgtgccgtt | 600 |
| ctggaattgt cacgcacatc cgcgggaaac gtggccgatg ccgacctggc tgaatggccc | 660 |
| ggttccgttg agcggattca acaacattac ccagaagcag aggtggtcat tcccgggcac | 720 |
| ggtctaccgg gcggtctaga cttgctccag cacacagcga acgttgtcaa agcacacaca | 780 |
| aatcgctcag tcgccgagta g | 801 |
| SEQ ID NO: 55 | |
| atgttcaaac ttttgagtaa gttattggtc tatttgaccg cgtctatcat ggctattgcg | 60 |
| agtccgctcg ctttttccgt agattctagc ggtgagtatc cgacagtcag cgaaattccg | 120 |
| gtcggggagg tccggcttta ccagattgcc gatggtgttt ggtcgcatat cgcaacgcgg | 180 |
| tcgtttgatg gcgcagtcta cccgtccaat ggtctcattg tccgtgatgg tgatgagttg | 240 |
| cttttgattg atacagcgtg gggtgcgaaa aacacagcgg cacttctcgc ggagattgag | 300 |
| aagcaaattg gacttcctgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagcga acgagattcc cacgcactct ctagaaggac tctcatcgag cggggacgca | 480 |
| gtgcgcttcg gtccagtaga actcttctat cctggtgctg cgcattcgac cgacaactta | 540 |
| gttgtgtacg tcccgtctgc gagtgtgctc tatggtggtt gtgcgattta tgagttgtca | 600 |
| cgcacgtctg cggggaacgt ggccgatgcc gatctggctg aatggcccac ctccattgag | 660 |
| cggattcaac aacactaccc ggaagcacag ttcgtcattc cggggcacgg cctgccgggc | 720 |
| ggtctagact tgctcaagca cacaacgaat gttgtaaaag cgcacacaaa tcgctcagtc | 780 |
| gttgagtag | 789 |
| SEQ ID NO: 56 | |
| atgttcaaac ttttgagtaa gttattggtc tatttgaccg cgtctatcat ggctattgcg | 60 |
| agtccgctcg ctttttccgt agattctagc ggtgagtatc cgacagtcag cgaaattccg | 120 |
| gtcggggagg tccggcttta ccagattgcc gatggtgttt ggtcgcatat cgcaacgcag | 180 |
| tcgtttgatg gcgcagtcta cccgtccaat ggtctcattg tccgtgatgg tgatgagttg | 240 |
| cttttgattg atacagcgtg gggtgcgaaa aacacagcgg cacttctcgc ggagattgag | 300 |
| aagcaaattg gacttcctgt aacgcgtgca gtctccacgc actttcatga cgaccgcgtc | 360 |
| ggcggcgttg atgtccttcg ggcggctggg gtggcaacgt acgcatcacc gtcggcacgc | 420 |
| cggctagccg aggtagaggg gaacgagatt cccacgcact ctctagaagg actctcatcg | 480 |
| agcggggacg cggtgcgctt cggtccagta gaactcttct atcctggtgc tgcgcattcg | 540 |
| accgacaact tagttgtgta cgtcccgtct gcgagtgtgc tctatggtgg ttgtgcgatt | 600 |
| tatgagttgt cacgcacgtc tgcggggaac gtggccgatg ccgatctggc tgaatggccc | 660 |
| acctccattg agcggattca acaacactac ccagaagcac agttcgtcat tccggggcac | 720 |
| ggcctgccgg gcggtctaga cttgctcaag cacacaacga atgttgtaaa agcgcacaca | 780 |
| aatcgctcag tcgttgagta g | 801 |
| SEQ ID NO: 57 | |
| atgttcaaac ttttgagtaa gttattggtc tatttgaccg cgtctatcat ggctattgcg | 60 |
| agtccgctcg ctttttccgt agattctagc ggtgagtatc cgacagtcag cgaaattccg | 120 |
| gtcggggagg tccggcttta ccagattgcc gatggtgttt ggtcgcatat cgcaacgaag | 180 |
| tcgtttgatg gcgcagtcta cccgtccaat ggtctcattg tccgtgatgg tgatgagttg | 240 |
| cttttgattg atacagcgtg gggtgcgaaa aacacagcgg cacttctcgc ggagattgag | 300 |
| aagcaaattg gacttcctgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggtagaggg gagcgagatt cccacgcact ctctagaagg actctcatcg | 480 |
| agcggggacg cagtgcgctt cggtccagta gaactcttct atcctggtgc tgcgcattcg | 540 |
| accgacaact tagttgtgta cgtcccgtct gcgagtgtgc tctatggtgg ttgtgcgatt | 600 |
| tatgagttgt cacgcacgtc tgcggggaac gtggccgatg ccgatctggc tgaatggccc | 660 |
| acctccattg agcggattca acaacactac ccggaagcac agttcgtcat tccggggcac | 720 |
| ggcctgccgg gcggtctaga cttgctcaag cacacaacga atgttgtaaa agcgcacaca | 780 |
| aatcgctcag tcgttgagta g | 801 |
| SEQ ID NO: 58 | |
| atgttcaaac ttttgagtaa gttattggtc tatttgaccg cgtctatcat ggctattgcg | 60 |
| agtccgctcg ctttttccgt agattctagc ggtgagtatc cgacagtcag cgaaattccg | 120 |
| gtcggggagg tccggcttta ccagattgcc gatggtgttt ggtcgcatat cgcaacgaag | 180 |
| tcgtttgatg gcgcagtcta cccgtccaat ggtctcattg tccgtgatgg tgatgagttg | 240 |
| cttttgattg atacagcgtg gggtgcgaaa aacacagcgg cacttctcgc ggagattgag | 300 |
| aagcaaattg gacttcctgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggtagaggg gagcgagatt cccacgcact ctctagaagg actctcatcg | 480 |
| agcggggacg cagtgcgctt cggtccagta gaactcttct atcctggtgc tgcgcattcg | 540 |
| accgacaact tagttgtgta cgtcccgtct gcgagtgtgc tctatggtgg ttgtgcgatt | 600 |
| tatgagttgt cacgcacgtc tgcggggaac gtggccgatg ccgatctggc tgaatggccc | 660 |
| acctccattg agcggattca acaacactac ccggaagcac agttcgtcat tccggggcac | 720 |
| ggcctgccgg gcggtctaga cttgctcaag cacacaacga atgttgtaaa agcgcacaca | 780 |
| aatcgctcag tcgttgagta g | 801 |
| SEQ ID NO: 59 | |
| atgttcaaac ttttgagtaa gttattggtc tatttgaccg cgtctatcat ggctattgcg | 60 |
| agtccgctcg ctttttccgt agattctagc ggtgagtatc cgacagtcag cgaaattccg | 120 |
| gtcggggagg tccggcttta ccagattgcc gatggtgttt ggtcgcatat cgcaacgcag | 180 |
| tcgtttgatg gcgcagtcta cccgtccaat ggtctcattg tccgtgatgg tgatgagttg | 240 |
| cttttgattg atacagcgtg gggtgcgaaa aacacagcgg cacttctcgc ggagattgag | 300 |
| aagcaaattg gacttcctgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggtagaggg gagcgagatt cccacgcact ctctagaagg actctcatcg | 480 |
| agcggggacg cagtgcgctt cggtccagta gaactcttct atcctggtgc tgcgcattcg | 540 |
| accgacaact tagttgtgta cgtcccgtct gcgagtgtgc tctatggtgg ttgtgcgatt | 600 |
| tatgagttgt cacgcacgtc tgcggggaac gtggccgatg ccgatctggc tgaatggccc | 660 |
| acctccattg agcggattca acaacactac ccggaagcac agttcgtcat tccggggcac | 720 |
| ggcctgccgg gcggtctaga cttgctcaag cacacaacga atgttgtaaa agcgcacaca | 780 |
| aatcgctcag tcgttgagta g | 801 |
| SEQ ID NO: 60 | |
| atgttcaaac ttttgagtaa gttattggtc tatttgaccg cgtctatcat ggctattgcg | 60 |
| agtccgctcg ctttttccgt agattctagc ggtgagtatc cgacagtcag cgaaattccg | 120 |
| gtcggggagg tccggcttta ccagattgcc gatggtgttt ggtcgcatat cgcaacgcag | 180 |
| tcgtttgatg gcgcagtcta cccgtccaat ggtctcattg tccgtgatgg tgatgagttg | 240 |
| cttttgattg atacagcgtg gggtgcgaaa aacacagcgg cacttctcgc ggagattgag | 300 |
| aagcaaattg gacttcctgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggtagaggg gagcgagatt cccacgcact ctctagaagg actctcatcg | 480 |
| agcggggacg cagtgcgctt cggtccagta gaactcttct atcctggtgc tgcgcattcg | 540 |
| accgacaact tagttgtgta cgtcccgtct gcgagtgtgc tctatggtgg ttgtgcgatt | 600 |
| tatgagttgt cacgcacgtc tgcggggaac gtggccgatg ccgatctggc tgaatggccc | 660 |
| acctccattg agcggattca acaacactac ccggaagcac agttcgtcat tccggggcac | 720 |
| ggcctgccgg gcggtctaga cttgctcaag cacacaacga atgttgtaaa agcgcacaca | 780 |
| aatcgctcag tcgttgagta g | 801 |
| SEQ ID NO: 61 | |
| atgttcaaac ttttgagtaa gttattggtc tatttgaccg cgtctatcat ggctattgcg | 60 |
| agtccgctcg ctttttccgt agattctagc ggtgagtatc cgacagtcag cgaaattccg | 120 |
| gtcggggagg tccggcttta ccagattgcc gatggtgttt ggtcgcatat cgcaacgcag | 180 |
| tcgtttgatg gcgcagtcta cccgtccaat ggtctcattg tccgtgatgg tgatgagttg | 240 |
| cttttgattg atacagcgtg gggtgcgaaa aacacagcgg cacttctcgc ggagattgag | 300 |
| aagcaaattg gacttcctgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggtagaggg gagcgagatt cccacgcact ctctagaagg actctcatcg | 480 |
| agcggggacg cagtgcgctt cggtccagta gaactcttct atcctggtgc tgcgcattcg | 540 |
| accgacaact tagttgtgta cgtcccgtct gcgagtgtgc tctatggtgg ttgtgcgatt | 600 |
| tatgagttgt cacgcacgtc tgcggggaac gtggccgatg ccgatctggc tgaatggccc | 660 |
| acctccattg agcggattca acaacactac ccggaagcac agttcgtcat tccggggcac | 720 |
| ggcctgccgg gcggtctaga cttgctcaag cacacaacga atgttgtaaa agcgcacaca | 780 |
| aatcgctcag tcgttgagta g | 801 |
| SEQ ID NO: 62 | |
| atgttcaaac ttttgagtaa gttattggtc tatttgaccg cgtctatcat ggctattgcg | 60 |
| agtccgctcg ctttttccgt agattctagc ggtgagtatc cgacagtcag cgaaattccg | 120 |
| gtcggggagg tccggcttta ccagattgcc gatggtgttt ggtcgcatat cgcaacgcag | 180 |
| tcgtttgatg gcgcagtcta cccgtccaat ggtctcattg tccgtgatgg tgatgagttg | 240 |
| cttttgattg atacagcgtg gggtgcgaaa aacacagcgg cacttctcgc ggagattgag | 300 |
| aagcaaattg gacttcctgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggtagaggg gaacgagatt cccacgcact ctctagaagg actctcatcg | 480 |
| agcggggacg cagtgcgctt cggtccagta gaactcttct atcctggtgc tgcgcattcg | 540 |
| accgacaact tagttgtgta cgtcccgtct gcgagtgtgc tctatggtgg ttgtgcgatt | 600 |
| catgagttgt cacgcacgtc tgcggggaac gtggccgatg ccgatctggc tgaatggccc | 660 |
| acctccattg agcggattca acaacgctac ccggaagcac agttcgtcat tccggggcac | 720 |
| ggcctgccgg gcggtctaga cttgctcaag cacacaacga atgttgtaaa agcgcacaca | 780 |
| aatcgctcag tcgttgagta g | 801 |
| SEQ ID NO: 63 | |
| atgttcaaac ttttgagtaa gttattggtc tatttgaccg cgtctatcat ggctattgcg | 60 |
| agtccgctcg ctttttccgt agattctagc ggtgagtatc cgacagtcag cgaaattccg | 120 |
| gtcggggagg tccggcttta ccagattgcc gatggtgttt ggtcgcatat cgcaacgcag | 180 |
| tcgtttgatg gcgcagtcta cccgtccaat ggtctcattg tccgtgatgg tgatgagttg | 240 |
| cttttgattg atacagcgtg gggtgcgaaa aacacagcgg cacttctcgc ggagattgag | 300 |
| aagcaaattg gacttcctgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggtagaggg gaacgagatt cccacgcact ctctagaagg actctcatcg | 480 |
| agcggggacg cagtgcgctt cggtccagta gaactcttct atcctggtgc tgcgcattcg | 540 |
| accgacaact tagttgtgta cgtcccgtct gcgagtgtgc tctatggtgg ttgtgcgatt | 600 |
| tatgagttgt cacgcacgtc tgcggggaac gtggccgatg ccgatctggc tgaatggccc | 660 |
| acctccattg agcggattca acaacgctac ccggaagcac agttcgtcat tccggggcac | 720 |
| ggcctgccgg gcggtctaga cttgctcaag cacacaacga atgttgtaaa agcgcacaca | 780 |
| aatcgctcag tcgttgagta g | 801 |
| SEQ ID NO: 64 | |
| atgttcaaac ttttgagtaa gttattggtc tatttgaccg cgtctatgat ggctattgcg | 60 |
| agtccgctcg ctttttccgt agattctagc ggtgagtatc cgacagtcag cgaaattccg | 120 |
| gtcggggagg tccggcttta ccagattgcc gatggtgttt ggtcgcatat cgcaacgcag | 180 |
| tcgtttgatg gcgcagtcta cccgtccaat ggtctcattg tccgtgatgg tgatgagttg | 240 |
| cttttgattg atacagcgtg gggtgcgaaa aacacagcgg cacttctcgc ggagattgag | 300 |
| aagcaaattg gacttcctgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggtagaggg gaacgagatt cccacgcact ctctagaagg actctcatcg | 480 |
| agcggggacg cagtgcgctt cggtccagta gaactcttct atcctggtgc tgcgcattcg | 540 |
| accgacaact tagttgtgta cgtcccgtct gcgagtgtgc tctatggtgg ttgtgcgatt | 600 |
| tatgagttgt cacgcacgtc tgcggggaac gtggccgatg ccgatctggc tgaatggccc | 660 |
| acctccattg agcggattca acaacactac ccggaagcac agttcgtcat tccggggcac | 720 |
| ggcctgccgg gcggtctaga cttgctcaag cacacaacga atgttgtaaa agcgcacaca | 780 |
| aatcgctcag tcgttgagta g | 801 |
| SEQ ID NO: 65 | |
| atgttcaaac ttttgagtaa gttattggtc tatttgaccg cgtctatcat ggctattgcg | 60 |
| agtccgctcg ctttttccgt agattctagc ggtgagtatc cgacagtcag cgaaattccg | 120 |
| gtcggggagg tccggcttta ccagattgcc gatggtgttt ggtcgcatat cgcaacgcag | 180 |
| tcgtttgatg gcgcagtcta cccgtccaat ggtctcattg tccgtgatgg tgatgagttg | 240 |
| cttttgattg atacagcgtg gggtgcgaaa aacacagcgg cacttctcgc ggagattgag | 300 |
| aagcaaattg gacttcctgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggtagaggg gaacgagatt cccacgcact ctctagaagg actctcatcg | 480 |
| agcggggacg cagtgcgctt cggtccagta gaactcttct atcctggtgc tgcgcattcg | 540 |
| accgacaact tagttgtgta cgtcccgtct gcgagtgtgc tctttggtgg ttgtgcgatt | 600 |
| tatgagttgt cacgcacgtc tgcggggaac gtggccgatg ccgatctggc tgaatggccc | 660 |
| acctccattg agcggattca acaacactac ccggaagcac agttcgtcat tccggggcac | 720 |
| ggcctgccgg gcggtctaga cttgctcaag cacacaacga atgttgtaaa agcgcacaca | 780 |
| aatcgctcag tcgttgagta g | 801 |
| SEQ ID NO: 66 | |
| atgttcaaac ttttgagtaa gttattggtc tatttgaccg cgtctatcat ggctattgcg | 60 |
| agtccgctcg ctttttccgt agattctagc ggtgagtatc cgacagtcag cgaaattccg | 120 |
| gtcggggagg tccggcttta ccagattgcc gatggtgttt ggttgcatat cgcaacgcag | 180 |
| tcgtttgatg gcgcagtcta cccgtccaat ggtctcattg tccgtgatgg tgatgagttg | 240 |
| cttttgattg atacagcgtg gggtgcgaaa aacacagcgg cacttctcgc ggagattgag | 300 |
| aagcaaattg gacttcctgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggtagaggg gaacgagatt cccacgcact ctctagaagg actctcatcg | 480 |
| agcggggacg cagtgcgctt cggtccagta gaactcttct atcctggtgc tgcgcattcg | 540 |
| accgacaact tagttgtgta cgtcccgtct gcgagtgtgc tctatggtgg ttgtgcgatt | 600 |
| tatgagttgt cacgcacgtc tgcggggaac gtggccgatg ccgatctggc tgaatggccc | 660 |
| acctccattg agcggattca acaacactac ccggaagcac agttcgtcat tccggggcac | 720 |
| ggcctgccgg gcggtctaga cttgctcaag cacacaacga atgttgtaaa agcgcacaca | 780 |
| aatcgctcag tcgttgagta g | 801 |
| SEQ ID NO: 67 | |
| atgttcaaac ttttgagtaa gttattggtc tatttgaccg cgtctatcat ggctattgcg | 60 |
| agtccgctcg ctttttccgt agattctagc ggtgagtatc cgacagtcag cgaaattccg | 120 |
| gtcggggagg tccggcttta ccagattgcc gatggtgttt ggtcgcatat cgcaacgcag | 180 |
| tcgtttgatg gcgcagtcta cccgtccaat ggtctcattg tccgtgatgg tgatgagttg | 240 |
| cttttgattg atacagcgtg gggtgcgaaa aacacagcgg cacttctcgc ggagattgag | 300 |
| aagcaaattg gacttcctgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggtagaggg gaacgagatt cccacgcact ctctagaagg actctcatcg | 480 |
| agcggggacg cagtgcgctt cggtccagta gaactcttct atcctggtgc tgcgcattcg | 540 |
| accgacaact tagttgtgta cgtcccgtct gcgagtgtgc tctatggtgg ttgtgcgatt | 600 |
| tatgagttgt caagcacgtc tgcggggaac gtggccgatg ccgatctggc tgaatggccc | 660 |
| acctccattg agcggattca acaacactac ccggaagcac agttcgtcat tccggggcac | 720 |
| ggcctgccgg gcggtctaga cttgctcaag cacacaacga atgttgtaaa agcgcacaca | 780 |
| aatcgctcag tcgttgagta g | 801 |
| SEQ ID NO: 68 | |
| atgttcaaac ttttgagtaa gttattggtc tatttgaccg cgtctatcat ggctattgcg | 60 |
| agtccgctcg ctttttccgt agattctagc ggtgagtatc cgacagtcag cgaaattccg | 120 |
| gtcggggagg tccggcttta ccagattgcc gatggtgttt ggtcgcatat cgcaacgcag | 180 |
| tcgtttgatg gcgcagtcta cccgtccaat ggtctcattg tccgtgatgg tgatgagttg | 240 |
| cttttgattg atacagcgtg gggtgcgaaa aacacagcgg cacttctcgc ggagattgag | 300 |
| aagcaaattg gacttcctgt aacgcgtgca gtctccacgc actttcatga cgaccgcgtc | 360 |
| ggcggcgttg atgtccttcg ggcggctggg gtggcaacgt acgcatcacc gtcgatacgc | 420 |
| cggctagccg aggtagaggg gaacgagatt cccacgcact ctctagaagg actctcatcg | 480 |
| agcggggacg cagtgcgctt cggtccagta gaactcttct atcctggtgc tgcgcattcg | 540 |
| accgacaact tagttgtgta cgtcccgtct gcgagtgtgc tctatggtgg ttgtgcgatt | 600 |
| tatgagttgt cacgcacgtc tgcggggaac gtggccgatg ccgatctggc tgaatggccc | 660 |
| acctccattg agcggattca acaacactac ccggaagcac agttcgtcat tccggggcac | 720 |
| ggcctgccgg gcggtctaga cttgctcaag cacacaacga atgttgtaaa agcgcacaca | 780 |
| aatcgctcag tcgttgagta g | 801 |
| SEQ ID NO: 69 | |
| atgttcaaac ttttgagtaa gttattggtc tatttgaccg cgtctatcat ggctattgcg | 60 |
| agtccgctcg ctttttccgt agattctagc ggtgagtatc cgacagtcag cgaaattccg | 120 |
| gtcggggagg tccggcttta ccagattgcc gatggtgttt ggtcgcatat cgcaacgcag | 180 |
| tcgtttgatg gcgcagtcta cccgtccaat ggtctcattg tccgtgatgg tgatgagttg | 240 |
| cttttgattg atacagcgtg gggtgcgaaa aacacagcgg cacttctcgc ggagattgag | 300 |
| aagcaaattg gacttcctgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggtagaggg gaacgagatt cccacgcact ctctagaagg actctcatcg | 480 |
| agcggggacg cagtgcgctt cggtccagta gaactcttct atcctggtgc tgcgcattcg | 540 |
| accgacaact tagttgtgta cgtcccgtct gcgagtgtgc tctatggtgg ttgtgcgatt | 600 |
| tatgagttgt cacgcacgtc tgcggggaac gtggccgatg ccgatctggc tgaatggccc | 660 |
| acctccattg agcggattca acaacactac ccggaagcac agtacgtcat tccggggcac | 720 |
| ggcctgccgg gcggtctaga cttgctcaag cacacaacga atgttgtaaa agcgcacaca | 780 |
| aatcgctcag tcgttgagta g | 801 |
| SEQ ID NO: 70 | |
| atgttcaaac ttttgagtaa gttattggtc tatttgaccg cgtctatcat ggctattgcg | 60 |
| agtccgctcg ctttttccgt agattctagc ggtgagtatc cgacagtcag cgaaattccg | 120 |
| gtcggggagg tccggcttta ccagattgcc gatggtgttt ggtcgcatat cgcaacgcgg | 180 |
| tcgtttgatg gcgcagtcta cccgtccaat ggtctcattg tccgtgatgg tgatgagttg | 240 |
| cttttgattg atacagcgtg gggtgcgaaa aacacagcgg cacttctcgc ggagattgag | 300 |
| aagcaaattg gacttcctgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggtagaggg gagcgagatt cccacgcact ctctagaagg actctcatcg | 480 |
| agcggggacg cagtgcgctt cggtccagta gaactcttct atcctggtgc tgcgcattcg | 540 |
| accgacaact tagttgtgta cgtcccgtct gcgagtgtgc tctatggtgg ttgtgcgatt | 600 |
| tatgagttgt cacgcacgtc tgcggggaac gtggccgatg ccgatctggc tgaatggccc | 660 |
| acctccattg agcggattca acaacactac ccggaagcac agttcgtcat tccggggcac | 720 |
| ggcctgccgg gcggtctaga cttgctcaag cacacaacga atgttgtaaa agcgcacaca | 780 |
| aatcgctcag tcgttgagta g | 801 |
| SEQ ID NO: 71 | |
| atgttcaaac ttttgagtaa gttattggtc tatttgaccg cgtctatcat ggctattgcg | 60 |
| agtccgctcg ctttttccgt agattctagc ggtgagtatc cgacagtcag cgaaattccg | 120 |
| gtcggggagg tccggcttta ccagattgcc gatggtgttt ggtcgcatat cgcaacgcgg | 180 |
| tcgtttgatg gcgcagtcta cccgtccaat ggtctcattg tccgtgatgg tgatgagttg | 240 |
| cttttgattg atacagcgtg gggtgcgaaa aacacagcgg cacttctcgc ggagattgag | 300 |
| aagcaaattg gacttcctgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggtagaggg gagcgagatt cccacgcact ctctagaagg actctcatcg | 480 |
| agcggggacg cagtgcgctt cggtccagta gaactcttct atcctggtgc tgcgcattcg | 540 |
| accgacaact tagttgtgta cgtcccgtct gcgagtgtgc tctatggtgg ttgtgcgatt | 600 |
| tatgagttgt cacgcacgtc tgcggggaac gtggccgatg ccgatctggc tgaatggccc | 660 |
| acctccattg agcggattca acaacactac ccggaagcac agttcgtcat tccggggcac | 720 |
| ggcctgccgg gcggtctaga cttgctcaag cacacaacga atgttgtaaa agcgcacaca | 780 |
| aatcgctcag tcgttgagta g | 801 |
| SEQ ID NO: 72 | |
| atgttcaaac ttttgagtaa gttattggtc tatttgaccg cgtctatcat ggctattgcg | 60 |
| agtccgctcg ctttttccgt agattctagc ggtgagtatc cgacagtcag cgaaattccg | 120 |
| gtcggggagg tccggcttta ccagattgcc gatggtgttt ggtcgcatat cgcaacgcag | 180 |
| tcgtttgatg gcgcagtcta cccgtccaat ggtctcattg tccgtgatgg tgatgagttg | 240 |
| cttttgattg atacagcgtg gggtgcgaaa aacacagcgg cacttctcgc ggagattgag | 300 |
| aagcaaattg gacttcctgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggtagaggg gaacgagatt cccacgcact ctctagaagg actctcatcg | 480 |
| agcggggacg cagtgcgctt cggtccagta gaactcttct atcctggtgc tgcgcattcg | 540 |
| accgacaact tagttgtgta cgtcccgtct gcgagtgtgc tctatggtgg ttgtgcgatt | 600 |
| tatgagttgt cactcacgtc tgcggggaac gtggccgatg ccgatctggc tgaatggccc | 660 |
| acctccattg agcggattca acaacactac ccggaagcac agttcgtcat tccggggcac | 720 |
| ggcctgccgg gcggtctaga cttgctcaag cacacaacga atgttgtaaa agcgcacaca | 780 |
| aatcgctcag tcgttgagta g | 801 |
| SEQ ID NO: 73 | |
| atgttcaaac ttttgagtaa gttattggtc tatttgaccg cgtctatcat ggctattgcg | 60 |
| agtccgctcg ctttttccgt agattctagc ggtgagtatc cgacagtcag cgaaattccg | 120 |
| gtcggggagg tccggcttta ccagattgcc gatggtgttt ggtcgcatat cgcaacgcgg | 180 |
| tcgtttgatg gcgcagtcta cccgtccaat ggtctcattg tccgtgatgg tgatgagttg | 240 |
| cttttgattg atacagcgtg gggtgcgaaa aacacagcgg cacttctcgc ggagattgag | 300 |
| aagcaaattg gacttcctgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggtagaggg gaacgagatt cccacgcact ctctagaagg actctcatcg | 480 |
| agcggggacg cagtgcgctt cggtccagta gaactcttct atcctggtgc tgcgcattcg | 540 |
| accgacaact tagttgtgta cgtcccgtct gcgagtgtgc tctatggtgg ttgtgcgatt | 600 |
| tatgagttgt cacgcacgtc tgcggggaac gtggccgatg ccgatctggc tgaatggccc | 660 |
| acctccattg agcggattca acaacactac ccggaagcac agttcgtcat tccggggcac | 720 |
| ggcctgccgg gcggtctaga cttgctcaag cacacaacga atgttgtaaa agcgcacaca | 780 |
| aatcgctcag tcgttgagta g | 801 |
| SEQ ID NO: 74 | |
| atgttcaaac ttttgagtaa gttattggtc tatttgaccg cgtctatcat ggctattgcg | 60 |
| agtccgctcg ctttttccgt agattctagc ggtgagtatc cgacagtcaa cgaaattccg | 120 |
| gtcggggagg tccggcttta ccagattgcc gatggtgttt ggtcgcatat cgcaacgcag | 180 |
| tcgtttgatg gcgcagtcta cccgtccaat ggtctcattg tccgtgatgg tgatgagttg | 240 |
| cttttgattg atacagcgtg gggtgcgaaa aacacagcgg cacttctcgc ggagattgag | 300 |
| aagcaaattg gacttcctgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggtagaggg gaacgagatt cccacgcact ctctagaagg actctcatcg | 480 |
| agcggggacg cagtgcgctt cggtccagta gaactcttct atcctggtgc tgcgcattcg | 540 |
| accgacaact tagttgtgta cgtcccgtct gcgagtgtgc tctatggtgg ttgtgcgatt | 600 |
| tatgagttgt cacgcacgtc tgcggggaac gtggccgatg ccgatctggc tgaatggccc | 660 |
| acctccattg agcggattca acaacactac ccggaagcac agttcgtcat tccggggcac | 720 |
| ggcctgccgg gcggtctaga cttgctcaag cacacaacga atgttgtaaa agcgcacaca | 780 |
| aatcgctcag tcgttgagta g | 801 |
| SEQ ID NO: 75 | |
| atgttcaaac ttttgagtaa gttattggtc tatttgaccg cgtctatcat ggctattgcg | 60 |
| agtccgctcg ctttttccgt agattctagc ggtgagtatc cgacagtcag cgaaattccg | 120 |
| gtcggggagg tccggcttta ccagattgcc gatggtgttt ggtcgcatat cgcaacgcag | 180 |
| tcgtttgatg gcgcagtcta cccgtccaat ggtctcattg tccgtgatgg tgatgagttg | 240 |
| cttttgattg atacagcgtg gggtgcgaaa aacacagcgg cacttctcgc ggagattgag | 300 |
| aagcaaattg gacttcctgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggtagaggg gaacgagatt cccacgcact ctctagaagg actctcatcg | 480 |
| agcggggacg cagtgcgctt cggtccagta gaactcttct atcctggtgc tgcgcattcg | 540 |
| accgacaact tagttgtgta cgtcccgtct gcgagtgtgc tctatggtgg ttgtgcgatt | 600 |
| tatgagttgt cacgcacgtc tgcggggaac gtggccgatg ccgatctggc tgaatggccc | 660 |
| acctccattg agcggattca acaacactac ccggaagcac agttcgtcat tccggggcac | 720 |
| ggcctgccgg gcggtctaga cttgctcaag cacacaacga atgttgtaaa agcgcacaca | 780 |
| aatcgctcag tcgttgagta g | 801 |
| SEQ ID NO: 76 | |
| atgttcaaac ttttgagtaa gttattggtc tatttgaccg cgtctatcat ggctattgcg | 60 |
| agtccgctcg ctttttccgt agattctagc ggtgagtatc cgacagtcag cgaaattccg | 120 |
| gtcggggagg tccggcttta ccagattgcc gatggtgttt ggtcgcatat cgcaacgcag | 180 |
| tcgtttgatg gcgcagtcta cccgtccaat ggtctcattg tccgtgatgg tgatgagttg | 240 |
| cttttgattg atacagcgtg gggtgcgaaa aacacagcgg cacttctcgc ggagattgag | 300 |
| aagcaaattg gacttcctgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggtagaggg gaacgagatt cccacgcact ctctagaagg actctcatcg | 480 |
| agcggggacg cagtgcgctt cggtccagta gaactcttct atcctggtgc tgcgcattcg | 540 |
| accgacaact tagttgtgta cgtcccgtct gcgagtgtgc tctatggtgg ttgtgcgatt | 600 |
| tatgagttgt cacgcacgtc tgcggggaac gtggccgatg ccgatctggc tgaatggccc | 660 |
| acctccattg agcggattca acaacactac ccggaagcac agttcgtcat tccggggcac | 720 |
| ggcctgccgg gcggtctaga cttgctcaag cacacaacga atgttgtaaa agcgcacaca | 780 |
| aatcgctcag tcgttgagta g | 801 |
| SEQ ID NO: 77 | |
| atgttaaaag ttattagtag tttattggtc tatttgaccg cgtctgtcat ggctgtagct | 60 |
| agtccgttag cccattccgg ggagccgagt ggtgagtatc cgacagtcaa cgaaattccg | 120 |
| gtcggagagg tccggcttta ccagattgct gatggtgttt ggtcgcatat cgcaacgcag | 180 |
| tcgtttgatg gcgcggtcta cccatccaat ggtctcattg tccgtgatgg tgatgagttg | 240 |
| cttttgattg atacagcgtg gggtgcgaaa aacacagcgg cccttctcgc ggagattgag | 300 |
| aagcaaattg gacttcccgt aacgcgtgca gtctccacgc actttcatga cgaccgcgtc | 360 |
| ggcggcgttg atgtccttcg gaaggctgga gtggcaacgt acgcatcacc gtcgacacgc | 420 |
| cggctagccg aggcagaggg gaacgagatt cccacgcact ctctagaagg actctcatcg | 480 |
| agcggggacg cagtgcgctt cggtccagta gagctcttct atcccggtgc tgcgcattcg | 540 |
| accgacaatc tggttgtata cgtcccgtca gcgaacgtgc tatacggtgg ttgtgccgtt | 600 |
| cttgcgttgt cacgcacgtc tgcggggaac gtggccgatg ccgatctggc tgaatggccc | 660 |
| acctccgttg agcggattca aaaacactac ccggaagcag aggtcgtcat tcccgggcac | 720 |
| ggtctaccgg gcggtctaga cttgctccag cacacagcga acgttgtcac agcacacaaa | 780 |
| aatcgctcag tcgttgagta g | 801 |
| SEQ ID NO: 78 | |
| atgttaaaag ttattagtag tttattggtc tacatgaccg cgtctgtcat ggctgtagct | 60 |
| agtccgttag cccattccgg ggagccgagt ggtgagtatc cgacagtcaa cgaaattccg | 120 |
| gtcggagagg tccggcttta ccagattgct gatggtgttt ggtcgcatat cgcaacgcag | 180 |
| tcgtttgatg gcgcggtcta cccatccaat ggtctcattg tccgtgatgg tgatgagttg | 240 |
| cttttgattg atacagcgtg gggtgcgaaa aacacagcgg cccttctcgc ggagattgag | 300 |
| aagcaaattg gacttcccgt aacgcgtgca gtctccacgc actttcatga cgaccgcgtc | 360 |
| ggcggcgttg atgtccttcg gaaggctgga gtggcaacgt acgcatcacc gtcgacacgc | 420 |
| cggctagccg aggcagaggg gaacgagatt cccacgcact ctctagaagg actctcatcg | 480 |
| agcggggacg cagtgcgctt cggtccagta gagctcttct atcccggtgc tgcgcattcg | 540 |
| accgacaatc tggttgtata cgtcccgtca gcgaacgtgc tatacggtgg ttgtgccgtt | 600 |
| cttgcgttgt cacgcacgtc tgcggggaac gtggccgatg ccgatctggc tgaatggccc | 660 |
| acctccgttg agcggattca aaaacactac ccggaagcag aggtcgtcat tcccgggcac | 720 |
| ggtctaccgg gcggtctaga cttgctccag cacacagcga acgttgtcac agcacacaaa | 780 |
| aatcgctcag tcgccgagta g | 801 |
| SEQ ID NO: 79 | |
| atgttaaaag ttattagtag tttattggtc tacatgaccg cgtctgtcat ggctgtcgca | 60 |
| agtccgttag cccattccgg ggagccgagt ggtgagtatc cgacagtcaa cgaaattccg | 120 |
| gtcggagagg tccgacttta ccagattgcc gatggtgttt ggtcgcatat cgcaacgcag | 180 |
| tcgtttgatg gcgcggtcta cccgtccaat ggtctcattg tccgtgatgg tgatgagttg | 240 |
| cttttgattg atacagcgtg gggtgcgaaa aacacagcgg cacttctcgc ggagattgaa | 300 |
| aagcaaattg gacttcccgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggcagaggg gaacgagatt cccacgcatt ctctagaagg actctcatcg | 480 |
| agcggggacg cagtgcgctt cggtccagta gagctcttct atcctggtgc tgcgcattcg | 540 |
| accgacaatc tggttgtata cgtcccgtca gcgaacgtgc tatacggtgg ttgtgccgtt | 600 |
| catgagttgt caagcacgtc tgcggggaac gtggccgatg ccgatctggc tgaatggccc | 660 |
| acctccattg agcggattca acaacactac ccggaagcac agttcgtcat tccggggcac | 720 |
| ggcctgccgg gcggtctaga cttgctcaag cacacaacga atgttgtaaa agcgcacaca | 780 |
| aatcgctcag tcgttgagta g | 801 |
| SEQ ID NO: 80 | |
| atgttaaaag ttattagtag tttattggtc tacatgaccg cgtctgtcat ggctgtcgca | 60 |
| agtccgttag cccattccgg ggagccgagt ggtgagtatc cgacagtcaa cgaaattccg | 120 |
| gtcggagagg tccgacttta ccagattgcc gatggtgttt ggtcgcatat cgcaacgcag | 180 |
| tcgtttgatg gcgcggtcta cccgtccaat ggtctcattg tccgtgatgg tgatgagttg | 240 |
| cttttgattg atacagcgtg gggtgcgaaa aacacagcgg cacttctcgc ggagattgaa | 300 |
| aagcaaattg gacttcccgt aacgcgtgca atctccacgc actttcatga cgaccgcgtc | 360 |
| ggcggcgttg atgtccttcg ggcggctggg gtggcaacgt acgcatcacc gtcgacacgc | 420 |
| cggctagccg aggcagaggg gaacgagatt cccacgcatt ctctagaagg actctcatcg | 480 |
| agcggggacg cagtgcgctt cggtccagta gagctcttct atcctggtgc tgcgcattcg | 540 |
| accgacaatc tggttgtata cgtcccgtca gcgaacgtgc tatacggtgg ttgtgccgtt | 600 |
| catgagttgt caagcacgtc tgcggggaac gtggccgatg ccgatctggc tgaatggccc | 660 |
| acctccgttg agcggattca aaaacactac ccggaagcag aggtcgtcat tcccgggcac | 720 |
| ggtctaccgg gcggtctaga cttgctccag cacacagcga acgttgtcaa agcacacaaa | 780 |
| aatcgctcag tcgccgagta g | 801 |
| SEQ ID NO: 81 | |
| atgttaaaag ttattagtag tttattggtc tacatgaccg cgtctgtcat ggctgtcgca | 60 |
| agtccgttag cccattccgg ggagccgagt ggtgagtatc cgacagtcaa cgaaattccg | 120 |
| gtcggagagg tccgacttta ccagattgcc gatggtgttt ggtcgcatat ctcaacgcag | 180 |
| tcgtttgatg gcgcggtcta cccgtccaat ggtctcattg tccgtgatgg tgatgagttg | 240 |
| cttttgattg atacagcgtg gggtgcgaaa aacacagcgg cacttctcgc ggagattgaa | 300 |
| aagcaaattg gacttcccgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggcagaggg gaacgagatt cccacgcatt ctctagaagg actctcatcg | 480 |
| agcggggacg cagtgcgctt cggtccagta gagctcttct atcctggtgc tgcgcattcg | 540 |
| accgacaatc tggttgtata cgtcccgtca gcgaacgtgc tatacggtgg ttgtgccgtt | 600 |
| catgagttgt cacgcacgtc tgcggggaac gtggccgatg ccgatctggc tgaatggccc | 660 |
| acctccgttg agcggattca aaaacactac ccggaagcag aggtcgtcat tcccgggcac | 720 |
| ggtctaccgg gcggtctaga cttgctccag cacacagcga acgttgtcaa agcacacaaa | 780 |
| aatcgctcag tcgccgagta g | 801 |
| SEQ ID NO: 82 | |
| atgttaaaag ttattagtag tttattggtc tacatgaccg cgtctgtcat ggctgtcgca | 60 |
| agtccgttag cccattccgg ggagccgagt ggtgagtatc cgacagtcaa cgaaattccg | 120 |
| gtcggagagg tccgacttta ccagattgcc gatggtgttt ggtcgcatat ctcaacgcag | 180 |
| tcgtttgatg gcgcggtcta cccgtccaat ggtctcattg tccgtgatgg tgatgagttg | 240 |
| cttttgattg atacagcgtg gggtgcgaaa aacacagcgg cacttctcgc ggagattgaa | 300 |
| aagcaaattg gacttcccgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggcagaggg gaacgagatt cccacgcatt ctctagaagg actctcatcg | 480 |
| agcggggacg cagtgcgctt cggtccagta gagctcttct atcctggtgc tgcgcattcg | 540 |
| accgacaatc tggttgtata cgtcccgtca gcgaacgtgc tatacggtgg ttgtgccgtt | 600 |
| catgagttgt caagcacgtc tgcggggaac gtggccgatg ccgatctggc tgaatggccc | 660 |
| acctccgttg agcggattca aaaacactac ccggaagcag aggtcgtcat tcccgggcac | 720 |
| ggtctaccgg gcggtctaga cttgctccag cacacagcga acgttgtcaa agcacacaaa | 780 |
| aatcgctcag tcgccgagta g | 801 |
| SEQ ID NO: 83 | |
| atgttaaaag ttattagtag tttattggtc tacatgaccg cgtctgtcat ggctgtcgca | 60 |
| agtccgttag cccattccgg ggagccgagt ggtgagtatc cgacagtcaa cgaaattccg | 120 |
| gtcggagagg tccgacttta ccagattgcc gatggtgttt ggtcgcatat ctcaacgcag | 180 |
| tcgtttgatg gcgcggtcta cccgtccaat ggtctcattg tccgtgatgg tgatgagttg | 240 |
| cttttgattg atacagcgtg gggtgcgaaa aacacagcgg cacttctcgc ggagattgaa | 300 |
| aagcaaattg gacttcccgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggcagaggg gaacgagatt cccacgcatt ctctagaagg actctcatcg | 480 |
| agcggggacg cagtgcgctt cggtccagta gagctcttct atcctggtgc tgcgcattcg | 540 |
| accgacaatc tggttgtata cgtcccgtca gcgaacgtgc tatacggtgg ttgtgccgtt | 600 |
| catgagttgt caagcacgtc tgcggggaac gtggccgatg ccgatctggc tgaatggccc | 660 |
| acctccgttg agcggattca aaaacactac ccggaagcag aggtcgtcat tcccgggcac | 720 |
| ggtctaccgg gcggtctaga cttgctccag cacacagcga acgttgtcaa agcacacaaa | 780 |
| aatcgctcag tcgccgagta g | 801 |
| SEQ ID NO: 84 | |
| atgttaaaag ttattagtag tttattggtc tacatgaccg cgtctgtcat ggctgtcgca | 60 |
| agtccgttag cccattccgg ggagccgagt ggtgagtatc cgacagtcaa cgaaattccg | 120 |
| gtcggagagg tccgacttta ccagattgcc gatggtgttt ggtcgcatat cgcaacgcag | 180 |
| tcgtttgatg gcgcggtcta cccgtccaat ggtctcattg tccgtgatgg tgatgagttg | 240 |
| cttttgattg atacagcgtg gggtgcgaaa aacacagcgg cacttctcgc ggagattgaa | 300 |
| aagcaaattg gacttcccgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggcagaggg gaacgagatt cccacgcatt ctctagaagg actctcatcg | 480 |
| agcggggacg cagtgcgctt cggtccagta gagctcttct atcctggtgc tgcgcattcg | 540 |
| accgacaatc tggttgtata cgtcccgtca gcgaacgtgc tatacggtgg ttgtgccgtt | 600 |
| cttgagttgt caagcacgtc tgcggggaac gtggccgatg ccgatctggc tgaatggccc | 660 |
| acctccgttg agcggattca aaaacactac ccggaagcag aggtcgtcat tcccgggcac | 720 |
| ggtctaccgg gcggtctaga cttgctccag cacacagcga acgttgtcaa agcacacaaa | 780 |
| aatcgctcag tcgccgagta g | 801 |
| SEQ ID NO: 85 | |
| atgttaaaag ttattagtag tttattggtc tacatgaccg cgtctgtcat ggctgtcgca | 60 |
| agtccgttag cccattccgg ggagccgagt agtgagtatc cgacagtcaa cgaaattccg | 120 |
| gtcggagagg tccgacttta ccagattgcc gatggtgttt ggtcgcatat cgcaacgcag | 180 |
| tcgtttgatg gcgcggtcta cccgtccaat ggtctcattg tccgtgatgg tgatgagttg | 240 |
| cttttgattg atacagcgtg gggtgcgaaa aacacagcgg cacttctcgc ggagattgaa | 300 |
| aagcaaattg gacttcccgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggcagaggg gaacgagatt cccacgcatt ctctagaagg actctcatcg | 480 |
| agcggggacg cagtgcgctt cggtccagta gagctcttct atcctggtgc tgcgcattcg | 540 |
| accgacaatc tggttgtata cgtcccgtca gcgaacgtgc tatacggtgg ttgtgccgtt | 600 |
| catgagttgt cacgcacgtc tgcggggaac gtggccgatg ccgatctggc tgaatggccc | 660 |
| acctccgttg agcggattca aaaacactac ccggaagcag aggtcgtcat tcccgggcac | 720 |
| ggtctaccgg gcggtctaga cttgctccag cacacagcga acgttgtcaa agcacacaaa | 780 |
| aatcgctcag tcgccgagta g | 801 |
| SEQ ID NO: 86 | |
| atgttaaaag ttattagtag tttattggtc tacatgaccg cgtctgtcat ggctgtcgca | 60 |
| agtccgttag cccattccgg ggagccgagt agtgagtatc cgacagtcaa cgaaattccg | 120 |
| gtcggagagg tccgacttta ccagattgcc gatggtgttt ggtcgcatat cgcaacgcag | 180 |
| tcgtttgatg gcgcggtcta cccgtccaat ggtctcattg tccgtgatgg tgatgagttg | 240 |
| cttttgattg atacagcgtg gggtgcgaaa aacacagcgg cacttctcgc ggagattgaa | 300 |
| aagcaaattg gacttcccgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggcagaggg gaacgagatt cccacgcatt ctctagaagg actctcatcg | 480 |
| agcggggacg cagtgcgctt cggtccagta gagctcttct atcctggtgc tgcgcattcg | 540 |
| accgacaatc tggttgtata cgtcccgtca gcgaacgtgc tatacggtgg ttgtgccgtt | 600 |
| catgagttgt cacgcacgtc tgcggggaac gtggccgatg ccgatctggc tgaatggccc | 660 |
| acctccgttg agcggattca aaaacactac ccggaagcag aggtcgtcat tcccgggcac | 720 |
| ggtctaccgg gcggtctaga cttgctccag cacacagcga acgttgtcaa agcacacaaa | 780 |
| aatcgctcag tcgccgagta g | 801 |
| SEQ ID NO: 87 | |
| atgttaaaag ttattagtag tttattggtc tacatgaccg cgtctgtcat ggctgtcgca | 60 |
| agtccgttag cccattccgg ggagccgagt ggtgagtatc cgacagtcaa cgaaattccg | 120 |
| gtcggagagg tccgacttta ccagattgcc gatggtgttt ggtcgcatat cgcaacgcag | 180 |
| tcgtttgatg gcgcggtcta cccgtccaat ggtctcattg tccgtgatgg tgatgagttg | 240 |
| cttttgattg atacagcgtg gggtgcgaaa aacacagcgg cacttctcgc ggagattgaa | 300 |
| aagcaaattg gacttcccgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggcagaggg gaacgagatt cccacgcatt ctctagaagg actctcatcg | 480 |
| agcggggacg cagtgcgctt cggtccagta gagctcttct atcctggtgc tgcgcattcg | 540 |
| accgacaatc tggttgtata cgtcccgtca gcgaaagtgc tatacggtgg ttgtgccgtt | 600 |
| catgagttgt cacgcacgtc tgcggggaac gtggccgatg ccgatctggc tgaatggccc | 660 |
| acctccgttg agcggattca aaaacactac ccggaagcag aggtcgtcat tcccgggcac | 720 |
| ggtctaccgg gcggtctaga cttgctccag cacacagcga acgttgtcaa agcacacaaa | 780 |
| aatcgctcag tcgccgagta g | 801 |
| SEQ ID NO: 88 | |
| atgttaaaag ttattagtag tttattggtc tacatgaccg cgtctgtcat ggctgtcgca | 60 |
| agtccgttag cccattccgg ggagccgagt ggtgagtatc cgacagtcaa cgaaattccg | 120 |
| gtcggagagg tccgacttta ccagattgcc gatggtgttt ggtcgcatat cgcaacgcag | 180 |
| tcgtttgatg gcgcggtcta cccgtccaat ggtctcattg tccgtgatgg tgatgagttg | 240 |
| cttttgattg atacagcgtg gggtgcgaaa aacacagcgg cacttctcgc ggagattgaa | 300 |
| aagcaaattg gacttcccgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggcagaggg gaacgagatt cccacgcatt ctctagaagg actctcatcg | 480 |
| agcggggacg cagtgcgctt cggtccagta gagctcttct atcctggtgc tgcgcattcg | 540 |
| accgacaatc tggttgtata cgtcccgtca gcgaaagtgc tatacggtgg ttgtgccgtt | 600 |
| catgagttgt cacgcacgtc tgcggggaac gtggccgatg ccgatctggc tgaatggccc | 660 |
| acctccgttg agcggattca aaaacactac ccggaagcag aggtcgtcat tcccgggcac | 720 |
| ggtctaccgg gcggtctaga cttgctccag cacacagcga acgttgtcaa agcacacaaa | 780 |
| aatcgctcag tcgccgagta g | 801 |
| SEQ ID NO: 89 | |
| atgttaaaag ttattagtag tttattggtc tacatgaccg cgtctgtcat ggctgtcgca | 60 |
| agtccgttag cccattccgg ggagccgagt ggtgagtatc cgacagtcaa cgaaattccg | 120 |
| gtcggagagg tccgacttta ccagattgcc gatggtgttt ggtcgcatat cgcaacgcag | 180 |
| tcgtttgatg gcgcggtcta cccgtccaat ggtctcattg tccgtgatgg tgatgagttg | 240 |
| cttttgattg atacagcgtg gggtgcgaaa aacacagcgg cacttctcgc ggagattgaa | 300 |
| aagcaaattg gacttcccgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggcagaggg gaacgagatt cccacgcatt ctctagaagg actctcatcg | 480 |
| agcggggacg cagtgcgctt cggtccagta gagctcttct atcctggtgc tgcgcattcg | 540 |
| accgacaatc tggttgtata cgtcccgtca gcgaacgtgc tatacggtgg ttgtgccgtt | 600 |
| cttgagttgt cacgcacgtc tgcggggaac gtggccgatg ccgatctggc tgaatggccc | 660 |
| acctccgttg agcggattca aaaacactac ccggaagcag aggtcgtcat tcccgggcac | 720 |
| ggtctaccgg gcggtctaga cttgctccag cacacagcga acgttgtcaa agcacacaaa | 780 |
| aatcgctcag tcgccgagta g | 801 |
| SEQ ID NO: 90 | |
| atgttaaaag ttattagtag tttattggtc tacatgaccg cgtctgtcat ggctgtcgca | 60 |
| agtccgttag cccattccgg ggagccgagt ggtgagtatc cgacagtcaa cgaaattccg | 120 |
| gtcggagagg tccgacttta ccagattgcc gatggtgttt ggtcgcatat cgcaacgcag | 180 |
| tcgtttgatg gcgcggtcta cccgtccaat ggtctcattg tccgtgatgg tgatgagttg | 240 |
| cttttgattg atacagcgtg gggtgcgaaa aacacagcgg cacttctcgc ggagattgaa | 300 |
| aagcaaattg gacttcccgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggcagaggg gaacgagatt cccacgcatt ctctagaagg actctcatcg | 480 |
| agcggggacg cagtgcgctt cggtccagta gagctcttct atcctggtgc tgcgcattcg | 540 |
| accgacaatc tggttgtata cgtcccgtca gcgaacgtgc tatacggtgg ttgtgccgtt | 600 |
| catgagttgt cacgcacgtc tgcggggaac gtggccgatg ccgatctggc tgaatggccc | 660 |
| acctccgttg agcggattca aaaacactac ccggaagcag aggtcgtcat tcccgggcac | 720 |
| ggtctaccgg gcggtctaga cttgctccag cacacagcga acgttgtcaa agcacacaaa | 780 |
| aatcgctcag tcgccgagta g | 801 |
| SEQ ID NO: 91 | |
| atgttaaaag ttattagtag tttattggtc tacatgaccg cgtctgtcat ggctgtcgca | 60 |
| agtccgttag cccattccgg ggagccgagt ggtgagtatc cgacagtcaa cgaaattccg | 120 |
| gtcggagagg tccgacttta ccagattgcc gatggtgttt ggtcgcatat cgcaacgcag | 180 |
| tcgtttgatg gcgcggtcta cccgtccaat ggtctcattg tccgtgatgg tgatgagttg | 240 |
| cttttgattg atacagcgtg gggtgcgaaa aacacagcgg cacttctcgc ggagattgaa | 300 |
| aagcaaattg gacttcccgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggcagaggg gaacgagatt cccacgcatt ctctagaagg actctcatcg | 480 |
| agcggggacg cagtgcgctt cggtccagta gagctcttct atcctggtgc tgcgcattcg | 540 |
| accgacaatc tggttgtata cgtcccgtca gcgaacgtgc tatacggtgg ttgtgccgtt | 600 |
| catgagttgt cacgcacgtc tgcggggaac gtggccgatg ccgatctggc tgaatggccc | 660 |
| acctccgttg agcggattca aaaacactac ccggaagcag aggtcgtcat tcccgggcac | 720 |
| ggtctaccgg gcggtctaga cttgctccag cacacagcga acgttgtcaa agcacacaaa | 780 |
| aatcgctcag tcgccgagta g | 801 |
| SEQ ID NO: 92 | |
| atgttaaaag ttattagtag tttattggtc tacatgaccg cgtctgtcat ggctgtcgca | 60 |
| agtccgttag cccattccgg ggagccgagt ggtgagtatc cgacagtcaa cgaaattccg | 120 |
| gtcggagagg tccgacttta ccagattgcc gatggtgttt ggtcgcatat cgcaacgcag | 180 |
| tcgtttgatg gcgcggtcta cccgtccaat ggtctcattg tccgtgatgg tgatgagttg | 240 |
| cttttgattg atacagcgtg gggtgcgaaa aacacagcgg cacttctcgc ggagattgaa | 300 |
| aagcaaattg gacttcccgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggcagaggg gaacgagatt cccacgcatt ctctagaagg actctcatcg | 480 |
| agcggggacg cagtgcgctt cggtccagta gagctcttct atcctggtgc tgcgcattcg | 540 |
| accgacaatc tggttgtata cgtcccgtca gcgaacgtgc tatacggtgg ttgtgccgtt | 600 |
| catgagttgt cacgcacgtc tgcggggaac gtggccgatg ccgatctggc tgaatggccc | 660 |
| acctccgttg agcggattca aaaacactac ccggaagcag aggtcgtcat tcccgggcac | 720 |
| ggtctaccgg gcggtctaga cttgctccag cacacagcga acgttgtcaa agcacacaaa | 780 |
| aatcgctcag tcgccgagta g | 801 |
| SEQ ID NO: 93 | |
| atgttaaaag ttattagtag tttattggtc tacatgaccg cgtctgtcat ggctgtcgca | 60 |
| agtccgttag cccattccgg ggagccgagt ggtgagtatc cgacagtcaa cgaaattccg | 120 |
| gtcggagagg tccgacttta ccagattgcc gatggtgttt ggtcgcatat cgcaacgcag | 180 |
| tcgtttgatg gcgcggtcta cccgtccaat ggtctcattg tccgtgatgg tgatgagttg | 240 |
| cttttgattg atacagcgtg gggtgcgaaa aacacagcgg cacttctcgc ggagattgaa | 300 |
| aagcaaattg gacttcccgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggcagaggg gaacgagatt cccacgcatt ctctagaagg actctcatcg | 480 |
| agcggggacg cagtgcgctt cggtccagta gagctcttct atcctggtgc tgcgcattcg | 540 |
| accgacaatc tggttgtata cgtcccgtca gcgaaagtgc tatacggtgg ttgtgccgtt | 600 |
| catgagttgt caagcacgtc tgcggggaac gtggccgatg ccgatctggc tgaatggccc | 660 |
| acctccgttg agcggattca aaaacgctac ccggaagcag aggtcgtcat tcccgggcac | 720 |
| ggtctaccgg gcggtctaga cttgctccag cacacagcga acgttgtcaa agcacacaaa | 780 |
| aatcgctcag tcgccgagta g | 801 |
| SEQ ID NO: 94 | |
| atgttaaaag ttattagtag tttattggtc tacatgaccg cgtctgtcat ggctgtcgca | 60 |
| agtccgttag cccattccgg ggagccgagt ggtgagtatc cgacagtcaa cgaaattccg | 120 |
| gtcggagagg tccgacttta ccagattgcc gatggtgttt ggtcgcatat cgcaacgcag | 180 |
| tcgtttgatg gcgcggtcta cccgtccaat ggtctcattg tccgtgatgg tgatgagttg | 240 |
| cttttgattg atacagcgtg gggtgcgaaa aacacagcgg cacttctcgc ggagattgaa | 300 |
| aagcaaattg gacttcccgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggcagaggg gaacgagatt cccacgcatt ctctagaagg actctcatcg | 480 |
| agcggggacg cagtgcgctt cggtccagta gagctcttct atcctggtgc tgcgcattcg | 540 |
| accgacaatc tggttgtata cgtcccgtca gcgaacgtgc tattcggtgg ttgtgccgtt | 600 |
| catgagttgt caagcacgtc tgcggggaac gtggccgatg ccgatctggc tgaatggccc | 660 |
| acctccgttg agcggattca aaaacactac ccggaagcag aggtcgtcat tcccgggcac | 720 |
| ggtctaccgg gcggtctaga cttgctccag cacacagcga acgttgtcaa agcacacaaa | 780 |
| aatcgctcag tcgccgagta | 800 |
| SEQ ID NO: 95 | |
| atgttaaaag ttattagtag tttattggtc tacatgaccg cgtctgtcat ggctgtcgca | 60 |
| agtccgttag cccattccgg ggagccgagt ggtgagtatc cgacagtcaa cgaaattccg | 120 |
| gtcggagagg tccgacttta ccagattgcc gatggtgttt ggtcgcatat cgcaacgcag | 180 |
| tcgtttgatg gcgcggtcta cccgtccaat ggtctcattg tccgtgatgg tgatgagttg | 240 |
| cttttgattg atacagcgtg gggtgcgaaa aacacagcgg cacttctcgc ggagattgaa | 300 |
| aagcaaattg gacttcccgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggcagaggg gaacgagatt cccacgcatt ctctagaagg actctcatcg | 480 |
| agcggggacg cagtgcgctt cggtccagta gagctcttct atcctggtgc tgcgcattcg | 540 |
| accgacaatc tggttgtata cgtcccgtca gcgaacgtgc tatacggtgg ttgtgccgtt | 600 |
| catgagttgt caagcacgtc tgcggggaac gtgaccgatg ccgatctggc tgaatggccc | 660 |
| acctccgttg agcggattca aaaacactac ccggaagcag aggtcgtcat tcccgggcac | 720 |
| ggtctaccgg gcggtctaga cttgctccag cacacagcga acgttgtcaa agcacacaaa | 780 |
| aatcgctcag tcgccgagta g | 801 |
| SEQ ID NO: 96 | |
| atgttaaaag ttattagtag tttattggtc tacatgaccg cgtctgtcat ggctgtcgca | 60 |
| agtccgttag cccattccgg ggagccgagt ggtgagtatc cgacagtcaa cgaaattccg | 120 |
| gtcggagagg tccgacttta ccagattgcc gatggtgttt ggtcgcatat cgcaacgcag | 180 |
| tcgtttgatg gcgcggtcta cccgtccaat ggtctcattg tccgtgatgg tgatgagttg | 240 |
| cttttgattg atacagcgtg gggtgcgaaa aacacagcgg cacttctcgc ggagattgaa | 300 |
| aagcaaattg gacttcccgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggcagcggg gaacgagatt cccacgcatt ctctagaagg actctcatcg | 480 |
| agcggggacg cagtgcgctt cggtccagta gagctcttct atcctggtgc tgcgcattcg | 540 |
| accgacaatc tggttgtata cgtcccgtca gcgaacgtgc tatacggtgg ttgtgccgtt | 600 |
| catgagttgt caagcacgtc tgcggggaac gtggccgatg ccgatctggc tgaatggccc | 660 |
| acctccgttg agcggattca aaaacactac ccggaagcag aggtcgtcat tcccgggcac | 720 |
| ggtctaccgg gcggtctaga cttgctccag cacacagcga acgttgtcaa agcacacaaa | 780 |
| aatcgctcag tcgccgagta g | 801 |
| SEQ ID NO: 97 | |
| atgttaaaag ttattagtag tttattggtc tacatgaccg cgtctgtcat ggctgtcgca | 60 |
| agtccgttag cccattccgg ggagccgagt ggtgagtatc cgacagtcaa cgaaattccg | 120 |
| gtcggagagg tccgacttta ccagattgcc gatggtgttt ggtcgcatat cgcaacgcag | 180 |
| tcgtttgatg gcgcggtcta cccgtccaat ggtctcattg tccgtgatgg tgatgagttg | 240 |
| cttttgattg atacagcgtg gggtgcgaaa aacacagcgg cacttctcgc ggagattgaa | 300 |
| aagcaaattg gacttcccgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggcagaggg gaacgagatt cccacgcatt ctctagaagg actctcatcg | 480 |
| agcggggacg cagtgcgctt cggtccagta gagctcttct atcctggtgc tgcgcattcg | 540 |
| accgacaatc tggttgtata cgtcccgtca gcgaacgtgc tatacggtgg ttgtgccgtt | 600 |
| catgagttgt caagcacgtc tgcggggaac gtggccgatg ccgatctggc tgaatggccc | 660 |
| acctccgttg agcggattca aaaacactac ccggaagcag aggtcgtcat tcccgggcac | 720 |
| ggtctaccgg gcggtctaga cttgctccag cacacagcga acgttgtcaa agcacacaaa | 780 |
| aatcgctcag tcgccgagta g | 801 |
| SEQ ID NO: 98 | |
| atgttaaaag ttattagtag tttattggtc tacatgaccg cgtctgtcat ggctgtcgca | 60 |
| agtccgttag cccattccgg ggagccgagt ggtgagtatc cgacagtcaa cgaaattccg | 120 |
| gtcggagagg tccgacttta ccagattgcc gatggtgttt ggtcgcatat cgcaacgcag | 180 |
| tcgtttgatg gcgcggtcta cccgtccaat ggtctcattg tccgtgatgg tgatgagttg | 240 |
| cttttgattg atacagcgtg gggtgcgaaa aacacagcgg cacttctcgc ggagattgaa | 300 |
| aagcaaattg gacttcccgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggcagaggg gaacgagatt cccacgcatt ctctagaagg actctcatcg | 480 |
| agcggggacg cagtgcgctt cggtccagta gagctcttct atcctggtgc tgcgcattcg | 540 |
| accgacaatc tggttgtata cgtcccgtca gcgaacgtgc tatacggtgg ttgtgccgtt | 600 |
| catgagttgt caagcacgtc tgcggggaac gtggccgatg ccgatctggc tgaatggccc | 660 |
| acctccgttg agcggattca aaaacactac ccggaagcag aggtcgtcat tcccgggcac | 720 |
| ggtctaccgg gcggtctaga cttgctccag cacacagcga acgttgtcaa agcacacaaa | 780 |
| aatcgctcag tcgccgagta g | 801 |
| SEQ ID NO: 99 | |
| atgttaaaag ttattagtag tttattggtc tacatgaccg cgtctgtcat ggctgtagct | 60 |
| agtccgttag cccattccgg ggagccgagt ggtgagtatc cgacagtcaa cgaaattccg | 120 |
| gtcggagagg tccggcttta ccagattgct gatggtgttt ggtcgcatat cgcaacgcag | 180 |
| tcgtttgatg gcgcggtcta cccatccaat ggtctcattg tccgtgatgg tgatgagttg | 240 |
| cttttgattg atacagcgtg gggtgcgaaa aacacagcgg cccttctcgc ggagattgag | 300 |
| aagcaaattg gacttcccgt aacgcgtgca gtctccacgc actttcatga cgaccgcgtc | 360 |
| ggcggcgttg atgtccttcg gaaggctgga gtggcaacgt acgcatcacc gtcgacacgc | 420 |
| cggctagccg aggcagaggg gaacgagatt cccacgcact ctctagaagg actctcatcg | 480 |
| agcggggacg cagtgcgctt cggtccagta gagctcttct atcccggtgc tgcgcattcg | 540 |
| accgacaatc tggttgtata cgtcccgtca gcgaacgtgc tatacggtgg ttgtgccgtt | 600 |
| cttgcgttgt cacgcacgtc tgcggggaac gtggccgatg ccgatctggc tgaatggccc | 660 |
| acctccgttg agcggattca aaaacactac ccggaagcac agttcgtcat tccggggcac | 720 |
| ggcctgccgg gcggtctaga cttgctcaag cacacaacga atgttgtaaa agcgcacaca | 780 |
| aatcgctcag tcgttgagta g | 801 |
| SEQ ID NO: 100 | |
| atgttaaaag ttattagtag tttattggtc tacatgaccg cgtctgtcat ggctgtagct | 60 |
| agtccgttag cccattccgg ggagccgagt ggtgagtatc cgacagtcaa cgaaattccg | 120 |
| gtcggagagg tccggcttta ccagattgct gatggtgttt ggtcgcatat cgcaacgcag | 180 |
| tcgtttgatg gcgcggtcta cccatccaat ggtctcattg tccgtgatgg tgatgagttg | 240 |
| cttttgattg atacagcgtg gggtgcgaaa aacacagcgg cccttctcgc ggagattgag | 300 |
| aagcaaattg gacttcccgt aacgcgtgca gtctccacgc actttcatga cgaccgcgtc | 360 |
| ggcggcgttg atgtccttcg gaaggctgga gtggcaacgt acgcatcacc gtcgacacgc | 420 |
| cggctagccg aggcagaggg gaacgagatt cccacgcact ctctagaagg actctcatcg | 480 |
| agcggggacg cagtgcgctt cggtccagta gagctcttct atcccggtgc tgcgcattcg | 540 |
| accgacaatc tggttgtata cgtcccgtca gcgaacgtgc tatacggtgg ttgtgccgtt | 600 |
| cttgcgttgt cacgcacgtc tgcggggaac gtggccgatg ccgatctggc tgaatggccc | 660 |
| acctccgttg agcggattca aaaacactac ccggaagcag aggtcgtcat tcccgggcac | 720 |
| ggtctaccgg gcggtctaga cttgctccag cacacagcga acgttgtcac agcacacaaa | 780 |
| aatcgctcag tcgccgagta g | 801 |
| SEQ ID NO: 101 | |
| atgttaaaag ttattagtag tttattggtc tacatgaccg cgtctgtcat ggctgtcgca | 60 |
| agtccgttag cccattccgg ggagccgagt ggtgagtatc cgacagtcaa cgaaattccg | 120 |
| gtcggagagg tccgacttta ccagattgcc gatggtgttt ggtcgcatat cgcaacgcag | 180 |
| tcgtttgatg gcgcggtcta cccgtccaat ggtctcattg tccgtgatgg tgatgagttg | 240 |
| cttttgattg atacagcgtg gggtgcgaaa aacacagcgg cacttctcgc ggagattgaa | 300 |
| aagcaaattg gacttcccgt aacgcgtgca gtctccacgc actttcatga cgaccgcgtc | 360 |
| ggcggcgttg atgtccttcg ggcggctggg gtggcaacgt acgcatcacc gtcgacacgc | 420 |
| cggctagccg aggcagaggg gaacgagatt cccacgcatt ctctagaagg actctcatcg | 480 |
| agcggggacg cagtgcgctt cggtccagta gagctcttct atcctggtgc tgcgcattcg | 540 |
| accgacaatc tggttgtata cgtcccgtca gcgaaagtgc tatacggtgg ttgtgccgtt | 600 |
| catgagttgt cacgcacgtc tgcggggaac gtggccgatg ccgatctggc tgaatggccc | 660 |
| acctccgttg agcggattca aaaacactac ccggaagcag aggtcgtcat tcccgggcac | 720 |
| ggtctaccgg gcggtctaga cttgctccag cacacagcga acgttgtcaa agcacacaaa | 780 |
| aatcgctcag tcgccgagta g | 801 |
| SEQ ID NO: 102 | |
| atgttaaaag ttattagtag tttattggtc tacatgaccg cgtctgtcat ggctgtagct | 60 |
| agtccgttag cccattccgg ggagccgagt ggtgagtatc cgacagtcaa cgaaattccg | 120 |
| gtcggagagg tccggcttta ccagattgct gatggtgttt ggtcgcatat cgcaacgcag | 180 |
| tcgtttgatg gcgcggtcta cccatccaat ggtctcattg tccgtgatgg tgatgagttg | 240 |
| cttttgattg atacagcgtg gggtgcgaaa aacacagcgg cccttctcgc ggagattgag | 300 |
| aagcaaattg gacttcccgt aacgcgtgca gtctccacgc actttcatga cgaccgcgtc | 360 |
| ggcggcgttg atgtccttcg gaaggctgga gtggcaacgt acgcatcacc gtcgacacgc | 420 |
| cggctagccg aggcagaggg gaacgagatt cccacgcact ctctagaagg actctcatcg | 480 |
| agcggggacg cagtgcgctt cggtccagta gagctcttct atcccggtgc tgcgcattcg | 540 |
| accgacaatc tggttgtata cgtcccgtca gcgaacgtgc tatacggtgg ttgtgccgtt | 600 |
| cttgcgttgt cacgcacgtc tgcggggaac gtggccgatg ccgatctggc tgaatggccc | 660 |
| acctccgttg agcggattca aaaacactac ccggaagcag aggtcgtcat tcccgggcac | 720 |
| ggtctaccgg gcggtctaga cttgctccag cacacagcga acgttgtcac agcacacaaa | 780 |
| aatcgctcag tcgccgagta g | 801 |
| SEQ ID NO: 103 | |
| cgtctagttc tgctgtcttg tctctcatgg ccgctggctg gcttttctgc caccgcgctg | 60 |
| accaacctcg tcgcggaacc attcgctaaa ctcgaacagg actttggcgg ctccatcggt | 120 |
| gtgtacgcga tggataccgg ctcaggcgca actgtaagtt accgcgctga ggagcgcttc | 180 |
| ccactgtgca gctcattcaa gggctttctt gctgccgctg tgctggctcg cagccagcag | 240 |
| caggccggct tgctggacac acccatccgt tacggcaaaa atgcgctggt tccgtggtca | 300 |
| cccatctcgg aaaaatatct gacaacaggc atgacggtgg cggagctgtc cgcggccgcc | 360 |
| gtgcaataca gtgataacgc cgccgccaat ttgttgctga aggagttggg cggcccggcc | 420 |
| gggctgacgg ccttcatgcg ctctatcggc gataccacgt tccgtctgga ccgctgggag | 480 |
| ctggagctga actccgccat cccaggcgat gcgcgcgata cctcatcgcc gcgcgccgtg | 540 |
| acggaaagct tacaaaaact gacactgggc tctgcactgg ctgcgccgca gcggcagcag | 600 |
| tttgttgatt ggctaaaggg aaacacgacc ggcaaccacc gcatccgcgc ggcggtgccg | 660 |
| gcagactggg cagtcggaga caaaaccgga acctgcggag tgtatggcac ggcaaatgac | 720 |
| tatgccgtcg tctggcccac tgggcgcgca cctattgtgt tggccgtcta cacccgggcg | 780 |
| cctaacaagg atgacaagca cagcgaggcc gtcatcgccg ctgcggctag actcgcgctc | 840 |
| gatgatgcc | 849 |
| SEQ ID NO: 104 | |
| atgtcactgt atcgccgtct agttctgctg tcttgtctct catggccgct ggctggcttt | 60 |
| tctgccaccg cgctgaccaa cctcgtcgcg gaaccattcg ctaaactcga acaggacttt | 120 |
| ggcggctcca tcggtgtgta cgcgatggat accggctcag gcgcaactgt aagttaccgc | 180 |
| gctgaggagc gcttcccact gtgcagctca ttcaagggct ttcttgctgc cgctgtgctg | 240 |
| gctcgcagcc agcagcaggc cggcttgctg gacacaccca tccgttacgg caaaaatgcg | 300 |
| ctggttcggt ggtcacccat ctcggaaaaa tatctgacaa caggcatgac ggttttggag | 360 |
| ctgtccgcgg ccgccgtgca atacagtgat aacgccgccg ccaatttgtt gctgaaggag | 420 |
| ttgggcggcc cggccaaact gacggccttc atgcgctcta tcggcgatac cacgttccgt | 480 |
| ctggaccgct gggagctgga gctgaactcc gccatcccag gcgatgcgcg cgatacctca | 540 |
| tcgccgcgcg ccgtgacgga aagcttacaa aaactgacac tgggctctgc actggctgcg | 600 |
| ccgcagcggc agcagtttgt tgattggcta aagggaaaca cgaccggcaa ccaccgcatc | 660 |
| cgcgcggcgg tgccggcaga ctgggcagtc ggagacaaaa ccggaacctg cggagggtat | 720 |
| ggcacggcaa atgactatgc cgtcgtctgg cccactgggc gcgcacctat tgtgttggcc | 780 |
| gtctacaccc gggcgcctaa caaggatgac aagtacagcg aggccgtcat cgccgctgcg | 840 |
| gctagactcg cgctcgaggg attgggcgtc aacgggcagt aa | 882 |
| SEQ ID NO: 105 | |
| atgtcactgt atcgccgtct agttctgctg tcttgtctct catggccgct ggctggcttt | 60 |
| tctgccaccg cgctgaccaa cctcgtcgcg gaaccattcg ctaaactcga acaggacttt | 120 |
| ggcggctcca tcggtgtgta cgcgatggat accggctcag gcgcaactgt aagttaccgc | 180 |
| gctgaggagc gcttcccact gtgcagctca ttcaagggct ttcttgctgc cgctgtgctg | 240 |
| gctcgcagcc agcagcaggc cggcttgctg gacacaccca tccgttacgg caaaaatgcg | 300 |
| ctggttccgt ggtcacccat ctcggaaaaa tatctgacaa caggcatgac ggtggcggag | 360 |
| ctgtccgcgg ccgccgtgca atacagtgat aacgccgccg ccaatttgtt gctgaaggag | 420 |
| ttgggcggcc cggccgggct gacggccttc atgcgctcta tcggcgatac cacgttccgt | 480 |
| ctggaccgct gggagctgga gctgaactcc gccatcccag gcgatgcgcg cgatacctca | 540 |
| tcgccgcgcg ccgtgacgga aagcttacaa aaactgacac tgggctctgc actggctgcg | 600 |
| ccgcagcggc agcagtttgt tgattggcta aagggaaaca cgaccggcaa ccaccgcatc | 660 |
| cgcgcggcgg tgccggcaga ctgggcagtc ggagacaaaa ccggaacctg cggagtgtat | 720 |
| gcaaatgact atgccgtcgt ctggcccact gggcgcgcac ctattgtgtt ggccgtctac | 780 |
| acccgggcgc ctaacaagga tgacaagcac agcgaggccg tcatcgccgc tgcggctaga | 840 |
| ctcgcgctcg agggattggg cgtcaacggg cagtaa | 876 |
| SEQ ID NO: 106 | |
| atgtcactgt atcgccgtct agttctgctg tcttgtctct catggccgct ggctggcttt | 60 |
| tctgccaccg cgctgaccaa cctcgtcgcg gaaccattcg ctaaactcga acaggacttt | 120 |
| ggcggctcca tcggtgtgta cgcgatggat accggttcag gcgcaactgt aagttaccgc | 180 |
| gctgaggagc gcttcccact gtgcagctca ttcaagggct ttcttgctgc cgctgtgctg | 240 |
| gctcgcagcc agcagcaggc cggcttgctg gacacaccca tccgttacgg caaaaatgcg | 300 |
| ctggttccgt ggtcacccat ctcggaaaaa tatctgacaa caggcatgac ggtggcggag | 360 |
| ctgtccgcgg ccgccgtgca atacagtgat aacgccgccg ccaatttgtt gctgaaggag | 420 |
| ttgggcggcc cggccgggct gacggccttc atgcgctcta tcggcgatac cacgttccgt | 480 |
| ctggaccgct gggagctgga gctgaactcc gccatcccag gcgatgcgcg cgatacctca | 540 |
| tcgccgcgcg ccgtgacgga aagcttacaa aaactgacac tgggctctgc actggctgcg | 600 |
| tcgcagcggc agcagcttgt tgattggcta aagggaaaca cgaccggcaa ccaccgcatc | 660 |
| cgcgcggcgg tgccggcaga ctgggcagtc ggagacaaaa ccggaacctg cggagtgtat | 720 |
| ggcacggcaa atgactatgc cgtcgtctgg cccactgggc gcgcacctat tgtgttggcc | 780 |
| gtctacaccc gggcgcctaa caaggatgac aagcacagcg aggccgtcat cgccgctgcg | 840 |
| gctagactcg cgctcgaggg attgggcgtc aacgggcagt aa | 882 |
| SEQ ID NO: 107 | |
| atgtcactgt atcgccgtct agttctgctg tcttgtctct catggccgct ggctggcttt | 60 |
| tctgccaccg cgctgaccaa cctcgtcgcg gaaccattcg ctaaactcga acaggacttt | 120 |
| ggcggctcca tcggtgtgta cgcgatggat accggctcag gcgcaactgt aagttaccgc | 180 |
| gctgaggagc gcttcccact gtgcagctca ttcaagggct ttcttgctgc cgctgtgctg | 240 |
| gctcgcagcc agcagcaggc cggcttgctg gacacaccca tccgttacgg caaaaatgcg | 300 |
| ctggttccgt ggtcacccat ctcggaaaaa tatctgacaa caggcatgac ggtggcggag | 360 |
| ctgtccgcgg ccgccgtgca atacagtgat aacgccgccg ccaatttgtt gctgaaggag | 420 |
| ttgggcggcc cggccgggct gacggccttc atgcgctcta tcggcgatac cacgttccgt | 480 |
| ctggaccgct gggagctgga gatgaactcc gccatcccag gcgatgcgcg cgatacctca | 540 |
| tcgccgcgcg ccgtgacgga aagcttacaa aaactgacac tgggctctgc actggctgcg | 600 |
| ccgcagcggc agcagtttgt tgattggcta aagggaaaca cgaccggcaa ccaccgcatc | 660 |
| cgcgcggcgg tgccggcaga ctgggcagtc ggagacaaaa ccggaacctg cggagtgtat | 720 |
| ggcacggcaa atgactatgc cgtcgtctgg cccactgggc gcgcacctat tgtgttggcc | 780 |
| gtctacaccc gggcgcctaa caaggatgac aagcacagcg aggccgtcat cgccgctgcg | 840 |
| gctagactcg cgctcgaggg attgggcgtc aacgggcagt aa | 882 |
| SEQ ID NO: 108 | |
| atgtcactgt atcgccgtct agttctgctg tcttgtctct catggccgct ggctggcttt | 60 |
| tctgccaccg cgctgaccaa cctcgtcgcg gaaccattcg ctaaactcga acaggacttt | 120 |
| ggcggctcca tcggtgtgta cgcgatggat accggctcag gcgcaactgt aagttaccgc | 180 |
| gctgaggagc gcttcccact gtgcagctca ttcaagggct ttcttgctgc cgctgtgctg | 240 |
| gctcgcagcc agcagcaggc cggcttgctg gacacaccca tccgttacgg caaaaatgcg | 300 |
| ctggttccgt ggtcacccat ctcggaaaaa tatctgacaa caggcatgac ggtggcggag | 360 |
| ctgtccgcgg ccgccgtgca atacagtgat aacgccgccg ccaatttgtt gctgaaggag | 420 |
| ttgggcggcc cggccgggct gacggccttc atgcgctcta tcggcgatac cacgttccgt | 480 |
| ctggaccgct gggagctgga gctgaactcc gccatcccaa gcgatgcgcg cgatacctca | 540 |
| tcgccgcgcg ccgtgacgga aagcttacaa aaactgacac tgggctctgc actggctgcg | 600 |
| ccgcagcggc agcagtttgt tgattggcta aagggaaaca cgaccggcaa ccaccgcatc | 660 |
| cgcgcggcgg tgccggcaga ctgggcagtc ggagacaaaa ccggaacctg cggagtgtat | 720 |
| ggcacggcaa atgactatgc cgtcgtctgg cccactgggc gcgcacctat tgtgttggcc | 780 |
| gtctacaccc gggcgcctaa caaggatgac aagcacagcg aggccgtcat cgccgctgcg | 840 |
| gctagactcg cgctcgaggg attgggcgtc aacgggcagt aa | 882 |
| SEQ ID NO: 109 | |
| atgtcactgt atcgccgtct agttctgctg tcttgtctct catggccgct ggctggcttt | 60 |
| tctgccaccg cgctgaccaa cctcgtcgcg gaaccattcg ctaaactcga acaggacttt | 120 |
| ggcggctcca tcggtgtgta cgcgatggat accggctcag gcgcaactgt aagttaccgc | 180 |
| gctgaggagc gcttcccact gtgcagctca ttcaagggct ttcttgctgc cgctgtgctg | 240 |
| gctcgcagcc agcagcaggc cggcttgctg gacacaccca tccgttacgg caaaaatgcg | 300 |
| ctggttccgt ggtcacccat ctcggaaaaa tatctgacaa caggcatgac ggtggcggag | 360 |
| ctgtccgcgg ccgccgtgca atacagtgat aacgccgccg ccaatttgtt gctgaaggag | 420 |
| ttgggcggcc cggccgggct gacggccttc atgcgctcta tcggcgatac cacgttccgt | 480 |
| ctggaccgct gggagctgga gctgaactcc gccatcccag gcgatgcgcg cgatacctca | 540 |
| tcgccgcgcg ccgtgacgga aagcttacaa aaactgacac tgggctctgc actggctgcg | 600 |
| ccgcagcggc agcagcttgt tgattggcta aagggaaaca cgaccggcaa ccaccgcatc | 660 |
| cgcgcggcgg tgccggcaga ctgggcagtc ggagacaaaa ccggaacctg cggagtgtat | 720 |
| ggcacggcaa atgactatgc cgtcgtctgg cccactgggc gcgcacctat tgtgttggcc | 780 |
| gtctacaccc gggcgcctaa caaggatgac aagcacagcg aggccgtcat cgccgctgcg | 840 |
| gctagactcg cgctcgaggg attgggcgtc aacgggcagt aa | 882 |
| SEQ ID NO: 110 | |
| atgtcactgt atcgccgtct agttctgctg tcttgtctct catggccgct ggctggcttt | 60 |
| tctgccaccg cgctgaccaa cctcgtcgcg gaaccattcg ctaaactcga acaggacttt | 120 |
| ggcggctcca tcggtgtgta cgcgatggat accggctcag gcgcaactgt aagttaccgc | 180 |
| gctgaggagc gcttcccact gtgcagctca ttcaagggct ttcttgctgc cgctgtgctg | 240 |
| gctcgcagcc agcagcaggc cggcttgctg gacacaccca tccgttacgg caaaaatgcg | 300 |
| ctggttccgt ggtcacccat ctcggaaaaa tatctgacaa caggcatgac ggtggcggag | 360 |
| ctgtccgcgg ccgccgtgca atacagtgat aacgccgccg ccaatttgtt gctgaaggag | 420 |
| ttgggcggcc cggccgggct gacggccttc atgcgctcta tcggcgatac cacgttccgt | 480 |
| ctggaccgct gggagctgga gctgaactcc gccatcccag gcgatgcgcg cgatacctca | 540 |
| tcgccgcgcg ccgtgacgga aagcttacaa aaactgacac tgggctctgc actggctgcg | 600 |
| ccgcagcggc agcagtttgt tgattggcta aagggaaaca cgaccggcaa ccaccgcatc | 660 |
| cgcgcggcgg tgccggcaga ctgggcagtc ggagacaaaa ccggaacctg cggagtgtat | 720 |
| ggcacggcaa atgactatgc cgtcgtctgg cccactgggc gcgcacctat tgtgttggcc | 780 |
| gtctacaccc gggcgcctaa caaggatgac aagcacagcg aggccgtcat cgccgctgcg | 840 |
| gctagactcg cgctcgaggg attgggcgtc aacgggcagt aa | 882 |
| SEQ ID NO: 111 | |
| tgtcactgta tcgccgtcta gttctgctgt cttgtctctc atggccgctg gctggctttt | 60 |
| ctgccaccgc gctgaccaac ctcgtcgcgg aaccattcgc taaactcgaa caggactttg | 120 |
| gcggctccat cggtgtgtac gcgatggata ccggctcagg cgcaactgta agttaccgcg | 180 |
| ctgaggagcg cttcccactg tgcagctcat tcaagggctt tcttgctgcc gctgtgctgg | 240 |
| ctcgcagcca gcagcaggcc ggcttgctgg acacacccat ccgttacggc aaaaatgcgc | 300 |
| tggttcggtg gtcacccatc tcggaaaaat atctgacaac aggcatgacg gtggcggagc | 360 |
| tgtccgcggc cgccgtgcaa tacagtgata acgccgccgc caatttgttg ctgaaggagt | 420 |
| tgggcggccc ggccgggctg acggccttca tgcgctctat cggcgatacc acgttccgtc | 480 |
| tggaccgctg ggagctggag ctgaactccg ccatcccagg cgatgcgcgc gatacctcat | 540 |
| cgccgcgcgc cgtgacggaa agcttacaaa aactgacact gggctctgca ctggctgcgc | 600 |
| cgcagcggca gcagtttgtt gattggctaa agggaaacac gaccggcaac caccgcatcc | 660 |
| gcgcggcggt gccggcagac tgggcagtcg gagacaaaac cggaacctgc ggagggtatg | 720 |
| gcacggcaaa tgactatgcc gtcgtctggc ccactgggcg cgcacctatt gtgttggccg | 780 |
| tctacacccg ggcgcctaac aaggatgaca agcacagcga ggccgtcatc gccgctgcgg | 840 |
| ctagactcgc gctcgaggga ttgggcgtca acgggcagta a | 881 |
| SEQ ID NO: 112 | |
| atgtcactgt atcgccgtct agttctgctg tcttgtctct catggccgct ggctggcttt | 60 |
| tctgccaccg cgctgaccaa cctcgtcgcg gaaccattcg ctaaactcga acaggacttt | 120 |
| ggcggctcca tcggtgtgta cgcgatggat accggctcag gcgcaactgt aagttaccgc | 180 |
| gctgaggagc gcttcccact gtgcagctca ttcaagggct ttcttgctgc cgctgtgctg | 240 |
| gctcgcagcc agcagcaggc cggcttgctg gacacaccca tccgttacgg caaaaatgcg | 300 |
| ctggttctgt ggtcacccat ctcggaaaaa tatctgacaa caggcatgac ggtggcggag | 360 |
| ctgtccgcgg ccgccgtgca atacagtgat aacgccgccg ccaatttgtt gctgaaggag | 420 |
| ttgggcggcc cggccgggct gacggccttc atgcgctcta tcggcgatac cacgttccgt | 480 |
| ctggaccgct gggagctgga gctgaactcc gccatcccag gcgatgcgcg cgatacctca | 540 |
| tcgccgcgcg ccgtgacgga aagcttacaa aaactgacac tgggctctgc actggctgcg | 600 |
| ccgcagcggc agcagtttgt tgattggcta aagggaaaca cgaccggcaa ccaccgcatc | 660 |
| cgcgcggcgg tgccggcaga ctgggcagtc ggagacaaaa ccggaacctg cggagtgtat | 720 |
| ggcacggcaa atgactatgc cgtcgtctgg cccactgggc gcgcacctat tgtgttggcc | 780 |
| gtctacaccc gggcgcctaa caaggatgac aagcacagcg aggccgtcat cgccgctgcg | 840 |
| gctagactcg cgctcgaggg attgggcgtc aacgggcagt aa | 882 |
| SEQ ID NO: 113 | |
| atgtcactgt atcgccgtct agttctgctg tcttgtctct catggccgct ggctggcttt | 60 |
| tctgccaccg cgctgaccaa cctcgtcgcg gaaccattcg ctaaactcga acaggacttt | 120 |
| ggcggctcca tcggtgtgta cgcgatggat accggctcag gcgcaactgt aagttaccgc | 180 |
| gctgaggagc gcttcccact gtgcagctca ttcaagggct ttcttgctgc cgctgtgctg | 240 |
| gctcgcagcc agcagcaggc cggcttgctg gacacaccca tccgttacgg caaaaatgcg | 300 |
| ctggttcggt ggtcacccat ctcggaaaaa tatctgacaa caggcatgac ggtggcggag | 360 |
| ctgtccgcgg ccgccgtgca atacagtgat aacgccgccg ccaatttgtt gctgaaggag | 420 |
| ttgggcggcc cggccgggct gacggccttc atgcgctcta tcggcgatac cacgttccgt | 480 |
| ctggaccgct gggagctgga gctgaactcc gccatcccag gcgatgcgcg cgatacctca | 540 |
| tcgccgcgcg ccgtgacgga aagcttacaa aaactgacac tgggctctgc actggctgcg | 600 |
| ccgcagcggc agcagtttgt tgattggcta aagggaaaca cgaccggcaa ccaccgcatc | 660 |
| cgcgcggcgg tgccggcaga ctgggcagtc ggagacaaaa ccggaacctg cggagtgtat | 720 |
| ggcacggcaa atgactatgc cgtcgtctgg cccactgggc gcgcacctat tgtgttggcc | 780 |
| gtctacaccc gggcgcctaa caaggatgac aagcacagcg aggccgtcat cgccgctgcg | 840 |
| gctagactcg cgctcgaggg attgggcgtc aacgggcagt aa | 882 |
| SEQ ID NO: 114 | |
| atgtcactgt atcgccgtct agttctgctg tcttgtctct catggccgct ggctggcttt | 60 |
| tctgccaccg cgctgaccaa cctcgtcgcg gaaccattcg ctaaactcga acaggacttt | 120 |
| ggcggctcca tcggtgtgta cgcgatggat accggctcag gcgcaactgt aagttaccgc | 180 |
| gctgaggagc gcttcccact gtgcagctca ttcaagggct ttcttgctgc cgctgtgctg | 240 |
| gctcgcagcc agcagcaggc cggcttgctg ggcacaccca tccgttacgg caaaaacgcg | 300 |
| ctggttccgt ggtcacccat ctcggaaaaa tatctgacaa caggcatgac ggtggcggag | 360 |
| ctgtccgcgg ccgccgtgca atacagtgat aacgccgccg ccaatttgtt gctgaaggag | 420 |
| ttgggcggcc cggccgggct gacggccttc atgcgctcta tcggcgatac cacgttccgt | 480 |
| ctggactgct gggagctgga gctgaactcc gctatcccag gcgatgcgcg cgatacctca | 540 |
| tcgccgcgcg ccgtgacgga aagcttacaa aaactgacac tgggctctgc actggctgcg | 600 |
| ccgcagcggc agcagtttgt tgattggcta aagggaaaca cgaccggcaa ccaccgcatc | 660 |
| cgcgcggcgg tgccggcaga ctgggcagtc ggagacaaaa ccggaacctg cggagtgtat | 720 |
| ggcacggcaa atgactatgc cgtcgtctgg cccactgggc gcgcacctat tgtgttggcc | 780 |
| gtctacaccc gggcgcctaa caaggatgac aagtacagcg aggccgtcat cgccgctgcg | 840 |
| gctggactcg cgctcgaggg attgggcgtc aacgggcagt aa | 882 |
| SEQ ID NO: 115 | |
| atgtcactgt atcgccgtct agttctgctg tcttgtctct catggccgct ggctggcttt | 60 |
| tctgccaccg cgctgaccaa cctcgtcgcg gaaccattcg ctaaactcga acaggacttt | 120 |
| ggcggctcca tcggtgtgta cgcgatggat accggctcag gcgcaactgt aagttaccgc | 180 |
| gctgaggagc gcttcccact gtgcagctca ttcaagggct ttcttgctgc cgctgtgctg | 240 |
| gctcgcagcc agcagcaggc cggcttgctg ggcacaccca tccgttacgg caaaaatgcg | 300 |
| ctggttccgt ggtcacccat ctcggaaaaa tatctgacaa caggcatgac ggtggcggag | 360 |
| ctgtccgcgg ccgccgtgca atacagtgat aacgccgccg ccaatttgtt gctgaaggag | 420 |
| ttgggcggcc cggccgggct gacggccttc atgcgctcta tcggcgatac cacgttccgt | 480 |
| ctggaccgct gggagctgga gctgaactcc gctatcccag gcgatgcgcg cgatacctca | 540 |
| tcgccgcgcg ccgtgacgga aagcttacaa aaactgacac tgggctctgc actggctgcg | 600 |
| ccgcagcggc agcagtttgt tgattggcta aagggaaaca cgaccggcaa ccaccgcatc | 660 |
| cgcgcggcgg tgccggcaga ctgggcagtc ggagacaaaa ccggaacctg cggagtgtat | 720 |
| ggcacggcaa atgactatgc cgtcgtctgg cccactgggc gcgcacctat tgtgttggcc | 780 |
| gtctacaccc gggcgcctaa caaggatgac aagtacagcg aggccgtcat cgccgctgcg | 840 |
| gctagactcg cgctcgaggg attgggcgtc aacgggcagt aa | 882 |
| SEQ ID NO: 116 | |
| gccgtctagt tctgctgtct tgtctctcat ggccgctggc tggcttttct gccaccgcgc | 60 |
| tgaccaacct cgtcgcggaa ccattcgcta aactcgaaca ggactttggc ggctccatcg | 120 |
| gtgtgtacgc gatggatacc ggctcaggcg caactgtaag ttaccgcgct gaggagcgct | 180 |
| tcccactgtg cagctcattc aagggctttc ttgctgccgc tgtgctggct cgcagccagc | 240 |
| agcaggccgg cttgctggac acacccatcc gttacggcaa aaatgcgctg gttccgtggt | 300 |
| cacccatctc ggaaaaatat ctgacaacag gcatgacggt ggcggagctg tccgcggccg | 360 |
| ccgtgcaata cagtgataac gccgccgcca atttgttgct gaaggagttg ggcggcccgg | 420 |
| ccgggctgac ggccttcatg cgctctatcg gcgataccac gttccgtctg gaccgctggg | 480 |
| agctggagct gaactccgcc atcccaggcg atgcgcgcga tacctcatcg ccgcgcgccg | 540 |
| tgacggaaag cttacaaaaa ctgacactgg gctctgcact ggctgcgccg cagcggcagc | 600 |
| agtttgttga ttggctaaag ggaaacacga ccggcaacca ccgcatccgc gcggcggtgc | 660 |
| cggcagactg ggcagtcgga gacaaaaccg gaacctgcgg agcgtatggc acggcaaatg | 720 |
| actatgccgt cgtctggccc actgggcgcg cacctattgt gttggccgtc tacacccggg | 780 |
| cgcctaacaa ggatgacaag tacagcgagg ccgtcatcgc cgctgcggct agactcgcgc | 840 |
| tcgagggatt gggc | 854 |
| SEQ ID NO: 117 | |
| atgtcactgt atcgccgtct agttctgctg tcttgtctct catggccgct ggctggcttt | 60 |
| tctgccaccg cgctgaccaa cctcgtcgcg gaaccattcg ctaaactcga acaggacttt | 120 |
| ggcggctcca tcggtgtgta cgcgatggat accggctcag gcgcaactgt aagttaccgc | 180 |
| gctgaggagc gcttcccact gtgcagctca ttcaagggct ttcttgctgc cgctgtgctg | 240 |
| gctcgcagcc agcagcaggc cggcttgctg gacacaccca tccgttacgg caaaaatgcg | 300 |
| ctggttccgt ggtcacccat ctcggaaaaa tatctgacaa caggcatgac ggtggcggag | 360 |
| ctgtccgcgg ccgccgtgca atacagtgat aacgccgccg ccaatttgtt gctgaaggag | 420 |
| ttgggcggcc cggccgggct gacggccttc atgcgctcta tcggcgatac cacgttccgt | 480 |
| ctggaccgct gggagctgga gctgaactcc gccatcccag gcgatgcgcg cgatacctca | 540 |
| tcgccgcgcg ccgtgacgga aagcttacaa aaactgacac tgggctctgc actggctgcg | 600 |
| ccgcagcggc agcagtttgt tgattggcta aagggaaaca cgaccggcaa ccaccgcatc | 660 |
| cgcgcggcgg tgccggcaga ctgggcagtc ggagacaaaa ccggaacctg cggagggtat | 720 |
| ggcacggcaa atgactatgc cgtcgtctgg cccactgggc gcgcacctat tgtgttggcc | 780 |
| gtctacaccc gggcgcctaa caaggatgac aagcacagcg aggccgtcat cgccgctgcg | 840 |
| gctagactcg cgctcgaggg attgggcgtc aacgggcagt aa | 882 |
| SEQ ID NO: 118 | |
| atgtcactgt atcgccgtct agttctgctg tcttgtctct catggccgct ggctggcttt | 60 |
| tctgccaccg cgctgaccaa cctcgtcgcg gaaccattcg ctaaactcga acaggacttt | 120 |
| ggcggctcca tcggtgtgta cgcgatggat accggctcag gcgcaactgt aagttaccgc | 180 |
| gctgaggagc gcttcccact gtgcagctca ttcaagggct ttcttgctgc cgctgtgctg | 240 |
| gctcgcagcc agcagcaggc cggcttgctg gacacaccca tccgttacgg caaaaatgcg | 300 |
| ctggttcggt ggtcacccat ctcggaaaaa tatctgacaa caggcatgac ggtggcggag | 360 |
| ctgtccgcgg ccgccgtgca atacagtgat aacgccgccg ccaatttgtt gctgaaggag | 420 |
| ttgggcggcc cggccgggct gacggccttc atgcgctcta tcggcgatac cacgttccgt | 480 |
| ctggaccgct gggagctgga gctgaactcc gccatcccag gcgatgcgcg cgatacctca | 540 |
| tcgccgcgcg ccgtgacgga aagcttacaa aaactgacac tgggctctgc actggctgcg | 600 |
| ccgcagcggc agcagtttgt tgattggcta aagggaaaca cgaccggcaa ccaccgcatc | 660 |
| cgcgcggcgg tgccggcaga ctgggcagtc ggagacaaaa ccggaacctg cggagtgtat | 720 |
| ggcacggcaa atgactatgc cgtcgtctgg cccactgggc gcgcacctat tgtgttggcc | 780 |
| gtctacaccc gggcgcctaa caaggatgac aagtacagcg aggccgtcat cgccgctgcg | 840 |
| gctagactcg cgctcgaggg attgggcgtc aacgggcagt aa | 882 |
| SEQ ID NO: 119 | |
| atgtcactgt atcgccgtct agttctgctg tcttgtctct catggccgct ggctggcttt | 60 |
| tctgccaccg cgctgaccaa cctcgtcgcg gaaccattcg ctaaactcga acaggacttt | 120 |
| ggcggctcca tcggtgtgta cgcgatggat accggctcag gcgcaactgt aagttaccgc | 180 |
| gctgaggagc gcttcccact gtgcagctca ttcaagggct ttcttgctgc cgctgtgctg | 240 |
| gctcgcagcc agcagcaggc cggcttgctg gacacaccca tccgttacgg caaaaatgcg | 300 |
| ctggttccgt ggtcacccat ctcggaaaaa tatctgacaa caggcatgac ggtggcggag | 360 |
| ctgtccgcgg ccgccgtgca atacagtgat aacgccgccg ccaatttgtt gctgaaggag | 420 |
| ttgggcggcc cggccgggct gacggccttc atgcgctcta tcggcgatac cacgttccgt | 480 |
| ctggaccgct gggagctgga gctgaactcc gccatcccag gcgatgcgcg cgatacctca | 540 |
| tcgccgcgcg ccgtgacgga aagcttacaa aaactgacac tgggctctgc actggctgcg | 600 |
| ccgcagcggc agcagtttgt tgattggcta aagggaaaca cgaccggcaa ccaccgcatc | 660 |
| cgcgcggcgg tgccggcaga ctgggcagtc ggagacaaaa ccggaacctg cggagggtat | 720 |
| ggcacggcaa atgactatgc cgtcgtctgg cccactgggc gcgcacctat tgtgttggcc | 780 |
| gtctacaccc gggcgcctaa caaggatgac aagtacagcg aggccgtcat cgccgctgcg | 840 |
| gctagactcg cgctcgaggg attgggcgtc aacgggcagt aa | 882 |
| SEQ ID NO: 120 | |
| atgtcactgt atcgccgtct agttctgctg tcttgtctct catggccgct ggctggcttt | 60 |
| tctgccaccg cgctgaccaa cctcgtcgcg gaaccattcg ctaaactcga acaggacttt | 120 |
| ggcggctcca tcggtgtgta cgcgatggat accggctcag gcgcaactgt aagttaccgc | 180 |
| gctgaggagc gcttcccact gtgcagctca ttcaagggct ttcttgctgc cgctgtgctg | 240 |
| gctcgcagcc agcagcaggc cggcttgctg gacacaccca tccgttacgg caaaaatgcg | 300 |
| ctggttccgt ggtcacccat ctcggaaaaa tatctgacaa caggcatgac ggtggcggag | 360 |
| ctgtccgcgg ccgccgtgca atacagtgat aacgccgccg ccaatttgtt gctgaaggag | 420 |
| ttgggcggcc cggccgggct gacggccttc atgcgctcta tcggcgatac cacgttccgt | 480 |
| ctggaccgct gggagctgga gctgaactcc gccatcccag gcgatgcgcg cgatacctca | 540 |
| tcgccgcgcg ccgtgacgga aagcttacaa aaactgacac tgggctctgc actggctgcg | 600 |
| ccgcagcggc agcagtttgt tgattggcta aagggaaaca cgaccggcaa ccaccgcatc | 660 |
| cgcgcggcgg tgccggcaga ctgggcagtc ggagacaaaa ccggaacctg cggagtgtat | 720 |
| ggcacggcaa atgactatgc cgtcgtctgg cccactgggc gcgcacctat tgtgttggcc | 780 |
| gtctacaccc gggcgcctaa caaggatgac aagtacagcg aggccgtcat cgccgctgcg | 840 |
| gctagactcg cgctcgaggg attgggcgtc aacgggcagt aa | 882 |
| SEQ ID NO: 121 | |
| atgtcactgt atcgccgtct agttctgctg tcttgtctct catggccgct ggctggcttt | 60 |
| tctgccaccg cgctgaccaa cctcgtcgcg gaaccattcg ctaaactcga acaggacttt | 120 |
| ggcggctcca tcggtgtgta cgcgatggat accggctcag gcgcaactgt aagttaccgc | 180 |
| gctgaggagc gcttcccact gtgcagctca ttcaagggct ttcttgctgc cgctgtgctg | 240 |
| gctcgcagcc agcagcaggc cggcttgctg gacacaccca tccgttacgg caaaaatgcg | 300 |
| ctggttccgt ggtcacccat ctcggaaaaa tatctgacaa caggcatgac ggtggcggag | 360 |
| ctgtccgcgg ccgccgtgca atacagtgat aacgccgccg ccaatttgtt gctgaaggag | 420 |
| ttgggcggcc cggccgggct gacggccttc atgcgctcta tcggcgatac cacgttccgt | 480 |
| ctggaccgct gggagctgga gctgaactcc gccatcccag gcgatgcgcg cgatacctca | 540 |
| tcgccgcgcg ccgtgacgga aagcttacaa aaactgacac tgggctctgc actggctgcg | 600 |
| ccgcagcggc agcagtttgt tgattggcta aagggaaaca cgaccggcaa ccaccgcatc | 660 |
| cgcgcggcgg tgccggcaga ctgggcagtc ggagacaaaa ccggaacctg cggagtgtat | 720 |
| ggcacggcaa atgactatgc cgtcgtctgg cccactgggc gcgcacctat tgtgttggcc | 780 |
| gtctacaccc gggcgcctaa caaggatgac aagtacagcg aggccgtcat cgccgctgcg | 840 |
| gctagactcg cgctcgaggg attgggcgtc aacgggcagt aa | 882 |
| SEQ ID NO: 122 | |
| atgtcactgt atcgccgtct agttctgctg tcttgtctct catggccgct ggctggcttt | 60 |
| tctgccaccg cgctgaccaa cctcgtcgcg gaaccattcg ctaaactcga acaggacttt | 120 |
| ggcggctcca tcggtgtgta cgcgatagat accggctcag gcgcaactgt aagttaccgc | 180 |
| gctgaggagc gcttcccact gtgcagctca ttcaagggct ttcttgctgc cgctgtgctg | 240 |
| gctcgcagcc agcagcaggc cggcttgctg gacacaccca tccgttacgg caaaaatgcg | 300 |
| ctggttccgt ggtcacccat ctcggaaaaa tatctgacaa caggcatgac ggtggcggag | 360 |
| ctgtccgcgg ccgccgtgca atacagtgat aacgccgccg ccaatttgtt gctgaaggag | 420 |
| ttgggcggcc cggccgggct gacggccttc atgcgctcta tcggcgatac cacgttccgt | 480 |
| ctggaccgct gggagctgga gctgaactcc gccatcccag gcgatgcgcg cgatacctca | 540 |
| tcgccgcgcg ccgtgacgga aagcttacaa aaactgacac tgggctctgc actggctgcg | 600 |
| ccgcagcggc agcagtttgt tgattggcta aagggaaaca cgaccggcaa ccaccgcatc | 660 |
| cgcgcggcgg tgccggcaga ctgggcagtc ggagacaaaa ccggaacctg cggagtgtat | 720 |
| ggcacggcaa atgactatgc cgtcgtctgg cccactgggc gcgcacctat tgtgttggcc | 780 |
| gtctacaccc gggcgcctaa caaggatgac aagtacagcg aggccgtcat cgccgctgcg | 840 |
| gctagactcg cgctcgaggg attgggcgtc aacgggcagt aa | 882 |
| SEQ ID NO: 123 | |
| atggaattgc ccaatattat gcacccggtc gcgaagctga gcaccgcatt agccgctgca | 60 |
| ttgatgctga gcgggtgcat gcccggtgaa atcagcccga cgattgacca gcaaatggaa | 120 |
| actggcgacc aacggtttgg cgatctggtt ttccgccagc tcgcaccgaa tgtctggcag | 180 |
| cacacttcct atctcgacat gccgagtttc ggggcagtca cttccaacgg tttgatcgtc | 240 |
| agggatggcg gccgcgtgct ggtggtcgat accgcctgga ccgatgacca gaccgcccag | 300 |
| atcctcaact ggatcaagca ggagatcaac ctgccggtcg cgctggcggt ggtgactcac | 360 |
| gcgcatcagg acaagatggg cggtatggac gcgctgcatg cggcggggat tgcgacttat | 420 |
| gccaatgcgt tgtcgaacca gcttgccccg caagagggga tggttgcggc gcaacacagc | 480 |
| ctgactttcg ccgccaatgg ctgggtcgaa ccagcaaccg cgcccaactt tggcccgctc | 540 |
| aaggtatttt accccggccc cggccacacc agtgacaata tcaccgttgg gatcgaccgc | 600 |
| accgacatcg cttttggtgg ctgcctgatc aaggacagca aggccaagtc gctcggcaat | 660 |
| ctcggtgatg ccgacactga gcactacgcc gcgtcagcgc gcgcgtttgg tgcggcgttc | 720 |
| cccaaggcca gcatgatcgt gatgagccat tccgcccccg atagccgcgc cgcaatcact | 780 |
| catacggccc gcatggccga caagctgcgc tga | 813 |
| SEQ ID NO: 124 | |
| atggaattgc ccaatattat gcacccggtc gcgaagctga gcaccgcatt agccgctgca | 60 |
| ttgatgctga gcgggtgcat gcccggtgaa atccgcccga cgattggcca gcaaatggaa | 120 |
| actggcgacc aacggtttgg cgatctggtt ttccgccagc tcgcaccgaa tgtctggcag | 180 |
| cacacttcct atctcgacat gccgggtttc ggggcagtcg cttccaacgg tttgatcgtc | 240 |
| agggatggcg gccgcgtgct ggtggtcgat accgcctgga ccgatgacca gaccgcccag | 300 |
| atcctcaact ggatcaagca ggagatcaac ctgccggtcg cgctggcggt ggtgactcac | 360 |
| gcgcatcagg acaagatggg cggtatgggc gcgctgcatg cggcggggat tgcgacttat | 420 |
| gccaatgcgt tgtcgaacca gcttgccccg caagaggggc tggttgcggc gcaacacagc | 480 |
| ctgactttcg ccgccaatgg ctgggtcgaa ccagcaaccg cgcccaactt tggcccgctc | 540 |
| aaggtatttt accccggccc cggccacacc agtgacaata tcaccgttgg gatcgacggc | 600 |
| accgacatcg cttttggtgg ctgcctgatc aaggacagca aggccaagtc gctcggcaat | 660 |
| ctcggtgatg ccgacactga gcactacgcc gcgtcagcgc gcgcgtttgg tgcggcgttc | 720 |
| cccaaggcca gcatgatcgt gatgagccat tccgcccccg atagccgcgc cgcaatcact | 780 |
| catacggccc gcatggccga caagctgcgc tga | 813 |
| SEQ ID NO: 125 | |
| atggaattgc ccaatattat gcacccggtc gcgaagctga gcaccgcatt agccgctgca | 60 |
| ttgatgctga gcgggtgcat gcccggtgaa atccgcccga cgattggcca gcaaatggaa | 120 |
| actggcgacc aacggtttgg cgatctggtt ttccgccagc tcgcaccgaa tgtctggcag | 180 |
| cacacttcct atctcgacat gccgggtttc ggggcagtcg cttccaacgg tttgatcgtc | 240 |
| agggatggcg gccgcgtgct ggtggtcgat accgcctgga ccgatgacca gaccgcccag | 300 |
| atcctcaact ggatcaagca ggagatcaac ctgccggtcg cgctggcggt ggtgactcac | 360 |
| gcgcatcagg acaagatggg cggtatgaac gcgctgcatg cggcggggat tgcgacttat | 420 |
| gccaatgcgt tgtcgaacca gcttgccccg caagaggggc tggttgcggc gcaacacagc | 480 |
| ctgactttcg ccgccaatgg ctgggtcgaa ccagcaaccg cgcccaactt tggcccgctc | 540 |
| aaggtatttt accccggccc cggccacacc agtgacaata tcaccgttgg gatcgacggc | 600 |
| accgacatcg cttttggtgg ctgcctgatc aaggacagca aggccaagtc gctcggcaat | 660 |
| ctcggtgatg ccgacactga gcactacgcc gcgtcagcgc gcgcgtttgg tgcggcgttc | 720 |
| cccaaggcca gcatgatcgt gatgagccat tccgcccccg atagccgcgc cgcaatcact | 780 |
| catacggccc gcatggccga caagctgcgc tga | 813 |
| SEQ ID NO: 126 | |
| atggaattgc ccaatattat gcacccggtc gcgaagctga gcaccgcatt agccgctgca | 60 |
| ttgatgctga gcgggtgcat gcccggtgaa atccgcccga cgattggcca gcaaatggaa | 120 |
| actggcgacc aacggtttgg cgatctggtt ttccgccagc tcgcaccgaa tgtctggcag | 180 |
| cacacttcct atctcgacat gccgggtttc ggggcagtcg cttccaacgg tttgatcgtc | 240 |
| agggatggcg gccgcgtgct gttggtcgat accgcctgga ccgatgacca gaccgcccag | 300 |
| atcctcaact ggatcaagca ggagatcaac ctgccggtcg cgctggcggt ggtgactcac | 360 |
| gcgcatcagg acaagatggg cggtatggac gcgctgcatg cggcggggat tgcgacttat | 420 |
| gccaatgcgt tgtcgaacca gcttgccccg caagaggggc tggttgcggc gcaacacagc | 480 |
| ctgactttcg ccgccaatgg ctgggtcgaa ccagcaaccg cgcccaactt tggcccgctc | 540 |
| aaggtatttt accccggccc cggccacacc agtgacaata tcaccgttgg gatcgacggc | 600 |
| accgacatcg cttttggtgg ctgcctgatc aaggacagca aggccaagtc gctcggcaat | 660 |
| ctcggtgatg ccgacactga gcactacgcc gcgtcagcgc gcgcgtttgg tgcggcgttc | 720 |
| cccaaggcca gcatgatcgt gatgagccat tccgcccccg atagccgcgc cgcaatcact | 780 |
| catacggccc gcatggccga caagctgcgc tgagccatgg ctgaccacgt cacccccaat | 840 |
| ctgccatcgc gcgatttcga tgtgacagag a | 871 |
| SEQ ID NO: 127 | |
| atggaattgc ccaatattat gcacccggtc gcgaagctga gcaccgcatt agccgctgca | 60 |
| ttgatgctga gcgggtgcat gcccggtgaa atccgcccga cgattggcca gcaaatggaa | 120 |
| actggcgacc aacggtttgg cgatctggtt ttccgccagc tcgcaccgaa tgtctggcag | 180 |
| cacacttcct atctcgacat gccgggtttc ggggcagtcg cttccaacgg tttgatcgtc | 240 |
| agggatggcg gccgcgtgct ggtggtcgat accgcctgga ccgatgacca gaccgcccag | 300 |
| atcctcaact ggatcaagca ggagatcaac ctgccggtcg cgctggcggt ggtgactcac | 360 |
| gcgcatcagg acaagatggg cggtatggac gcgctgcatg cggcggggat tgcgacttat | 420 |
| gccaatgcgt tgtcgaacca gcttgccccg caagaggggc tggttgcggc gcaacacagc | 480 |
| ctgactttcg ccgccaatgg ctgggtcgaa ccagcaaccg cgcccaactt tggcccgctc | 540 |
| aaggtatttt accccggccc cggccacacc agtgacaata tcaccgttgg gatcgacggc | 600 |
| accgacatcg cttttggtgg ctgcctgatc aaggacagca aggccaagtc gctcggcaat | 660 |
| ctcggtgatg ccgacactga gcactacgcc gcgtcagcgc gcgcgtttgg tgcggcgttc | 720 |
| cccaaggcca gcatgatcgt gatgagccat tccgcccccg atagccgcgc cgcaatcact | 780 |
| catacggccc gcatggccga caagctgcgc tga | 813 |
| SEQ ID NO: 128 | |
| atggaattgc ccaatattat gcacccggtc gcgaagctga gcaccgcatt agccgctgca | 60 |
| ttgatgctga gcgggtgcat gcccggtgaa atccgcccga cgattggcca gcaaatggaa | 120 |
| actggcgacc aacggtttgg cgatctggtt ttccgccagc tcgcaccgaa tgtctggcag | 180 |
| cacacttcct atctcgacat gccgggtttc ggggcagtcg cttccaacgg tttgatcgtc | 240 |
| agggatggcg gccgcgtgct ggtggtcgat accgcctgga ccgatgacca gaccgcccag | 300 |
| atcctcaact ggatcaagca ggagatcaac ctgccggtcg cgctggcggt ggtgactcac | 360 |
| gcgcatcagg acaagatggg cggtatggac gcgctgcatg cggcggggat tgcgacttat | 420 |
| gccaatgcgt tgtcgaacca gcttgccccg caaaagggga tggttgcggc gcaacacagc | 480 |
| ctgactttcg ccgccaatgg ctgggtcgaa ccagcaaccg cgcccaactt tggcccgctc | 540 |
| aaggtatttt accccggccc cggccacacc agtgacaata tcaccgttgg gatcgacggc | 600 |
| accgacatcg cttttggtgg ctgcctgatc aaggacagca aggccaagtc gctcggcaat | 660 |
| ctcggtgatg ccgacactga gcactacgcc gcgtcagcgc gcgcgtttgg tgcggcgttc | 720 |
| cccaaggcca gcatgatcgt gatgagccat tccgcccccg atagccgcgc cgcaatcact | 780 |
| catacggccc gcatggccga caagctgcgc tga | 813 |
| SEQ ID NO: 129 | |
| atggaattgc ccaatattat gcacccggtc gcgaagctga gcaccgcatt agccgctgca | 60 |
| ttgatgctga gcgggtgcat gcccggtgaa atccgcccga cgattggcca gcaaatggaa | 120 |
| actggcgacc aacggtttgg cgatctggtt ttccgccagc tcgcaccgaa tgtctggcag | 180 |
| cacacttcct atctcgacat gccgggtttc ggggcagtcg cttccaacgg tttgatcgtc | 240 |
| agggatggcg gccgcgtgct ggtggtcgat accgcctgga ccaatgacca gaccgcccag | 300 |
| atcctcaact ggatcaagca ggagatcaac ctgccggtcg cgctggcggt ggtgactcac | 360 |
| gcgcatcagg acaagatggg cggtatggac gcgctgcatg cggcggggat tgcgacttat | 420 |
| gccaatgcgt tgtcgaacca gcttgccccg caagagggga tggttgcggc gcaacacagc | 480 |
| ctgactttcg ccgccaatgg ctgggtcgaa ccagcaaccg cgcccaactt tggcccgctc | 540 |
| aaggtatttt accccggccc cggccacacc agtgacaata tcaccgttgg gatcgacggc | 600 |
| accgacatcg cttttggtgg ctgcctgatc aaggacagca aggccaagtc gctcggcaat | 660 |
| ctcggtgatg ccgacactga gcactacgcc gcgtcagcgc gcgcgtttgg tgcggcgttc | 720 |
| cccaaggcca gcatgatcgt gatgagccat tccgcccccg atagccgcgc cgcaatcact | 780 |
| catacggccc gcatggccga caagctgcgc tga | 813 |
| SEQ ID NO: 130 | |
| atggaattgc ccaatattat gcacccggtc gcgaagctga gcaccgcatt agccgctgca | 60 |
| ttgatgctga gcgggtgcat gcccggtgaa atccgcccga cgattggcca gcaaatggaa | 120 |
| actggcgacc aacggtttgg cgatctggtt ttccgccagc tcgcaccgaa tgtctggcag | 180 |
| cacacttcct atctcgacat gccgggtttc ggggcagtcg cttccaacgg tttgatcgtc | 240 |
| agggatggcg gccgcgtgct ggtggtcgat accgcctgga ccgatgacca gaccgcccag | 300 |
| atcctcaact ggatcaagca ggagatcaac ctgccggtcg cgctggcggt ggtgactcac | 360 |
| gcgcatcagg acaagatggg cggtatggac gcgctgcatg cggcggggat tgcgacttat | 420 |
| gccaatgcgt tgtcgaacca gcttgccccg caagagggga tggttgcggc gcaacacagc | 480 |
| ctgactttcg ccgccaatgg ctgggtcgaa ccagcaaccg cgcccaactt tggcccgctc | 540 |
| aaggtatttt accccggccc cggccacacc agtgacaata tcaccgttgg gatcgacggc | 600 |
| accgacatcg cttttggtgg ctgcctgatc aaggacagca aggccaagtc gctcggcaat | 660 |
| ctcggtgatg ccgacactga gcactacgcc gcgtcagtgc gcgcgtttgg tgcggcgttc | 720 |
| cccaaggcca gcatgatcgt gatgagccat tccgcccccg atagccgcgc cgcaatcact | 780 |
| catacggccc gcatggccga caagctgcgc tga | 813 |
| SEQ ID NO: 131 | |
| atggaattgc ccaatattat gcacccggtc gcgaagctga gcaccgcatt agccgctgca | 60 |
| ttgatgctga gcgggtgcat gcccggtgaa atccgcccga cgattggcca gcaaatggaa | 120 |
| actggcgacc aacggtttgg cgatctggtt ttccgccagc tcgcaccgaa tgtctggcag | 180 |
| cacacttcct atctcgacat gccgggtttc ggggcagtcg cttccaacgg tttgatcgtc | 240 |
| agggatggcg gccgcgtgct ggtggtcgat accgcctgga ccgatgacca gaccgcccag | 300 |
| atcctcaact ggatcaagca ggagatcaac ctgccggtcg cgctggcggt ggtgactcac | 360 |
| gcgcatcagg acaagatggg cggtatggac gcgctgcatg cggcggggat tgcgacttat | 420 |
| gccaatgcgt tgtcgaacca gcttgccccg caagagggga tggttgcggc gcaacacagc | 480 |
| ctgactttcg ccgccaatgg ctgggtcgaa ccagcaaccg cgcccaactt tggcccgctc | 540 |
| aaggtatttt accccggccc cggccacacc agtgacaata tcaccgttgg gatcgacggc | 600 |
| accgacatcg cttttggtgg ctgcctgatc aaggacagca aggccaagtc gctcggcaat | 660 |
| ctcggtgatg ccgacactga gcactacgcc gcgtcagcgc gcgcgtttgg tgcggcgttc | 720 |
| cccaaggcca gcatgatcgt gatgagccat tccgcccccg atagccgcgc cgcaatcact | 780 |
| catacggccc gcatggccga caagctgcgc tga | 813 |
| SEQ ID NO: 132 | |
| atggaattgc ccaatattat gcacccggtc gcgaagctga gcaccgcatt agccgctgca | 60 |
| ttgatgctga gcgggtgcat ggccggtgaa atccgcccga cgattggcca gcaaatggaa | 120 |
| actggcgacc aacggtttgg cgatctggtt ttccgccagc tcgcaccgaa tgtctggcag | 180 |
| cacacttcct atctcgacat gccgggtttc ggggcagtcg cttccaacgg tttgatcgtc | 240 |
| agggatggcg gccgcgtgct ggtggtcgat accgcctgga ccgatgacca gaccgcccag | 300 |
| atcctcaact ggatcaagca ggagatcaac ctgccggtcg cgctggcggt ggtgactcac | 360 |
| gcgcatcagg acaagatggg cggtatggac gcgctgcatg cggcggggat tgcgacttat | 420 |
| gccaatgcgt tgtcgaacca gcttgccccg caagagggga tggttgcggc gcaacacagc | 480 |
| ctgactttcg ccgccaatgg ctgggtcgaa ccagcaaccg cgcccaactt tggcccgctc | 540 |
| aaggtatttt accccggccc cggccacacc agtgacaata tcaccgttgg gatcgacggc | 600 |
| accgacatcg cttttggtgg ctgcctgatc aaggacagca aggccaagtc gctcggcaat | 660 |
| ctcggtgatg ccgacactga gcactacgcc gcgtcagcgc gcgcgtttgg tgcggcgttc | 720 |
| cccaaggcca gcatgatcgt gatgagccat tccgcccccg atagccgcgc cgcaatcact | 780 |
| catacggccc gcatggccga caagctgcgc tga | 813 |
| SEQ ID NO: 133 | |
| atgaaaaaat tatttgtttt atgtgtattc ttcctttgca acattgctgc tgcagatgat | 60 |
| tctttgcctg atttaaaaat tgagaagctt gaaaaaggcg tttatgttca tacttcgttt | 120 |
| gaagaagtta aaggttgggg tgtagtcaca aaacacggtt tagtggttct tgtaaagaat | 180 |
| gatgcttatc tgatagatac tccaattacc gctaaagata ctgaaaaatt agttaattgg | 240 |
| tttattgagc acggctatag aatcaaaggc agtatttcca cacatttcca tggcgacagt | 300 |
| acggctggaa tagagtggct taattctcaa tctatctcca cgtatgcctc tgaattaaca | 360 |
| aatgaacttc taaaaaaaga caataaggtg caagctacaa attcttttag tggagttagt | 420 |
| tattcactta tcaaaaacaa aattgaagtt ttctatccag gtccaggaca cactcaagat | 480 |
| aacgtagtgg tttggttacc tgaaaagaaa attttattcg gtggttgctt tgttaaaccg | 540 |
| gacggtcttg gaaatttagg ggatgcaaat ttagaagctt ggccaaagtc cgctaaaata | 600 |
| ttaatgtcta aatatggtaa agcaaaactg gttgtttcaa gtcatagtga aattggaaac | 660 |
| gcatcactct tgcagcgcac atgggagcag gctgttaaag ggttaaatga aagtaaaaaa | 720 |
| ccgttacagc caagtagcta a | 741 |
| SEQ ID NO: 134 | |
| atgaaaaaat tatttgtttt atgtgtattc ttcctttgca acattgctgc tgcagatgat | 60 |
| tctttgcctg atttaaaaat tgagaagctt gaaaaaggcg tttatgttca tacttcgttt | 120 |
| gaagaagtta aaggttgggg tgtagtcaca aaacacggtt tagtggttct tgtaaagaat | 180 |
| gatgcttatc tgatagatac tccaattacc gctaaagata ctgaaaaatt agttaattgg | 240 |
| tttattgagc acggctatag aatcaaaggc agtatttcca cacatttcca tggcgacagt | 300 |
| acggctggaa tagagtggct taattctcaa tctatctcca cgtatgcctc tgaattaaca | 360 |
| aatgaacttc taaaaaaaga caataaggtg caagctacaa attcttttag tggagttagt | 420 |
| tattcactta tcaaaaacaa aattgaagtt ttctatccag gtccaggaca cactcaagat | 480 |
| aacgtagtgg tttggttacc tgaaaagaaa attttattcg gtggttgctt tgttaaaccg | 540 |
| gacggtcttg gaaatttagg ggatgcaaat ttagaagctt ggccaaagtc cgctaaaata | 600 |
| ttaatgtcta aatatggtaa agcaaaactg gttgtttcaa gtcatagtga aattggaaac | 660 |
| gcatcactct tgcagcgcac atgggagcag gctgttaaag ggttaaatga aagtaaaaaa | 720 |
| ccgttacagc caagtagcta a | 741 |
| SEQ ID NO: 135 | |
| atgaaaaaat tatttgtttt atgtgtattc ttcctttgca acattgctgc tgcagatgat | 60 |
| tctttgcctg atttaaaaat tgagaagctt gaaaaaggcg tttatgttca tacttcgttt | 120 |
| gaagaagtta aaggttgggg tgtagtcaca aaacacggtt tagtggttct tgtaaagaat | 180 |
| gatgcttatc tgatagatac tccaattacc gctaaagata ctgaaaaatt agttaattgg | 240 |
| tttattgagc acggctatag aatcaaaggc agtatttcca cacatttcca tggcgacagt | 300 |
| acggctggaa tagagtggct taattctcaa tctatctcca cgtatgcctc tgaattaaca | 360 |
| aatgaacttc taaaaaaaga caataaggtg caagctacaa attcttttag tggagttagt | 420 |
| tattcactta tcaaaaacaa aattgaagtt ttctatccag gtccaggaca cactcaagat | 480 |
| aacgtagtgg tttggttacc tgaaaagaaa attttattcg gtggttgctt tgttaaaccg | 540 |
| gacggtcttg gaaatttagg ggatgcaaat ttagaagctt ggccaaagtc cgctaaaata | 600 |
| ttaatgtcta aatatggtaa agcaaaactg gttgtttcaa gtcatagtga aattggaaac | 660 |
| gcatcactct tgcagcgcac atgggagcag gctgttaaag ggttaaatga aagtaaaaaa | 720 |
| ccgttacagc caagtagcta a | 741 |
| SEQ ID NO: 136 | |
| atgaaaaaat tatttgtttt atgtgtattc ttcctttgca acattgctgc tgcagatgat | 60 |
| tctttgcctg atttaaaaat tgagaagctt gaaaaaggcg tttatgttca tacttcgttt | 120 |
| gaagaagtta aaggttgggg tgtagtcaca aaacacggtt tagtggttct tgtaaagaat | 180 |
| gatgcttatc tgatagatac tccaattacc gctaaagata ctgaaaaatt agttaattgg | 240 |
| tttattgagc acggctatag aatcaaaggc agtatttcca cacatttcca tggcgacagt | 300 |
| acggctggaa tagagtggct taattctcaa tctatctcca cgtatgcctc tgaattaaca | 360 |
| aatgaacttc taaaaaaaga caataaggtg caagctacaa attcttttag tggagttagt | 420 |
| tattcactta tcaaaaacaa aattgaagtt ttctatccag gtccaggaca cactcaagat | 480 |
| aacgtagtgg tttggttacc tgaaaagaaa attttattcg gtggttgctt tgttaaaccg | 540 |
| gacggtcttg gaaatttagg ggatgcaaat ttagaagctt ggccaaagtc cgctaaaata | 600 |
| ttaatgtcta aatatggtaa agcaaaactg gttgtttcaa gtcatagtga aattggaaac | 660 |
| gcatcactct tgcagcgcac atgggagcag gctgttaaag ggttaaatga aagtaaaaaa | 720 |
| ccgttacagc caagtagcta a | 741 |
| SEQ ID NO: 137 | |
| atgaaaaaaa tatttgtgtt atttgtattt ttgttttgca gtattactgc cgccggagag | 60 |
| tctttgcctg atataaaaat tgagaaactt gacgaagatg tttatgttca tacttctttt | 120 |
| gaaaagataa ccggctgggg tgttattact aaacacggct tggtggttct tgtaaatact | 180 |
| gatgcctata taattgacac tccatttaca gctaaagata ctgaaaaatt agtccgctgg | 240 |
| tttgtggggc gtggttataa aatcaaaggc agtatttcct cacattttca tagcgatagc | 300 |
| gcaggtggaa ttgagtggct taattctcaa tctatcccca catatgcatc taaattaaca | 360 |
| aatgagcttc ttaaaaagaa cggtaatgcg caagccgaaa actcatttag tggcgttagc | 420 |
| tattggctag ttaaacataa aattgaagtt ttctatccag gaccagggca cactcaggat | 480 |
| aatgtagtgg tttggttgcc tgaaaagaaa attttatttg gcggttgttt tattaagccg | 540 |
| gacggtcttg gttatttggg agacgcaaat ctagaagcat ggcctaagtc cgcagaaaca | 600 |
| ttaatgtcta agtatggtaa tgcaaaactg gttgtttcga gtcatagtga aattgggggc | 660 |
| gcatcactat tgaagcgcac ttgggagcag gctgttaagg ggctaaaaga aagtaaaaac | 720 |
| catcacagcc ccaaataa | 738 |
| SEQ ID NO: 138 | |
| atgaaaaaat tatttgtttt atgtgtattc ttcttctgca acattgcagt tgcagaagaa | 60 |
| tctttgcctg atttaaaaat tgagaagctt gaagaaggcg tttatgttca tacttcgttt | 120 |
| gaagaagtta aaggttggag tgtggtcact aaacacggtt tggtggttct tgtgaaaaat | 180 |
| gacgcctatc tgattgatac tccaattact gctaaagata ctgaaaaatt agtcaattgg | 240 |
| tttgttgagc ggggctataa aatcaaaggc agtatttcca cacatttcca tggtgacagt | 300 |
| acggctggaa tagagtggct taattctcaa tctatcccca catatgcttc tgaattaaca | 360 |
| aatgaacttc ttaaaaaaga caataaggta caagctaaac actcttttta tggggttagt | 420 |
| tattcactaa taaaaaacaa aattgaagtt ttttatccag gcccagggca cactcaagat | 480 |
| aacgtagtgg tttggttacc tgaaaagaaa attttattcg gtggttgctt tgttaaaccg | 540 |
| gacggtcttg gctatttggg ggacgcaaat ttagaagctt ggccaaagtc cgctaaaata | 600 |
| ttaatgtcta aatatggtaa agcaaaacta gttgtgtcga gtcatagtga tattggagat | 660 |
| gtatcactct tgaaacgtac atgggagcag gctgttaaag ggctgaatga aagtaaaaaa | 720 |
| tcatcacagc caagcgacta a | 741 |
| SEQ ID NO: 139 | |
| atgaaaaaat tatttgtttt atgtgtattc ttcttctgca acattgcagt tgcagaagaa | 60 |
| tctttgcctg atttaaaaat tgagaagctt gaagaaggcg tttatgttca tacttcgttt | 120 |
| gaagaagtta aaggttggag tgtggtcact aaacacggtt tggtggttct tgtgaaaaat | 180 |
| gacgcctatc tgattgatac tccaattact gctaaagata ctgaaaaatt agtcaattgg | 240 |
| tttgttgagc ggggctataa aatcaaaggc agtatttcca cacatttcca tggtgacagt | 300 |
| acggctggaa tagagtggct taattctcaa tctatcccca catatgcttc tgaattaaca | 360 |
| aatgaacttc ttaaaaaaga caataaggta caagctaaac actcttttaa tggggttagt | 420 |
| tattcactaa ttaaaaacaa aattgaagtt ttttatccag gcccagggca cactcaagat | 480 |
| aacgtagtgg tttggttacc tgaaaagaaa attttattcg gtggttgctt tgttaaaccg | 540 |
| gacggtcttg gctatttggg ggacgcaaat ttagaagctt ggccaaagtc cgctaaaata | 600 |
| ttaatgtcta aatatggtaa agcaaaacta gttgtgtcga gtcatagtga tattggagat | 660 |
| gtatcactct tgaaacgtac atgggagcag gctgttaaag ggctgaatga aagtaaaaaa | 720 |
| tcatcacagc caagcgacta a | 741 |
| SEQ ID NO: 140 | |
| atgaaaaaat tatttgtttt atgtgtattc ttcttctgca acattgcagt tgcagaagaa | 60 |
| tctttgcctg atttaaaaat tgagaagctt gaagaaggcg tttatgttca tacttcgttt | 120 |
| gaagaagtta aaggttggag tgtggtcact aaacacggtt tggtggttct tgtgaaaaat | 180 |
| gacgcctatc tgattgatac tccaattact gctaaagata ctgaaaaatt agtcaattgg | 240 |
| tttgttgagc ggggctataa aatcaaaggc agtatttcca cacatttcca tggtgacagt | 300 |
| acggctggaa tagagtggct taattctcaa tctatcccca catatgcttc tgaattaaca | 360 |
| aatgaacttc ttaaaaaaga caataaggta caagctaaac actcttttaa tggggttagt | 420 |
| tattcactaa ttaaaaacaa aattgaagtt ttttatccag gcccagggca cactcaagat | 480 |
| aacgtagtgg tttggttacc tgaaaagaaa attttattcg gtggttgctt tgttaaaccg | 540 |
| gacggtcttg gctatttggg ggacgcaaat ttagaagctt ggccaaagtc cgctaaaata | 600 |
| ttaatgtcta aatatggtaa agcaaaacta gttgtgtcga gtcatagtga tattggagat | 660 |
| gtatcactct tgaaacgtac atgggagcag gctgttaaag ggctgaatga aagtaaaaaa | 720 |
| tcatcacagc caagcgacta a | 741 |
| SEQ ID NO: 141 | |
| atgaaaaaat tatttgtttt atgtgtattc ttcttctgca acattgcagt tgcagaagaa | 60 |
| tctttgcctg atttaaaaat tgagaagctt gaagaaggcg tttatgttca tacttcgttt | 120 |
| gaagaagtta aaggttggag tgtggtcact aaacacggtt tggtggttct tgtgaaaaat | 180 |
| gacgcctatc tgattgatac tccaattact gctaaagata ctgaaaaatt agtcaattgg | 240 |
| tttgttgagc ggggctataa aatcaaaggc agtatttcaa cacatttcca tggtgacagt | 300 |
| acggctggaa tagagtggct taattctcaa tctatcccca catatgcttc tgaattaaca | 360 |
| aatgaacttc ttaaaaaaga caataaggta caagctaaac actcttttaa tggggttagt | 420 |
| tattcactaa ttaaaaacaa aattgaagtt ttttatccag gcccagggca cactcaagat | 480 |
| aacgtagtgg tttggttacc tgaaaagaaa attttattcg gtggttgctt tgttaaaccg | 540 |
| gacggtcttg gctatttggg ggacgcaaat ttagaagctt ggccaaagtc cgctaaaata | 600 |
| ttaatgtcta aatatggtaa agcaaaacta gttgtgtcga gtcatagtga tattggagat | 660 |
| gtatcactct tgaaacgtac atgggagcag gctgttaaag ggctgaatga aagtaaaaaa | 720 |
| tcatcacagc caagcgacta a | 741 |
| SEQ ID NO: 142 | |
| atgaaaaaac tatttgtttt atgtatattt ttgttttgta gcattactgc cgcaggagcg | 60 |
| tctttgcctg atttaaaaat tgagaagctt gaagagggtg tttatgttca tacatcgttt | 120 |
| gaagaagtta acggctgggg tgttgcttct aaacacggtt tggtggttct tgtaaatact | 180 |
| gacgcctatc tgattgacac tccatttact gctaaagata ctgaaaagtt agtcaattgg | 240 |
| tttgtggagc gcggctataa aatcaaaggc agtatttcct cacatttcca tagcgacagc | 300 |
| acgggtggaa tagagtggct taattctcaa tctattccca cgtatgcatc tgtattaaca | 360 |
| aatgaacttc tcaaaaaaga cggtaaggtg caagctaaaa actcatttag cggagttagc | 420 |
| tattggctag ttaaaaataa aattgaagtt ttttatccag gcccagggca cactcaagat | 480 |
| aacgtagtgg tttggctacc taaaaataaa atcttatttg gtggttgttt tgttaaacca | 540 |
| tatggtcttg gtaatctaga tgacgcaaat gttgaagcat ggccacattc ggctgaaaaa | 600 |
| ttaatatcta agtatggtaa tgcaaaactg gttgttccaa gccatagtga cataggagat | 660 |
| gcgtcgctct tgaagcttac gtgggaacag gcggtaaaag ggcttaatga aagcaaaaaa | 720 |
| agtaacactg ttcattaa | 738 |
| SEQ ID NO: 143 | |
| atgaaaaaac tatttgtttt atgtatattt ttgttttgta gcattactgc cgcaggagcg | 60 |
| tctttgcctg atttaaaaat tgagaagctt gaagagggtg tttatgttca tacatcgttt | 120 |
| gaagaagtta acggctgggg tgttgtttct aaacacggtt tggtggttct tgtaaatact | 180 |
| gacgcctatc tgattgacac tccatttact gctaaagata ctgaaaagtt agtcaattgg | 240 |
| tttgtggagc gcggctataa aatcaaaggc agtatttcct cacatttcca tagcgacagc | 300 |
| acgggtggaa tagagtggct taattctcaa tctattccca cgtatgcatc tgtattaaca | 360 |
| aatgaacttc tcaaaaaaga cggtaaggtg caagctaaaa actcatttag cggagttagc | 420 |
| tattggctag ttaaaaataa aattgaagtt ttttatccag gcccagggca cactcaagat | 480 |
| aacgtagtgg tttggctacc taaaaataaa atcttatttg gtggttgttt tgttaaacca | 540 |
| tatggtcttg gtaatctaga tgacgcaaat gttgaagcat ggccacattc ggctgaaaaa | 600 |
| ttaatatcta agtatggtaa tgcaaaactg gttgttccaa gccatagtga cataggagat | 660 |
| gcgtcgctct tgaagcttac gtgggaacag gcggtaaaag ggcttaatga aagcaaaaaa | 720 |
| agtaacactg ttcattaacc aaatttctaa caagtcgctg aagcatcgca ccttcggtgc | 780 |
| tggacagttt ttaagtcgcg cttttgtggt tttgctacgc aaaaggtttc cacaaaatca | 840 |
| caacttaaaa actgccgctt agctcggcg | 869 |
| SEQ ID NO: 144 | |
| atgaaaaaac tatttgtttt atgtatattt ttgttttgta gcattactgc cgcaggagcg | 60 |
| tctttgcctg atttaaaaat tgagaagctt gaagagggtg tttatgttca tacatcgttt | 120 |
| gaagaagtta acggctgggg tgttttttct aaacacggtt tggtggttct tgtaaatact | 180 |
| gacgcctatc tgattgacac tccatctact gctaaagata ctgaaaagtt agtcaattgg | 240 |
| tttgtggagc gcggctataa aatcaaaggc agtatttcct cacatttcca tagcgacagc | 300 |
| acgggtggaa tagagtggct taattctcaa tctattccca cgtatgcatc tgtattaaca | 360 |
| aatgaacttc tcaaaaaaga cggtaaggtg caagctaaaa actcatttag cggagttagc | 420 |
| tattggctag ttaaaaataa aattgaagtt ttttatccag gcccagggca cactcaagat | 480 |
| aacgtagtgg tttggctacc taaaaataaa atcttatttg gtggttgttt tgttaaacca | 540 |
| tatggtcttg gtaatctaga tgacgcaaat gttgaagcat ggccacattc ggctgaaaaa | 600 |
| ttaatatcta agtatggtaa tgcaaaactg gttgttccaa gccatagtga cataggagat | 660 |
| gcgtcgctct tgaagcttac gtgggaacag gcggtaaaag ggcttaatga aagcaaaaaa | 720 |
| agtaacactg ttcattaa | 738 |
| SEQ ID NO: 145 | |
| atgaaaaaac tatttgtttt atgtatattt ttgttttgta gcattactgc cgcaggagcg | 60 |
| tctttgcctg atttaaaaat tgagaagctt gaagagggtg tttatgttca tacatcgttt | 120 |
| gaagaagtta acggctgggg tgttttttct aaacacggtt tggtggttct tgtaaatact | 180 |
| gacgcctatc tgattgacac tccatttact gctaaagata ctgaaaagtt agtcaattgg | 240 |
| tttgtggagc gcggctataa aatcaaaggc agtatttcct cacatttcca tagcgacagc | 300 |
| acgggtggaa tagagtggct taattctcaa tctattccca cgtatgcatc tgtattaaca | 360 |
| aatgaacttc tcaaaaaaga cggtaaggtg caagctaaaa actcatttag cggagttagc | 420 |
| tattggctag ttaaaaataa aattgaagtt ttttatccag gcccagggca cactcaagat | 480 |
| aacgtagtgg tttggctacc taaaaataaa atcttatttg gtggttgttt tgttaaacca | 540 |
| tatggtcttg gtaatctaga tgacgcaaat gttgaagcat ggccacattc ggctgaaaaa | 600 |
| ttaatatcta agtatggtaa tgcaaaactg gttgttccaa gccatagtga cataggagat | 660 |
| gcgtcgctct tgaagcttac gtgggaacag gcggtaaaag ggcttaatga aagcaaaaaa | 720 |
| agtaacactg ttcattaa | 738 |
| SEQ ID NO: 146 | |
| atgaagaaat tatttgtttt atgtgtgttt ttgttttgta gcattactgc cgcaggagag | 60 |
| tctttgcccg atttaaaaat tgaaaagctt gaagaaggtg tttatgttca tacatcgttt | 120 |
| gaagaagtta atggttgggg cgttgtttct aaacacggtt tggttattct tgtgaatact | 180 |
| gacgcctatc tgattgacac tccattcacg gctaaagata ctgaaaagtt agtcacctgg | 240 |
| tttgtggagc gcggctataa aatcaaaggt agcatttcct cacatttcca tagcgacagc | 300 |
| acgggtggaa tagagtggct taattctcaa tcaattccca cgtatgcatc tgaattaaca | 360 |
| aatgaccttc ttaaacaaaa cggtaaggta caagctaaaa actcatttag cggagttagt | 420 |
| tattggttag ttaaaaataa aattgaagtt ttctatcccg gccccgggca cactcaagat | 480 |
| aacgtagtgg tttggttgcc tgaaaagaaa attttatttg gtgggtgctt tgttaaaccg | 540 |
| tacggtcttg gaaatctcga tgacgcaaat gttgtagcat ggccacattc tgctgaaata | 600 |
| ttaatgtcta ggtatggtaa tgcaaaactg gttgttccaa gccatagtga catcggagat | 660 |
| gcgtcgctct tgaagcttac atgggagcag gctgttaaag ggctaaaaga aagtaaaaaa | 720 |
| ccatcagagc caagtaacta a | 741 |
| SEQ ID NO: 147 | |
| atgaaaaaat tatttgtttt atgtatcttt ttgttttgta gcattactgc cgcaggagag | 60 |
| tctttgcctg atttaaaaat tgagaagctt gaagacggtg tttatgttca tacatcgttt | 120 |
| gaagaagtta acggttgggg tgttgttact aaacacggtt tggtgtttct tgtaaacaca | 180 |
| gacgcctatc tgattgacac tccatttgct gctaaagaca ctgaaaagtt agtaaattgg | 240 |
| tttgtggagc gcggttataa aataaaaggc agtatttcct cacattttca tagcgacagc | 300 |
| tcgggtggaa tagaatggct taactctcaa tctattccca cgtatgcatc tgaattaaca | 360 |
| aacgaacttc ttaaaaagaa cggtaaggtg caagctaaaa actcatttag cggagttagt | 420 |
| tattggctac ttaaaaataa aattgaaatt ttttatccgg gccctgggca cactcaagat | 480 |
| aacgtagtgg tttggttgcc tgaaaagaaa attttatttg gtgggtgttt tgttaaaccg | 540 |
| tacggtcttg gaaatctcga tgatgcaaat gttgaagcgt ggccacattc tgctgaaata | 600 |
| ttaatgtcta ggtatggtaa tgcaaaactg gttgttccaa gccatagtga cgtcggagat | 660 |
| gcgtcgctct tgaagcttac atgggagcag gctgttaaag ggctaaaaga aagtaaaaaa | 720 |
| ccatcacagc caagtaacta a | 741 |
| SEQ ID NO: 148 | |
| atgagcaagt tatttgtatt ctttatgttt ttgttttgta gcattactgc cgcaggagag | 60 |
| tctttgccag atttaaaaat tgagaagctt gacgaaggcg tttatgttca tacttcgttt | 120 |
| gaagaagtta acggttgggg tgttattcct aaacacggct tggtggttct tgtaaatact | 180 |
| gatgcctatc tgatagacac tccatttact gctaaagata ctgaaaattt agttaattgg | 240 |
| tttgttgagc gcggctatag aataaaaggc agtatttcct cacatttcca tagcgacagc | 300 |
| acgggtggaa tagagtggct taattctcaa tctatcccca cgtatgcatc tgaattaaca | 360 |
| aatgaacttc ttaaaaaaga cggtaaggta caagctaaat attcatttag cggagttagc | 420 |
| tattggctag ttaagaaaaa gattgaagtt ttttatcctg gtccagggca cgctccagat | 480 |
| aacgtagtgg tttggctgcc tgaaaataga gttttgttcg gtggttgttt tgttaaaccc | 540 |
| tacggtctag gtaatttggg tgacgcaaat ttagaagctt ggccaaaatc cgccaaatta | 600 |
| ttaatgtcaa aatatagtaa ggcaaaactg gttgtaccaa gtcatagtga cataggagat | 660 |
| tcgtcgctct tgaagcttac atgggagcag acggtaaaag gattcaatga aagcaaaaaa | 720 |
| agtaccactg cacattaa | 738 |
| SEQ ID NO: 149 | |
| atgagcaagt tatttgtatt ctttatgttt ttgttttgta gcattactgc cgcaggagag | 60 |
| tctttgccag atttaaaaat tgagaagctt gacgaaggcg tttatgttca tacttcgttt | 120 |
| gaagaagtta acggttgggg tgttattcct aaacacggct tggtggttct tgtaaatact | 180 |
| gatgcctatc tgatagacac tccatttact gctaaagata ctgaaaattt agttaattgg | 240 |
| tttgttgagc gcggctatag aataaaaggc agtatttcct cacatttcca tagcgacagc | 300 |
| acgggtggaa tagagtggct taattctcaa tctatcccca cgtatgcatc tgaattaaca | 360 |
| aatgaacttc ttaaaaaaga cggtaaggta caagctaaat attcatttag cggagttagc | 420 |
| tattggctag ttaagaaaaa gattgaagtt ttttatcctg gttcagggca cgctccagat | 480 |
| aacgtagtgg tttggctgcc tgaaaataga gttttgttcg gtggttgttt tgttaaaccc | 540 |
| tacggtctag gtaatttggg tgacgcaaat ttagaagctt ggccaaaatc cgccaaatta | 600 |
| ttaatgtcaa aatatagtaa ggcaaaactg gttgtaccag gtcatagtga cataggagat | 660 |
| tcgtcgctct tgaagcttac atgggagcag acggtaaaag gattcaatga aagcaaaaaa | 720 |
| agtaccactg cacattaa | 738 |
| SEQ ID NO: 150 | |
| atgagcaagt tatttgtatt ctttatgttt ttgttttgta gcattactgc cgcaggagag | 60 |
| tctttgccag atttaaaaat tgagaagctt gacgaaggcg tttatgttca tacttcgttt | 120 |
| gaagaagtta acggttgggg tgttattcct aaacacggct tggtggttct tgtaaatact | 180 |
| gatgcctatc tgatagacac tccatttact gctaaagata ctgaaaattt agttaattgg | 240 |
| tttgttgagc gcggctatag aataaaaggc agtatttcct cacatttcca tagcgacagc | 300 |
| acgggtggaa tagagtggct taattctcaa tctatcccca cgtatgcatc tgaattaaca | 360 |
| aatgaacttc ttaaaaaaga cggtaaggta caagctaaat attcatttag cggagttagc | 420 |
| tattggctag ttaagaaaaa gattgaagtt ttttatcctg gtccagggca cgctccagat | 480 |
| aacgtagtgg tttggctgcc tgaaaataga gttttgttcg gtggttgttt tgttaaaccc | 540 |
| tacggtctag gtaatttggg tgacgcaaat ttagaagctt ggccaaaatc cgccaaatta | 600 |
| ttaatgtcaa aatatagtaa ggcaaaactg gttgtaccag gtcatagtga cataggagat | 660 |
| tcgtcgctct tgaagcttac atgggagcag acggtaaaag gattcaatga aagcaaaaaa | 720 |
| agtaccactg cacattaa | 738 |
| SEQ ID NO: 151 | |
| atgagcaagt tatttgtatt ccttattttt ttgttttgta gcattactgc cgcagcagag | 60 |
| tctttgccag atttaaaaat tgagaagctc gacgaaggcg tttatgttca tacttcgttt | 120 |
| gaagaagtta acggttgggg tgttgttcct aaacatggct tggtggttct tgtaaatact | 180 |
| gaggcctatc tgattgacac tccatttacg gctaaagata ctgaaaagtt agtcacttgg | 240 |
| tttgtggaac gcggctataa aataaaaggc agtatttcct ctcattttca tagcgacagc | 300 |
| acaggcggaa tagagtggct taattctcaa tctatcccca cgtatgcatc tgaattaaca | 360 |
| aatgaacttc ttaaaaaagg cggtaaagta caagctaaaa attcatttag cggagttagc | 420 |
| tattggctag ttaagaaaaa gattgaagtt ttttatcctg gtccagggca cactccagat | 480 |
| aacgtagtgg tttggctacc tgaaaataga gttttgttcg gtggttgttt tgttaaaccg | 540 |
| tacggtcttg gaaatctcga tgacgcaaat gttgaagcat ggccacattc tgctgaaata | 600 |
| ttaatgtcta ggtatggtaa tgcaaaactg gttgttccaa gccatagtga catcggaaat | 660 |
| gcgtcgctct tgaagcttac atgggagcag gctgttaaag ggctaaaaga aagtaaaaaa | 720 |
| ccatcacagc caagtaacta a | 741 |
| SEQ ID NO: 152 | |
| atgaacaagt tatctgtatt ctttatgttt atgttttgta gcattactgc cgcaggagag | 60 |
| tctttgccag atttaaaaat tgagaagctt gacgaaggtg tttatgttca tacttcgttt | 120 |
| gaagaagtta acggttgggg tgttgttcct aaacacggct tggtggttct tgtaaatact | 180 |
| gaggcctatc tgattgacac tccatttacg gcaaaagata ctgaaaagtt agtcacttgg | 240 |
| tttgtggagc gcggctataa aataaaaggc agtatttcct ctcattttca tagcgacagc | 300 |
| acgggcggaa tagagtggct taattctcaa tctatcccca cgtatgcatc tgaattaaca | 360 |
| aatgaacttc ttaaaaaaga cggtaaggta caagctaaaa attcatttag cggaggtagc | 420 |
| tattggctag ttaataataa gattgaagtt ttttatcctg gtccagggca cactccagat | 480 |
| aacgtagtgg tttggctacc tgaaaataga gttttgttcg gtggttgttt tgttaaaccg | 540 |
| tacggtcttg gtaatttggg tgacgcaaat ttagaagctt ggccaaagtc cgccaaaata | 600 |
| ttaatgtcta aatatggtaa agcaaagttg gttgtttcaa gtcatagtga aactgggaac | 660 |
| gcatcactct tgaaacttac ttgggagcag gctgttaaag ggctaaaaga aagtaaaaaa | 720 |
| ccatcactgc caagtaacta a | 741 |
| SEQ ID NO: 153 | |
| atgaacaagt tatctgtatt ctttatgttt atgttttgta gcattactgc cgcaggagag | 60 |
| tctttgccag atttaaaaat tgagaagctt gacgaaggtg tttatgttca tacttcgttt | 120 |
| gaagaagtta acggttgggg tgttgttcct aaacacggct tggtggttct tgtaaatact | 180 |
| gaggcctatc tgattgacac tccatttacg gcaaaagata ctgaaaagtt agtcacttgg | 240 |
| tttgtggagc gcggctataa aataaaaggc agtatttcct ctcattttca tagcgacagc | 300 |
| acgggcggaa tagagtggct taattctcaa tctatcccca cgtatgcatc tgaattaaca | 360 |
| aatgaacttc ttaaaaaaga cggtaaggta caagctaaaa attcatttag cggaggtagc | 420 |
| tattggctag ttaataataa gattgaagtt ttttatcctg gtccagggca cactccagat | 480 |
| aacgtagtgg tttggctacc tgaaaataga gttttgttcg gtggttgttt tgttaaaccg | 540 |
| tacggtcttg gtaatttggg tgacgcaaat ttagaagctt ggccaaagtc cgccaaaata | 600 |
| ttaatgtcta aatatggtaa agcaaagttg gttgtttcaa gtcatagtga aactgggaac | 660 |
| gcatcactct tgaaacttac ttgggagcag gctgttaaag ggctaaaaga aagtaaaaaa | 720 |
| ccatcactgc caagtaacta a | 741 |
| SEQ ID NO: 154 | |
| atgagcaagt tatttgtatt ctttatgttt ttgttttgta gcattactgc cgcagcagag | 60 |
| tctttgccag atttaaaaat tgagaagctt gacgaaggcg tttatgttca tacttcgttt | 120 |
| gaagaagtta acggttgggg tgttgttcct aaacacggct tggtggttct tgtaaatact | 180 |
| gaggcctatc tgattgacac tccatttacg gctaaagata ctgaaaagtt agtcacttgg | 240 |
| tttgtggaac gcggctataa aataaaaggc agtatttcct ctcattttca tagcgacagc | 300 |
| acgggcggaa tagagtggct taattctcaa tctatcccca cgtatgcatc tgaattaaca | 360 |
| aatgaacttc ttaaaaaaga cggtaaagta caagctaaaa attcatttag cggagctagc | 420 |
| tattggctag ttaagaaaaa gattgaagtt ttttatcctg gtccagggca cactccagat | 480 |
| aacgtagtgg tttggctacc tgaaaataga gttttgttcg gtggttgttt tgttaaaccg | 540 |
| tacggtctag gtaatttggg tgacgcaaat gtagaagctt ggccaaagtc cgccaaatta | 600 |
| ttaatgtcca aatatggtaa ggcaaaactg gtagttccaa gtcacagtga agttggagac | 660 |
| gcatcactct tgaaacgtac gttagaacag gcggttaaag ggttaaacga aagtaaaaaa | 720 |
| ccatcaaaac caagtaacta a | 741 |
| SEQ ID NO: 155 | |
| atgagcaagt tatttgtatt ctttatgttt ttgttttgta gcattactgc cgcagcagag | 60 |
| tctttgccag atttaaaaat tgagaagctt gacgaaggcg tttatgttca tacttcgttt | 120 |
| gaagaagtta acggttgggg tgttgttcct aaacacggct tggtggttct tgtaaatact | 180 |
| gaggcctatc tgattgacac tccatttacg gctaaagata ctgaaaagtt agtcacttgg | 240 |
| tttgtggaac gcggctataa aataaaaggc agtatttcct ctcattttca tagcgacagc | 300 |
| acgggcggaa tagagtggct taattctcaa tctatcccca cgtatgcatc tgaattaaca | 360 |
| aatgaacttc ttaaaaaaga cggtaaagta caagctaaaa attcatttag cggagctagc | 420 |
| tattggctag ttaagaaaaa gattgaagtt ttttatcctg gtccagggca cactccagat | 480 |
| aacgtagtgg tttggctacc tgaaaataga gttttgttcg gtggttgttt tgttaaaccg | 540 |
| tacggtctag gtaatttggg tgacgcaaat gtagaagctt ggccaaagtc cgccaaatta | 600 |
| ttaatgtcca aatatggtaa ggcaaaactg gtagttccaa gtcacagtga agttggagac | 660 |
| gcatcactct tgaaacgtac gttagaacag gcggttaaag ggttaaacga aagtaaaaaa | 720 |
| ccatcaaaac caagtaacta a | 741 |
| SEQ ID NO: 156 | |
| atgagcaagt tatttgtatt ctttatgttt ttgttttgta gcattactgc cgcagcagag | 60 |
| tctttgccag atttaaaaat tgagaggctt gatgaaggcg tttatgttca tacttcgttt | 120 |
| gaagaagtta acggttgggg tgttgttcct aaacacggct tggtggttct tgtaaatact | 180 |
| gaggcctatc tgattgacac tccatttacg gctaaagata ctgaaaagtt agtcacttgg | 240 |
| tttgtgggac gcggctataa aataaaaggc agtatttcct ctcattttca tagcgacagc | 300 |
| acgggcggaa tagagtggct taattctcaa tctatcccca cgtatgcatc tgaattaaca | 360 |
| aatgaacttc ttaaaaaaga cggtaaggta caagctaaaa attcatttgg cggagttagc | 420 |
| tattggctag ttaagaataa gattgaagtt ttttatcctg gtccagggca cactccagat | 480 |
| aacgtagtgg tttggctacc tgaaaataga gttttgttcg gtggttgttt tgttaaaccg | 540 |
| tacggtcttg gtaatttggg tgacgcaaat ttagaagctt ggccaaagtc cgccaaatta | 600 |
| ttaatgtcca aatatggtaa ggcaaaactg gttgttccaa gtcacagtga agttggagac | 660 |
| gcatcactct tgaagcgaac attagaacat gcggttaaag ggttaaatga aagtaaaaaa | 720 |
| ccatcaaaac caagtaacta a | 741 |
| SEQ ID NO: 157 | |
| acttcgtttg aggaagttaa cggctggggc gtggttccta aacacggctt ggtggttctt | 60 |
| gtaaatactg acgcttattt gattgacact ccatttacag ctaaagatac tgaaaagtta | 120 |
| gttacttggt ttgtagagcg cggctataaa ataaaaggca gtatctcctc tcattttcat | 180 |
| agcgacagca cgggcggaat agagtggctt aattctcaat ctattccaac atatgcatct | 240 |
| gaattaacaa atgaacttct taaaaaagac ggtaaggtac aagctaaaaa ttcatttagc | 300 |
| ggagccagct attggttagt taagaaaaag attgaaattt tttatcctgg cccagggcac | 360 |
| actccagata acgtagtggt ttggctacct gaacatagag ttttgtttgg tggttgtttt | 420 |
| gttaaaccgt atggtctagg taatttgggt gacgcaaatt tagaagcttg gccaaagtct | 480 |
| gccaaattat tagtgtccaa atatggtaag gcaaaactgg ttccaagtca cagtgaagtt | 540 |
| gga | 543 |
| SEQ ID NO: 158 | |
| atgaaaaagt tatcagtatt ctttatgttt ttgttttgta gcattgctgc ctcaggagag | 60 |
| gctttgccag atttaaaaat tgagaagctt gacgaaggcg tttatgttca tacttcgttt | 120 |
| gaggaagtta acggctgggg cgtgtttcct aaacacggct tggtggttct tgtaaatact | 180 |
| gacgcttatt tgattgacac tccatttaca gctaaagata ctgaaaagtt agttacttgg | 240 |
| tttgtagagc gcggctataa aataaaaggc agtatctcct ctcattttca tagcgacagc | 300 |
| acgggcggaa tagagtggct taattctcaa tctattccaa catatgcatc tgaattaaca | 360 |
| aatgaacttc ttaaaaaaga cggtaaggta caagctaaaa attcatttag cggagccagc | 420 |
| tattggttag ttaagaaaaa gattgaaatt ttttatcctg gcccagggca cactccagat | 480 |
| aacgtagtgg tttggctacc tgaacataga gttttgtttg gtggttgttt tgttaaaccg | 540 |
| tatggtctag gtaatttggg tgacgcaaat ttagaagctt ggccaaagtc tgccaaatta | 600 |
| ttagtgtcca aatatggtaa ggcaaaactg gttgttccaa gtcacagtga agttggagat | 660 |
| gcatcactct tgaaacgtac attagaacag gctgttaaag gattaaacga aagtaaaaag | 720 |
| ctatcaaaac caagtaacta a | 741 |
| SEQ ID NO: 159 | |
| atgaaaaagt tatcagtatt ctttatgttt ttgttttgta gcattgctgc ctcaggagag | 60 |
| gctttgccag atttaaaaat tgagaagctt gacgaaggcg tttatgttca tacttcgttt | 120 |
| gaggaagtta acggctgggg cgtggttcct aaacacggct tggtggttct tgtaaatact | 180 |
| gacgcttatt tgattgacac tccatttaca gctaaagata ctgaaaagtt agttacttgg | 240 |
| tttgtagagc gcggctataa aataaaaggc agtatctcct ctcattttca tagcgacagc | 300 |
| acgggcggaa tagagtggct taattctcaa tctattccaa catatgcatc tgaattaaca | 360 |
| aatgaacttc ttaaaaaaga cggtaaggta caagctaaaa attcatttag cggagccagc | 420 |
| tattggttag ttaagaaaaa gattgaaatt ttttatcctg gcccagggca cactccagat | 480 |
| aacgtagtgg tttggctacc tgaacataga gttttgtttg gtggttgttt tgttaaaccg | 540 |
| tatggtctag gtaatttggg tgacgcaaat ttagaagctt ggccaaagtc tgccaaatta | 600 |
| ttagtgtcca aatatggtaa ggcaaaactg gttgttccaa gtcacagtga agttggagat | 660 |
| gcatcactct tgaaacgtac attagaacag gctgttaaag gattaaacga aagtaaaaag | 720 |
| ctatcaaaac caagtaacta a | 741 |
| SEQ ID NO: 160 | |
| atgaaaaaat tatttgtttt atgtgtcttt gtcttttgta gtattactgt cgcaggtgag | 60 |
| actttgccta atttgagagt tgaaaagctt gaagaaggtg tttatgttca tacatcgtat | 120 |
| gaagaagtta aaggttgggg tgttgttact aaacacggtt tggtggttct cataggcgct | 180 |
| gacgcctatc tgattgatac tccatttact gctaaagata ctgaaaagtt agtcaattgg | 240 |
| tttgtggagc gcggctataa aataaaaggc actgtttcct cacatttcca tagcgacagt | 300 |
| acggggggaa tagagtggct taactctcag tctatcccca cgtatgcgtc tgaattaacg | 360 |
| aatgaacttc tgaaaaaaga cggtaaggtt caagccaaaa actcatttga cggggttagt | 420 |
| tattggctgg cgaaagataa aatagaagtg ttttatcctg gccctggcca cactcaagac | 480 |
| aacgtagtag tttggctgcc tgaaaaggaa atattatttg gcggttgctt tgttaagcct | 540 |
| cacggccttg gtaatttggg tgacgcaaat ttagaggctt ggccagagtc cgccaaaata | 600 |
| ttgatggaaa aatatggtaa agcaaagctg gttgtttcag gtcatagcga aaccggagac | 660 |
| gcgacacact tgaagcgtac ctgggagcag gctgttaaag gacttaaaga aagtaaaaag | 720 |
| acattgcagc caagcaacta a | 741 |
| SEQ ID NO: 161 | |
| atgagcaagt tatctgtatt ctttatattt ttgttttgta gcattgctac cgcagcagag | 60 |
| cctttgccag atttaaaaat tgaaaaactt gatgaaggcg tttatgttca tacttcgttt | 120 |
| gaagaagtta acgggtgggg cgtttttcct aaacatggtt tggttgttct tgtagatgct | 180 |
| gaagcttatc taattgacac tccatttacg gctaaagata ctgaaaagtt agtcacttgg | 240 |
| tttgtggaac gtggctataa aataaaaggc agtatttcct ctcattttca tagtgacagc | 300 |
| acgggcggaa tagagtggct taattctcaa tccatcccca cgtatgcgtc tgaattaact | 360 |
| aatgagctgc ttaaaaaaga cggtaaggtt caagctaaaa attcatttgg cggggttaac | 420 |
| tattggctag ttaaaaataa aattgaagtt ttttatccag gcccaggaca cactccagat | 480 |
| aacctagtag tttggctgcc tgaaaggaaa atattattcg gtggttgttt tattaaaccg | 540 |
| tacggtctag gtaatttggg tgacgcaaat ttagaagctt ggccaaagtc cgctaaatta | 600 |
| ttaatatcca aatatggtaa ggcaaaactg gttgttccaa gtcacagtga agctggagac | 660 |
| gcatcactct tgaaacttac attagagcag gcggttaaag ggttaaacga aagtaaaaaa | 720 |
| ccatcaaaac taagcaatta a | 741 |
| SEQ ID NO: 162 | |
| aagaagttaa cgggtggggc gtttttccta aacatggttt ggttgttctt gtagatgctg | 60 |
| aagcttatct aattgacact ccatttacgg ctaaagatac tgaaaagtta gtcacttggt | 120 |
| ttgtggaacg tggctataaa ataaaaggca gtatttcctc tcattttcat agtgacagca | 180 |
| cgggcggaat agagtggctt aattctcaat ccatccccac gtatgcgtct gaattaacta | 240 |
| atgagctgct taaaaaagac ggtaaggttc aagctaaaaa ttcatttggc ggggttaact | 300 |
| attggctagt taaaaataaa attgaagttt tttatccagg cccaggacac actccagata | 360 |
| acctagtagt ttggctgcct gaaaggaaaa tattattcgg tggttgtttt attaaaccgt | 420 |
| acggtctagg taatttgggt gacgcaaatt tagaagcttg gccaaagtcc gctaaattat | 480 |
| taatatccaa atatggtaag gcaaaactgg ttgttccaag tcacagtgaa gctgg | 535 |
| SEQ ID NO: 163 | |
| atgagcaagt tatctgtatt ctttatattt ttgttttgta gcattgctac cgcagcagag | 60 |
| cctttgccag atttaaaaat tgaaaaactt gatgaaggcg tttatgttca tacttcgttt | 120 |
| gaagaagtta acgggtgggg cgtttttcct aaacatggtt tggttgttct tgtagatgct | 180 |
| gaagcttatc taattgacac tccatttacg gctaaagata ctgaaaagtt agtcacttgg | 240 |
| tttgtggaac gtggctataa aataaaaggc agtatttcct ctcattttca tagtgacagc | 300 |
| acgggcggaa tagagtggct taattctcaa tccatcccca cgtatgcgtc tgaattaact | 360 |
| aatgagctgc ttaaaaaaga cggtaaggtt caagctaaaa attcatttgg cggggttaac | 420 |
| tattggctag ttaaaaataa aattgaagtt ttttatccag gcccaggaca cactccagat | 480 |
| aacctagtag tttggctgcc tgaaaggaaa atattattcg gtggttgttt tattaaaccg | 540 |
| tacggtctag gtaatttggg tgacgcaaat ttagaagctt ggccaaagtc cgctaaatta | 600 |
| ttaatatcca aatatggtaa ggcaaaactg gttgttccaa gtcacagtga agctggagac | 660 |
| gcatcactct tgaaacttac attagagcag gcggttaaag ggttaaacga aagtaaaaaa | 720 |
| ccatcaaaac taagcaacta a | 741 |
| SEQ ID NO: 164 | |
| atgagcaagt tatctgtatt ctttatattt ttgttttgta gcattgctac cgcagcagag | 60 |
| cctttgccag atttaaaaat tgaaaaactt gatgaaggcg tttatgttca tacttcgttt | 120 |
| gaagaagtta acgggtgggg cgttgttcct aaacatggtt tggttgttct tgtagatgct | 180 |
| gaagcttatc taattgacac tccatttacg gctaaagata ctgaaaagtt agtcacttgg | 240 |
| tttgtggaac gtggctataa aataaaaggc agtatttcct ctcattttca tagtgacagc | 300 |
| acgggcggaa tagagtggct taattctcaa tccatcccca cgtatgcgtc tgaattaact | 360 |
| aatgagctgc ttaaaaaaga cggtaaggtt caagctaaaa attcatttgg cggggttaac | 420 |
| tattggctag ttaaaaataa aattgaagtt ttttatccag gcccaggaca cactccagat | 480 |
| aacctagtag tttggctgcc tgaaaggaaa atattattcg gtggttgttt tattaaaccg | 540 |
| tacggtctag gtaatttggg tgacgcaaat ttagaagctt ggccaaagtc cgctaaatta | 600 |
| ttaatatcca aatatggtaa ggcaaaactg gttgttccag gtcacagtga agctggagac | 660 |
| gcatcactct tgaaacttac attagagcag gcggttaaag ggttaaacga aagtaaaaaa | 720 |
| ccatcaaaac taagcaacta a | 741 |
| SEQ ID NO: 165 | |
| atgagcaagt tatctgtatt ctttatattt ttgttttgta gcattgctac cgcagcagag | 60 |
| tctttgccag atttaaaaat tgaaaaactt gatgaaggcg tttatgttca tacttcgttt | 120 |
| gaagaagtta acgggtgggg cgttgttcct aaacatggtt tggttgttct tgtagatgct | 180 |
| gaagcttatc taattgacac tccatttacg gctaaagata ctgaaaagtt agtcacttgg | 240 |
| tttgtggaac gtggctataa aataaaaggc agtatttcct ctcattttca tagtgacagc | 300 |
| acgggcggaa tagagtggct taattctcaa tccatcccca cgtatgcgtc tgaattaact | 360 |
| aatgagctgc ttaaaaaaga cggtaaggtt caagctaaaa attcatttgg cggggttaac | 420 |
| tattggctag ttaaaaataa aattgaagtt ttttatccag gcccaggaca cactccagat | 480 |
| aacctagtag tttggctgcc tgaaaggaaa atattattcg gtggttgttt tattaaaccg | 540 |
| tacggtctag gtaatttggg tgacgcaaat ttagaagctt ggccaaagtc cgctaaatta | 600 |
| ttaatatcca aatatggtaa ggcaaaactg gttgttccaa gtcacagtga agctggagac | 660 |
| gcatcactct tgaaacttac attagagcag gcggttaaag ggttaaacga aagtaaaaaa | 720 |
| ccatcaaaac taagcaacta a | 741 |
| SEQ ID NO: 166 | |
| atgagcaagt tatctgtatt ctttatattt ttgttttgta gcattgctac cgcagcagag | 60 |
| cctttgccag atttaaaaat tgaaaaactt gatgaaggcg tttatgttca tacttcgttt | 120 |
| gaagaagtta acgggtgggg cgttgttcct aaacatggtt tggttgttct tgtagatgct | 180 |
| gaagcttatc taattgacac tccatttacg gctaaagata ctgaaaagtt agtcacttgg | 240 |
| tttgtggaac gtggctataa aataaaaggc agtatttcct ctcattttca tagtgacagc | 300 |
| acgggcggaa tagagtggct taattctcaa tccatcccca cgtatgcgtc tgaattaact | 360 |
| aatgagctgc ttaaaaaaga cggtaaggtt caagctaaaa attcatttgg cggggttaac | 420 |
| tattggctag ttaaaaataa aattgaagtt ttttatccag gcccaggaca cactccagat | 480 |
| aacctagtag tttggctgcc tgaaaggaaa atattattcg gtggttgttt tattaaaccg | 540 |
| tacggtctag gtaatttggg tgacgcaaat ttagaagctt ggccaaagtc cgctaaatta | 600 |
| ttaatatcca aatatggtaa ggcaaaactg gttgttccaa gtcacagtga agctggagac | 660 |
| gcatcactct tgaaacttac attagagcag gcggttaaag ggttaaacga aagtaaaaaa | 720 |
| ccatcaaaac taagcaacta a | 741 |
| SEQ ID NO: 167 | |
| atgagcaagt tatctgtatt ctttatattt ttgttttgta gcattgctac cgcagcagag | 60 |
| cctttgccag atttaaaaat tgaaaaactt gatgaaggcg tttatgttca tacttcgttt | 120 |
| gaagaagtta acgggtgggg cgttgttcct aaacatggtt tggttgttct tgtagatgct | 180 |
| gaagcttatc taattgacac tccatttacg gctaaagata ctgaaaagtt agtcacttgg | 240 |
| tttgtggaac gtggctataa aataaaaggc agtatttcct ctcattttca tagtgacagc | 300 |
| acgggcggaa tagagtggct taattctcaa tccatcccca cgtatgcgtc tgaattaact | 360 |
| aatgagctgc ttaaaaaaga cggtaaggtt caagctaaaa attcatttgg cggggttaac | 420 |
| tattggctag ttaaaaataa aattgaagtt ttttatccag gcccaggaca cactccagat | 480 |
| aacctagtag tttggctgcc tgaaaggaaa atattattcg gtggttgttt tattaaaccg | 540 |
| tacggtctag gtaatttggg tgacgcaaat ttagaagctt ggccaaagtc cgctaaatta | 600 |
| ttaatatcca aatatggtaa ggcaaaactg gttgttccaa gtcacagtga agctggagac | 660 |
| gcatcactct tgaaacttac attagagcag gcggttaaag ggttaaacga aagtaaaaaa | 720 |
| ccatcaaaac taagcaacta a | 741 |
| SEQ ID NO: 168 | |
| atgaagaaat tatttgtttt atgcattttt ttgtttttaa gtattactgc ctcaggtgag | 60 |
| gttttgcctg atttgaaaat tgagaagctt gaagagggtg tttatcttca tacatctttt | 120 |
| gaagaggtta gcggttgggg tgttgttact aaacatggtt tggtagttct tgtaaataat | 180 |
| gacgcctatc taattgacac tccatttaca aataaagata ctgaaaaatt agttgcttgg | 240 |
| tttgtagggc gcggctttac aataaaggga agtgtttcct cacattttca tagcgacagt | 300 |
| acgggtggaa tagagtggct taattctcaa tctattccca cgtatgcatc tgagttaaca | 360 |
| aatgaacttc tgaaaaagaa cggtaaggtg caagctacaa attcatttag cggggttagt | 420 |
| tattggctag ttaaaaataa aattgaaatt ttttatcccg gcccaggaca tactcaagat | 480 |
| aacgtagtgg tttggctacc tgaaaacaaa attttattcg gtggttgttt tgttaaaccg | 540 |
| gacggtcttg gtaatttgga tgacgcaaat ttaaaagctt ggccaaagtc cgcaaaaata | 600 |
| ttaatgtcta aatatggtaa agcaaagtta gttgtttcag gtcatagtga aattgggaac | 660 |
| gcatcactct tgaaacttac ttgggagcag gctgttaaag ggctaaaaga aagtaaaaaa | 720 |
| ccattactgc caagtaacta a | 741 |
| SEQ ID NO: 169 | |
| atgaagaaat tatttgtttt atgtgtatgc ttcctttgta gcattactgc cgcgggagcg | 60 |
| cgtttgcctg atttaaaaat cgagaagctt gaagaaggtg tttatgttca tacatcgttc | 120 |
| gaagaagtta acggttgggg tgttgtttct aaacacggtt tggtggttct tgtaaacact | 180 |
| gacgcctatc tgattgacac tccatttact gctacagata ctgaaaagtt agtcaattgg | 240 |
| tttgtggagc gcggctataa aatcaaaggc actatttcct cacatttcca tagcgacagc | 300 |
| acagggggaa tagagtggct taattctcaa tctattccca cgtatgcatc tgaattaaca | 360 |
| aatgaacttc ttaaaaaaga cggtaaggtg caagctaaaa actcatttag cggagttagt | 420 |
| tattggctag ttaaaaataa aattgaagtt ttttatcccg gcccggggca cactcaagat | 480 |
| aacgtagtgg tttggttacc tgaaaagaaa attttattcg gtggttgttt tgttaaaccg | 540 |
| gacggtcttg gtaatttggg tgacgcaaat ttagaagctt ggccaaagtc cgccaaaata | 600 |
| ttaatgtcta aatatgttaa agcaaaactg gttgtttcaa gtcatagtga aattggggac | 660 |
| gcatcactct tgaaacgtac atgggaacag gctgttaaag ggctaaatga aagtaaaaaa | 720 |
| ccatcacagc caagtaacta a | 741 |
| SEQ ID NO: 170 | |
| atgaagaaat tatttgtttt atgtgtatgc ttcctttgta gcattactgc cgcaggagcg | 60 |
| gctttgcctg atttaaaaat cgagaagctt gaagaaggtg tttatgttca tacatcgttc | 120 |
| gaagaagtta acggttgggg tgttgtttct aaacacggtt tggtggttct tgtaaacact | 180 |
| gacgcctatc tgattgacac tccatttact gctacagata ctgaaaagtt agtcaattgg | 240 |
| tttgtggagc gcggctataa aatcaaaggc actatttcct cacatttcca tagcgacagc | 300 |
| acagggggaa tagagtggct taattctcaa tctattccca cgtatgcatc tgaattaaca | 360 |
| aatgaacttc ttaaaaaaga cggtaaggtg caagctaaaa actcatttag cggagttagt | 420 |
| tattggctag ttaaaaataa aattgaagtt ttttatcccg gcccggggca cactcaagat | 480 |
| aacgtagtgg tttggttacc tgaaaagaaa attttattcg gtggttgttt tgttaaaccg | 540 |
| gacggtcttg gtaatttggg tgacgcaaat ttagaagctt ggccaaagtc cgccaaaata | 600 |
| ttaatgtcta aatatggtaa agcaaaactg gttgtttcaa gtcatagtga aattggggac | 660 |
| gcatcactct tgaaacgtac atgggaacag gctgttaaag ggctaaatga aagtagaaaa | 720 |
| ccatcacagc caagtaacta a | 741 |
| SEQ ID NO: 171 | |
| atgaagaaat tatttgtttt atgtgtatgc ttcctttgta gcattactgc cgcaggagcg | 60 |
| gctttgcctg atttaaaaat cgagaagctt gaagaaggtg tttatgttca tacatcgttc | 120 |
| gaagaagtta acggttgggg tgttttttct aaacacggtt tggtggttct tgtaaacact | 180 |
| gacgcctatc tgattgacac tccatttact gctacagata ctgaaaagtt agtcaattgg | 240 |
| tttgtggagc gcggctataa aatcaaaggc actatttcct cacatttcca tagcgacagc | 300 |
| acagggggaa tagagtggct taattctcaa tctattccca cgtatgcatc tgaattaaca | 360 |
| aatgaacttc ttaaaaaaga cggtaaggtg caagctaaaa actcatttag cggagttagt | 420 |
| tattggctag ttaaaaataa aattgaagtt ttttatcccg gcccggggca cactcaagat | 480 |
| aacgtagtgg tttggttacc tgaaaagaaa attttattcg gtggttgttt tgttaaaccg | 540 |
| gacggtcttg gtaatttggg tgacgcaaat ttagaagctt ggccaaagtc cgccaaaata | 600 |
| ttaatgtcta aatatgttaa agcaaaactg gttgtttcaa gtcatagtga aattggggac | 660 |
| gcatcactct tgaaacgtac atgggaacag gctgttaaag ggctaaatga aagtaaaaaa | 720 |
| ccatcacagc caagtaacta a | 741 |
| SEQ ID NO: 172 | |
| atgaagaaat tatttgtttt atgtgtatgc ttcctttgta gcattactgc cgcaggagcg | 60 |
| gctttgcctg atttaaaaat cgagaagctt gaagaaggtg tttatgttca tacatcgttc | 120 |
| gaagaagtta acggttgggg tgttgtttct aaacacggtt tggtggttct tgtaaacact | 180 |
| gacgcctatc tgattgacac tccatttact gctacagata ctgaaaagtt agtcaattgg | 240 |
| tttgtggagc gcggctataa aatcaaaggc actatttcct cacatttcca tagcgacagc | 300 |
| acagggggaa tagagtggct taattctcaa tctattccca cgtatgcatc tgaattaaca | 360 |
| aatgaacttc ttaaaaaaga cggtaaggtg caagctaaaa actcatttag cggagttagt | 420 |
| tattggctag ttaaaaataa aattgaagtt ttttatcccg gcccggggca cactcaagat | 480 |
| aacgtagtgg tttggttacc tgaaaagaaa attttattcg gtggttgttt tgttaaaccg | 540 |
| gacggtcttg gtaatttggg tgacgcaaat ttagaagctt ggccaaagtc cgccaaaata | 600 |
| ttaatgtcta aatatgttaa agcaaaactg gttgtttcaa gtcatagtga aattggggac | 660 |
| gcatcactct tgaaacgtac atgggaacag gctgttaaag ggctaaatga aagtaaaaaa | 720 |
| ccatcacagc caagtaacta a | 741 |
| SEQ ID NO: 173 | |
| atgaagaaat tatttgtttt atgtgtatgc ttcctttgta gcattactgc cgcaggagcg | 60 |
| gctttgcctg atttaaaaat cgagaagctt gaagaaggtg tttatgttca tacatcgttc | 120 |
| gaagaagtta acggttgggg tgttgtttct aaacacggtt tggtggttct tgtaaacact | 180 |
| gacgcctatc tgattgacac tccatttact gctacagata ctgaaaagtt agtcaattgg | 240 |
| tttgtggagc gcggctataa aatcaaaggc actatttcct cacatttcca tagcgacagc | 300 |
| acagggggaa tagagtggct taattctcaa tctattccca cgtatgcatc tgaattaaca | 360 |
| aatgaacttc ttaaaaaaga cggtaaggtg caagctaaaa actcatttag cggagttagt | 420 |
| tattggctag ttaaaaataa aattgaagtt ttttatcccg gcccggggca cactcaagat | 480 |
| aacgtagtgg tttggttacc tgaaaagaaa attttattcg gtggttgttt tgttaaaccg | 540 |
| gacggtcttg gtaatttggg tgacgcaaat ttagaagctt ggccaaagtc cgccaaaata | 600 |
| ttaatgtcta aatatggtaa agcaaaactg gttgtttcaa gtcatagtga aattggggac | 660 |
| gcatcactct tgaaacgtac atgggaacag gctgttaaag ggctaaatga aagtaaaaaa | 720 |
| ccatcacagc caagtaacta a | 741 |
| SEQ ID NO: 174 | |
| atgaagaaat tatttgtttt atgtgtatgc ttcctttgta gcattactgc cgcaggagcg | 60 |
| gctttgcctg atttaaaaat cgagaagctt gaagaaggtg tttatgttca tacatcgttc | 120 |
| gaagaagtta acggttgggg tgttgtttct aaacacggtt tggtggttct tgtaaacact | 180 |
| gacgcctatc tgattgacac tccatttact gctacagata ctgaaaagtt agtcaattgg | 240 |
| tttgtggagc gcggctataa aatcaaaggc actatttcct cacatttcca tagcgacagc | 300 |
| acagggggaa tagagtggct taattctcaa tctattccca cgtatgcatc tgaattaaca | 360 |
| aatgaacttc ttaaaaaaga cggtaaggtg caagctaaaa actcatttag cggagttagt | 420 |
| tattggctag ttaaaaataa aattgaagtt ttttatcccg gcccggggca cactcaagat | 480 |
| aacgtagtgg tttggttacc tgaaaagaaa attttattcg gtggttgttt tgttaaaccg | 540 |
| gacggtcttg gtaatttggg tgacgcaaat ttagaagctt ggccaaagtc cgccaaaata | 600 |
| ttaatgtcta aatatggtaa agcaaaactg gttgtttcaa gtcatagtga aattggggac | 660 |
| gcatcactct tgaaacgtac atgggaacag gctgttaaag ggctaaatga aagtaaaaaa | 720 |
| ccatcacagc caagtaacta a | 741 |
| SEQ ID NO: 175 | |
| atgaagaaat tatttgtttt atgtgtatgc ttcttttgta gcattactgc cgcaggatcg | 60 |
| tctttacctg atttaaaaat tgagaagctt gaagaaggtg tttttgttca tacatcgttc | 120 |
| gaagaagtta acggttgggg ggttgttact aaacacggtt tggtggtgct tgtaaacaca | 180 |
| gacgcctatc taattgacac tccatttact gctacagaca ctgaaaaatt agtcaattgg | 240 |
| tttgtggagc gcggctataa aatcaaaggc actatttcat cacatttcca tagcgacagc | 300 |
| acaggaggaa tagagtggct taattctcaa tctattccca cgtatgcatc tgaattaaca | 360 |
| aatgaacttt tgaaaaaatc cggtaaggta caagctaaat attcatttag cgaagttagc | 420 |
| tattggctag ttaaaaataa aattgaagtt ttttatcctg gcccaggtca cactcaagat | 480 |
| aacctagtgg tttggttgcc tgaaagtaaa attttattcg gtggttgctt tgttaaacct | 540 |
| cacggtcttg gcaatttagg tgacgcaaat ttagaagctt ggccaaagtc cgccaaaata | 600 |
| ttaatgtcta aatatggcaa agcaaagctt gttgtttcaa gtcatagtga gaaaggggac | 660 |
| gcatcactat tgaaacgtac atgggaacaa gctcttaaag ggcttaaaga aagtaaaaaa | 720 |
| acatcatcac caagtaacta a | 741 |
| SEQ ID NO: 176 | |
| atgaagaaat tatttgtttt atgtgtatgc ttcttttgta gcattactgc cgcaggagcg | 60 |
| gctttacctg atttaaaaat cgagaagctt gaagaaggtg tttttgttca tacatcgttc | 120 |
| gaagaggtta acggttgggg ggttgttact aaacacggtt tagtggtgct tgtaaacaca | 180 |
| gacgcctatc taattgacac tccatttact gctacagaca ctgaaaaatt agtcaattgg | 240 |
| tttgtggagc gcggctatga aatcaaaggc actatttcat cacatttcca tagcgacagc | 300 |
| acaggaggaa tagagtggct taattctcaa tctattccca cgtatgcatc tgaattaaca | 360 |
| aatgaacttt tgaaaaaatc cggtaaggta caagctaaat attcatttag cgaagttagc | 420 |
| tattggctag ttaaaaataa aattgaagtt ttctaccctg gcccaggtca cactcaagat | 480 |
| aacctagtgg tttggttgcc tgaaagtaaa attttattcg gtggttgctt tattaaacct | 540 |
| cacggtcttg gcaatttagg tgacgcaaat ttagaagctt ggccaaagtc cgccaaaata | 600 |
| ttaatgtcta aatatggcaa agcaaagctt gttgtttcaa gtcatagtga aaaaggggac | 660 |
| gcatcactaa tgaaacgtac atgggaacaa gcccttaaag ggcttaaaga aagtaaaaaa | 720 |
| acatcatcac aaagtacagc atcgtga | 747 |
| SEQ ID NO: 177 | |
| atgaagaaat tatttgtttt atgtgtatgc ttcttttgta gcattactgc cgcaggagcg | 60 |
| gctttacctg atttaaaaat cgagaagctt gaagaaggtg tttttgttca tacatcgttc | 120 |
| gaagaggtta acggttgggg ggttgttact aaacacggtt tagtggtgct tgtaaacaca | 180 |
| gacgcctatc taattgacac tccatttact gctacagaca ctgaaaaatt agtcaattgg | 240 |
| tttgtggagc gcggctatga aatcaaaggc actatttcat cacatttcca tagcgacagc | 300 |
| acaggaggaa tagagtggct taattctcaa tctattccca cgtatgcatc tgaattaaca | 360 |
| aatgaacttt tgaaaaaatc cggtaaggta caagctaaat attcatttag cgaagttagc | 420 |
| tattggctag ttaaaaataa aattgaagtt ttctaccctg gcccaggtca cactcaagat | 480 |
| aacctagtgg tttggttgcc tgaaagtaaa attttattcg gtggttgctt tattaaacct | 540 |
| cacggtcttg gcaatttagg tgacgcaaat ttagaagctt ggccaaagtc cgccaaaata | 600 |
| ttaatgtcta aatatggcaa agcaaagctt gttgtttcaa gtcatagtga aaaaggggac | 660 |
| gcatcactaa tgaaacgtac atgggaacaa gcccttaaag ggcttaaaga aagtaaaaaa | 720 |
| acatcatcac caagtaacta a | 741 |
| SEQ ID NO: 178 | |
| atgaagaaat tatttgtttt atgtgtatgc ttcttttgta gcattactgc cgcaggagcg | 60 |
| gctttacctg atttaaaaat cgagaagctt gaagaaggtg tttttgttca tacatcgttc | 120 |
| gaagaggtta acggttgggg ggttgttact aaacacggtt tagtggtgct tgtaaacaca | 180 |
| gacgcctatc taattgacac tccatttact gctacagaca ctgaaaaatt agtcaattgg | 240 |
| tttgtggagc gcggctatga aatcaaaggc actatttcat cacatttcca tagcgacagc | 300 |
| acaggaggaa tagagtggct taattctcaa tctattccca cgtatgcatc tgaattaaca | 360 |
| aatgaacttt tgaaaaaatc cggtaaggta caagctaaat attcatttag cgaagttagc | 420 |
| tattggctag ttaaaaataa aattgaagtt ttctaccctg gcccaggtca cactcaagat | 480 |
| aacctagtgg tttggttgcc tgaaagtaaa attttattcg gtggttgctt tattaaacct | 540 |
| cacggtcttg gcaatttagg tgacgcaaat ttagaagctt ggccaaagtc cgccaaaata | 600 |
| ttaatgtcta aatatggcaa agcaaagctt gttgtttcaa gtcatagtga aaaaggggac | 660 |
| gcatcactaa tgaaacgtac atgggaacaa gcccttaaag ggcttaaaga aagtaaaaaa | 720 |
| acatcatcac caagtaacta a | 741 |
| SEQ ID NO: 179 | |
| atgaagaaat tatttgtttt atgtgtatgc ttcttttgta gcattactgc cgcaggagcg | 60 |
| gctttacctg atttaaaaat cgagaagctt gaagaaggtg tttttgttca tacatcgttc | 120 |
| gaagaggtta acggttgggg ggttgttact aaacacggtt tagtggtgct tgtaaacaca | 180 |
| gacgcctatc taattgacac tccatttact gctacagaca ctgaaaaatt agtcaattgg | 240 |
| tttgtggagc gcggctatga aatcaaaggc actatttcat cacatttcca tagcgacagc | 300 |
| acaggaggaa tagagtggct taattctcaa tctattccca cgtatgcatc tgaattaaca | 360 |
| aatgaacttt tgaaaaaatc cggtaaggta caagctaaat attcatttag cggagttagc | 420 |
| tattggctag ttaaaaataa aattgaagtt ttctaccctg gcccaggtca cactcaagat | 480 |
| aacctagtgg tttggttgcc tgaaagtgaa attttattcg gtggttgctt tattaaacct | 540 |
| cacggtcttg gcaatttagg tgacgcaaat ttagaagctt ggccaaagtc cgccaaaata | 600 |
| ttaatgtcta aatatggcaa agcaaagctt gttgtttcaa gtcatagtga aaaaggggac | 660 |
| gcatcactaa tgaaacgtac atgggaacaa gcccttaaag ggcttaaaga aagtaaaaaa | 720 |
| acatcatcac caagtaacta a | 741 |
| SEQ ID NO: 180 | |
| atgagcaagt tatctgtatt ctttatattt ttgttttgca gcattgctac cgcagcagag | 60 |
| tctttgccag atttaaaaat tgaaaagctt gatgaaggcg tttatgttca tacttcgttt | 120 |
| gaagaagtta acgggtgggg cgttgttcct aaacatggtt tggtggttct tgtaaatgct | 180 |
| gaggcttatt taattgacac tccatttacg gctaaagata ctgaaaagtt agtcacttgg | 240 |
| tttgtggagc gtggctataa aataaaaggc agtatttcct ctcattttca tagcgacagc | 300 |
| acgggcggaa tagggtggct taattctcga tctatcccca cgtatgcatc tgaattaaca | 360 |
| aatgaactgc ttaaaaaaga cggtaaggtt caagccacaa attcatttag cggagttaac | 420 |
| tattggctag ttaaaaataa aattgaagtt ttttatccag gcccgggaca cactccagat | 480 |
| aacgtagtgg tttggctgcc tgaaaggaaa atattattcg gtggttgttt tattaaaccg | 540 |
| tacggtttag gcaatttggg tgacgcaaat atagaagctt ggccaaagtc cgccaaatta | 600 |
| ttaaagtcca aatatggtaa ggcaaaactg gttgttccaa gtcacagtga agttggagac | 660 |
| gcatcactct tgaaacttac attagagcag gcggttaaag gattaaacga aagtaaaaaa | 720 |
| ccatcaaaac caagcaacta a | 741 |
| SEQ ID NO: 181 | |
| atgagcaagt tatctgtatt ctttatattt ttgttttgca gcattgctac cgcagcagag | 60 |
| tctttgccag atttaaaaat tgaaaagctt gatgaaggcg tttatgttca tacttcgttt | 120 |
| gaagaagtta acgggtgggg cgttgttcct aaacatggtt tggtggttct tgtaaatgct | 180 |
| gaggcttatt taattgacac tccatttacg gctaaagata ctgaaaagtt agtcacttgg | 240 |
| tttgtggagc gtggctataa aataaaaggc agtatttcct ctcattttca tagcgacagc | 300 |
| acgggcggaa tagggtggct taattctcga tctatcccca cgtatgcatc tgaattaaca | 360 |
| aatgaactgc ttaaaaaaga cggtaaggtt caagccacaa attcatttag cggagttaac | 420 |
| tattggctag ttaaaaataa aattgaagtt ttttatccag gcccgggaca cactccagat | 480 |
| aacgtagtgg tttggctgcc tgaaaggaaa atattattcg gtggttgttt tattaaaccg | 540 |
| tacggtttag gcaatttggg tgacgcaaat atagaagctt ggccaaagtc cgccaaatta | 600 |
| ttaaagtcca aatatggtaa ggcaaaactg gttgttccag gtcacagtga agttggagac | 660 |
| gcatcactct tgaaacttac attagagcag gcggttaaag gattaaacga aagtaaaaaa | 720 |
| ccatcaaaac caagcaacta a | 741 |
| SEQ ID NO: 182 | |
| atgagcaagt tatctgtatt ctttatattt ttgttttgca gcattgctac cgcagcagag | 60 |
| tctttgccag atttaaaaat tgaaaagctt gatgaaggcg tttatgttca tacttcgttt | 120 |
| gaagaagtta acgggtgggg cgttgttcct aaacatggtt tggtggttct tgtaaatgct | 180 |
| gaggcttacc taattgacac tccatttacg gctaaagata ctgaaaagtt agtcacttgg | 240 |
| tttgtggagc ctggctataa aataaaaggc agcatttcct ctcattttca tagcgacagc | 300 |
| acgggcggaa tagagtggct taattctcga tctatcccca cgtatgcatc tgaattaaca | 360 |
| aatgaactgc ttaaaaaaga cggtaaggtt caagccacaa attcatttag cggagttaac | 420 |
| tattggctag ttaaaaataa aattgaagtt ttttatccag gcccgggaca cactccagat | 480 |
| aacgtactgg tttggttgcc tgaaaggaaa atattattcg gtggttgttt tattaaaccg | 540 |
| tacggtttag gcaatttggg tgacgcaaat atagaagctt ggccaaagtc cgccaaatta | 600 |
| ttaaagtcca aatatggtaa ggcaaaactg gttgttccaa gtcacagtga agttggagac | 660 |
| gcatcactct tgaaacttac attagagcag gcggttaaag ggttaaacga aagtaaaaaa | 720 |
| ccatcaaaac caagcaacta a | 741 |
| SEQ ID NO: 183 | |
| atgagcaagt tatctgtatt ctttatattt ttgttttgca gcattgctac cgcagcagag | 60 |
| tctttgccag atttaaaaat tgaaaagctt gatgaaggcg tttatgttca tacttcgttt | 120 |
| gaagaagtta acgggtgggg cgttgttcct aaacatggtt tggtggttct tgtaaatgct | 180 |
| gaggcttacc taattgacac tccatttacg gctaaagata ctgaaaagtt agtcacttgg | 240 |
| tttgtggagc gtggctataa aataaaaggc agcatttcct ctcattttca tagcgacagc | 300 |
| acgggcggaa tagagtggct taattctcga tctatcccca cgtatgcatc tgaattaaca | 360 |
| aatgaactgc ttaaaaaaga cggtaaggtt caagccacaa attcatttag cggagttaac | 420 |
| tattggctag ttaaaaataa aattgaagtt ttttatccag gcccgggaca cactccagat | 480 |
| aacgtagtgg tttggttgcc tgaaaggaaa atattattcg gtggttgttt tattaaaccg | 540 |
| tacggtttag gcaatttgag tgacgcaaat atagaagctt ggccaaagtc cgccaaatta | 600 |
| ttaaagtcca aatatggtaa ggcaaaactg gttgttccag gtcacagtga agttggagac | 660 |
| gcatcactct tgaaacttac attagagcag gcggttaaag ggttaaacga aagtaaaaaa | 720 |
| ccatcaaaac caagcaacta a | 741 |
| SEQ ID NO: 184 | |
| atgagcaagt tatctgtatt ctttatattt ttgttttgca gcattgctac cgcagcagag | 60 |
| tctttgccag atttaaaaat tgaaaagctt gatgaaggcg tttatgttca tacttcgttt | 120 |
| gaagaagtta acaggtgggg cgttgttcct aaacatggtt tggtggttct tgtaaatgct | 180 |
| gaggcttacc taattgacac tccatttacg gctaaagata ctgaaaagtt agtcacttgg | 240 |
| tttgtggagc gtggctataa aataaaaggc agcatttcct ctcattttca tagcgacagc | 300 |
| acgggcggaa tagagtggct taattctcga tctatcccca cgtatgcatc tgaattaaca | 360 |
| aatgaactgc ttaaaaaaga cggtaaggtt caagccacaa attcatttag cggagttaac | 420 |
| tattggctag ttaaaaataa aattgaagtt ttttatccag gcccgggaca cactccagat | 480 |
| aacgtagtgg tttggttgcc tgaaaggaaa atattattcg gtggttgttt tattaaaccg | 540 |
| tacggtttag gcaatttggg tgacgcaaat atagaagctt ggccaaagtc cgccaaatta | 600 |
| ttaaagtcca aatatggtaa ggcaaaactg gttgttccaa gtcacagtga agttggagac | 660 |
| gcatcactct tgaaacttac attagagcag gcggttaaag ggttaaacga aagtaaaaaa | 720 |
| ccatcaaaac caagcaacta a | 741 |
| SEQ ID NO: 185 | |
| atgagcaagt tatctgtatt ctttatattt ttgttttgca gcattgctac cgcagcagag | 60 |
| tctttgccag atttaaaaat tgaaaagctt gatgaaggcg tttatgttca tacttcgttt | 120 |
| gaagaagtta acgggtgggg cgtttttcct aaacatggtt tggtggttct tgtaaatgct | 180 |
| gaggcttacc taattgacac tccatctacg gctaaagata ctgaaaagtt agtcacttgg | 240 |
| tttgtggagc gtggctataa aataaaaggc agcatttcct ctcattttca tagcgacagc | 300 |
| acgggcggaa tagagtggct taattctcga tctatcccca cgtatgcatc tgaattaaca | 360 |
| aatgaactgc ttaaaaaaga cggtaaggtt caagccacaa attcatttag cggagttaac | 420 |
| tattggctag ttaaaaataa aattgaagtt ttttatccag gcccgggaca cactccagat | 480 |
| aacgtagtgg tttggttgcc tgaaaggaaa atattattcg gtggttgttt tattaaaccg | 540 |
| tacggtttag gcaatttggg tgacgcaaat atagaagctt ggccaaagtc cgccaaatta | 600 |
| ttaaagtcca aatatggtaa ggcaaaactg gttgttccaa gtcacagtga agttggagac | 660 |
| gcatcactct tgaaacttac attagagcag gcggttaaag ggttaaacga aagtaaaaaa | 720 |
| ccatcaaaac caagcaacta a | 741 |
| SEQ ID NO: 186 | |
| atgagcaagt tatctgtatt ctttatattt ttgttttgca gcattgctac cgcagcagag | 60 |
| tctttgccag atttaaaaat tgaaaagctt gatgaaggcg tttatgttca tacttcgttt | 120 |
| gaagaagtta acgggtgggg cgtttttcct aaacatggtt tggtggttct tgtaaatgct | 180 |
| gaggcttacc taattgacac tccatttacg gctaaagata ctgaaaagtt agtcacttgg | 240 |
| tttgtggagc gtggctataa aataaaaggc agcatttcct ctcattttca tagcgacagc | 300 |
| acgggcggaa tagagtggct taattctcga tctatcccca cgtatgcatc tgaattaaca | 360 |
| aatgaactgc ttaaaaaaga cggtaaggtt caagccacaa attcatttag cggagttaac | 420 |
| tattggctag ttaaaaataa aattgaagtt ttttatccag gcccgggaca cactccagat | 480 |
| aacgtagtgg tttggttgcc tgaaaggaaa atattattcg gtggttgttt tattaaaccg | 540 |
| tacggtttag gcaatttggg tgacgcaaat atagaagctt ggccaaagtc cgccaaatta | 600 |
| ttaaagtcca aatatggtaa ggcaaaactg gttgttccaa gtcacagtga agttggagac | 660 |
| gcatcactct tgaaacttac attagagcag gcggttaaag ggttaaacga aagtaaaaaa | 720 |
| ccatcaaaac caagcaacta a | 741 |
| SEQ ID NO: 187 | |
| atgagcaagt tatctgtatt ctttatattt ttgttttgca gcattgctac cgcagcagag | 60 |
| tctttgccag atttaaaaat tgaaaagctt gatgaaggcg tttatgttca tacttcgttt | 120 |
| gaagaagtta acgggtgggg cgtttttcct aaacatggtt tggtggttct tgtaaatgct | 180 |
| gaggcttacc taattgacac tccatttacg gctaaagata ctgaaaagtt agtcacttgg | 240 |
| tttgtggagc gtggctataa aataaaaggc agcatttcct ctcattttca tagcgacagc | 300 |
| acgggcggaa tagagtggct taattctcga tctatcccca cgtatgcatc tgaattaaca | 360 |
| aatgaactgc ttaaaaaaga cggtaaggtt caagccacaa attcatttag cggagttaac | 420 |
| tattggctag ttaaaaataa aattgaagtt ttttatccag gcccgggaca cactccagat | 480 |
| aacgtagtgg tttggttgcc tgaaaggaaa atattattcg gtggttgttt tattaaaccg | 540 |
| tacggtttag gcaatttggg tgacgcaaat atagaagctt ggccaaagtc cgccaaatta | 600 |
| ttaaagtcca aatatggtaa ggcaaaactg gttgttccaa gtcacagtga agttggagac | 660 |
| gcatcactct tgaaacttac attagagcag gcggttaaag ggttaaacga aagtaaaaaa | 720 |
| ccatcaaaac caagcaacta aatttctaac aagtcgttgc agcacgccac tacgtggctg | 780 |
| gacagtttgt aagttgcgct tttgtggttt gcttcgcaaa gtattccaca acgcgcaact | 840 |
| ta | 842 |
| SEQ ID NO: 188 | |
| atgagcaagt tatctgtatt ctttatattt ttgttttgca gcattgctac cgcagcagag | 60 |
| tctttgccag atttaaaaat tgaaaagctt gatgaaggcg tttatgttca tacttcgttt | 120 |
| gaagaagtta acgggtgggg cgttgttcct aaacatggtt tggtggttct tgtaaatgct | 180 |
| gaggcttacc taattgacac tccatttacg gctaaagata ctgaaaagtt agtcacttgg | 240 |
| tttgtggagc gtggctataa aataaaaggc agcatttcct ctcattttca tagcgacagc | 300 |
| acgggcggaa tagagtggct taattctcga tctatcccca cgtatgcatc tgaattaaca | 360 |
| aatgaactgc ttaaaaaaga cggtaaggtt caagccacaa attcatttag cggagttaac | 420 |
| tattggctag ttaaaaataa aattgaagtt ttttatccag gcccgggaca cactccagat | 480 |
| aacgtagtgg tttggttgcc tgaaaggaaa atattattcg gtggttgttt tattaaaccg | 540 |
| tacggtttag gcaatttggg tgacgcaaat atagaagctt ggccaaagtc cgccaaatta | 600 |
| ttaaagtcca aatatggtaa ggcaaaactg gttgttccag gtcacagtga agttggagac | 660 |
| gcatcactct tgaaacttac attagagcag gcggttaaag ggttaaacga aagtaaaaaa | 720 |
| ccatcaaaac caagcaacta a | 741 |
| SEQ ID NO: 189 | |
| atgagcaagt tatctgtatt ctttatattt ttgttttgca gcattgctac cgcagcagag | 60 |
| tctttgccag atttaaaaat tgaaaagctt gatgaaggcg tttatgttca tacttcgttt | 120 |
| gaagaagtta acgggtgggg cgttgttcct aaacatggtt tggtggttct tgtaaatgct | 180 |
| gaggcttacc taattgacac tccatttacg gctaaagata ctgaaaagtt agtcacttgg | 240 |
| tttgtggagc gtggctataa aataaaaggc agcatttcct ctcattttca tagcgacagc | 300 |
| acgggcggaa tagagtggct taattctcga tctatcccca cgtatgcatc tgaattaaca | 360 |
| aatgaactgc ttaaaaaaga cggtaaggtt caagccacaa attcatttag cggagttaac | 420 |
| tattggctag ttaaaaataa aattgaagtt ttttatccag gcccgggaca cactccagat | 480 |
| aacgtagtgg tttggttgcc tgaaaggaaa atattattcg gtggttgttt tattaaaccg | 540 |
| tacggtttag gcaatttggg tgacgcaaat atagaagctt ggccaaagtc cgccaaatta | 600 |
| ttaaagtcca aatatggtaa ggcaaaactg gttgttccag gtcacagtga agttggagac | 660 |
| gcatcactct tgaaacttac attagagcag gcggttaaag ggttaaacga aagtaaaaaa | 720 |
| ccatcaaaac caagcaacta a | 741 |
| SEQ ID NO: 190 | |
| atgagcaagt tatctgtatt ctttatattt ttgttttgca gcattgctac cgcagcagag | 60 |
| tctttgccag atttaaaaat tgaaaagctt gatgaaggcg tttatgttca tacttcgttt | 120 |
| aaagaagtta acgggtgggg cgttgttcct aaacatggtt tggtggttct tgtaaatgct | 180 |
| gaggcttacc taattgacac tccatttacg gctaaagata ctgaaaagtt agtcacttgg | 240 |
| tttgtggagc gtggctataa aataaaaggc agcatttcct ctcattttca tagcgacagc | 300 |
| acgggcggaa tagagtggct taattctcga tctatcccca cgtatgcatc tgaattaaca | 360 |
| aatgaactgc ttaaaaaaga cggtaaggtt caagccacaa attcatttag cggagttaac | 420 |
| tattggctag ttaaaaataa aattgaagtt ttttatccag gcccgggaca cactccagat | 480 |
| aacgtagtgg tttggttgcc tgaaaggaaa atattattcg gtggttgttt tattaaaccg | 540 |
| tacggtttag gcaatttggg tgacgcaaat atagaagctt ggccaaagtc cgccaaatta | 600 |
| ttaaagtcca aatatggtaa ggcaaaactg gttgttccaa gtcacagtga agttggagac | 660 |
| gcatcactct tgaaacttac attagagcag gcggttaaag ggttaaacga aagtaaaaaa | 720 |
| ccatcaaaac caagcaacta a | 741 |
| SEQ ID NO: 191 | |
| atgagcaagt tatctgtatt ctttatattt ttgttttgca gcattgctac cgcagcagag | 60 |
| tctttgccag atttaaaaat tgaaaagctt gatgaaggcg tttatgttca tacttcgttt | 120 |
| gaagaagtta acgggtgggg cgttgttcct aaacatggtt tggtggttct tgtaaatgct | 180 |
| gaggcttacc taattgacac tccatttacg gctaaagata ctgaaaagtt agtcacttgg | 240 |
| tttgtggagc gtggctataa aataaaaggc agcatttcct ctcattttca tagcgacagc | 300 |
| acgggcggaa tagagtggct taattctcga tctatcccca cgtatgcatc tgaattaaca | 360 |
| aatgaactgc ttaaaaaaga cggtaaggtt caagccacaa attcatttag cggagttaac | 420 |
| tattggctag ttaaaaataa aattgaagtt ttttatccag gcccgggaca cactccagat | 480 |
| aacgtagtgg tttggttgcc tgaaaggaaa atattattcg gtggttgttt tattaaaccg | 540 |
| tacggtttag gcaatttggg tgacgcaaat atagaagctt ggccaaagtc cgccaaatta | 600 |
| ttaaagtcca aatatggtaa ggcaaaactg gttgttccaa gtcacagtga agttggagac | 660 |
| gcatcactct tgaaacttac attagagcag gcggttaaag ggttaaacga aagtaaaaaa | 720 |
| ccatcaaaac caagcaacta a | 741 |
| SEQ ID NO: 192 | |
| atgagcaagt tatctgtatt ctttatattt ttgttttgca gcattgctac cgcagcagag | 60 |
| tctttgccag atttaaaaat tgaaaagctt gatgaaggcg tttatgttca tacttcgttt | 120 |
| gaagaagtta acgggtgggg cgttgttcct aaacatggtt tggtggttct tgtaaatgct | 180 |
| gaggcttacc taattgacac tccatttacg gctaaagata ctgaaaagtt agtcacttgg | 240 |
| tttgtggagc gtggctataa aataaaaggc agcatttcct ctcattttca tagcgacagc | 300 |
| acgggcggaa tagagtggct taattctcga tctatcccca cgtatgcatc tgaattaaca | 360 |
| aatgaactgc ttaaaaaaga cggtaaggtt caagccacaa attcatttag cggagttaac | 420 |
| tattggctag ttaaaaataa aattgaagtt ttttatccag gcccgggaca cactccagat | 480 |
| aacgtagtgg tttggttgcc tgaaaggaaa atattattcg gtggttgttt tattaaaccg | 540 |
| tacggtttag gcaatttggg tgacgcaaat atagaagctt ggccaaagtc cgccaaatta | 600 |
| ttaaagtcca aatatggtaa ggcaaaactg gttgttccaa gtcacagtga agttggagac | 660 |
| gcatcactct tgaaacttac attagagcag gcggttaaag ggttaaacga aagtaaaaaa | 720 |
| ccatcaaaac caagcaacta a | 741 |
| SEQ ID NO: 193 | |
| atgaaaaaaa tatttgtgtt atttgtattt ttgttttgca gtattactgc cgccggagag | 60 |
| tctttgcctg atataaaaat tgagaaactt gacgaagatg tttatgttca tacttctttt | 120 |
| gaagaagata acggctgggg tgttattact aaacacggct tggtggttct tgtaaatact | 180 |
| gatgcctata taattgacac tccatttaca gctaaagata ctgaaaaatt agtccgctgg | 240 |
| tttgtggggc gtggttataa aatcaaaggc agtatttcct cacattttca tagcgatagc | 300 |
| gcaggtggaa ttgagtggct taattctcaa tctatcccca catatgcatc taaattaaca | 360 |
| aatgagcttc ttaaaaagaa cggtaatgcg caagccgaaa actcatttag tggcgttagc | 420 |
| tattggctag ttaaacataa aattgaagtt ttctatccag gaccagggca cactcaggat | 480 |
| aatgtagtgg tttggttgcc tgaaaagaaa attttatttg gcggttgttt tattaagccg | 540 |
| gacggtcttg gttatttggg agacgcaaat ctagaagcat ggcctaagtc cgcagaaaca | 600 |
| ttaatgtcta agtatggtaa tgcaaaactg gttgtttcga gtcatagtga aattgggggc | 660 |
| gcatcactat tgaagcgcac ttgggagcag gctgttaagg ggctaaaaga aagtaaaaaa | 720 |
| ccatcacagc caaataacta a | 741 |
| SEQ ID NO: 194 | |
| gcaactatga gtagtgggag taatttac | 28 |
| SEQ ID NO: 195 | |
| ttcattgcct gaattgaaga catgag | 26 |
| SEQ ID NO: 196 | |
| acggagtagt agtaaaacct acagtcctaa agc | 33 |

The following Examples illustrate the invention.

### EXAMPLES

### Example 1—Design and Generation of Primers and Probes

DNA sequences of the carbapenemase genes of interest were identified from public databases (see the above tables in the sequence information section) and DNA sequence alignments generated.

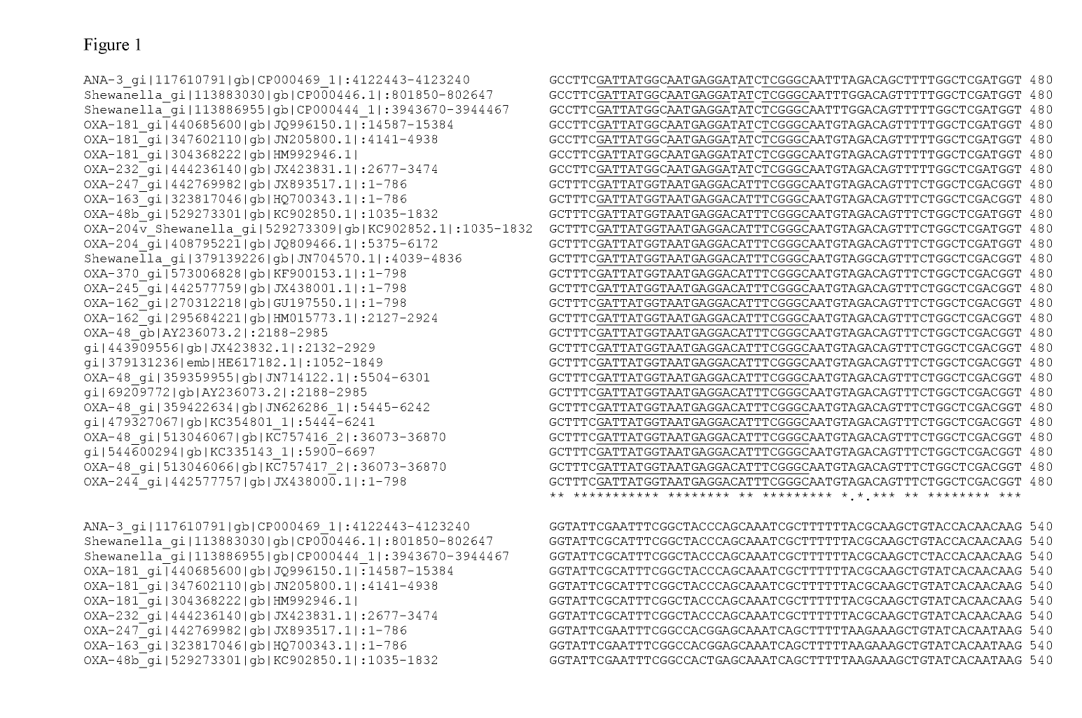

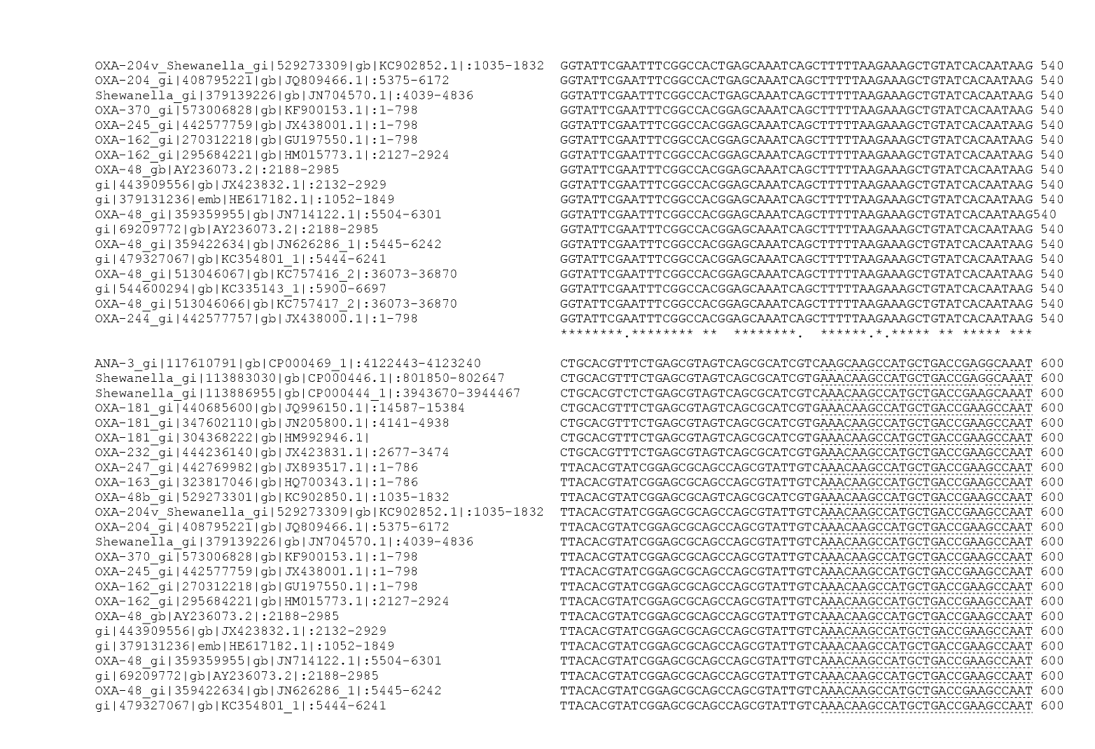

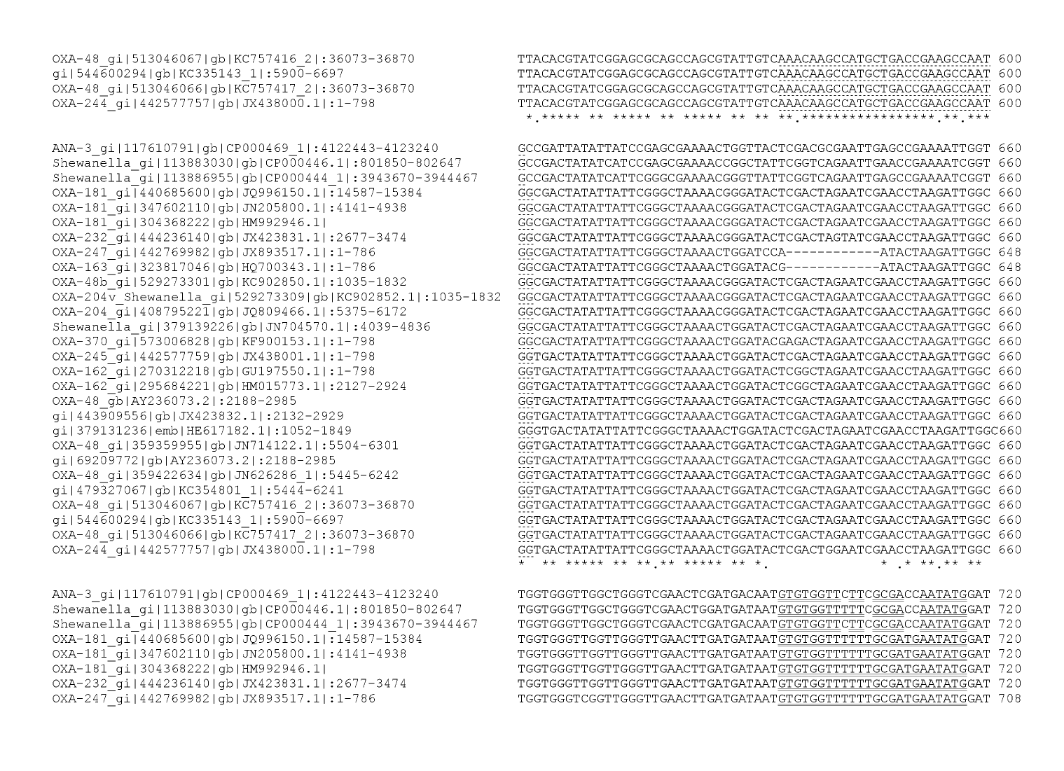

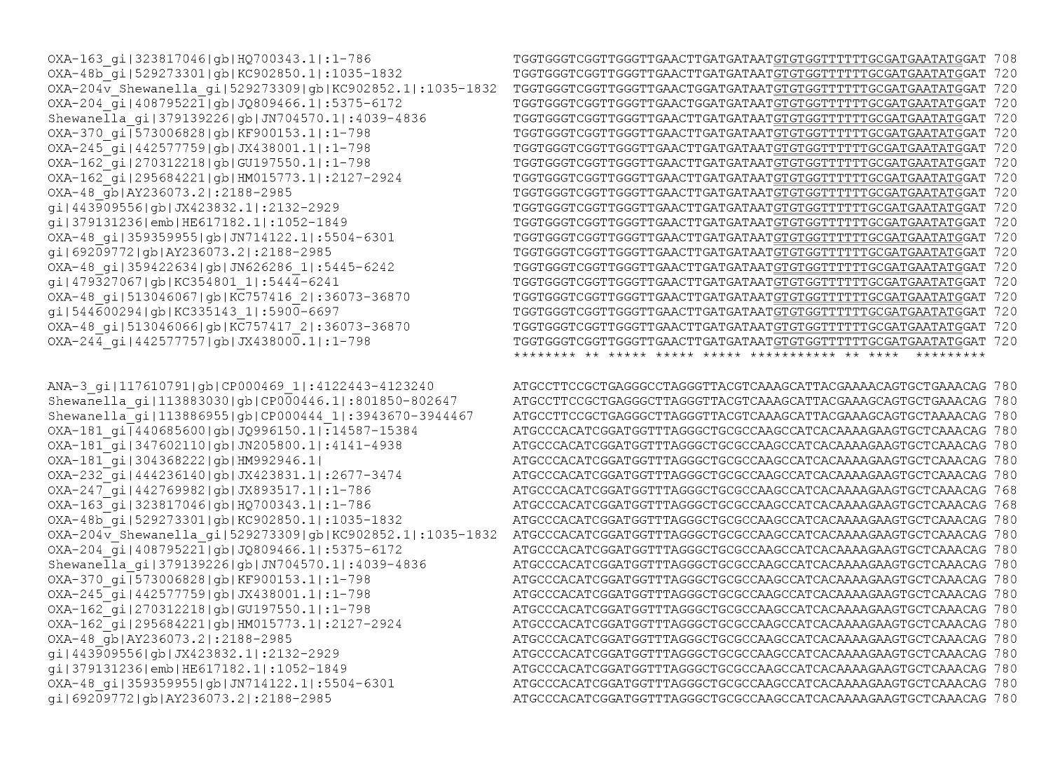

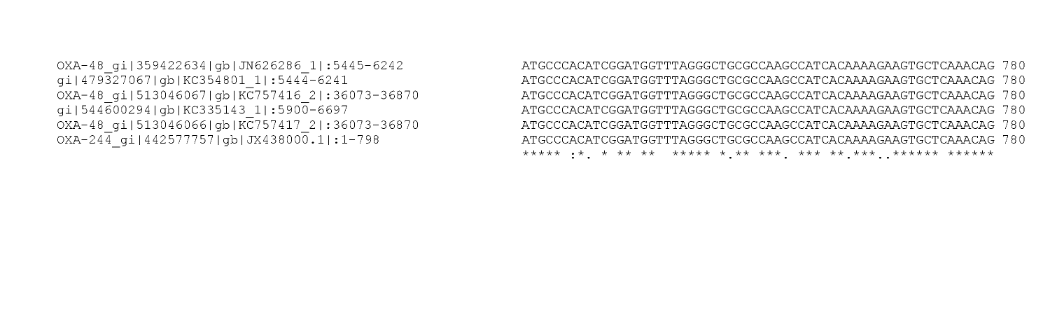

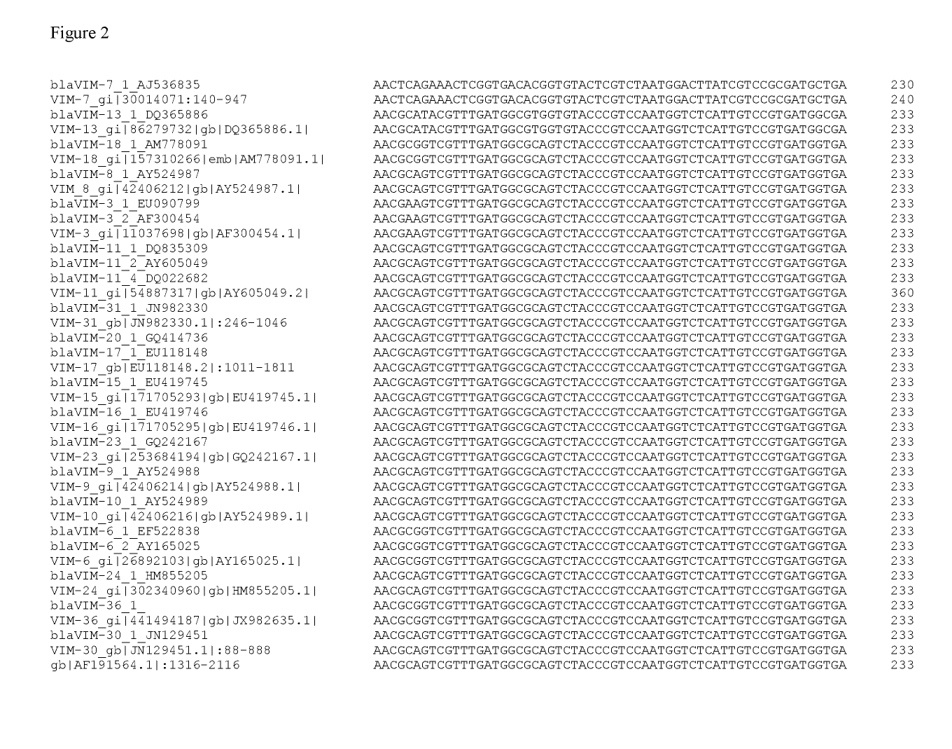

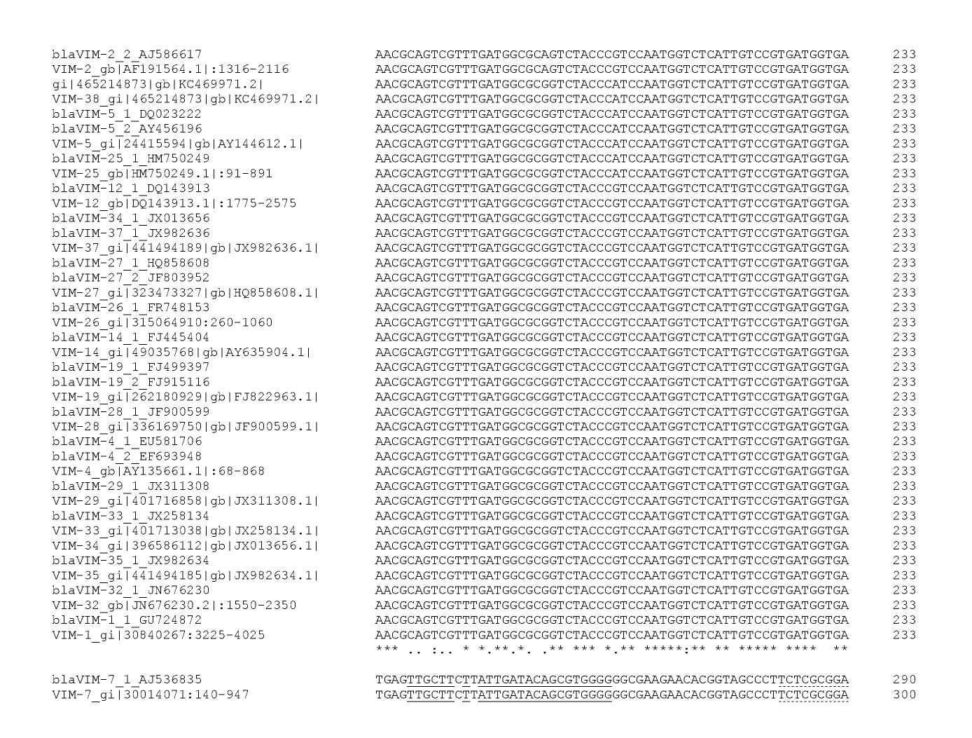

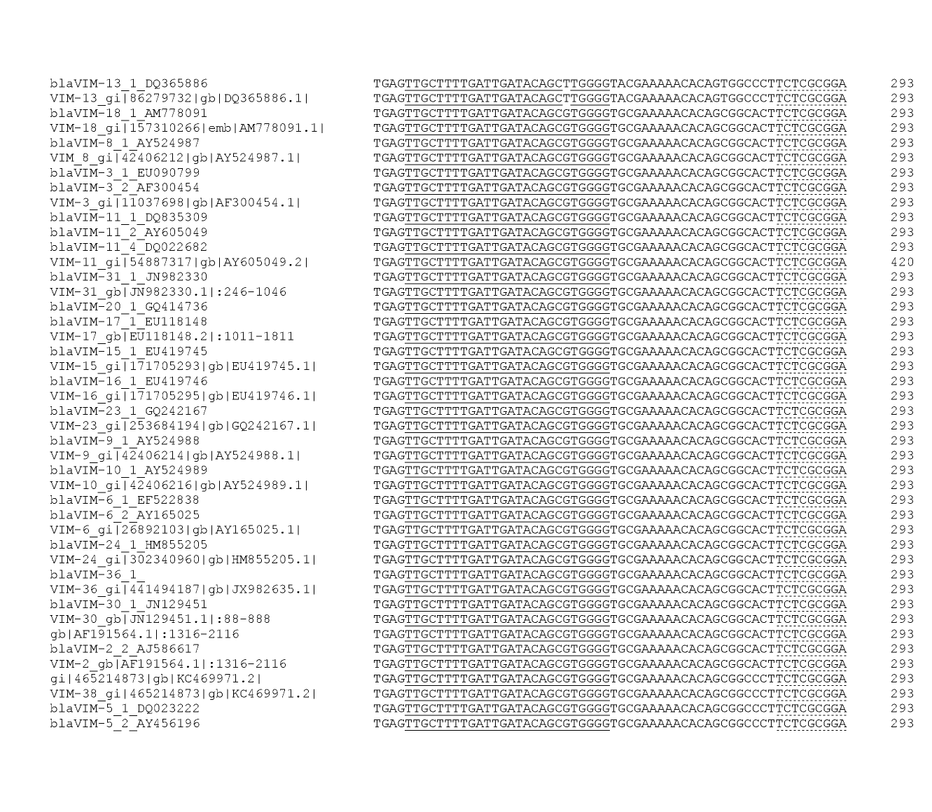

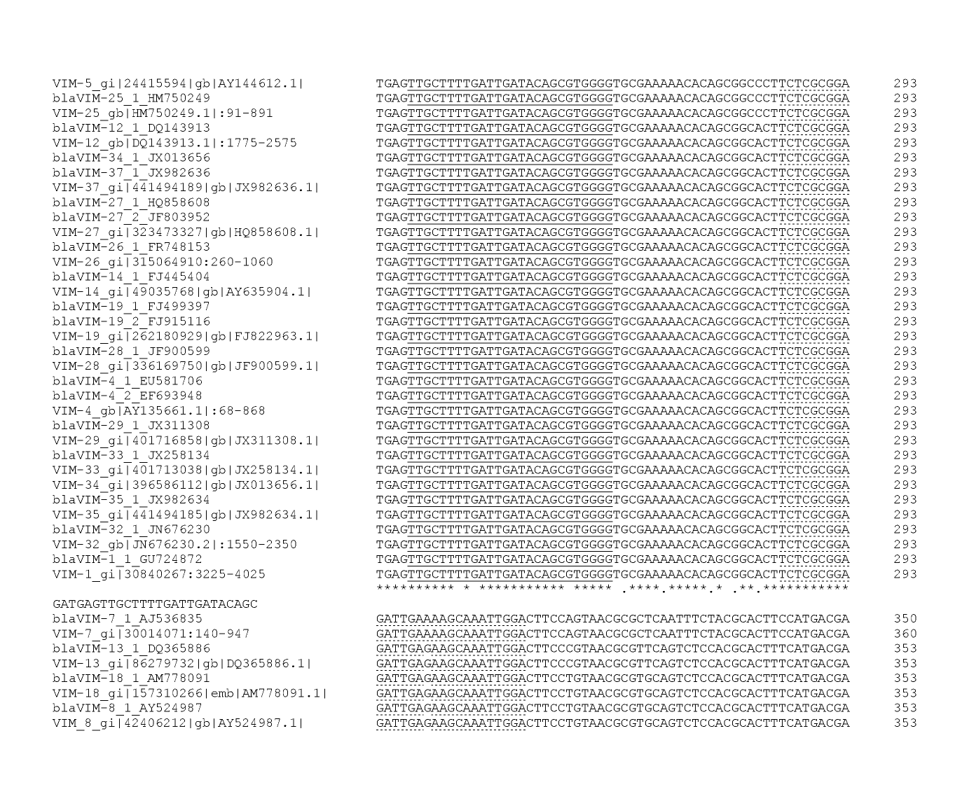

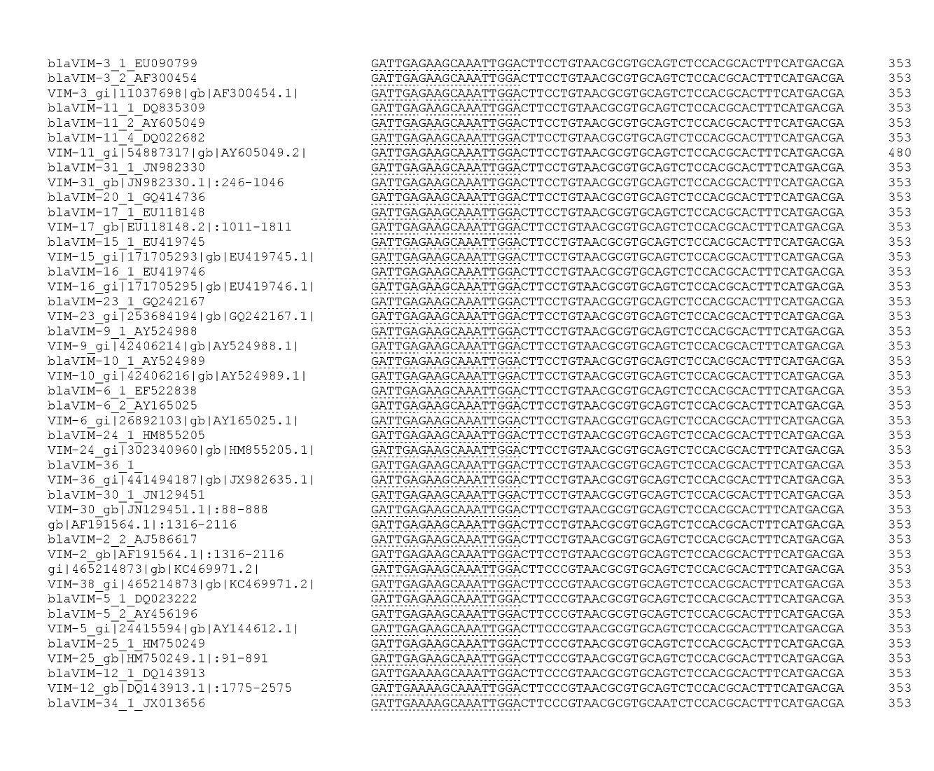

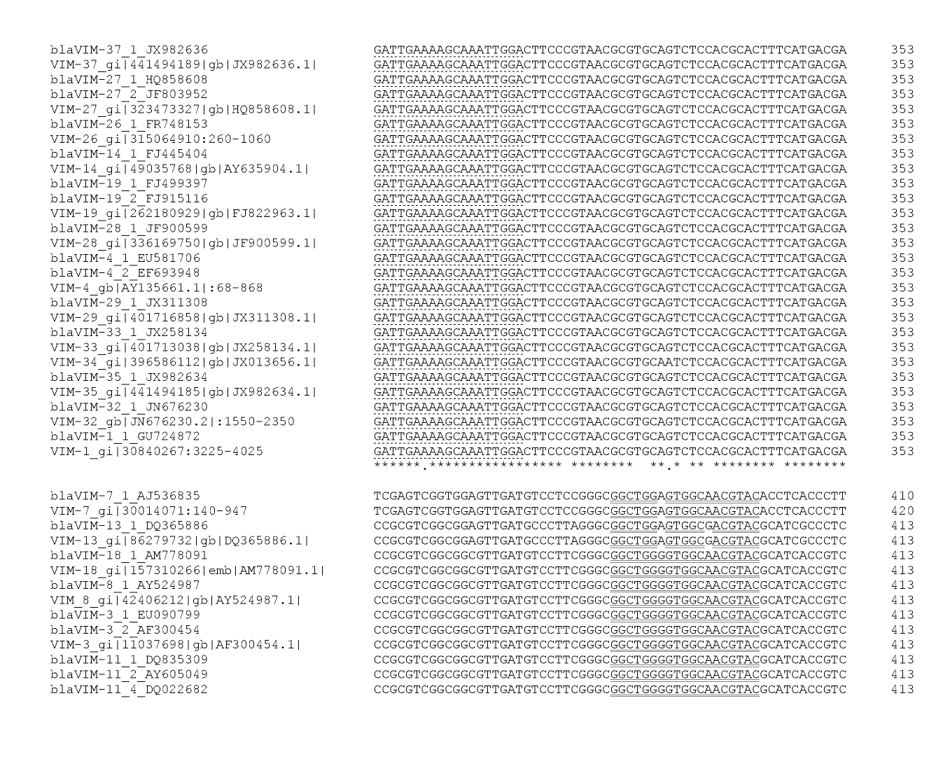

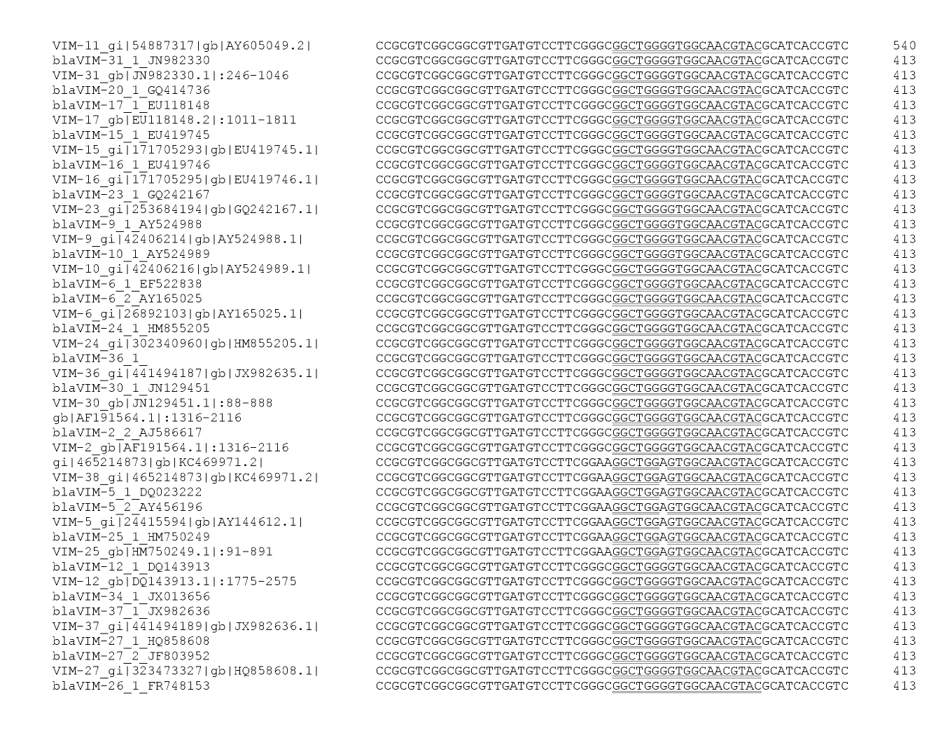

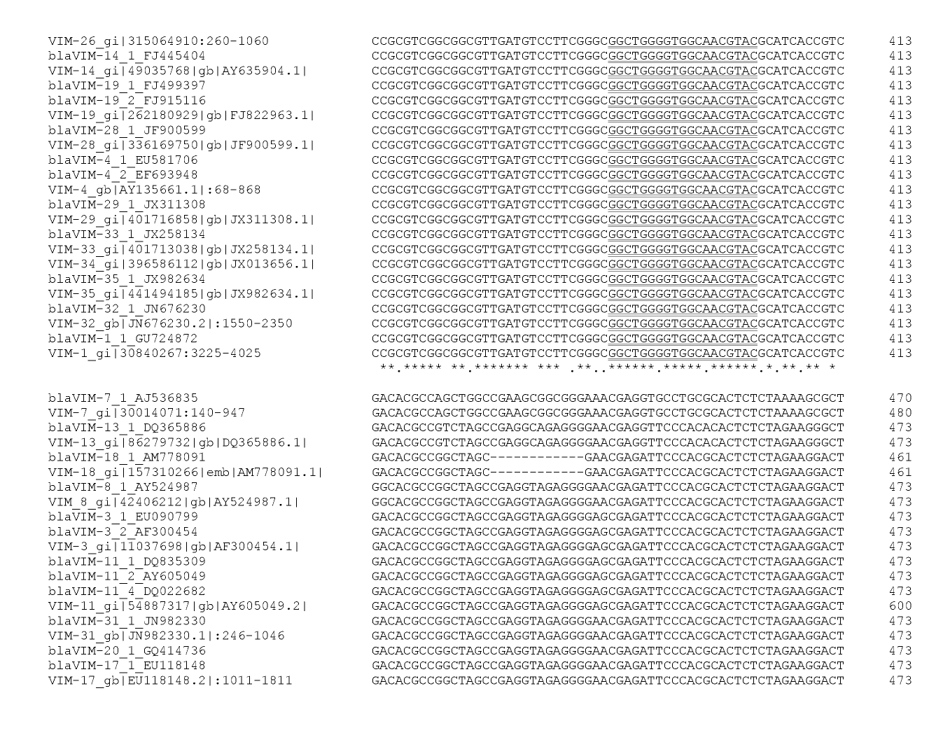

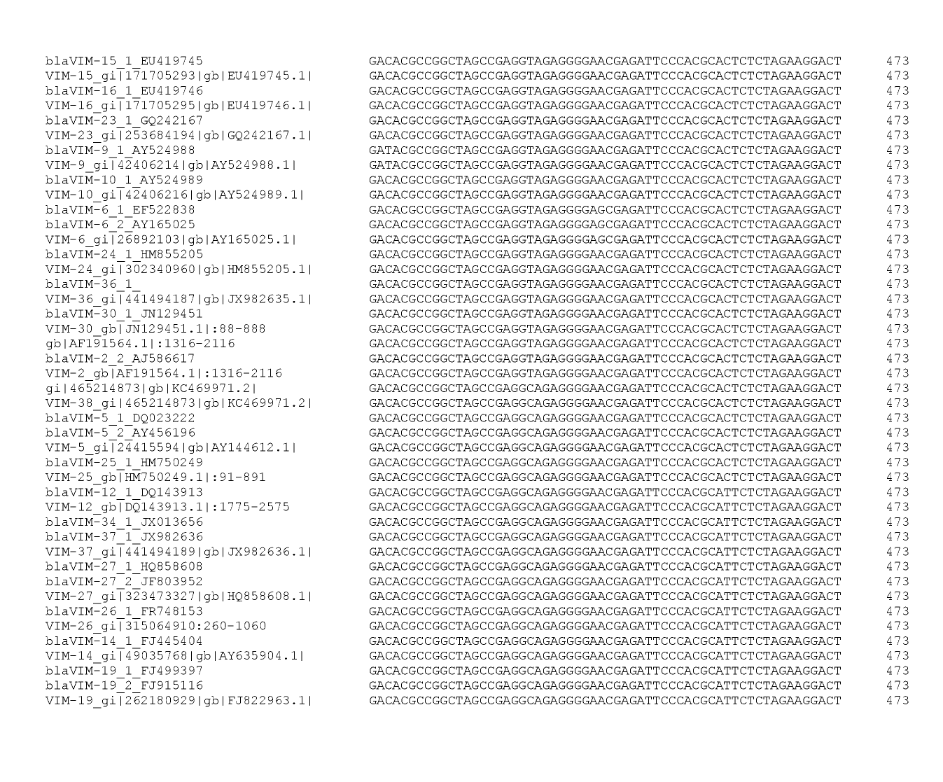

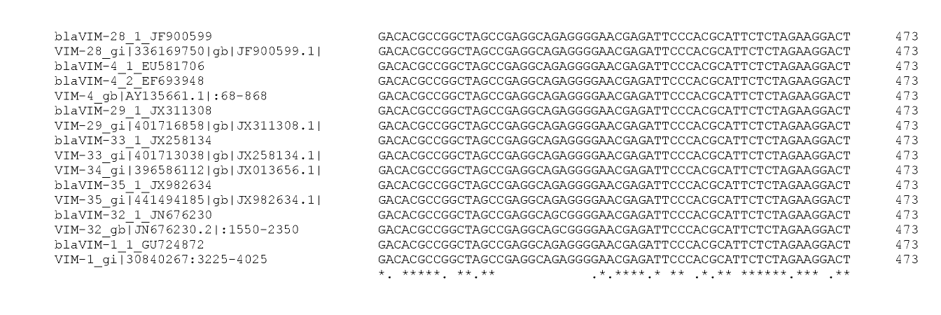

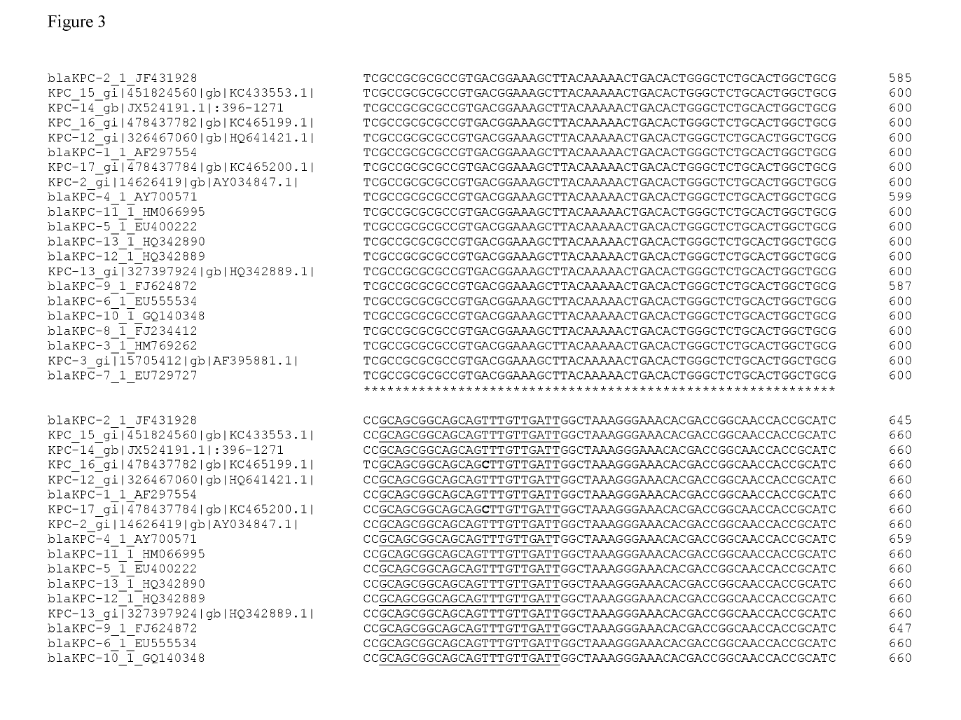

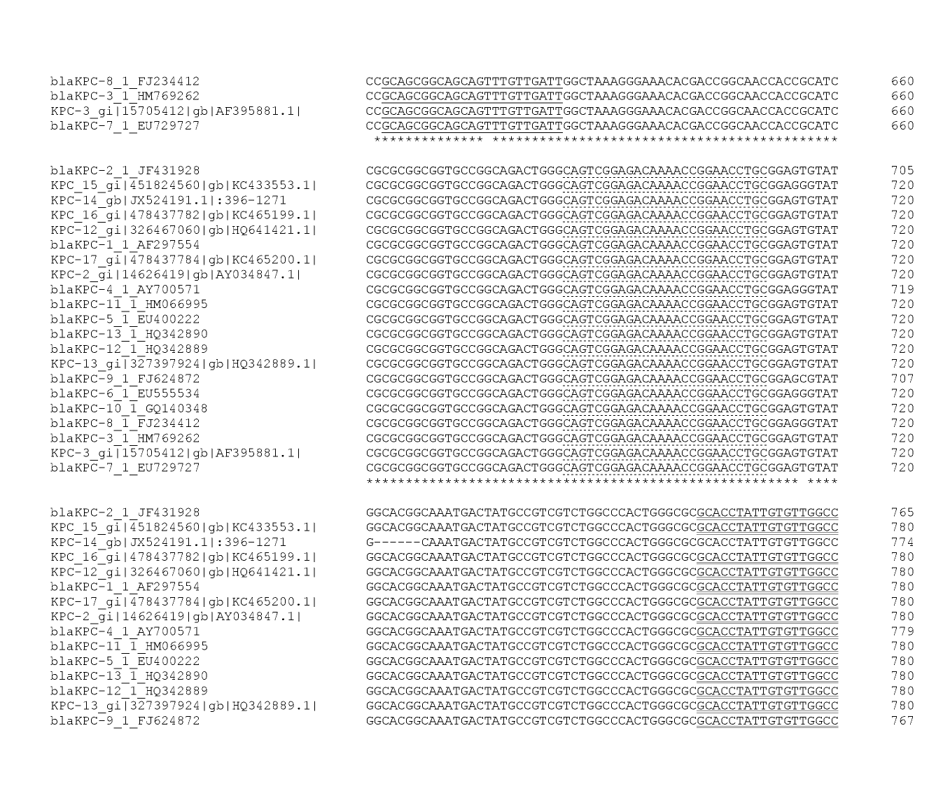

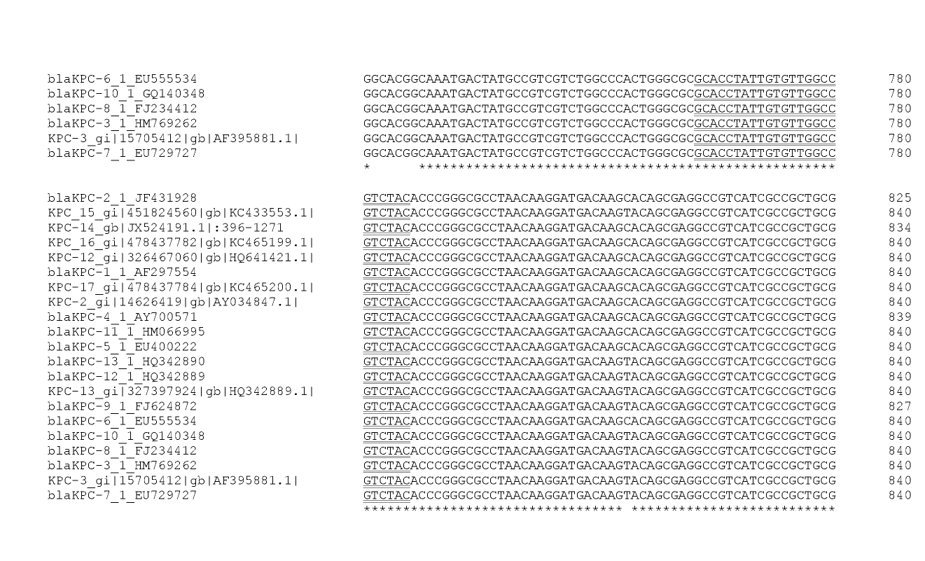

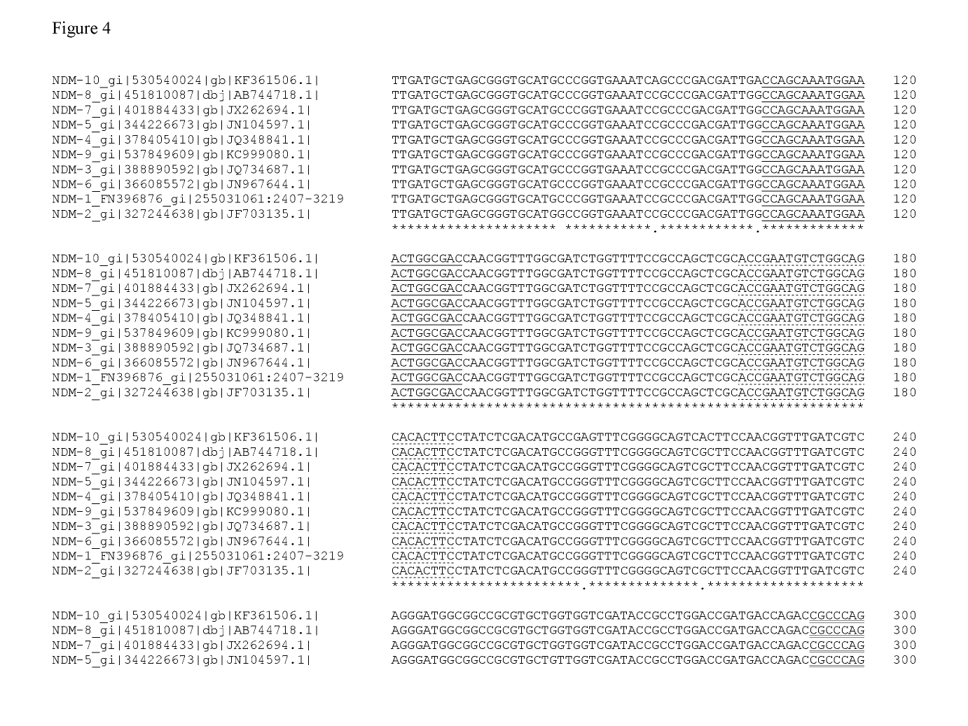

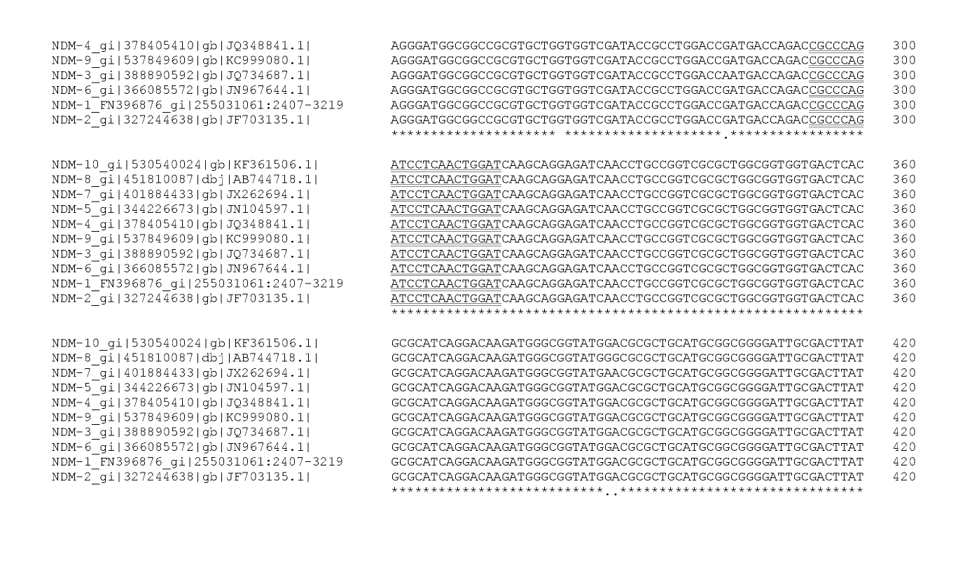

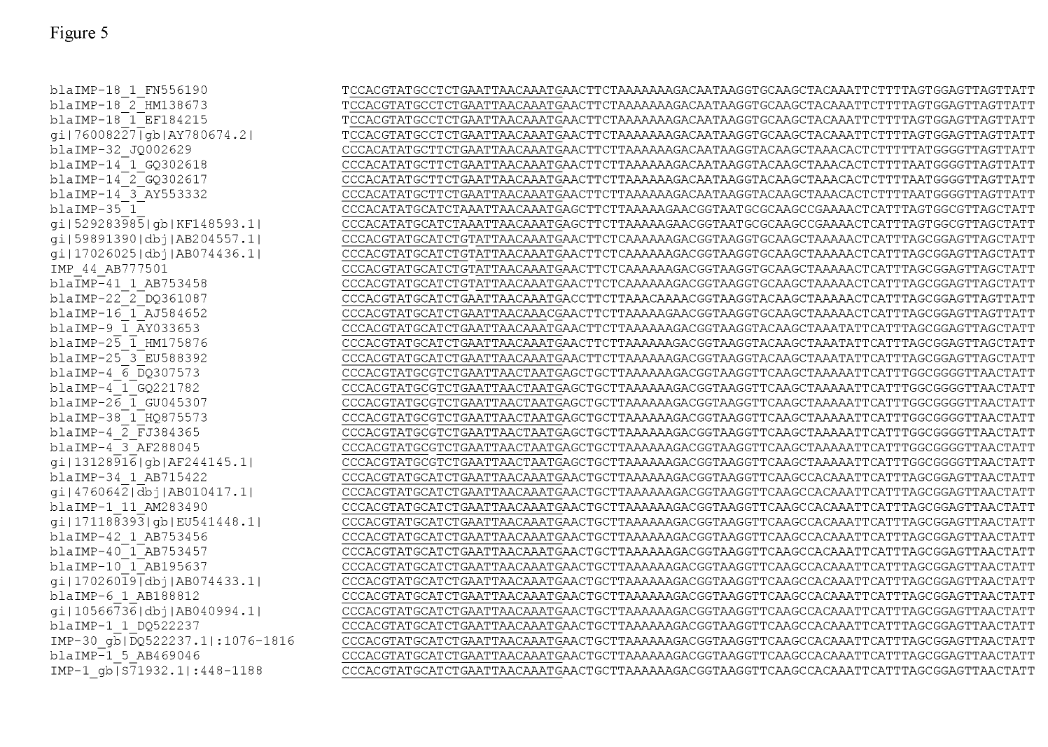

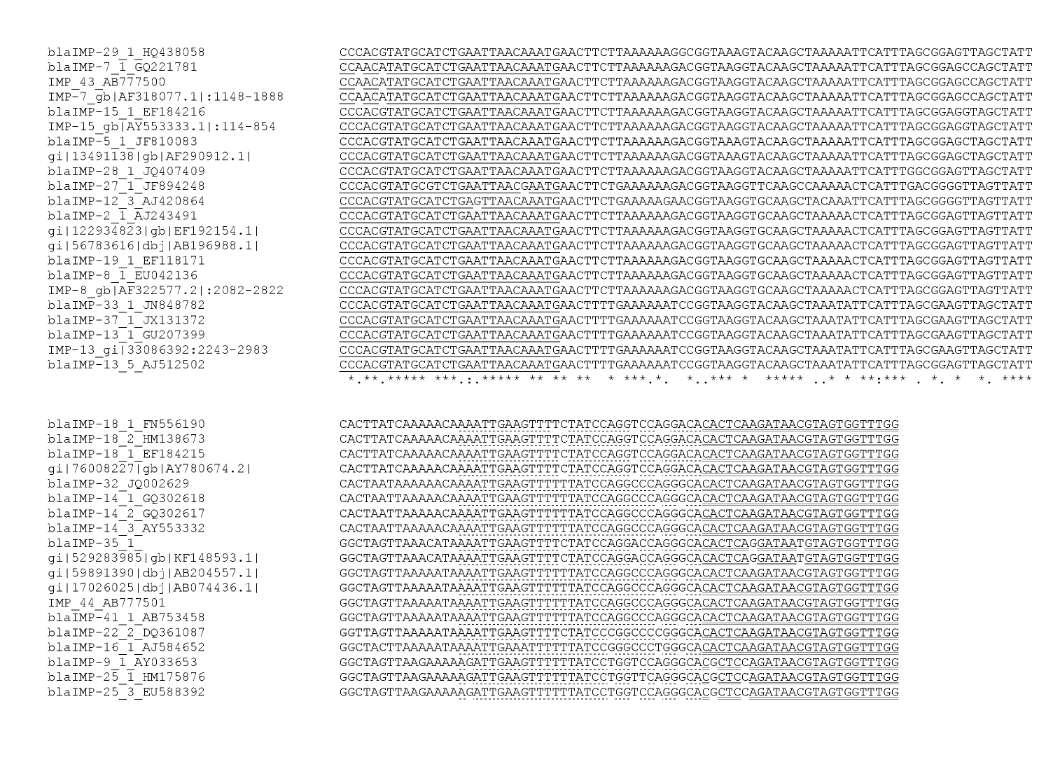

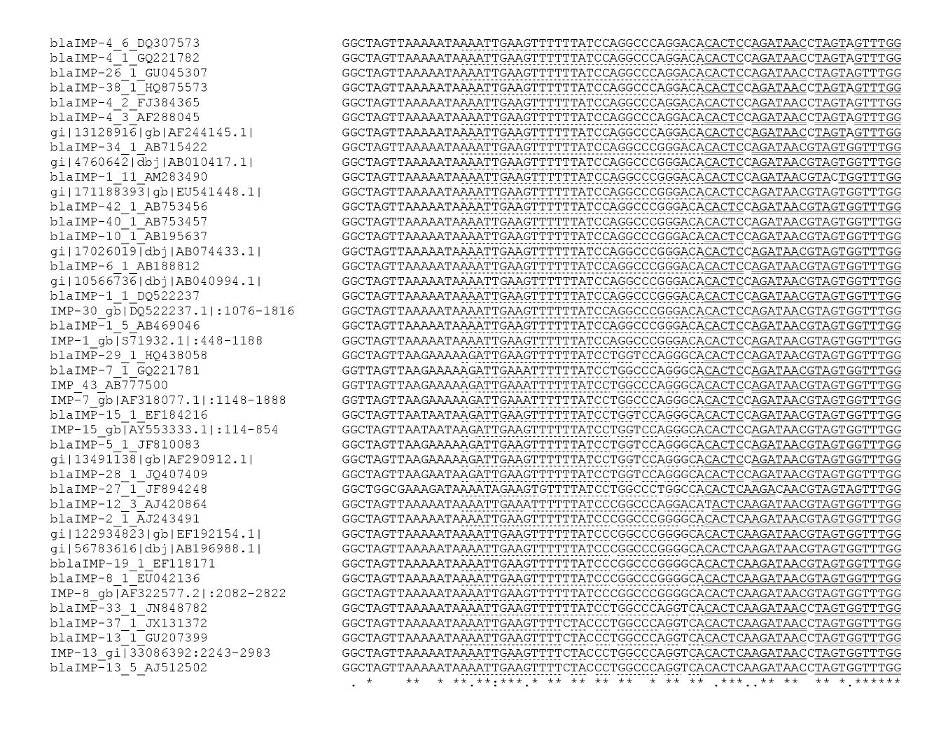

The DNA sequence alignments were analysed and primer and probe sequences were designed to maximize coverage of the different gene variants. Alignments from the OAX carbapenemase genes, VIM carbapenemase genes, KPC carbapenemase genes, NDM carbapenemase genes and IMP carbapenemase genes are shown in FIGS. 1 to 5 respectively. Newly designed primers and probes for OXA, VIM, NDM and IMP are given in the Table below (Table 7):

| | | SEQ |
|---|---|---|
| Name | Sequence | ID NO |
| NDM_fwd_ | CCAGCAAATGGAAACT | 7 |
| | GGCGAC | |
| NDM_R | ATCCAGTTGAGGATCT | 8 |
| | GGGCG | |
| OXA-48_like fwd | GATTATGGTAATGAGG | 16 |
| | ACATTTCGGGC | |
| OXA-48_like_rev | CATATCCATATTCATC | 2 |
| | GCAAAAAACCACAC | |
| OXA-48_like probe | CCATTGGCTTCGGTCA | 3 |
| | GCATGGCTTGTTT | |
| VIM_fwd_ | TTGCTTTTGATTGATA | 17 |
| | CAGCGTGGGG | |
| VIM_rev_ | GTACGTTGCCACCCCA | 18 |
| | GCC | |
| IMP-Fwd | CCCACGTATGCATCTG | 22 |
| | AATTAACAAATG | |
| IMP-Rev | CCAAACCACTACGTTA | 23 |
| | TCTKGAGTG | |
| IMPprobe1 | TGICCTGGICCAGGAT | 24 |
| | AAAAAACTTCAATIT | |
| IMPprobe2 | TGICCIGGGCCIGGAT | 25 |
| | AAAAAACTTCAATTT | |

Primers and probes were synthesised using standard techniques.

The primers and probes were prepared in water and added to the Reaction tube along with the appropriate amount of standard manufacturer supplied Taq enzyme, dNTP, MgCl2 mix (Invitrogen Platinum qPCR, SuperMix-UDG. Cat#11730-25, although other enzyme mixes could be used).

Bacterial suspensions of test organisms were prepared with an internal, whole process, positive control (IPC) as follows:

A suspension of the vegetative cells of the IPC organism (Bacillus thuringiensis) was prepared from an overnight agar culture by resuspending the organism in water to a standard Optical Density (in this case McFarland standard 0.5—but this can be adjusted). This standard suspension was aliquoted in to smaller volumes tubes (in this case 100 μl in each tube). 1-5 bacterial colonies from agar culture of test organisms were picked and resuspended into the tubes containing the suspension of the IPC.

Lysis of the cells in the suspension was performed by heating the suspension for 30 minutes (99° C. in this case—but temp can vary)

An aliquot of the lysate (1-5 μl in this case) was added to the enzyme/probe reaction mix in PCR tubes/plates/reaction vials and mixed.

Samples were tested using the primers and probes shown in the table below (Table 8):

| | | SEQ |
|---|---|---|
| Name | Sequence | ID NO |
| KPC-Fwd | GCAGCGGCAGCAGTTTG | 21 |
| | TTGATT | |
| KPC-Rev | GTAGACGGCCAACACAA | 11 |
| | TAGGTGC | |
| KPC-probe | CAGTCGGAGACAAAACC | 12 |
| | GGAACCTGC | |
| NDM_fwd_ | CCAGCAAATGGAAACTG | 7 |
| | GCGAC | |
| NDM_R | ATCCAGTTGAGGATCTG | 8 |
| | GGCG | |
| NDM_probe | ACCGAATGTCTGGCAGC | 9 |
| | ACACTTC | |
| OXA-48_like fwd | GATTATGGTAATGAGGA | 16 |
| | CATTTCGGGC | |
| OXA-48_like_rev | CATATCCATATTCATCG | 2 |
| | CAAAAAACCACAC | |
| OXA-48_like probe | CCATTGGCTTCGGTCAG | 3 |
| | CATGGCTTGTTT | |
| VIM_fwd_ | TTGCTTTTGATTGATAC | 17 |
| | AGCGTGGGG | |
| VIM_rev_ | GTACGTTGCCACCCCAG | 18 |
| | CC | |
| VIM_II_probe | TCTCGCGGAGATTGAAA | 19 |
| | AGCAAATTGGACTTCC | |
| IMP-Fwd | CCCACGTATGCATCTGA | 22 |
| | ATTAACAAATG | |
| IMP-Rev | CCAAACCACTACGTTAT | 23 |
| | CTKGAGTG | |
| IMPprobe1 | TGICCTGGICCAGGATA | 24 |
| | AAAAACTTCAATIT | |
| IMPprobe2 | TGICCIGGGCCIGGATA | 25 |
| | AAAAACTTCAATTT | |
| B. thuringiensis | | |
| controls | | |
| Bt_fwd | GCAACTATGAGTAGTGG | 194 |
| | GAGTAATTTAC | |
| Bt_rev | TTCATTGCCTGAATTGA | 195 |
| | AGACATGAG | |
| Bt_probe | ACGGAGTAGTAGTAAAA | 196 |
| | CCTACAGTCCTAAAGC | |

The reactions were placed on to an RT-PCR instrument (AB7500 Fast or Rotorgene) and thermal cycled using standard reaction conditions (in this case on the AB7500Fast=60° C.—1 min, 95° C.—10 mins followed by 50 cycles of: 95° C.—15 secs, 60° C.—1 min, and a final a single step of: 60° C.—1 min. On the Rotorgene the parameters=60° C.—1 min, 95° C.—10 mins followed by 50 cycles of: 95° C.—15 secs, 58° C.—1 min, and a final a single step of: 60° C.—1 min).

Results were read on screen looking for an exponential signal from the Taqman probes in each channel. Positive results were called when a sigmoidally increasing fluorescent signal was detected and breached a threshold (crossing threshold or Ct). Using an Applied Biosystems 7500Fast machine this was standardly 50000 Fluorescence units, on the Rotorgene was usually around the normalised fluorescence level ‘0.2’. (These values are typically malleable on a run by run basis judged against the performance of the positive and negative controls).

Controls were as follows: 1 each of known positives for KPC-3, NDM-1, OXA-48 and VIM-10 (although any known positive for each gene family could theoretically be substituted), these were used to judge the performance of the assay for each of the gene families. Two tubes of negative controls were used to judge cross-tube contamination with carbapenemases, these contained no added carbapenemase template but did contain the IPC and were positive for that on test. To judge the background fluorescence of the probes/enzyme and risk of cross-tube contamination two further tubes of No-Template-Control (NTC) were used which contained no IPC or carbapenemase gene template and were standardly negative.

Tests were carried out for 100 isolates of each gene family from culture and 50 samples that were negative for these four target DNA sequences. The positive and negative controls all tested as expected. Of the isolates that were positive for KPC, OXA-48-like, NDM or VIM genes (n=400) all were detected and identified with the correct carbapenemaxse gene family and did not cross react with other gene families (i.e. did not detect any additional, false-positive, genes). The 50 isolates that were known to be negative for any of these four carbapenmease gene types all tested negative as expected. This indicates 100% sensitivity and specificity for the test when testing from cultured organism.

### Example 2—Further Validation of Primers and Probes

### Bacterial Isolates, Identification and Carbapenemases

The isolates (n=502) were comprised of 490 Enterobacteriaceae and 12 Pseudomonas spp. (see Table 9) that had been submitted to PHE's Antimicrobial Resistance and Healthcare Associated Infections (AMRHAI) Reference Unit, for investigation of ‘unusual’ resistance, mostly to carbapenems.

The isolates had been identified using either API-20E tests (bioMerieux SA, Marcy-l'Etoile, France) or MALDI-ToF MS; Bruker Microflex LT (Bruker Daltonik GmbH, Bremen, Germany). Antibiotic susceptibilities (minimum inhibitory concentrations—MICs) had been determined by BSAC agar dilution and interpreted using BSAC breakpoints, where available.

The isolates included 426 carbapenemase positives, comprised of 100 each of KPC, NDM, VIM and OXA-48-like producers and two isolates producing both NDM and an OXA-48-like enzyme. The remaining 24 carbapenemase-producers isolates were IMP-positive. A further 24 isolates had carbapenem resistance contingent upon ESBL and/AmpC activity plus porin loss (Table 2) and 52 isolates were carbapenem susceptible with extended-spectrum

TABLE 9
Species distribution and carbapenemase status of 502 and 100 isolates used
for validation or multi-centre evaluation of the carbapenemase gene detection
real-time PCR assay. Dividers separate isolates from the validation and evaluation panels.

| | Carbapenemase gene family | | | | | | | |
|---|---|---|---|---|---|---|---|---|
| | | | | OXA-48- | NDM + | | | |
| Species | KPC | NDM | VIM | like | OXA-48-like | IMP | None | Totals |
| Klebsiella pneumoniae | 62/10 | 52/11 | 55/9 | 59/9 | 2/0 | 0/0 | 25/2 | 255/41 |
| Klebsiella oxytoca | 6/2 | 0/0 | 12/5 | 3/1 | 0/0 | 1/1 | 0/0 | 22/9 |
| Klebsiella sp. | 0/0 | 0/0 | 0/0 | 0/0 | 0/0 | 1/1 | 0/0 | 1/1 |
| Escherichia coli | 9/2 | 28/5 | 8/4 | 29/8 | 0/0 | 2/1 | 25/2 | 101/22 |
| Enterobacter spp. | 18/4 | 14/3 | 16/1 | 8/3 | 0/0 | 8/2 | 23/2 | 87/15 |
| Pseudomonas spp. | 0/0 | 0/0 | 0/0 | 0/0 | 0/0 | 11/0 | 1/0 | 12/0 |
| Others | 5/4 | 6/3 | 9/3 | 1/1 | 0/0 | 1/1 | 2/0 | *24/12^{#} |
| Totals | 100/22 | 100/22 | 100/22 | 100/22 | 2/0 | 24/6 | ^{a}76/6^{b} | 502/100 |

^{a}ESBL producing organisms (n = 52) that were carbapenem susceptible and carbapenem resistant isolates (n=) 24 that did not produce a carbapenemase.
^{b}isolates that were carbapenem resistant but did not produce a carbapenemase.
*comprised of Citrobacter spp. (n = 16), Raoutella spp. (n = 3), Leclercia adecarboxylata (n = 2), Serratia marcesens (n = 2), Kluyvera georgiana (n = 1).
^{#}comprised of Citrobacter spp. (n = 6), Raoutella spp. (n = 2), Leclercia adecarboxylata (n = 2), Serratia marcesens (n = 1), Kluyvera georgiana (n = 1).

beta-lactamases (ESBLs) only. The latter three isolate groups represented predicted negatives for the detection range of this assay. The carbapenemase gene content of the isolates had been detected previously using in-house PCR assays and/or a commercial microarray (Check-MDR CT102; Check-Points, Wageningen, The Netherlands).

### Inter Laboratory Evaluation

To compare the performance of the assay across different laboratories a sub-set of 100 geographically diverse isolates were chosen from the validation isolates (see above and Table 9) and included: 22 isolates producing each of KPC, NDM VIM and OXA-48-like enzymes (including four OXA-181). Of twelve isolates that were predicted to be negative for this assay six produced an IMP enzyme and six that were porin deficient beta-lactamase producers (Table 9).

Overall, 502 isolates were tested in full on both Rotor-Gene Q and ABI 7500 instruments within AMRHAI. The sub-set of 100 isolates was tested five times in four laboratories, three times on ABI 7500 instruments and twice on Rotor-Gene Q instruments. All tests were carried out as described below.

### Bacterial Culture and DNA Template Preparation

Isolates were cultured on to MacConkey, Columbia blood or CLED agar plates with a 10 μg ertapenem or meropenem disc (Oxoid, Basingstoke, UK). One to five colonies of test organism were picked using a 1 μl loop and suspended in 100 μl of molecular grade water containing a 0.5 McFarland standard suspension of Bacillus thuringiensis ATCC 29730. The suspension was incubated for 30 minutes at 98° C. and 5 μl used in the RT-PCR assay.

### Real-Time PCR Assay and Data Analysis

For the detection assay a PCR enzyme and dNTP mixture (Platinum qPCR SuperMix-UDG, Invitrogen Paisley, UK) was mixed with template DNA and primers and probes (Table 8) for KPC, OXA-48-like, NDM, VIM and the B. thuringiensis cry1 gene. Rotor-Gene Q (5-plexHRM) instruments (QIAGEN, Crawley, UK) were thermal cycled: 95° C. for 10 min; 95 C for 15 seconds and 58 C for 1 minute (50 cycles) and 60° C. for 5 minutes and 4° C. hold. The same parameters were used for ABI 7500 (Life Technologies, Paisley, UK) instruments excepting the extension step was at 60° C. Results from RT-PCR assays were all interpreted using a cut-off value (Ct≦36) to identify carbapenemase-positive isolates. Rotor-Gene Q v2.0.3 software and ABI 7500 v2.0.4 were used to analyse data.

### Results

### Assay Performance

The assay successfully detected the correct carbapenemase gene in all 402 isolates with a KPC, NDM or VIM or OXA-48-like enzyme and achieved 100% sensitivity for these targets (Table 10). The OXA-48-like positive isolates included 18 isolates that encoded blaOXA-181 (one also encoded an NDM enzyme). The assay also demonstrated 100% specificity, indicating the 100 samples that were negative for the targets detected by this assay (Table 10), including 24 IMP producers that encoded enzymes closely related to IMP-4, IMP-7, IMP-8, IMP-13, IMP-14.

The internal control failed to amplify for 94 (18.7%) of 502 tests on ABI 7500 instruments and for 99 (19.7%) tests on Rotor-Gene Q instruments, likely due to successful amplification of a carbapenemase gene amplicon. For this evaluation amplification of the internal control was only deemed critical for carbapenemase-negative isolates in order to demonstrate the correct function of the PCR. Indeed, for carbapenemase negative isolates the internal positive control demonstrated 100% sensitivity on both platforms.

TABLE 10
Performance of CPE RT-PCR assay in four different centres
each using Rotor Gene Q and/or ABI 7500 instrument(s)

| | | | | Centre | Centre |
|---|---|---|---|---|---|
| | Centre | Centre | Centre | 1.Col | 4.Cam |
| | 1.Col | 2.Bham | 3.Leeds | Rotor- | Rotor- |
| | ABI 7500 | ABI 7500 | ABI 7500 | Gene Q | Gene-Q |
| KPC | 100% | 100% | 100% | 100% | 100% |
| (n = 22) | | | | | |
| OXA- | 100% | 100% | 100% | 100% | 100% |
| 48-like | | | | | |
| (n = 22)^{a} | | | | | |
| NDM | 100% | 100% | 100% | 100% | 100% |
| (n = 22) | | | | | |
| VIM | 100% | 100% | 100% | 100% | 100% |
| (n = 22) | | | | | |
| Non-carba | 100% | 100% | 100% | 100% | 100% |
| (n = 12) | | | | | |

^{a}Sequencing identified OXA-181 in four isolates.

### Multi-Centre Evaluation

Tests of the 100 evaluation isolates using ABI 7500 instruments located in three laboratories and Rotor-Gene Q instruments located in two laboratories the assay demonstrated 100% sensitivity and specificity (Table 10). Of the 22 correctly identified OXA-48-like positive isolates four were OXA-181. As expected, the assay also accurately identified the 12 samples that were negative for the genes detected by this assay (including six IMP producers).

### SUMMARY

Thus, the above validation data obtained using both the Rotor-Gene Q and ABI 7500 instruments demonstrated 100% sensitivity for the assay amongst the 402 isolates that were positive for KPC, NDM, OXA-48-like (including OXA-181) and VIM carbapenemase genes, whilst the 100 assay negative samples were correctly identified indicating 100% specificity. The same 100% level of sensitivity and specificity, was observed in each of the four centres that participated. Neither invalid nor false-positive results were observed.

In addition, the assay also detected isolates that were positive for two targets: NDM and OXA-48 or NDM and OXA-181, which present a theoretical detection risk due to possible competition for reaction resources between the different positive amplicons. The assay was able to detect both combinations.

### Example 3—Detection of Combinations of Carbapenemase Genes

Further testing of the assay for the detection of two carbapenemase genes in the same isolate was carried out using the same methodology as described above in Examples 1 and 2, using isolates known to have combinations of genes as set out as follows in Table 11:

TABLE 11

| | Number of | % of genes |
|---|---|---|
| Genes present | isolates tested | detected |
| NDM-1; OXA-48 | 5 | 100% |
| NDM-1; OXA-181 | 5 | 90% |
| NDM-1; OXA-232 | 7 | 100% |
| KPC-2; OXA-48 | 3 | 100% |
| KPC-2; NDM-1 | 1 | 100% |

The numbers of isolates tested here are small reflecting the scarcity of these isolate types. The data does indicate that where these isolates do occur the assay can reliably detect the presence of a two carbapenemases in these cases and with the exception of isolates harbouring NDM-land OXA-181 can do so with 100% accuracy. The reason for the failure of the assay to detect OXA-181 in one isolate with NDM-1 and OXA-181 remains unknown.

1. A method for determining the presence and/or amount of one or more carbapenemase-producing bacteria in a sample comprising determining the presence and/or amount of a carbapenemase gene selected from an OXA-48-like gene and/or a VIM gene in said sample, in which the presence and/or amount of the OXA-48-like gene is determined using at least one oligonucleotide specific for the OXA-48-like gene and/or the presence and/or amount of the VIM gene is determined using at least one oligonucleotide specific for the VIM gene, wherein:
(a) the at least one oligonucleotide specific for the OXA-48-like gene comprises a region of at least 24 contiguous bases from SEQ ID NO: 3, or from a nucleic acid sequence having at least 80% sequence identity to the full-length of the nucleic acid sequence of SEQ ID NO: 3 and hybridises to a target region of the OXA-48-like gene comprising bases 570 to 670 of the OXA-48-like gene; and/or
(b) the at least one oligonucleotide specific for the VIM gene is 15 to 35 bases in length and hybridises to a nucleic acid sequence within a target region of the VIM gene comprising bases 235 to 400, 238 to 403 and/or 245 to 410 of the VIM gene.

2. The method of claim 1, wherein the at least one oligonucleotide sequence specific for the VIM gene hybridises to a target region of the VIM gene comprising bases 350 to 410 of the VIM gene.

3. The method of claim 1 or 2, wherein the at least one oligonucleotide sequence specific for the VIM gene comprises a region of at least 15 contiguous bases from SEQ ID NO: 18, or from a nucleic acid sequence having at least 80% sequence identity to the full-length of the nucleic acid sequence of SEQ ID NO: 18.

4. The method of any one of the preceding claims, wherein the oligonucleotide specific for the OXA-48-like gene is a probe.

5. The method of any one of the preceding claims, wherein the oligonucleotide specific for the VIM gene is a reverse primer.

6. The method of any one of the preceding claims, wherein the presence and/or amount of the OXA-48-like gene and the VIM gene is determined.

7. The method of any one of the preceding claims, wherein the at least one oligonucleotide specific for the VIM gene hybridises to a target region of the VIM gene comprising bases:
(i) 235 to 280, 238 to 283 or 240 to 290; and/or
(ii) 360 to 410, 363 to 413 or 270 to 420;
of the VIM gene.

8. The method of any one of the preceding claims, wherein one or more additional oligonucleotide specific for the OXA-48-like gene, said one or more additional oligonucleotide specific for the OXA-48-like gene comprising a nucleic acid sequence of SEQ ID NO: 2 and/or 16, or a nucleic acid sequence having at least 80% sequence identity to the full-length of the nucleic acid sequence of SEQ ID NO: 2 and/or 16, is used to determine the presence and/or amount of the OXA-48-like gene.

9. The method of any one of the preceding claims, wherein one or more additional oligonucleotide specific for the VIM gene, said one or more additional oligonucleotide specific for the VIM gene comprising a nucleic acid sequence of SEQ ID NO: 19 and/or 17, or a nucleic acid sequence having at least 80% sequence identity to the full-length of the nucleic acid sequence of SEQ ID NO: 19 and/or 17, is used to determine the presence and/or amount of the VIM gene.

10. The method of any one of the preceding claims, wherein
(i) an additional oligonucleotide specific for the OXA-48-like gene hybridises to a target region of the OXA-48-like gene comprising bases 421 to 490 of the OXA-48-like gene; or
(ii) an additional oligonucleotide specific for the OXA-48-like gene hybridises to a target region of the OXA-48-like gene comprising bases 650 to 717 of the OXA-48-like gene; or
(iii) a first additional oligonucleotide specific for the OXA-48-like gene hybridises to a target region of the OXA-48-like gene comprising bases 421 to 490 of the OXA-48-like gene, and a second additional oligonucleotide specific for the OXA-48-like gene hybridises to a target region of the OXA-48-like gene comprising bases 650 to 717 of the OXA-48-like gene.

11. The method of claim 11, wherein:
(i) the additional oligonucleotide specific for the OXA-48-like gene which hybridises to a target region of the OXA-48-like gene comprising bases 421 to 490 of the OXA-48-like gene is a forward primer;
(ii) the additional oligonucleotide specific for the OXA-48-like gene which hybridises to a target region of the OXA-48-like gene comprising bases 650 to 717 of the OXA-48-like gene a reverse primer.

12. The method of claim 10 or 11, wherein:
(i) the additional oligonucleotide specific for the OXA-48-like gene which hybridises to a target region of the OXA-48-like gene comprising bases 421 to 490 of the OXA-48-like gene comprises or consists of a nucleic acid sequence of SEQ ID NO: 16, or a nucleic acid sequence having at least 80% sequence identity to the full-length of the nucleic acid sequence of SEQ ID NO: 16; and/or
(ii) the additional oligonucleotide specific for the OXA-48-like gene which hybridises to a target region of the OXA-48-like gene comprising bases 650 to 717 of the OXA-48-like gene comprises or consists of a nucleic acid sequence of SEQ ID NO: 2, or a nucleic acid sequence having at least 80% sequence identity to the full-length of the nucleic acid sequence of SEQ ID NO: 2.

13. The method of any one of the preceding claims, wherein:
(i) an additional oligonucleotide specific for the VIM gene hybridises to a target region of the VIM gene comprising bases 235 to 400, 238 to 403 and/or 245 to 410 of the VIM gene, preferably to a target region of the VIM gene comprising bases 235 to 270 of the VIM gene; or
(ii) an additional oligonucleotide specific for the VIM gene hybridises to a target region of the VIM gene comprising bases 235 to 400, 238 to 403 and/or 245 to 410 of the VIM gene, preferably to a target region of the VIM gene comprising bases 275 to 330 of the VIM gene; or
(iii) a first additional oligonucleotide specific for the VIM gene hybridises to a target region of the VIM gene comprising bases 235 to 400, 238 to 403 and/or 245 to 410 of the VIM gene, preferably to a target region of the VIM gene comprising bases 235 to 270 of the VIM gene, and a second additional oligonucleotide specific for the VIM gene hybridises to a target region of the VIM gene comprising bases 235 to 400, 238 to 403 and/or 245 to 410 of the VIM gene, preferably to a target region of the VIM gene comprising bases 275 to 330 of the VIM gene.

14. The method of claim 13, wherein:
(i) the additional oligonucleotide specific for the VIM gene which hybridises to a target region of the VIM gene comprising bases 235 to 400, 238 to 403 and/or 245 to 410 of the VIM gene, preferably to a target region of the VIM gene comprising bases 235 to 270 of the VIM gene, is a forward primer; and/or
(ii) the additional oligonucleotide specific for the VIM gene which hybridises to a target region of the VIM gene comprising bases 235 to 400, 238 to 403 and/or 245 to 410 of the VIM gene, preferably to a target region of the VIM gene comprising bases 275 to 330 of the VIM gene, is a probe.

15. The method of claim 13 or 14, wherein:
(i) the additional oligonucleotide specific for the VIM gene which hybridises to a target region of the VIM gene comprising bases 235 to 400, 238 to 403 and/or 245 to 410 of the VIM gene, preferably to a target region of the VIM gene comprising bases 235 to 270 of the VIM gene, comprises or consists of a nucleic acid sequence of SEQ ID: 17, or a nucleic acid sequence having at least 80% sequence identity to the full-length of the nucleic acid sequence of SEQ ID NO: 17; and/or
(ii) the additional oligonucleotide specific for the VIM gene which hybridises to a target region of the VIM gene comprising bases 235 to 400, 238 to 403 and/or 245 to 410 of the VIM gene, preferably to a target region of the VIM gene comprising bases 275 to 330 of the VIM gene, comprises or consists of a nucleic acid sequence of SEQ ID: 19, or a nucleic acid sequence having at least 80% sequence identity to the full-length of the nucleic acid sequence of SEQ ID NO: 19.

16. The method of any one of the preceding claims, further comprising determining the presence and/or amount of one or more additional carbapenemase gene.

17. The method of claim 16, wherein the one or more additional carbapenemase gene is selected from a KPC gene, an NDM gene, an IMP gene, an IMI gene, a GES gene and an SPM gene.

18. The method of claim 16 or 17, wherein the one or more additional carbapenemase gene is:
(i) a KPC gene;
(ii) an NDM gene;
(iii) an IMP gene;
(iv) a KPC gene and an NDM gene;
(v) a KPC gene and an IMP gene;
(vi) an NDM gene and an IMP gene; or
(vii) a KPC gene, an NDM gene and an IMP gene.

19. The method of any one of claims 16 to 18, wherein the presence and/or amount of:
(i) an OXA-48-like gene and an NDM gene;
(ii) an OXA-48-like gene, a VIM gene and an NDM gene;
(iii) an OXA-48-like gene, a VIM gene, a KPC gene and an NDM gene; or
(iv) an OXA-48-like gene, a VIM gene, a KPC gene an NDM gene and an IMP gene;
is determined.

20. The method of any one of claims 16 to 19, wherein the presence and/or amount of the one or more additional carbapenemase gene is determined using at least one oligonucleotide specific for said one or more additional carbapenemase gene.

21. The method of claim 20, wherein the at least one oligonucleotide specific for the NDM gene hybridises to a target region of the NDM gene comprising bases 108 to 320 of the NDM gene.

22. The method of claim 20 or 21, wherein the at least one oligonucleotide specific for the KPC gene hybridises to a target region of the KPC gene comprising bases 580 to 800 or 600 to 810 of the KPC gene.

23. The method of any one of claims 20 to 22, wherein the at least one oligonucleotide specific for the IMP gene hybridises to a target region of the IMP gene comprising bases 337 to 515 of the IMP gene.

24. The method of any one of claims 20 to 23, wherein the at least one oligonucleotide specific for the NDM gene hybridises to a target region of the NDM gene comprising bases 108 to 150, 294 to 320 and/or 160 to 200 of the NDM gene.

25. The method of any one of claims 20 to 24, wherein the at least one oligonucleotide specific for the KPC gene hybridises to a target region of the KPC gene comprising bases:
(i) 580 to 620 or 600 to 650;
(ii) 745 to 800 or 760 to 815; and/or
(iii) 660 to 710 or 675 to 725;
of the KPC gene.

26. The method of any one of claims 20 to 25, wherein the at least one oligonucleotide specific for the IMP gene hybridises to a target region of the IMP gene comprising bases 337 to 363, 440 to 514 and/or 471 to 515 of the IMP gene.

27. The method of any one of claims 20 to 26, wherein the at least one oligonucleotide specific for the NDM gene comprises a nucleic acid sequence of SEQ ID NO: 7, 8 and/or 9, or a nucleic acid sequence having at least 80% sequence identity to the full-length of the nucleic acid sequence of SEQ ID NO: 7, 8 and/or 9.

28. The method of any one of claims 20 to 27, wherein the at least one oligonucleotide specific for the KPC gene comprises a nucleic acid sequence of SEQ ID NO: 21, 11 and/or 12, or a nucleic acid sequence having at least 80% sequence identity to the full-length of the nucleic acid sequence of SEQ ID NO: 21, 11 and/or 12.

29. The method of any one of claims 20 to 28, wherein the at least one oligonucleotide specific for the IMP gene comprises a nucleic acid sequence of SEQ ID NO: 22, 23, 24 and/or 25, or a nucleic acid sequence having at least 70% sequence identity to the full-length of the nucleic acid sequence of SEQ ID NO: 22, 23, 24 and/or 25.

30. The method of any one of the preceding claims, wherein the presence and/or amount of the OXA-48-like gene, the VIM gene and/or the one or more additional carbapenemase gene is determined by PCR and/or hybridisation.

31. The method of claim 30, wherein the presence and/or amount of the OXA-48-like, the VIM gene and/or the one or more additional carbapenemase gene is determined by real-time PCR.

32. The method of any one of the preceding claims, wherein:
(i) the presence and/or amount the OXA-48-like gene is compared with the presence and/or amount of the OXA-48-like in a control sample;
(ii) the presence and/or amount the VIM gene is compared with the presence and/or amount of the VIM gene in a control sample; and/or
(iii) the presence and/or amount the one or more additional carbapenemase gene is compared with the presence and/or amount of the one or more additional carbapenemase gene in a control sample.

33. A method for diagnosing a carbapenem-resistant bacteria infection in an individual comprising carrying out a method according to any one of the preceding claims on a sample obtained from the individual.

34. The method of any one of the preceding claims, wherein the sample is a sample of blood, cerebral spinal fluid, saliva, urine, cells, a cellular extract, a stool sample, a tissue sample or a tissue biopsy, or a swab from an individual, such as a rectal swab, or a swab from the environment.

35. A device for use in the method of any one of the preceding claims, which comprises one or more oligonucleotide specific for the OXA-48-like gene, the VIM gene and/or the one or more additional carbapenemase gene.

36. The device of claim 35, wherein the one or more oligonucleotide specific for the OXA-48-like gene, the VIM gene and/or the one or more additional carbapenemase gene is an oligonucleotide as defined in any one of claims 1 to 15 and/or 21 to 29.

37. The device of claim 35 or 36, wherein the one or more oligonucleotide is immobilised on a surface.
